(19) United States
(12) Patent Application Publication
(10) Pub. No.: US 2009/0137557 A1
(43) Pub. Date: May 28, 2009

(54) Calcilytic Compounds

(76) Inventors: Thomas Wen Fu Ku, Collegeville, PA (US); Hong Lin, Collegeville, PA (US); Juan I. Luengo, Collegeville, PA (US); Robert W. Marquis Jr., Collegeville, PA (US); Joshi M. Ramanjulu, Collegeville, PA (US); Robert Trout, Collegeville, PA (US); Dennis S. Yamashita, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/094,665

(22) PCT Filed: Nov. 21, 2006
(86) PCT No.: PCT/US06/61150

§ 371 Date: May 22, 2008

Related U.S. Application Data
(60) Provisional application No. 60/739,067, filed on Nov. 22, 2005.
(60) Provisional application No. 60/738,731, filed on Nov. 22, 2005.

(51) Int. Cl. A61K 31/513; C07D 239/22; C07D 413/04; A61K 31/55; A61P 19/10; A61K 31/5377; C07D 487/04

(52) U.S. Cl. 514/215; 544/319; 544/123; 540/578; 514/269; 514/235.8

(57) ABSTRACT

Novel calcilytic compounds, pharmaceutical compositions, methods of synthesis and methods of using them are provided.

### FIELD OF INVENTION

The present invention relates to novel calcilytic compounds, pharmaceutical compositions containing these compounds, processes for their preparation and their use as calcium receptor antagonists.

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evoked by extracellular Ca²⁺. Calcilytics are useful as lead molecules in the discovery, development, design, modification and/or construction of useful calcium modulators, which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

### SUMMARY OF THE INVENTION

The present invention comprises novel calcium receptor antagonists represented by Formula (I) and Formula (II) hereinbelow, formulations comprising the present compounds, and their use as calcium receptor antagonists in the treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

The present invention further provides a method for antagonizing calcium receptors in an animal, including humans, which comprises administering to an animal in need thereof an effective amount of a compound of Formula (I) or (II), indicated hereinbelow.

The present invention further provides a method for increasing serum parathyroid levels in an animal, including humans, which comprises administering to an animal in need thereof an effective amount of a compound of Formula (I) or (II), indicated hereinbelow.

### DETAILED DESCRIPTION OF THE INVENTION

The present invention involves novel compounds according to formula (I) hereinbelow:

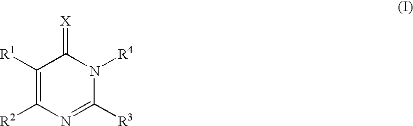

wherein:

X is O or S;

R¹ and R² are, independently, selected from the group consisting of H, halogen, CN, C₁₋₁₀alkyl, C₂₋₆alkenyl, cycloalkyl, cycloalkylC₁₋₆alkyl, aryl, arylC₁₋₆alkyl, heterocyclyl, heteroaryl, (CR₁₀R₁₁)ₓNR₅R₆, C(O)OR₅, C(O)NR₅R₆, NR₅C(O)R₆, (CR₁₀R₁₁)ₓOR₅ and NC(O)R₅, optionally substituted, except for H, halogen and CN, one to three times, independently, by halogen, CN, C₁₋₄alkyl, aryl, heteroaryl, C(O)OR₁₉, O—(CR₁₉R₂₀)_{q}—O, C(O)R₁₉, CF₃, OCF₃, NO₂, C(O)NR₁₉R₂₀, (CR₁₀R₁₁)_{z}OR₁₉, (CR₁₀R₁₁)_{z}NR₁₉R₂₀, and (CR₁₀R₁₁)ₓS(O)ₘR₁₉;

or R¹ and R² together form an optionally substituted 5 to 8 membered ring, optionally containing one to three heteroatoms selected from N, O and S, wherein the optional substituents are selected, independently, at each occurrence, one to three times, from the group consisting of halogen, C₁₋₄alkyl, (CR₁₀R₁₁)_{z}S(O)ₘR₅, (CR₁₀R₁₁)_{z}OR₅, (CR₁₀R₁₁)_{z}NR₅R₆, C(O)R₅ and C(O)OR₅;

or R¹ and R² together form an optionally substituted heteroaryl ring, wherein the optional substituents are selected, independently, at each occurrence, one to three times, from the group consisting of halogen, C₁₋₄alkyl, (CR₁₀R₁₁)_{z}S(O)ₘR₅, (CR₁₀R₁₁)_{z}OR₅, (CR₁₀R₁₁)_{z}NR₅R₆, C(O)R₅ and C(O)OR₅;

or, when R¹ is NR₅R₆, R₅ and R₆ can join together to form a 5 to 7 membered ring, optionally substituted by C₁₋₄alkyl or halogen;

R₅ and R₆ represent, independently, at each occurrence, H, C₁₋₄alkyl, cycloalkyl, cycloalkylC₁₋₆alkyl, C₂₋₆alkenyl, heterocyclyl, heterocyclylC₁₋₆alkyl, aryl, arylC₁₋₆alkyl, heteroaryl or heteroarylC₁₋₆alkyl, wherein each moiety, except H, is optionally substituted, independently, one to three times, by halogen or C₁₋₄alkyl;

R₁₀ and R₁₁, represent, independently, at each occurrence, H or C₁₋₄alkyl;

R₁₉ and R₂₀ represent, independently, at each occurrence, H, C₁₋₄alkyl, cycloalkyl, cycloalkylC₁₋₆alkyl, C₂₋₆alkenyl, heterocyclyl, heterocyclylC₁₋₄alkyl, aryl, arylC₁₋₆alkyl, heteroaryl or a heteroarylC₁₋₆alkyl moiety, wherein each moiety, except H, may be substituted, independently, one to three times, by halogen or C₁₋₄alkyl;

R³ represents aryl or heteroaryl, optionally substituted, independently, one to three times, by C₁₋₄alkyl, halogen, CN or CF₃;

R⁴ is selected from the group consisting of cycloalkylC₁₋₄alkyl, heteroaryl, heterocyclyl, aryl, heteroarylC₁₋₂alkyl, heterocyclylC₁₋₂alkyl, cycloalkylC₂alkenyl, arylC₂alkenyl, heteroarylC₂alkenyl and heterocyclylC₂alkenyl, wherein each moiety is optionally substituted, independently, one to three times, by C₁₋₄alkyl, F, CF₃ or Cl;

m is 0, 1 or 2;

x is 0, 1, 2 or 3;

q is 1, 2 or 3; and

z is 0, 1, 2, 3 or 4;

or a pharmaceutically acceptable salt thereof.

As used herein, “alkyl” refers to a linear or branched saturated hydrocarbon group containing from 1 to 10 carbon atoms. Examples of such groups include methyl, ethyl, n-propyl, isopropyl, n-butyl, isobutyl, sec-butyl, tert butyl, n-pentyl, isopentyl, neopentyl or hexyl and the like.

As used herein “cycloalkyl” refers to a saturated monocyclic hydrocarbon ring of 3 to 7 carbon atoms. Examples of such groups include cyclopropyl, cyclobutyl, cyclopentyl, cyclohexyl, cycloheptyl and the like.

As used herein, “heterocyclyl” refers to a 4-8 membered monocyclic ring or a fused 8-12 membered bicyclic ring which may be saturated or partially unsaturated containing 1 to 4 heteroatoms selected from oxygen, nitrogen or sulphur. Examples of such monocyclic rings include pyrrolidinyl, pyrazolidinyl, piperidinyl, piperazinyl, morpholinyl, thiazolidinyl, and the like. Examples of heterocyclyl bicyclic rings include indolinyl, isoindolinyl, benzopyranyl, tetrahydrobenzazepinyl, tetrahydrobenzothienyl, tetrahydroisoquinolinyl and the like.

As used herein “heterocyclylalkyl” refers to a heterocyclyl-C₁₋₂alkyl group, wherein heterocyclyl and C₁₋₂ alkyl are as defined herein.

As used herein, “aryl” refers to a C₆ monocyclic or C₅₋₁₂bicyclic hydrocarbon ring wherein at least one ring is aromatic. Examples of such groups include phenyl, naphthyl, indenyl or tetrahydronaphthyl and the like.

As used herein, “arylakyl” refers to an aryl-C₁₋₆alkyl group wherein aryl and C₁₋₆alkyl are as defined herein.

As used herein “heteroaryl” refers to a 5-6 membered monocyclic aromatic or a fused 8-10 membered bicyclic aromatic ring containing 1 to 4 heteroatoms selected from oxygen, nitrogen and sulphur. Examples of such monocyclic aromatic rings include thienyl, furyl, furazanyl, pyrrolyl, thiazolyl, isoxazolyl, thiadiazolyl, pyranyl, pyrazolyl, pyrimidyl, pyridazinyl, pyrazinyl, pyridyl, and the like. Examples of such fused aromatic rings include isoquinolinyl, quinazolinyl, quinoxalinyl, indolyl, isoindolyl, indazolyl, benzofuranyl, isobenzofuranyl, benzothienyl, benzoxazolyl, benzothiazolyl, benzoisothiazolyl, and the like.

As used herein “heteroarylalkyl” refers to a heteroaryl-C₁₋₂alkyl group wherein heteroaryl and C₁₋₂alkyl are as defined herein.

As used herein “alkenyl” refers to a linear or branched hydrocarbon group containing one or more carbon-carbon double bonds having from 2 to 6 carbon atoms. Examples of such groups include ethenyl, propenyl, butenyl, pentenyl, or hexenyl and the like.

As used herein, “alkoxy” refers to an —O—C₁₋₄ alkyl group wherein C₁₋₄ alkyl is as defined herein. Examples of such groups include methoxy, ethoxy, propoxy, butoxy, pentoxy or hexoxy and the like.

As used herein, “halogen” or “halo” refers to F, Cl, Br or I.

As used herein, “optionally substituted,” unless specifically defined, means substituted, independently, at each occurrence, one to three times, by such groups as halogen, CN, C₁₋₄alkyl, C₂₋₄alkenyl, cycloC₁₋₆alkyl, heterocyclyl, aryl, heteroaryl, CO(O)R_{5′}, O—(CH₂)_{n′}—O, C(O)R_{5′}, CF₃, OCF₃, NO₂, C(O)NR_{5′}R_{6′}, (CR_{10′}R_{11′})_{2′}—OR_{5′}, (CR_{10′}R_{11′})_{z′}NR_{5′}R_{6′}, and (CR_{10′}R_{11′})_{z′}S(O)_{m′}R_{5′} such that the optional substituents may be further substituted, except for halogen and CN, one to three times, independently, by halogen or C₁₋₄alkyl.

As used herein, R_{5′} and R_{6′} represent, independently, at each occurrence, a H, C₁₋₃alkyl, cycloalkyl, cycloalkylC₁₋₃alkyl, C₂alkenyl, heterocyclyl, heterocyclylC₁₋₄alkyl, aryl, arylC₁₋₃alkyl, heteroaryl or a heteroarylC₁₋₃alkyl moiety, wherein each moiety, except H, is optionally substituted, independently, one to three times, by halogen or C₁₋₃alkyl.

As used herein R_{10′} and R_{11′} represent, at each occurrence, independently, H or C₁₋₄alkyl.

As used herein m′ is 0, 1 or 2.

As used herein n′ is 1, 2 or 3.

As used herein z′ is 0, 1, 2 or 3.

Suitably, X is O or S.

Preferably, X is O.

Suitably, R¹ and R² are, independently, selected from the group consisting of H, halogen, —CN, C₁₋₁₀alkyl, —C₂₋₆alkenyl, cycloalkyl, cycloalkylC₁₋₆alkyl, aryl, arylC₁₋₆alkyl, heterocyclyl, heteroaryl, (CR₁₀R₁₁)ₓNR₅R₆, C(O)OR₅, C(O)NR₅R₆, NR₅C(O)R₆, (CR₁₀R₁₁)ₓOR₅ and NC(O)R₅, optionally substituted, except for H, halogen and CN, one to three times, independently, by halogen, CN, C₁₋₄alkyl, aryl, heteroaryl, C(O)OR₁₉, O—(CR₁₉R₂₀)_{q}—O, C(O)R₁₉, CF₃, OCF₃, NO₂, C(O)NR₁₉R₂₀, (CR₁₀R₁₁)_{z}OR₁₉, (CR₁₀R₁₁)_{z}NR₁₉R₂₀, and (CR₁₀R₁₁)ₓS(O)ₘR₁₉.

In one embodiment, R¹ and R² are, independently, selected from the group consisting of H, halogen, CN, C₁₋₁₀alkyl, C₂₋₆alkenyl, cycloC₁₋₄alkyl, cycloalkylC₁₋₃alkyl, aryl, arylC₁₋₃alkyl, heterocyclyl, heteroaryl, (CR₁₀R₁₁)ₓNR₅R₆, NH₂, C(O)OR₅, NR₅C(O)C₁₋₄alkyl, C₁₋₄alkoxy, and (CR₁₀R₁₁)ₓOR₅, optionally substituted, one to three times, except for H, halogen and CN, independently, by halogen, CN, C₁₋₂alkyl, aryl, heteroaryl, C(O)OR₁₉, —O—(CH₂)_{q}—O, C(O)R₁₉, CF₃, OCF₃, NO₂, C(O)NR₁₉R₂₀, (CH₂)ₓOR₁₉, (CH₂)ₓNR₁₉R₂₀, and (CR₁₀R₁₁)ₓS(O)ₘR₁₉, such that the optional substituents, except for halogen and CN, may be further substituted, once or twice, independently, by halogen or C₁₋₂alkyl.

In another embodiment, R¹ and R² are, independently, selected from the group consisting of H, halogen, CN, C₂₋₃alkenyl, C(O)OH, phenethyl, pyrrolidinyl, N-propyl, NHC(O)C₁₋₃alkyl and C(O)CH₃.

In another embodiment, R¹ and R² are, independently, selected from the group consisting of a C₁₋₆alkyl, C₃₋₅cycloalkyl and C₃₋₄cycloalkylC₁₋₂alkyl, wherein each moiety is optionally substituted, independently, one to three times, by C₁₋₂alkyl or halogen.

In another embodiment, R¹ and R² represent, independently, phenyl, optionally substituted, independently, one to three times, by an optional substituent selected from the group consisting of F, OH, methyl, CN, OCF₃, CF₃, NH₂, CH₂OH, N-dimethyl, ethoxy, phenyl, NO₂, methylsulfonyl, isopropoxy and CH₂NC₁₋₂alkyl.

In another embodiment, R¹ and R² represent, independently, piperidinyl, optionally substituted by C₁₋₃alkyl.

In another embodiment, R¹ and R² represent, independently, an amine moiety, optionally substituted by C₁₋₄alkyl.

In another embodiment, R¹ and R² represent, independently, an ether moiety, substituted by C₁₋₃alkyl or benzyl.

In another embodiment, R¹ and R² represent, independently, a heteroaryl moiety selected from the group consisting of furyl, pyrizinyl, pyridyl, indanyl, thienyl, pyrrolyl, and thiazolyl, wherein the heteroaryl moiety is optionally substituted, independently, once or twice, by a substituent selected from the group consisting of methyl, chloro, CH₂NH₂, CN, CH₂OH, phenyl, CH₂NHCH₃ and 1,3,4-oxadiazolyl.

In another embodiment, R¹ and R² represent, independently, a heterocyclyl bicyclic moiety selected from the group consisting of quinolinyl, tetrahydroquinolinyl, methyltetrahydroquinolinyl, dihydrobenzodioxinyl, 3-benzothiophenyl, benzodioxolyl, benzothienyl, benzothiophenyl, benzofuranyl, indolyl, and thiazolyl, wherein the bicyclic moiety may be optionally substituted, independently, one to three times, by a substituent selected from the group consisting of methyl, phenyl, chloro and thiazolyl.

In another embodiment, R¹ and R² are selected, independently from the group consisting of hydrogen, I, Cl, Br, F, ON, methyl, ethyl, isobutyl, propyl, butyl, isopropyl, hexyl, 2-methylbutyl, 3-methylbutyl, 2-hydroxyethyl, 2-methyl-2-propenyl, 2-methyl-1-propenyl, 1-propenyl, cyclopentyl, cyclopropyl, cyclobutylethyl, cyclobutylmethyl, cyclopropylmethyl, phenyl, 2,4-difluorophenyl, 3,4-difluorophenyl, 4-fluorophenyl, 2-fluorophenyl, 3-fluorophenyl, 3-methylphenyl, 4-hydroxyphenyl, 2-cyanophenyl, 4-cyanophenyl, 4-trifluoromethylphenyl, 3-hydroxymethylphenyl, 3-hydroxyphenyl, 4-N,N-dimethylphenyl, 4-ethoxyphenyl, 4-biphenyl, 4-isopropoxyphenyl, 5-methylsulfonylphenyl, 3-ethoxyphenyl, 2-ethoxyphenyl, 3-cyanophenyl, 4-trifluoromethoxyphenyl, 3-trifluoromethoxyphenyl, 3-dimethylaminomethylphenyl, 3-N,N-dimethylbenzamidyl, 4-t-butylphenyl, 4-isopropylphenyl, 3-N,N-dimethylmethylphenyl, 3-nitrophenyl, carboxylic acid, pyrrolidinyl, morpholinyl, azetidinyl, phenpropyl, phenethyl, 3,4-dichlorophenethyl, ethylamino, methylethylisobutylamino, diethylamino, dimethylamino, 2,2-dimethylpropanamide, NH₂, N—N-dimethylamino, aniline, N-propyl, methylmethylether, benzylethylether, methylethylether, ethylether, isopropylether, N,2-dimethylpropanamide, 2-methylpropanamide, pyrazinyl, 3-pyridyl, 2-furyl, 3-furyl, 2-indanyl, 3-methyl-2-thienyl, 4-methyl-2-thienyl, 5-methyl-2-thienyl, 3-thienyl, 2-thienyl, 5-chloro-2-thienyl, 2-pyrrolyl, 1-methyl-2-pyrrolyl, 5-methyl-3-thienyl, 5-methylamino-2-thienyl, 5-hydroxymethyl-2-thienyl, 4,5-dimethyl-2-thienyl, 5-cyano-2-thienyl, 5-phenyl-2-thienyl, 2-methyl-1,3-thiazol-5-yl, 1,3-thiazol-2-yl, 5-acetyl-2-thienyl, 4,5-dimethyl-1,3-thiazol-2-yl, 4-methyl-1,3-thiazol-2-yl, 5-methyl-1,3,4-oxadiazol-2-yl, quinolinyl, 1,2,3,4-tetrahydroquinolinyl, 1,2,3,4-methyltetrahydroquinolinyl, 2,3-dihydro-1,4-benzodioxinyl, 3-benzothiophenyl, 1,3-benzodioxol-5-yl, 4-benzothienyl, 2-benzofuranyl, 4,5,6,7-tetrahydrobenzothienyl, 1-methylindol-5-yl, 5-(2-phenyl-1,3-thiazol-5-yl), 5-chloro-3-methyl-1-benzothien-2-yl, 2-benzothiophenyl, 1-methylindol-2-yl, and 5-(2-methyl-1,3-thiazol-4-yl)-2-thienyl.

In another embodiment, R¹ is selected from the group consisting of isobutyl, ethyl, phenyl, furanyl, quinolinyl, halogen, tetrahydroquinolinyl, pyrrolidinyl, thiophenyl, morpholinyl, cyclopentyl, isopropyl, amino, pyrazinyl, indolyl, thiazolyl, piperidinyl, N-acyl, benzothiophenyl and benzothiazolyl, all of which moieties may be optionally substituted, independently, one to three times, by C₁₋₄-alkyl or halogen.

Preferably, R¹ is selected from the group consisting of isobutyl, phenyl, thiazolyl and thienyl, optionally substituted by methyl.

In another embodiment, R² is selected from the group consisting of methyl, methoxymethyl, piperidinyl, ethyl, methoxyethyl, benzyloxyethyl, phenyl, pyrrolidinyl, amino, alkylamino, propyl, phenethyl, phenpropyl, butyl, isobutyl, cyclobutylethyl, 3-methylbutyl, dimethylaminomethyl, piperidinylmethyl, and alkylaminomethyl, all of which moieties may be optionally substituted, independently, one to three times, by C₁₋₄alkyl or halogen.

Preferably, R² is methyl, ethyl or propyl.

More preferably, R² is methyl.

Suitably, R¹ and R² together form an optionally substituted 5 to 8 membered ring, optionally containing one to three heteroatoms selected from N, O and S, wherein the optional substituents are selected, independently, at each occurrence, one to three times, from the group consisting of halogen, C₁₋₄alkyl, (CR₁₀R₁₁)_{z}S(O)ₘR₅, (CR₁₀R₁₁)_{z}OR₅, (CR₁₀R₁₁)_{z}NR₅R₆, C(O)R₅ and C(O)OR₅.

Suitably, R¹ and R² together form an optionally substituted heteroaryl ring, wherein the optional substituents are selected, independently, at each occurrence, one to three times, from the group consisting of halogen, C₁₋₄alkyl, (CR₁₀R₁₁)_{z}S(O)ₘR₅, (CR₁₀R₁₁)_{z}OR₅, (CR₁₀R₁₁)_{z}NR₅R₆, C(O)R₅ and C(O)OR₅.

Suitably, when R¹ is NR₅R₆, R₅ and R₆ join together to form a 5 to 7 membered ring, optionally substituted by C₁₋₄alkyl or halogen.

In one embodiment, R¹ and R² combine to form, together with the adjacent ring, a moiety selected from the group consisting of pyrimidinonyl, quinazolinyl, pyrridolpyrimidinecarboxylyl, pyrimidoazepinyl, cyclooctapyrimidinonyl, tetrahydropyrrolopyrimidinecarboxylyl, and pyrrazolopyrimidinonyl, optionally substituted, independently, once or twice, by a substituent selected from the group consisting of methyl, ethyl, benzyl, acetyl, methylsulfonyl, COCH₂C(CH₃)₂ and C(O)OCH₂C(CH₃)₂.

In another embodiment, R¹ and R² combine to form, together with the adjacent ring, a moiety selected from the group consisting of azepinyl, cyclohexyl, cycloheptyl, tetrahydrooxepinyl, tetrahydropyridynyl, tetrahydropyrrolidinyl, pyrazolyl and cyclooctyl, all of which moieties may be optionally substituted, independently, one to three times, by C₁₋₄alkyl or halogen.

In another embodiment, R¹ and R² combine to form, along with the adjacent ring, a moiety selected from the group consisting of 6-phenylmethyl-5,6,7,8-tetrahydropyrido[4,3-d]pyrimidin-4(3H)-onyl, 5,6,7,8-tetrahydro-4(3H)-quinazolinonyl, 6,6-dimethyl-4a,5,6,7,8,8a-hexahydro-4(3H)-quinazolinonyl, 3,5,6,7,8,9-hexahydrocycloheptapyrimidin-4-onyl, ethyl 3,5,7,8-tetrahydropyridolpyrimidine-6-carboxylyl, 3,5,6,7,8,9-hexahydropyrimido-4,5-azepin-4-onyl, 7-benzyl-3,5,6,7,8,9-hexahydropyrimido-4,5-azepin-4-onyl, 7-acetyl-3,5,6,7,8,9-hexahydropyrimido-4,5-azepine-4-onyl, 7-methylsulfonyl-3,5,6,7,8,9-hexahydropyrimido-4,5-azepin-4-onyl, 6(3-methylbutanoyl)-5,6,7,8-tetrahydropyrimido-4-onyl, 3-methylbutyryl-3,5,7,8-tetrahydropyridopyrimidine-6-carboxylyl, 5,6,7,8-tetrahydropyridopyrimidin-4(3H)-onyl, 5-methyl-5,6,7,8-tetrahydropyridopyrimidin-4(3H)-onyl, 5-ethyl-5,6,7,8-tetrahydropyridopyrimidin-4(3H)-onyl, 1,1′-dimethylethyl-3,4,5,7-tetrahydropyrrolopyrimidine-6-carboxylate, 1-methyl-1,5-dihydro-4-pyrrazolopyrimidin-4(3H)-onyl, and 5,6,7,8,9,10-hexahydrocyclooctapyrimidin-4(3H)-onyl.

Preferably, when R¹ and R² form a ring, the ring is cyclohexyl or dimethylcyclohexyl.

Suitably, R³ represents aryl or heteroaryl, optionally substituted, independently, one to three times, by C₁₋₄alkyl, halogen, CN or CF₃.

In one embodiment, R³ represents an aryl or heteroaryl moiety, optionally substituted, independently, one to three times, by a substituent selected from F, OH and Cl.

In another embodiment, R³ is selected from the group consisting of phenyl, pyrrolyl, thienyl, pyrridolyl, furanyl, imidazolyl, and furyl, optionally substituted, independently, once or twice, with a substituent selected from the group consisting of OH, F, methoxy and OCH₂phenyl.

In another embodiment, R³ is selected from the group consisting of 2-hydroxy-4-fluorophenyl, 2-hydroxy-3-fluorophenyl, 3-hydroxy-2-fluorophenyl, 2-hydroxyphenyl, 3-hydroxyphenyl, 2,3-dihydroxyphenyl, 2-fluorophenyl, 2-hydroxy-3-fluorophenyl, 2-methoxyphenyl, 3-fluoro-2(phenylmethyl)oxyphenyl, 2-pyrrolyl, 2-thienyl, 2-pyrridolyl, 2-furanyl, 2-imidazolyl, 2-furyl, and thienyl.

Suitably, R⁴ is selected from the group consisting of cycloalkyl₁₋₄alkyl, heteroaryl, heterocyclyl, aryl, heteroarylC₁₋₂alkyl, heterocyclylC₁₋₂alkyl, cycloalkylC₂alkenyl, arylC₂alkenyl, heteroarylC₂alkenyl and heterocyclylC₂alkenyl, wherein each moiety is optionally substituted, independently, one to three times, by C₁₋₄alkyl, F, CF₃ or Cl.

In one embodiment, R⁴ is selected from the group consisting of phenylC₁₋₂alkyl, cyclohexylC₁₋₂alkyl, cyclopentylC₁₋₂alkyl, thienylC₁₋₂alkyl, pyranylC₁₋₂alkyl, indenylC₁₋₂alkyl and piperidinylC₁₋₂alkyl, optionally substituted, independently, once or twice, by F, CF₃ or Cl.

In another embodiment, R⁴ is selected from the group consisting of phenylC₂alkenyl, cyclohexylC₂alkenyl, cyclopentylC₂alkenyl, thienylC₂alkenyl, pyranylC₂alkenyl, indenylC₂alkenyl and indenylC₂alkenyl.

In another embodiment, R⁴ is selected from the group consisting of 3-fluorophenylC₁₋₂alkyl, phenylC₁₋₂alkyl, 2-fluorophenylC₁₋₂alkyl, 3-trifluoromethylphenylC₁₋₂alkyl, 2-chlorophenylC₁₋₂alkyl, cyclopentylC₁₋₂alkyl, cyclohexylC₁₋₂alkyl, 2-thienylC₁₋₂alkyl, 3-thienylC₁₋₂alkyl, pyranylC₁₋₂alkyl, indenylC₁₋₂alkyl and piperidinylC₁₋₂alkyl.

Preferably, R⁴ is selected from the group consisting of phenethyl, 3-fluorophenethyl, 4-fluorophenethyl, 2-fluorophenethyl, 2-thienylethyl, phenylethyl, dihydroindenyl, cyclohexylethyl, 3-chlorophenethyl, 3-trifluoromethylphenethyl, cyclopentylethyl and tetrahydropyranylethyl.

More preferably, R⁴ is phenethyl, optionally substituted, once or twice, independently, by F.

Suitably, R₁₀ and R₁₁, represent, independently, hydrogen or C₁₋₄alkyl.

In one embodiment, O—(CR₅R₆)_{q}—O, represents 1,3-benzodioxinyl or 1,4-benzodioxinyl.

In another embodiment, q is 2 or 3.

An alternative embodiment of the present invention involves a compound according to formula (II) hereinbelow:

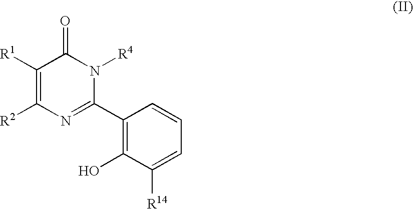

wherein:

R¹ and R² are, independently, selected from the group consisting of H, halogen, C₁₋₈alkyl, aryl, heterocyclyl, and heteroaryl, optionally substituted, except for H, and halogen, one to three times, independently, by halogen, ON, C₁₋₄alkyl, aryl, heteroaryl, —O—(CH₂)ₙ—O, CF₃, and OCF₃;

or R¹ and R² together form a 5 to 8 membered ring, optionally containing one to three heteroatoms selected from N, O and S, optionally substituted, independently, once or twice, by methyl;

R¹⁴ represents F or H;

R⁴ is represents arylC₁₋₂alkyl, optionally substituted, independently, one to three times, by F, CF₃ or Cl; and

n is 1, 2, or 3;

or a pharmaceutically acceptable salt thereof.

Unless otherwise specified, all definitions pertaining to formula (I) are applicable to formula (II).

Suitably, R¹ and R² are, independently, selected from the group consisting of H, halogen, C₁₋₈alkyl, aryl, heterocyclyl, and heteroaryl, optionally substituted, except for H, and halogen, one to three times, independently, by halogen, CN, C₁₋₄alkyl, aryl, heteroaryl, —O—(CH₂)ₙ—O, CF₃, and OCF₃.

In one embodiment, R¹ is selected from the group consisting of C₁₋₄alkyl, halogen, dihydrobenzodioxy, N-pyrrolyl, benzothienyl, benzothiazolyl.

In another embodiment, R¹ represents phenyl, optionally substituted, independently, once or twice, by F, Cl, and ON.

In another embodiment, R¹ represents thienyl, optionally substituted, independently, once or twice, by F, methyl, or ON.

In another embodiment, R¹ represents thiazolyl, optionally substituted by methyl.

Preferably, R¹ is selected from the group consisting of chloro, propyl, isobutyl, 2-thienyl, 5-methyl-2-thienyl, 3-cyano-2-thienyl, 4-methyl-2-thienyl, 3-cyano-2-thienyl, 2-cyanophenyl, 3-cyanophenyl, 3,5-difluorophenyl, dihydrobenzodioxyl, benzothienyl, benzothiazolyl, 2-methylthiazolyl, N-pyrrolyl and 2-methylthiazolyl.

More preferably, R¹ is selected from the group consisting of isobutyl, thienyl, 4-methyl-2-thienyl, phenyl and thiazolyl.

Suitably, R¹ and R² together form a 5 to 8 membered ring, optionally containing one to three heteroatoms selected from N, O and S, optionally substituted, independently, once or twice, by methyl.

Preferably, when R¹ and R² form a ring, the ring is selected from the group consisting of cyclohexyl and dimethylcyclohexyl.

Preferably, R² is methyl, ethyl or propyl.

More preferably, R² is methyl.

Suitably, R¹⁴ represents F or H.

Preferably, R¹⁴ is F.

Suitably, R⁴ is represents arylC₁₋₂alkyl, optionally substituted, independently, one to three times, by F, CF₃ or Cl.

Preferably, R⁴ is phenethyl, optionally substituted by F.

More preferably, R⁴ is 3-fluorophenethyl.

Suitably, n is 1, 2 or 3.

Preferably, n is 1 or 2.

Preferred compounds of the present invention include but are not limited to:
2-(2-Fluoro-3-hydroxyphenyl)-6-methyl-5-(2-methylpropyl)-3-(2-phenylethyl)-4(3H)-pyrimidinone;
2-(3-Hydroxyphenyl)-6-methyl-5-(2-methylpropyl)-3-(2-phenylethyl)-4(3H)-pyrimidinone;
2-(2,3-Dihydroxyphenyl)-6-methyl-5-(2-methylpropyl)-3-(2-phenylethyl)-4(3H)-pyrimidinone;
6-Methyl-5-(2-methylpropyl)-3-(2-phenylethyl)-2-(1H-pyrrol-2-yl)-4(3H)-pyrimidinone;
6-Methyl-5-(2-methylpropyl)-3-(2-phenylethyl)-2-(2-thienyl)-4(3H)-pyrimidinone;
6-Methyl-5-(2-methylpropyl)-3-(2-phenylethyl)-2-(2-pyridinyl)-4(3H)-pyrimidinone;
2-(2-Furanyl)-6-methyl-5-(2-methylpropyl)-3-(2-phenylethyl)-4(3H)-pyrimidinone;
2-(1H-imidazol-2-yl)-6-methyl-5-(2-methylpropyl)-3-(2-phenylethyl)-4(3H)-pyrimidinone;
5-Ethyl-2-(2-fluoro-3-hydroxyphenyl)-3-[2-(3-fluorophenyl)ethyl]-6-methyl-4(3H)-pyrimidinone;
5-Ethyl-3-[2-(3-fluorophenyl)ethyl]-6-methyl-2-(1H-pyrrol-2-yl)-4(3H)-pyrimidinone;
5-Bromo-2-{3-fluoro-2-[(phenyl methyl)oxy]phenyl}-6-methyl-3-(2-phenylethyl)-4(3H)-pyrimidinone;
5-Bromo-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(6-quinolinyl)-4(3H)-pyrimidinone;
2-(3-Fluoro-2-hydroxyphenyl)-6-methyl-3-(2-phenylethyl)-5-(1,2,3,4-tetrahydro-6-quinolinyl)-4(3H)-pyrimidinone;
2-(3-Fluoro-2-hydroxyphenyl)-6-methyl-5-(1-methyl-1,2,3,4-tetrahydro-6-quinolinyl)-3-(2-phenylethyl)-4(3H)-pyrimidinone;
2-(3-Fluoro-2-hydroxyphenyl)-5-(2-furanyl)-6-methyl-3-(2-phenylethyl)-4(3H)-pyrimidinone;
2-(3-Fluoro-2-hydroxyphenyl)-6-methyl-5-phenyl-3-[2-(2-thienyl)ethyl]-4(3H)-pyrimidinone;
2-(3-Fluoro-2-hydroxyphenyl)-6-methyl-3-(2-phenylethyl)-5-(1-pyrrolidinyl)-4(3H)-pyrimidinone;
5-(5-Chloro-2-thienyl)-2-(3-fluoro-2-hydroxyphenyl)-6-methyl-3-(2-phenylethyl)-4(3H)-pyrimidinone;
5-bromo-6-methyl-3-(2-phenylethyl)-2-{2-[(phenylmethyl)oxy]phenyl}-4(3H)-pyrimidinone;
2-(2-Hydroxyphenyl)-3-(2-phenylethyl)-6-(1-piperidinylmethyl)-4(3H)-pyrimidinone;
2-(2-Hydroxyphenyl)-6-{[methyl(2-methylpropyl)amino]methyl}-3-(2-phenylethyl)-4(3H)-pyrimidinone;
2-(2-Hydroxyphenyl)-6-methyl-5-[(1-methylethyl)oxy]-3-(2-phenylethyl)-4(3H)-pyrimidinone;
5-(2-Furanyl)-2-(2-hydroxyphenyl)-6-methyl-3-(2-phenylethyl)-4(3H)-pyrimidinone;
2-(2-Hydroxyphenyl)-6-methyl-3-(2-phenylethyl)-5-(2-thienyl)-4(3H)-pyrimidinone;
2-(2-Hydroxyphenyl)-6-methyl-5-(4-morpholinyl)-3-(2-phenylethyl)-4(3H)-pyrimidinone;
5-Ethyl-2-(3-fluoro-2-hydroxyphenyl)-3-[2-(3-fluorophenyl)ethyl]-6-(1-piperidinyl)-4(3H)-pyrimidinone;
5-Ethyl-1-[2-(3-fluorophenyl)ethyl]-2-[2-(methyloxy)phenyl]-6-oxo-1,6-dihydro-4-pyrimidinecarboxylic acid;
5-Ethyl-2-(2-hydroxyphenyl)-6-methyl-3-[(E)-2-phenylethenyl]-4(3H)-pyrimidinone;
2-(3,6-Difluoro-2-hydroxyphenyl)-5-ethyl-3-[2-(3-fluorophenyl)ethyl]-6-methyl-4(3H)-pyrimidinone;
2-(3-Fluoro-2-hydroxyphenyl)-6-methyl-5-propyl-3-[2-(2-thienyl)ethyl]-4(3H)-pyrimidinone;
2-(2-Hydroxyphenyl)-5,5-dimethyl-3-[2-(2-thienyl)ethyl]-5,6,7,8-tetrahydro-4(3H)-quinazolinone;
3-[2-(2-Fluorophenyl)ethyl]-2-(2-hydroxyphenyl)-5,6-dimethyl-5,6,7,8-tetrahydro-4(3H)-quinazolinone;
2-(2-Hydroxyphenyl)-3-(2-phenylethyl)-3,5,6,7,8,9-hexahydro-4H-cyclohepta[d]pyrimidin-4-one;
2-(3-Fluoro-2-hydroxyphenyl)-3-(2-phenylethyl)-5,6,7,8-tetrahydro-4(3H)-quinazolinone;
5-Cyclopentyl-2-(3-fluoro-2-hydroxyphenyl)-6-methyl-3-(2-phenylethyl)-4(3H)-pyrimidinone;
5-(2,3-Dihydro-1,4-benzodioxin-6-yl)-6-methyl-3-(2-phenylethyl)-2-(2-thienyl)-4(3H)-pyrimidinone;
2-(2-Hydroxyphenyl)-6-[(methyloxy)methyl]-3-(2-phenylethyl)-4(3H)-pyrimidinone;
2-(2-Hydroxyphenyl)-6-[(methyloxy)methyl]-5-(2-methylpropyl)-3-(2-phenylethyl)-4(3H)-pyrimidinone;
2-{3-Fluoro-2-[(phenylmethyl)oxy]phenyl}-5-(2-hydroxyethyl)-6-methyl-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinethione;
2-(3-Fluoro-2-hydroxyphenyl)-6-methyl-5-phenyl-3-(2-phenylethyl))-4(3H)-pyrimidimethione;
2-(3-Fluoro-2-hydroxyphenyl)-6-methyl-5-(2-methylpropyl)-3-(2-phenylethyl)-4(3H)-pyrimidinethione;
3-(2,3-Dihydro-1H-inden-2-yl)-2-(2-hydroxyphenyl)-6-methyl-5-(1-methylethyl)-4(3H)-pyrimidinone;
5,6-Diethyl-2-(3-fluoro-2-hydroxyphenyl)-3-[2-(2-fluorophenyl)ethyl]-4(3H)-pyrimidinone;
6-(2-Cyclohexylethyl)-5-ethyl-2-(3-fluoro-2-hydroxyphenyl)-3-[2-(2-fluorophenyl)ethyl]-4(3H)-pyrimidinone;
6-[2-(3,4-Dichlorophenyl)ethyl]-5-ethyl-2-(3-fluoro-2-hydroxyphenyl)-3-[2-(2-fluorophenyl)ethyl]-4(3H)-pyrimidinone;
2-(3-Fluoro-2-hydroxyphenyl)-3-[2-(2-fluorophenyl)ethyl]-6-methyl-5-(2-methylpropyl)-4(3H)-pyrimidinone;
2-(3-Fluoro-2-hydroxyphenyl)-6-methyl-5-(2-methylpropyl)-3-[2-(2-thienyl)ethyl]-4(3H)-pyrimidinone;
2-(3-Fluoro-2-hydroxyphenyl)-3-[2-(4-fluorophenyl)ethyl]-6-methyl-5-(2-methylpropyl)-4(3H)-pyrimidinone;
2-(3-Fluoro-2-hydroxyphenyl)-3-[2-(3-fluorophenyl)ethyl]-6-methyl-5-(2-methylpropyl)-4(3H)-pyrimidinone;
2-(2-Hydroxyphenyl)-7-methyl-3-(2-phenylethyl)-3,5,6,7,8,9-hexahydro-4H-pyrimido[4,5-d]azepin-4-one;
7-acetyl-2-(2-hydroxyphenyl)-3-(2-phenylethyl)-3,5,6,7,8,9-hexahydro-4H-pyrimido[4,5-d]azepin-4-one;
2-(2-Hydroxyphenyl)-7-(methylsulfonyl)-3-(2-phenylethyl))-3,5,6,7,8,9-hexahydro-4H-pyrimido[4,5-d]azepin-4-one;
5-Bromo-2-(2-hydroxyphenyl)-6-methyl-3-(2-phenylethyl)-4(3H)-pyrimidinone;
2-(2-Hydroxyphenyl)-5-iodo-6-methyl-3-(2-phenylethyl)-4(3H)-pyrimidinone;
5-Chloro-3-(2-cyclohexylethyl)-2-(2-hydroxyphenyl)-6-methyl-4(3H)-pyrimidinone;
5-Chloro-2-(2-hydroxyphenyl)-6-methyl-3-(2-(2-thienyl)ethyl-4(3H)-pyrimidinone;
5-Chloro-2-(2-hydroxyphenyl)-6-methyl-3-(2-phenylethyl)-4(3H)-pyrimidinethione;
5-Bromo-2-(3-hydroxyphenyl)-6-methyl-3-(2-phenylethyl)-4(3H)-pyrimidinone;
2-(3-Hydroxyphenyl)-6-methyl-5-phenyl-3-(2-phenylethyl)-4(3H)-pyrimidinone;
2-(2-hydroxyphenyl)-6-methyl-5-(phenylamino)-3-(2-phenylethyl)-4 (3H)-pyrimidinone;
5-(1-Azetidinyl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(propylamino)-4(3H)-pyrimidinone;
2-(2-Fluoro-3-hydroxyphenyl)-6-methyl-5-phenyl-3-(2-phenylethyl)-4(3H)-pyrimidinone;
2-(2-Hydroxyphenyl)-6-methyl-3-(2-phenylethyl)-5-(3-thienyl)-4(3H)-pyrimidinone;
5-(3-Furanyl)-2-(2-hydroxyphenyl)-6-methyl-3-(2-phenylethyl)-4(3H)-pyrimidinone;
5-(4-Biphenylyl)-2-(2-hydroxyphenyl)-6-methyl-3-(2-phenylethyl)-4(3H)-pyrimidinone;
5-(1,3-Benzodioxol-5-yl)-2-(2-hydroxyphenyl)-6-methyl-3-(2-phenylethyl)-4(3H)-pyrimidinone;
5-(2-fluorophenyl)-2-(2-hydroxyphenyl)-6-methyl-3-(2-phenylethyl)-4(3H)-pyrimidinone;
2-(2-Hydroxyphenyl)-6-methyl-3-(2-phenylethyl)-5-[4-(trifluoromethyl)phenyl]-4(3H)-pyrimidinone;
5-(3-Fluorophenyl)-2-(2-hydroxyphenyl)-6-methyl-3-(2-phenylethyl)-4(3H)-pyrimidinone;

### 5-(2,4-Difluorophenyl)-2-(2-hydroxyphenyl)-6-methyl-3-(2-phenylethyl)-4(3H)-pyrimidinone;

5-[4-(Dimethylamino)phenyl]-2-(3-fluoro-2-hydroxyphenyl)-6-methyl-3-(2-phenylethyl)-4(3H)-pyrimidinone;
5-[4-(Ethyloxy)phenyl]-2-(3-fluoro-2-hydroxyphenyl)-6-methyl-3-(2-phenylethyl)-4(3H)-pyrimidinone;
5-(1-Benzothien-3-yl)-2-(3-fluoro-2-hydroxyphenyl)-6-methyl-3-(2-phenylethyl)-4(3H)-pyrimidinone;
5-(1-Benzothien-4-yl)-2-(3-fluoro-2-hydroxyphenyl)-6-methyl-3-(2-phenylethyl)-4(3H)-pyrimidinone;
2-[2-(3-Fluoro-2-hydroxyphenyl)-4-methyl-6-oxo-1-(2-phenylethyl)-1,6-dihydro-5-pyrimidinyl]benzonitrile;
4-[2-(3-Fluoro-2-hydroxyphenyl)-4-methyl-6-oxo-1-(2-phenylethyl)-1,6-dihydro-5-pyrimidinyl]benzonitrile;
5-[2-(Ethyloxy)phenyl]-2-(3-fluoro-2-hydroxyphenyl)-6-methyl-3-(2-phenylethyl)-4(3H)-pyrimidinone;
5-[3-(Ethyloxy)phenyl]-2-(3-Fluoro-2-hydroxyphenyl)-6-methyl-3-(2-phenylethyl)-4(3H)-pyrimidinone;
5-(1-Benzofuran-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(1H-pyrrol-2-yl)-4(3H)-pyrimidinone;
2-(3-Fluoro-2-hydroxyphenyl)-5-[3-(hydroxymethyl)phenyl]-6-methyl-3-(2-phenylethyl)-4(3H)-pyrimidinone;
2-(3-Fluoro-2-hydroxyphenyl)-6-methyl-5-[3-(methylsulfonyl)phenyl]-3-(2-phenylethyl)-4(3H)-pyrimidinone;
2-(3-Fluoro-2-hydroxyphenyl)-6-methyl-3-(2-phenylethyl)-5-[3-(trifluoromethyl)phenyl]-4(3H)-pyrimidinone;
5-(3,4-Difluorophenyl)-2-(3-fluoro-2-hydroxyphenyl)-6-methyl-3-(2-phenylethyl)-4(3H)-pyrimidinone;
5-[4-(1,1-Dimethylethyl)phenyl]-2-(3-fluoro-2-hydroxyphenyl)-6-methyl-3-(2-phenylethyl)-4(3H)-pyrimidinone;
5-(5-Acetyl-2-thienyl)-2-(3-fluoro-2-hydroxyphenyl)-G-methyl-3-(2-phenylethyl)-4(3H)-pyrimidinone;
2-(3-Fluoro-2-hydroxyphenyl)-6-methyl-3-(2-phenylethyl)-5-{3-[(trifluoromethyl)oxy]phenyl}-4(3H)-pyrimidinone;
5-{3-[(Dimethylamino)methyl]phenyl}-2-(3-fluoro-2-hydroxyphenyl)-6-methyl-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]-N,N-dimethylbenzamide;
5-(4,5-Dimethyl-2-thienyl)-2-(3-fluoro-2-hydroxyphenyl)-6-methyl-3-(2-phenylethyl)-4(3H)-pyrimidinone;
5-[2-(3-Fluoro-2-hydroxyphenyl)-4-methyl-6-oxo-1-(2-phenylethyl)-1,6-dihydro-5-pyrimidinyl]-2-thiophenecarbonitrile;
2-(3-Fluoro-2-hydroxyphenyl)-6-methyl-5-(1-methyl-1H-pyrrol-2-yl)-3-(2-phenylethyl)-4(3H)-pyrimidinone;
2-(3-fluoro-2-hydroxyphenyl)-6-methyl-5-(1-methyl-1H-indol-2-yl)-3-(2-phenylethyl)-4(3H)-pyrimidinone;
2-(3-Fluoro-2-hydroxyphenyl)-6-methyl-3-(2-phenylethyl)-5-(1,3-thiazol-2-yl)-4(3H)-pyrimidinone;
2-(2-Hydroxyphenyl)-6-methyl-3-(2-phenylethyl)-5-(3-pyridinyl)-4(3H)-pyrimidinone;
2-(2-Hydroxyphenyl)-6-methyl-3-(2-phenylethyl)-5-(2-pyrazinyl)-4(3H)-pyrimidinone;
6-Methyl-2-[2-(methyloxy)phenyl]-3-(2-phenylethyl)-5-(2-thienyl)-4(3H)-pyrimidinone;
2-(2-Hydroxyphenyl)-6-methyl-5-phenyl-3-(2-phenylethyl)-4(3H)-pyrimidinone;
5-(4-Fluorophenyl)-2-(2-hydroxyphenyl)-6-methyl-3-(2-phenylethyl)-4(3H)-pyrimidinone;
2-(2-Hydroxyphenyl)-6-methyl-5-(3-methylphenyl)-3-(2-phenylethyl)-4(3H)-pyrimidinone;
2-(3-Fluoro-2-hydroxyphenyl)-6-methyl-5-(1-methyl-1H-indol-5-yl)-3-(2-phenylethyl)-4(3H)-pyrimidinone;
2-(3-Fluoro-2-hydroxyphenyl)-6-methyl-3-(2-phenylethyl)-5-{4-[(trifluoromethyl)oxy]phenyl}-4(3H)-pyrimidinone;
2-(3-Fluoro-2-hydroxyphenyl)-6-methyl-5-{4-[(1-methylethyl)oxy]phenyl}-3-(2-phenylethyl)-4(3H)-pyrimidinone;
2-(3-Fluoro-2-hydroxyphenyl)-6-methyl-3-(2-phenylethyl)-5-(6-quinolinyl)-4(3H)-pyrimidinone;
5-(2,3-Dihydro-1,4-benzodioxin-6-yl)-2-(2-hydroxyphenyl)-6-methyl-3-(2-phenylethyl)-4(3H)-pyrimidinone;
5-(5-Chloro-3-methyl-1-benzothien-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-[5-(1,3-oxazol-5-yl)-2-thienyl]-3-(2-3-phenylethyl)-4(3H)-pyrimidinone;
5-Fluoro-2-(2-hydroxyphenyl)-6-methyl-3-(2-phenylethyl)-4(3H)-pyrimidinone;
2-(2-Hydroxyphenyl)-6-methyl-5-(2-methylpropyl)-3-(2-phenylethyl)-4(3H)-pyrimidinone;
2-(2-Hydroxyphenyl)-6-methyl-5-(2-methyl-2-propen-1-yl)-3-(2-phenylethyl)-4(3H)-pyrimidinone;
5-(Cyclobutylmethyl)-6-methyl-2-[2-(methyloxy)phenyl]-3-(2-phenylethyl)-4(3m-pyrimidinone;
5-(Cyclobutylmethyl)-2-(2-hydroxyphenyl)-6-methyl-3-(2-phenylethyl)-4(3H)-pyrimidinone;
2-(2-Hydroxyphenyl)-6,6-dimethyl-3-(2-phenylethyl)-4a,5,6,7,8,8a-hexahydro-4(3H)-quinazolinone;
5-(Cyclopropylmethyl)-2-(3-fluoro-2-hydroxyphenyl)-6-methyl-3-(2-phenylethyl)-4(3H)-pyrimidinone;
5-(Cyclopropyl-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(3-methylbutyl)-3-(2-phenylethyl)-4(3-pyrimidinone;
5-(2-Cyclohexylethyl)-2-(3-fluoro-2-hydroxyphenyl)-6-methyl-3-(2-phenylethyl)-4(3H)-pyrimidinone;
5-(Cyclohexylmethyl)-2-(2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(phenylmethyl)-4(3H)-pyrimidinone;
5-Amino-2-(2-hydroxyphenyl)-6-methyl-3-(2-phenylethyl)-4(3H)-pyrimidinone;
2-(2-Hydroxyphenyl)-6-methyl-3-(2-phenylethyl)-5-(1-piperidinyl)-4(3H)-pyrimidinone;
5-(Dimethylamino)-2-(2-hydroxyphenyl)-6-methyl-3-(2-phenylethyl)-4(3H-pyrimidinone;
N-[2-(2-Hydroxyphenyl)-4-methyl-6-oxo-1-(2-phenylethyl)-1,6-dihydro-5-pyrimidinyl]-2,2-dimethylpropanamide;
N-[2-(2-Hydroxyphenyl)-4-methyl-6-oxo-1-(2-phenylethyl)-1,6-dihydro-5-pyrimidinyl]-2-methylpropanamide;
N-[2-(2-hydroxyphenyl)-4-methyl-6-oxo-1-(2-phenylethyl)-1,6-dihydro-5-pyrimidinyl]-N,2-dimethylpropanamide;
5-(Dipropylamino)-2-(2-hydroxyphenyl)-6-methyl-3-(2-phenylethyl)-4(3H)-pyrimidinone;
5-(Diethylamino)-2-(2-hydroxyphenyl)-6-methyl-3-(2-phenylethyl)-4(3H)-pyrimidinone;
5-(Ethylamino)-2-(2-hydroxyphenyl)-6-methyl-3-(2-phenylethyl)-4(3H)-pyrimidinone;
2-(2-Hydroxyphenyl)-5-(2-methylpropyl)-3-(2-phenylethyl)-6-propyl)-4(3H)-pyrimidinone;
6-Ethyl-2-(2-hydroxyphenyl)-5-(2-methylpropyl)-3-(2-phenylethyl)-4(3H)-pyrimidinone;
6-Butyl-2-(2-hydroxyphenyl)-5-(2-methylpropyl)-3-(2-phenylethyl)-4(3H)-pyrimidinone;
2-(2-Hydroxyphenyl)-5-(2-methylpropyl)-3-(2-phenylethyl)-6-{2-[(phenylmethyl)oxy]ethyl}-4(3H)-pyrimidinone;
6-(2-Hydroxyethyl)-2-(2-hydroxyphenyl)-5-(2-methylpropyl)-3-(2-phenylethyl)-4(3H)-pyrimidinone;
6-[2-(methyloxy)ethyl]-5-(2-methyl-1-propen-1-yl)-3-(2-phenylethyl)-4(3H)-pyrimidinone;
2-(2-hydroxyphenyl)-6-[2-(methyloxy)ethyl]-5-(2-methylpropyl)-3-(2-phenylethyl)-4(3H)-pyrimidinone;
5-(dimethylamino)-2-(3-fluoro-2-hydroxyphenyl)-6-methyl-3-(2-phenylethyl)-4(3H)-pyrimidinone;
5-(Dimethylamino)-2-(2-fluoro-3-hydroxyphenyl)-B-methyl-3-(2-phenylethyl)-4(3H)-pyrimidinone;
6-Methyl-2,5-diphenyl-3-(2-phenylethyl)-4(3H)-pyrimidinone;
2-(2-Fluorophenyl)-6-methyl-5-phenyl-3-(2-phenylethyl)-4(3H)-pyrimidinone;
3-[2-(2-chlorophenyl)ethyl]-2-(2-hydroxyphenyl)-5,6,7,8-tetrahydro-4(3H)-quinazolinone;
3-[2-(3-fluorophenyl)ethyl]-2-(2-hydroxyphenyl)-5,5-dimethyl-5,6,7,8-tetrahydro-4(3H)-quinazolinone;
3-(2-cyclohexylethyl)-2-(2-hydroxyphenyl)-5,5-dimethyl-5,6,7,8-tetrahydro-4(3H)-quinazolinone;
3-[2-(3-fluorophenyl)ethyl]-2-(2-furanyl)-5,6,7,8-tetrahydro-4(3H)-quinazolinone;
3-[2-(3-fluorophenyl)ethyl]-2-(2-thienyl)-5,6,7,8-tetrahydro-4(3H)-quinazolinone;
ethyl 2-(2-hydroxyphenyl)-4-methyl-6-oxo-1-(2-phenylethyl)-1,6-dihydro-5-pyrimidinecarbonitrile;
Ethyl 2-(2-hydroxyphenyl)-4-methyl-6-oxo-1-(2-phenylethyl)-1,6-dihydro-5-pyrimidinecarboxylate;
2-(2-hydroxyphenyl)-6-methyl-5-(1-methylpropyl)-3-[2-(2-thienyl)ethyl]-4(3H)-pyrimidinone;
2-(2-hydroxyphenyl)-6-methyl-5-(1-methylpropyl)-3-(2-phenylethyl)-4(3H)-pyrimidinone;
2-(3-fluoro-2-hydroxyphenyl)-6-methyl-5-(1-methylpropyl)-3-(2-phenylethyl)-4(3H)-pyrimidinone;
5-butyl-2-(2-hydroxyphenyl)-6-methyl-3-(2-phenylethyl)-4(3H)-pyrimidinone;
2-(2-hydroxyphenyl)-6-methyl-5-pentyl-3-(2-phenylethyl)-4(3H)-pyrimidinone;
5-hexyl-2-(2-hydroxyphenyl)-6-methyl-3-(2-phenylethyl)-4(3H)-pyrimidinone;
5-butyl-2-(2-hydroxyphenyl)-6-methyl-3-[2-(2-thienyl)ethyl]-4(3H)-pyrimidinone,
2-(2-hydroxyphenyl)-6-methyl-5-pentyl-3-[2-(2-thienyl)ethyl]-4(3H)-pyrimidinone;
5-hexyl-2-(2-hydroxyphenyl)-6-methyl-3-[2-(2-thienyl)ethyl]-4(3H)-pyrimidinone;
2-(3-fluoro-2-hydroxyphenyl)-3-[2-(3-fluorophenyl)ethyl]-5,6,7,8-tetrahydro-4(3H)-quinazolinone;
2-(3-fluoro-2-hydroxyphenyl)-3-[2-(3-fluorophenyl)ethyl]-3,5,6,7,8,9-hexahydro-4H-cyclohepta[d]pyrimidin-4-one;
Ethyl 2-(2-hydroxyphenyl)-4-oxo-3-(2-phenylethyl)-3,5,7,8-tetrahydropyrido[4,3-d]pyrimidine-6(4H)-carboxylate;
(2-hydroxyphenyl)-6-(3-methylbutanoyl)-3-(2-phenylethyl)-5,6,7,8-tetrahydropyrido[4,3-d]pyrimidin-4(3H)-one;
5-ethyl-2-(2-hydroxyphenyl)-6-methyl-3-[2-(2-thienyl)ethyl]-4(3H)-pyrimidinone;
5-Isopropyl-2-(2-hydroxy-phenyl)-6-methyl-3-(2-thiophen-2-yl-ethyl)-3H-pyrimidin-4-one;
5-Isopropyl-2-(2-hydroxy-phenyl)-6-methyl-3-(2-cyclohexyl-ethyl)-3H-pyrimidin-4-one;
5-Ethyl-2-(2-hydroxy-3-fluorophenyl)-6-methyl-3-(2-fluorophenylethyl)-3H-pyrimidin-4-one;
5-propenyl-2-(2-hydroxy-3-fluorophenyl)-6-methyl-3-(3-fluorophenylethyl)-3H-pyrimidin-4-one;
3-(2-cyclohexylethyl)-2-(2-hydroxyphenyl)-5,6,7,8-tetrahydro-4(3H)-quinazolinone;
3-(2-thiophen-2-yl-ethyl)-2-(2-hydroxy-phenyl)-5,6,7,8-tetrahydro-3H-quinazolin-4-one;
3-(2-thiophen-2-yl-ethyl)-2-(2-hydroxy-3-fluorophenyl)-5,6,7,8-tetrahydro-3H-quinazolin-4-one;
3-(2-thiophen-3-yl-ethyl)-2-(2-hydroxy-phenyl)-5,6,7,8-tetrahydro-3H-quinazolin-4-one;
3-(3-chlorophenethyl)-2-(2-hydroxy-phenyl)-5,6,7,8-tetrahydro-3H-quinazolin-4-one;
3-(2-cyclopentylethyl)-2-(2-hydroxy-phenyl)-5,6,7,8-tetrahydro-3H-quinazolin-4-one;
3-(3-trifluoromethylphenethyl)-2-(2-hydroxy-phenyl)-5,6,7,8-tetrahydro-3H-quinazolin-4-one;
2-(2-Hydroxyphenyl)-3-(2-phenylethyl)-5,6,8,9-tetrahydrooxepino[4,5-d]pyrimidin-4(3H)-one;
3-(2-Cyclohexyl-ethyl)-2-(2-hydroxy-phenyl)-3,5,6,7,8,9-hexahydro-cycloheptapyrimidin-4-one;
2-(2-hydroxyphenyl)-3-(2-phenylethyl)-6-(phenylmethyl)-5,6,7,8-tetrahydropyrido[4,3-d]pyrimidin-4(3H)-one;
2-Methylpropyl-2-(2-hydroxyphenyl-4-oxo-3-(2-phenylethyl)-3,5,7,8-tetrahydropyrido[4,3-d]pyrimidine-6(4H)-carboxylate;
2-(3-Fluoro-2-hydroxyphenyl)-6-methyl-5-[5-(2-methyl-1,3-thiazol-4-yl)-2-thienyl]-3-(2-phenylethyl)-4(3H)-pyrimidinone;
2-[2-(hydroxy)phenyl]-3-(2-phenylethyl)-5,6,7,8-tetrahydropyrido[3,2-d]pyrimidin-4(3H)-one;
2-(2-hydroxyphenyl)-5-methyl-3-(2-phenylethyl)-5,6,7,8-tetrahydropyrido[3,2-d]pyrimidin-4(3H)-one;
5-ethyl-2-[2-hydroxyphenyl]-3-(2-phenylethyl)-5,6,7,8-tetrahydropyrido[3,2-d]pyrimidin-4(3H)-one;
1,1-dimethylethyl 2-(2-hydroxyphenyl)-4-oxo-3-(2-phenylethyl)-3,4,5,7-tetrahydro-6H-pyrrolo[3,4-d]pyrimidine-6-carboxylate;
5-(2-methyl propyl-2-yl)-2-(2-hydroxy-phenyl)-6-methyl-3-(2-phenethyl)-3H-pyrimidin-4-one;
5-[2-(3-fluorophenyl)ethyl]-6-(2-hydroxyphenyl)-1-methyl-1,5-dihydro-4H-pyrazolo[3,4-d]pyrimidin-4-one;
5-ethyl-2-(3-fluoro-2-hydroxyphenyl)-3-[2-(3-fluorophenyl)ethyl]-6-phenyl-4(3H)-pyrimidinone;
6-[3,4-bis(methyloxy)phenyl]-5-ethyl-2-(3-fluoro-2-hydroxyphenyl)-3-[2-(3-fluorophenyl)ethyl]-4(3H)-pyrimidinone;
5-ethyl-2-(3-fluoro-2-hydroxyphenyl)-3-[2-(3-fluorophenyl)ethyl]-6-(3-nitrophenyl)-4(3H)-pyrimidinone;
5-ethyl-3-[2-(3-fluorophenyl)ethyl]-2-(2-hydroxyphenyl)-6-(1-pyrrolidinyl)-4(3H)-pyrimidinone;
6-(dimethylamino)-5-ethyl-3-[2-(3-fluorophenyl)ethyl]-2-(2-hydroxyphenyl)-4(3H)-pyrimidinone;
5-ethyl-3-[2-(3-fluorophenyl)ethyl]-2-(2-hydroxyphenyl)-6-(methylamino)-4(3H)-pyrimidinone;
5-cyclopentyl-3-[2-(3-fluorophenyl)ethyl]-2-(2-hydroxyphenyl)-6-methyl-4(3H)-pyrimidinone;
2-(2-hydroxyphenyl)-6-methyl-5-(2-methylpropyl)-3-(2-phenylethyl)-4(3H)-pyrimidinone;
2-(2-hydroxyphenyl)-6-methyl-5-(2-methylpropyl)-3-[2-(2-thienyl)ethyl]-4(3H)-pyrimidinone;
5-Ethyl-2-(2-hydroxy-phenyl)-6-Ethyl-3-phenylethyl-3H-pyrimidin-4-one;
5-Ethyl-2-(2-hydroxy-phenyl)-6-Propyl-3-phenylethyl-3H-pyrimidin-4-one;
5-Ethyl-2-(3-fluoro-2-hydroxy-phenyl)-6-(2-phenylethyl)-3-(2-fluoro-phenylethyl)-3H-pyrimidin-4-one;
5-Ethyl-2-(3-fluoro-2-hydroxy-phenyl)-6-propyl-3-(2-fluoro-phenyl ethyl)-3H-pyrimidin-4-one;
5-Ethyl-2-(3-fluoro-2-hydroxy-phenyl)-6-(3-phenyl-propyl)-3-(2-fluoro-phenylethyl)-3H-pyrimidin-4-one;
5-Ethyl-2-(3-fluoro-2-hydroxy-phenyl)-6-butyl-3-(2-fluoro-phenylethyl)-3H-pyrimidin-4-one;
5-Ethyl-2-(3-fluoro-2-hydroxy-phenyl)-6-(2-methyl-propyl)-3-(2-fluoro-phenylethyl)-3H-pyrimidin-4-one;
5-Ethyl-2-(3-fluoro-2-hydroxy-phenyl)-6-(3-methyl-butyl)-3-(2-fluoro-phenylethyl)-3H-pyrimidin-4-one;
5-Ethyl-2-(3-fluoro-2-hydroxy-phenyl)-6-(2-cyclobutyl-ethyl)-3-(2-fluoro-phenylethyl)-3H-pyrimidin-4-one;
5-Ethyl-2-(3-fluoro-2-hydroxy-phenyl)-6-(3,4-dichlorophenethyl)-3-(2-fluoro-phenylethyl)-3H-pyrimidin-4-one;
5-ethyl-2-(4-fluoro-2-hydroxyphenyl)-6-methyl-3-(2-phenylethyl)-4(3H)-pyrimidinone;
6-methyl-5-(2-methyl-1,3-thiazol-5-yl)-3-(2-phenylethyl)-2-{2-[(phenylmethyl)oxy]phenyl}-4(3H)-pyrimidinone;
2-(2-hydroxyphenyl)-6-methyl-5-(5-methyl-2-thienyl)-3-(2-phenylethyl)-4(3H)-pyrimidinone;
2-(2-hydroxyphenyl)-6-methyl-5-(5-methyl-3-thienyl)-3-(2-phenylethyl)-4(3H)-pyrimidinone;
2-(3-fluoro-2-hydroxyphenyl)-6-methyl-5-(5-methyl-3-thienyl)-3-(2-phenylethyl)-4(3H)-pyrimidinone;
5-(4,5-dimethyl-2-thienyl)-2-(2-hydroxyphenyl)-6-methyl-3-(2-phenylethyl)-4(3H)-pyrimidinone;
2-(2-hydroxyphenyl)-6-methyl-5-[5-(1,3-oxazol-5-yl)-2-thienyl]-3-(2-phenylethyl)-4(3H)-pyrimidinone;
2-(2-hydroxyphenyl)-6-methyl-5-(4-methyl-2-thienyl)-3-(2-phenylethyl)-4(3H)-pyrimidinone;
5-[2-(2-hydroxyphenyl)-4-methyl-6-oxo-1-(2-phenylethyl)-1,6-dihydro-5-pyrimidinyl]-2-thiophenecarbonitrile;
2-(2-hydroxyphenyl)-6-methyl-3-(2-phenylethyl)-5-[5-(1H-tetrazol-5-yl)-2-thienyl]-4(3H)-pyrimidinone;
5-[5-(Aminomethyl)-2-thienyl]-2-(2-hydroxyphenyl)-6-methyl-3-(2-phenylethyl)-4(3H)-pyrimidinone;
2-(2-hydroxyphenyl)-6-methyl-5-{5-[(methylamino)methyl]-2-thienyl}-3-(2-phenylethyl)-4(3H)-pyrimidinone;
5-[5-(hydroxymethyl)-2-thienyl]-2-(2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(4,5,6,7-tetrahydro-1-benzothien-2-yl)-4(3H)-pyrimidinone;
2-(3-Fluoro-2-hydroxyphenyl)-6-methyl-3-(2-phenylethyl)-5-(2-phenyl-1,3-thiazol-5-yl)-4(3H)-pyrimidinone;
2-(3-Fluoro-2-hydroxyphenyl)-5-(4-hydroxyphenyl)-6-methyl-3-(2-phenylethyl)-4(3H)-pyrimidinone;
2-(3-Fluoro-2-hydroxyphenyl)-5-(2-hydroxyphenyl)-6-methyl-3-(2-phenylethyl)-4(3H)-pyrimidinone;
2-(3-Fluoro-2-hydroxyphenyl)-5-(3-hydroxyphenyl)-6-methyl-3-(2-phenylethyl)-4(3H)-pyrimidinone;
2-(2-Hydroxyphenyl)-6-methyl-5-(2-methylpropyl)-3-[2-(1-piperidinyl)ethyl]-4(3H)-pyrimidinone;
5-Ethyl-3-[2-(2-fluorophenyl)ethyl]-2-(3-hydroxyphenyl)-6-methyl-4(3H)-pyrimidinone;
2-(2-Hydroxyphenyl)-6-methyl-5-[5-(5-methyl-1,3,4-oxadiazol-2-yl)-2-thienyl]-3-(2-phenylethyl)-4(3H)-pyrimidinone;
5-(2,3-Dihydro-1,4-benzodioxin-6-yl)-2-(2-hydroxyphenyl)-6-[(methyloxy)methyl]-3-(2-phenylethyl)-4(3H)-pyrimidinone;
2-(2-Hydroxyphenyl)-6-[(methyloxy)methyl]-5-(4-methyl-2-thienyl)-3-(2-phenylethyl)-4(3H)-pyrimidinone;
2-(2-Hydroxyphenyl)-6-[(methyloxy)methyl]-5-(5-methyl-2-thienyl)-3-(2-phenylethyl)-4(3H)-pyrimidinone;
5-Bromo-6-[(dimethylamino)methyl]-2-(2-hydroxyphenyl)-3-(2-phenylethyl)-4(3H)-pyrimidinone;
6-[(Dimethylamino)methyl]-2-(2-hydroxyphenyl)-3-(2-phenylethyl)-4(3H)-pyrimidinone;
2-(3-Fluoro-2-hydroxyphenyl)-6-methyl-5-(5-methyl-3-thienyl)-3-(2-phenylethyl)-4(3H)-pyrimidinone;
5-(4,5-Dimethyl-1,3-thiazol-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(4-methyl-1,3-thiazol-2-yl) 3-(2-phenylethyl)-4(3H)-pyrimidinone;
5-(1,3-Benzodioxol-5-yl)-2-(3-fluoro-2-hydroxyphenyl)-6-methyl-3-(2-phenylethyl)-4(3H)-pyrimidinone;
3-(2,3-Dihydro-1H-inden-2-yl)-2-(2-hydroxyphenyl)-6-methyl-5-(5-methyl-2-thienyl)-4(3H)-pyrimidinone;
3-[1-(2,3-Dihydro-1H-inden-2-yl)-2-(2-hydroxyphenyl)-4-methyl-6-oxo-1,6-dihydro-5-pyrimidinyl]benzonitrile;
3-(2,3-Dihydro-1H-inden-2-yl)-5-(4,5-dimethyl-1,3-thiazol-2-yl)-2-(2-hydroxyphenyl)-6-methyl-4(3H)-pyrimidinone;
3-(2-Cyclohexylethyl)-2-(3-fluoro-2-hydroxyphenyl)-6-methyl-5-(5-methyl-2-thienyl)-4(3H)-pyrimidinone;
3-(2-Cyclohexylethyl)-5-(4,5-dimethyl-1,3-thiazol-2-yl)-2-(3-fluoro-2-hydroxyphenyl)-6-methyl-4(3H)-pyrimidinone;
3-(2-Cyclohexylethyl)-2-(3-fluoro-2-hydroxyphenyl)-6-methyl-5-(2-methyl-1,3-thiazol-5-yl)-4(3H)-pyrimidinone;
2-(3-Fluoro-2-hydroxyphenyl)-6-methyl-5-(5-methyl-2-thienyl)-3-[2-(2-thienyl)ethyl]-4(3H)-pyrimidinone;
5-(4,5-Dimethyl-1,3-thiazol-2-yl)-2-(3-fluoro-2-hydroxyphenyl)-6-methyl-3-[2-(2-thienyl)ethyl]-4(3H)-pyrimidinone;
2-(3-Fluoro-2-hydroxyphenyl)-6-methyl-5-(5-methyl-2-thienyl)-3-[2-(tetrahydro-2H-pyran-4-yl)ethyl]-4(3H)-pyrimidinone;
2-(3-Fluoro-2-hydroxyphenyl)-3-[2-(2-fluorophenyl)ethyl]-6-methyl-5-(5-methyl)-2-thienyl)-4(3H)-pyrimidinone;
2-(3-Fluoro-2-hydroxyphenyl)-3-[2-(3-fluorophenyl)ethyl]-6-methyl-5-(5-methyl-2-thienyl)-4(3H)-pyrimidinone;
2-(3-Fluoro-2-hydroxyphenyl)-3-[2-(4-fluorophenyl)ethyl]-6-methyl-5-(5-methyl-2-thienyl)-4(3H)-pyrimidinone;
2-(3-Fluoro-2-hydroxyphenyl)-6-methyl-b-(3-methyl-2-thienyl)-3-(2-phenylethyl)-4(3H)-pyrimidinone;
2-(2-Hydroxyphenyl)-3-(2-phenylethyl)-5,6,7,8,9,10-hexahydrocycloocta[d]pyrimidin-4(3H)-one;
5-(1-Benzothien-2-yl)-3-(2,3-dihydro-1H-inden-2-yl)-2-(2-hydroxyphenyl)-6-methyl-4(3H)-pyrimidinone;
2-(3-Fluoro-2-hydroxyphenyl)-6-methyl-5-(2-methylpropyl)-3-(2-phenylethyl)-4(3H)-pyrimidinone;
2-(2-Hydroxyphenyl)-5,5-dimethyl-3-(2-phenylethyl)-5,6,7,8-tetrahydro-4(3H)-quinazolinone;
5-Chloro-2-(2-hydroxyphenyl)-6-methyl-3-(2-phenylethyl)-4(3H)-pyrimidinone;
3-[2-(3-Fluorophenyl)ethyl]-2-(2-hydroxyphenyl)-5,6,7,8-tetrahydro-4(3H)-quinazolinone;
2-(3-Fluoro-2-hydroxyphenyl)-6-methyl-5-(5-methyl-2-thienyl)-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]benzonitrile;
5-(2,3-Dihydro-1,4-benzodioxin-6-yl)-2-(3-fluoro-2-hydroxyphenyl)-6-methyl-3-(2-phenylethyl)-4(3H)-pyrimidinone;
5-(3,5-Difluorophenyl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(4-methyl-2-thionyl)-3-(2-phenylethyl)-4(3H)-pyrimidinone;
5-(1-Benzothien-2-yl)-2-(3-Fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(3-thienyl)-4(3H)-pyrimidinone;
2-(3-Fluoro-2-hydroxyphenyl)-6-methyl-3-(2-phenylethyl)-5-(5-phenyl-2-thienyl)-4(3H)-pyrimidinone;
2-(3-Fluoro-2-hydroxyphenyl)-6-methyl-3-(2-phenylethyl)-5-(2-thienyl)-4(3H)-pyrimidinone;
5-(1,3-Benzothiazol-2-yl)-2-(3-fluoro-2-hydroxyphenyl)-6-methyl-3-(2-phenylethyl)-4(3H)-pyrimidinone;
5-(1-Benzothien-2-yl)-2-(2-hydroxyphenyl)-6-methyl-3-(2-phenylethyl)-4(3H)-pyrimidinone;
2-(2-Hydroxyphenyl)-6-methyl-5-(2-methyl-1,3-thiazol-5-yl)-3-(2-phenylethyl)-4(3H)-pyrimidinone;
5-[2-(2-Hydroxyphenyl)-4-methyl-6-oxo-1-(2-phenylethyl)-1,6-dihydro-5-pyrimidinyl]-2-thiophenecarbonitrile;
3-[2-(2-Hydroxyphenyl)-4-methyl-6-oxo-1-(2-phenylethyl)-1,6-dihydro-5-pyrimidinyl]benzonitrile;
2-(3-Fluoro-2-hydroxyphenyl)-6-methyl-5-phenyl-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinone;
2-(3-Fluoro-2-hydroxyphenyl)-6-methyl-5-(2-methyl-1,3-thiazol-5-yl)-3-(2-phenylethyl)-4(3H)-pyrimidinone; and
2-(3-Fluoro-2-hydroxyphenyl)-6-methyl-3-(2-phenylethyl)-5-(1H-pyrrol-1-yl)-4(3H)-pyrimidinone;

or a pharmaceutically acceptable salt thereof.

More preferred compounds useful in the present invention include but are not limited to:
2-(3-fluoro-2-hydroxyphenyl)-6-methyl-5-(2-methylpropyl)-3-(2-phenylethyl)-4(3H)-pyrimidinone;
2-(2-hydroxyphenyl)-5,5-dimethyl-3-(2-phenylethyl)-5,6,7,8-tetrahydro-4(3H)-quinazolinone;
5-chloro-2-(2-hydroxyphenyl)-6-methyl-3-(2-phenylethyl)-4(3H)-pyrimidinone;
3-[2-(3-Fluorophenyl)ethyl]-2-(2-hydroxyphenyl)-5,6,7,8-tetrahydro-4(3H)-quinazolinone;
2-(3-Fluoro-2-hydroxyphenyl)-6-methyl-5-(5-methyl-2-thienyl)-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]benzonitrile;
5-(2,3-dihydro-1,4-benzodioxin-6-yl)-2-(3-fluoro-2-hydroxyphenyl)-6-methyl-3-(2-phenylethyl)-4(3H)-pyrimidinone;
5-(3,5-Difluorophenyl)-2-(3-fluoro-2-hydroxyphenyl)-6-methyl-3-(2-phenylethyl)-4(3H)-pyrimidinone;
2-(3-fluoro-2-hydroxyphenyl)-6-methyl-5-(4-methyl-2-thienyl)-3-(2-phenylethyl)-4(3H)-pyrimidinone;
5-(1-benzothien-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(2-thienyl)-4(3H)-pyrimidinone;
5-(1,3-Benzothiazol-2-yl)-2-(3-fluoro-2-hydroxyphenyl)-6-methyl-3-(2-phenylethyl)-4(3H)-pyrimidinone;
5-(1-Benzothien-2-yl)-2-(2-hydroxyphenyl)-6-methyl-3-(2-phenylethyl)-4(3H)-pyrimidinone;
2-(2-Hydroxyphenyl)-6-methyl-5-(2-methyl-1,3-thiazol-5-yl)-3-(2-phenylethyl)-4(3H)-pyrimidinone;
5-[2-(2-Hydroxyphenyl)-4-methyl-6-oxo-1-(2-phenylethyl)-1,6-dihydro-5-pyrimidinyl]-2-thiophenecarbonitrile;
3-[2-(2-hydroxyphenyl)-4-methyl-6-oxo-1-(2-phenylethyl)-1,6-dihydro-5-pyrimidinyl]benzonitrile;
2-(3-Fluoro-2-hydroxyphenyl)-6-methyl-5-phenyl-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinone;

and
2(3-Fluoro-2-hydroxyphenyl)-6-methyl-3-(2-phenylethyl)-5-(1H-pyrrol-1-yl)-4(3H)-pyrimidinone;

or a pharmaceutically acceptable salt thereof.

As used herein, the term “pharmaceutically acceptable” means a compound which is suitable for pharmaceutical use. Salts and solvates of compounds of the invention which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

With regard to stereoisomers, the present compounds may have one or more asymmetric carbon atom and may occur as racemates, racemic mixtures and as individual enantiomers or diastereomers. All such isomeric forms are included within the present invention, including mixtures thereof.

Furthermore, some of the crystalline forms of the present compounds may exist as polymorphs, which are included in the present invention.

Because of their potential use in medicine, the salts of the compounds of formula (I) and (II) are preferably pharmaceutically acceptable. Suitable pharmaceutically acceptable salts can include acid or base addition salts.

A pharmaceutically acceptable acid addition salt can be formed by reaction of a compound of formula (I) or (II) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamaic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated for example by crystallisation and filtration. A pharmaceutically acceptable acid addition salt of a compound of formula (I) or (II) can comprise or be for example a hydrobromide, hydrochloride, sulfate, nitrate, phosphate, succinate, maleate, formate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

A pharmaceutically acceptable base addition salt can be formed by reaction of a compound of formula (I) or (II) with a suitable inorganic or organic base (e.g. triethylamine, ethanolamine, triethanolamine, choline, arginine, lysine or histidine), optionally in a suitable solvent such as an organic solvent, to give the base addition salt which is usually isolated for example by crystallisation and filtration.

Other suitable pharmaceutically acceptable salts include pharmaceutically acceptable metal salts, for example pharmaceutically acceptable alkali-metal or alkaline-earth-metal salts such as sodium, potassium, calcium or magnesium salts; in particular pharmaceutically acceptable metal salts of one or more carboxylic acid moieties that may be present in the compound of formula (I) or (II).

Other non-pharmaceutically acceptable salts, e.g. oxalates, may be used, for example in the isolation of compounds of the invention, and are included within the scope of this invention.

The invention includes within its scope all possible stoichiometric and non-stoichiometric forms of the salts of the compounds of formula (I) or (II).

### Synthetic Schemes:

General synthetic methods are detailed below in Schemes 1-4. The present schemes are intended to be illustrative of the present invention and not limiting in any way. While particular substituents are disclosed, other variables can be made with the present schemes.

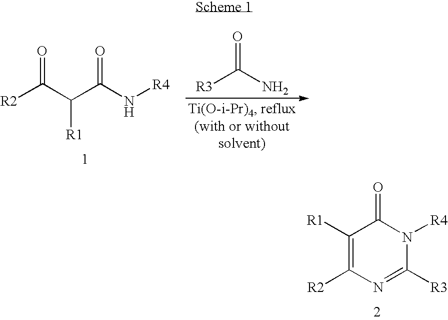

Treatment of a β-keto amide such as 1 with an amide in the presence of a Lewis acid such as Ti(O-i-Pr)₄ provides the pyrimidinone 2 as outlined in Scheme 1.

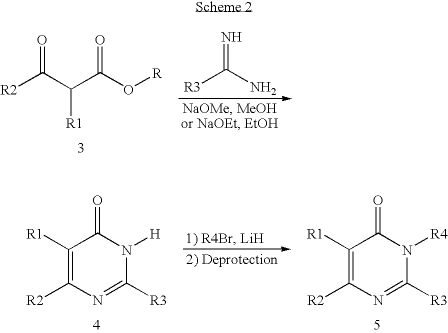

Alternatively, as outlined in Scheme 2, treatment of a β-keto ester such as 3 with an amidine in the presence of a base such as sodium methoxide or sodium ethoxide provides the pyrimidinone 4. Alkylation of 4 with and alkylating agent in the presence of a base common to the art such as lithium hydride provides the protected pyrimidinone which upon deprotection of the phenol protecting group, common to the art, may be achieved by a variety of methods also common to the art to provide the desired analogs 5.

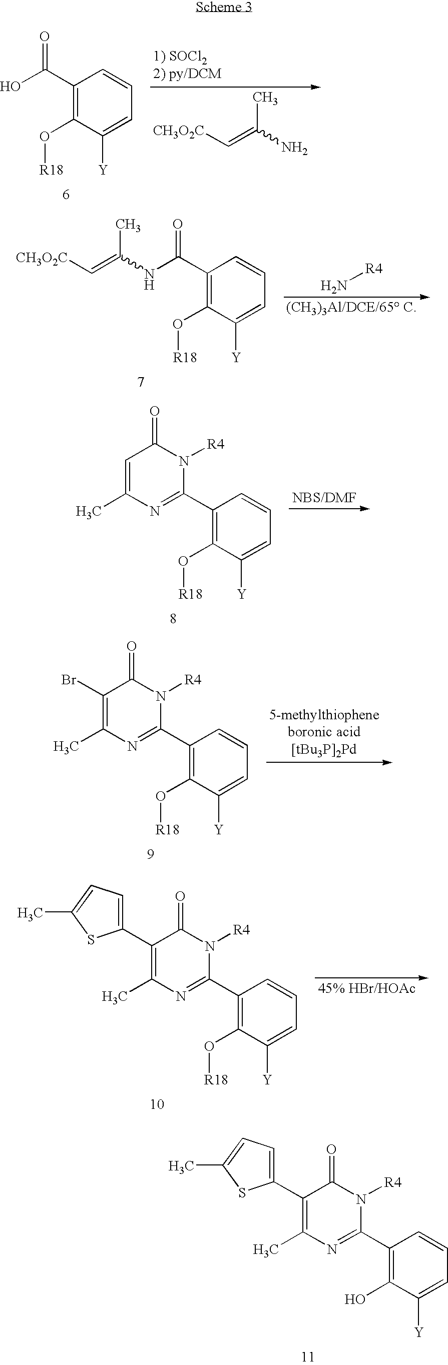

wherein:

R₁₈ represents a C₁₋₂alkyl, benzyl or acetyl protecting group in Scheme 3.

Y is a displacing group selected from F, Cl, Br and I.

R⁴ is selected from the group consisting of phenethyl, 3-fluorophenethyl, 4-fluorophenethyl, 2-fluorophenethyl, 2-thienyl ethyl, phenylethyl, dihydroindenyl, cyclohexylethyl, 3-chlorophenethyl, 3-trifluoromethylphenethyl, cyclopentyl ethyl and tetrahydropyranylethyl.

As described in Scheme 3, treatment of an appropriately protected 3-fluoro-2-alkoxybenzoic acid 6 with thionyl chloride provides corresponding acylchloride which with methyl 3-aminocrotonate in presence of a base common to the art such as pyridine provides mixtures of geometrical isomers such as methyl (2E,Z)-3-{[2-benzyloxy)-3-fluorobenzoyl]amino}but-2-enoate 7. Cyclization of 7 in presence of Me₃Al and amine provides the protected pyrimidinone 8. Selective bromination of 8 at the C-5 position under conditions common to the art such as NBS provides 9. Bromide 9 can then be coupled with 5-methyl-2-thiophene boronic acid under standard Suzuki reaction conditions to generate 10 which upon deprotection of the phenol protecting group using HBr in acetic acid, common to the art, provides the desired compound 11.

Scheme 3 discloses a novel method of converting an enamide according to 7 to a pyrimidinone according to 8. Novel intermediates include compounds 7, 8, 9 and 10 in Scheme 3.

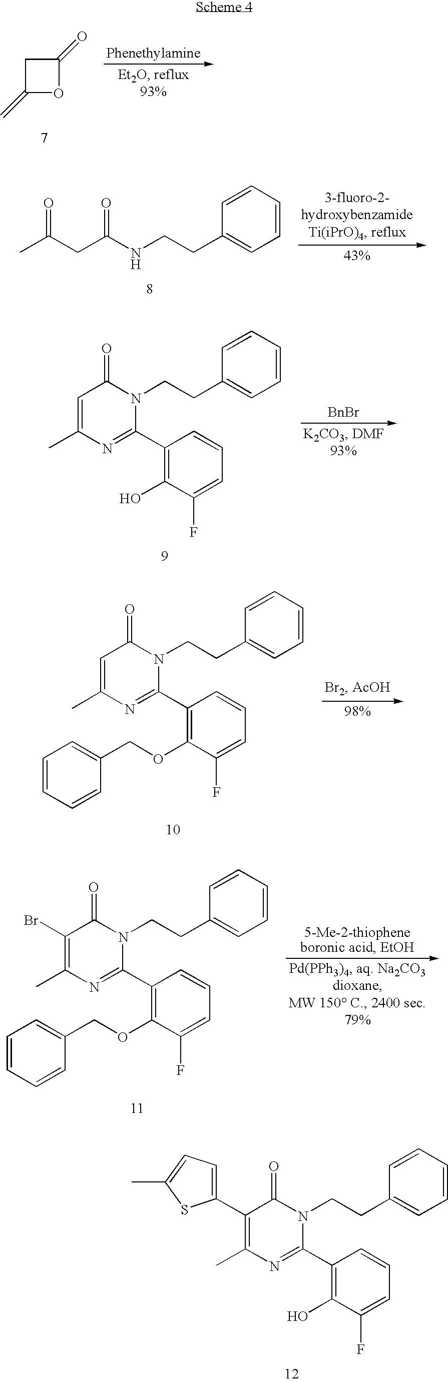

As shown in Scheme 4 \acute{α}-Substituted-β-keto-amides such as 14 can be prepared by a microwave-assisted thermal addition of an amine to a β-keto-ester such as 13. The enol triflate 15 is formed under conditions common to the art such as trifluoromethanesulfonic acid anhydride in the presence of a base such as triethyl amine. Subsequent treatment of the enol triflate 15 with a benzamide in the presence of a palladium catalyst and inorganic base such as cesium carbonate provides the enamide 16. Standard conditions common to the art are utilized to affect cyclization to the fully functionalized pyrimidinone 17.

In order to use a compound of Formula (I) of (II) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., for intramuscular, intravenous, intraperitoneal, and subcutaneous administration. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably, the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or (II) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I) or (II). A topical formulation contains suitably 0.01 to 5.0% of a compound of Formula (I) or (II). The active ingredient may be administered, for example, from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

Another aspect of the present invention describes a method of treating a patient comprising administering to the patient an amount of a present compound sufficient to increase the serum PTH level. Preferably, the method is carried out by administering an amount of the compound effective to cause an increase in duration and/or quantity of serum PTH level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In an alternative embodiment of the present invention, the compound administered to a patient causes an increase in serum PTH having a duration of more than about twenty-four hours provided that it is co-administered with an anti resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

In a preferred embodiment of the present invention, the present compound is co-administered with an anti-resorptive agent. Suitable anti-resorptive agents for co-administration include, but are not limited to estrogen, 1α,25-(OH)₂D₃, 1α-(OH)D₃, calcitonin, selective estrogen receptor modulators, vitronectin receptor antagonists, V-H+-ATPase inhibitors, src SH₂ antagonists, bisphosphonates and cathepsin K inhibitors.

Composition of Formula (I) or (II) and their pharmaceutically acceptable salts, which are active when given orally, can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or (II) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) and (II) are demonstrated by the following tests:

### (I) Calcium Receptor Inhibitor Assay

Calcilytic activity was measured by determining the IC₅₀ of the test compound for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 Suppl. 1:3483, 1995 (hereby incorporated by reference herein). Intracellular Ca²⁺ increases were elicited by increasing extracellular Ca²⁺ from 1 to 1.75 mM. Intracellular Ca²⁺ was measured using fluo-3, a fluorescent calcium indicator.

The procedure was as follows:

1. Cells were maintained in T-150 flasks in selection media (DMEM supplemented with 10% fetal bovine serum and 200 ug/mL hygromycin B), under 5% CO₂:95% air at 37° C. and were grown up to 90% confluency.

2. The medium was decanted and the cell monolayer was washed twice with phosphate-buffered saline (PBS) kept at 37° C. After the second wash, 6 mL of 0.02% EDTA in PBS was added and incubated for 4 minutes at 37° C. Following the incubation, cells were dispersed by gentle agitation.

3. Cells from 2 or 3 flasks were pooled and pelleted (100×g). The cellular pellet was resuspended in 10-15 mL of SPF-PCB+ and pelleted again by centrifugation. This washing was done twice.

Sulfate- and phosphate-free parathyroid cell buffer (SPF-PCB) contains 20 mM Na-Hepes, pH 7.4, 126 mM NaCl, 5 mM KCl, and 1 mM MgCl₂. SPF-PCB was made up and stored at 4° C. On the day of use, SPF-PCB was supplemented with 1 mg/mL of D-glucose and 1 mM CaCl₂ and then split into two fractions. To one fraction, bovine serum albumin (BSA; fraction V, ICN) was added at 5 mg/mL (SPF-PCB+). This buffer was used for washing, loading and maintaining the cells. The BSA-free fraction was used for diluting the cells in the cuvette for measurements of fluorescence.

4. The pellet was resuspended in 10 mL of SPF-PCB+ containing 2.2 uM fluo-3 (Molecular Probes) and incubated at room temperature for 35 minutes.

5. Following the incubation period, the cells were pelleted by centrifugation. The resulting pellet was washed with SPF-PCB+. After this washing, cells were resuspended in SPF-PCB+ at a density of 1-2×106 cells/mL.

6. For recording fluorescent signals, 300 uL of cell suspension were diluted in 1.2 mL of SPF buffer containing 1 mM CaCl₂ and 1 mg/mL of D-glucose. Measurements of fluorescence were performed at 37° C. with constant stirring using a spectrofluorimeter. Excitation and emission wavelengths were measured at 485 and 535 nm, respectively. To calibrate fluorescence signals, digitonin (5 mg/mL in ethanol) was added to obtain Fmax, and the apparent Fmin was determined by adding Tris-EGTA (2.5 M Tris-Base, 0.3 M EGTA). The concentration of intracellular calcium was calculated using the following equation:

Intracellular calcium=(F−Fₘᵢₙ/Fₘₐₓ)×K_{d}; where K_{d}=400 nM.

7. To determine the potential calcilytic activity of test compounds, cells were incubated with test compound (or vehicle as a control) for 90 seconds before increasing the concentration of extracellular Ca²⁺ from 1 to 2 mM. Calcilytic compounds were detected by their ability to block, in a concentration-dependent manner, increases in the concentration of intracellular Ca²⁺ elicited by extracellular Ca²⁺.

In general, those compounds having lower IC₅₀ values in the Calcium Receptor Inhibitor Assay are more preferred compounds. Compounds having an IC₅₀ greater than 30 uM were considered to be inactive. Preferred compounds are those having an IC₅₀ of 10 uM or lower. The present examples were tested except for Examples 11, 20, 28, 44 and 107. All compounds tested were found to be active, except for Examples 27, 46, 100, 123, 127, 214, 215 and 216 at the concentrations used.

### (II) Calcium Receptor Binding Assay

HEK 293 4.0-7 cells stably transfected with the Human Parathyroid Calcium Receptor (“HuPaR”) were scaled up in T180 tissue culture flasks. Plasma membrane is obtained by polytron homogenization or glass douncing in buffer (50 mM Tris-HCl pH 7.4, 1 mM EDTA, 3 mM MgCl₂) in the presence of a protease inhibitor cocktail containing 1 uM Leupeptin, 0.04 uM Pepstatin, and 1 mM PMSF. Aliquoted membrane was snap frozen and stored at −80° C. ³H labeled compound was radiolabeled to a radiospecific activity of 44 Ci/mmole and was aliquoted and stored in liquid nitrogen for radiochemical stability.

A typical reaction mixture contains 2 nM ³H compound ((R,R)—N-4′-Methoxy-t-3-3′methyl-1′-ethylphenyl-1-(1-naphthyl)ethylamine), or ³H compound (R)—N-[2-Hydroxy-3-(3-chloro-2-cyanophenoxy)propyl]-1,1-dimethyl-2-(4-methoxyphenyl)ethylamine 4-10 ug membrane in homogenization buffer containing 0.1% gelatin and 10% EtOH in a reaction volume of 0.5 mL. Incubation is performed in 12×75 polyethylene tubes in an ice water bath. To each tube 25 uL of test sample in 100% EtOH is added, followed by 400 uL of cold incubation buffer, and 25 uL of 40 nM ³H-compound in 100% EtOH for a final concentration of 2 nM. The binding reaction is initiated by the addition of 50 uL of 80-200 ug/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting. Preferred compounds are those having an IC₅₀ of 10 uM or lower. The present examples were tested except for Examples 11, 20, 28, 44 and 107. All compounds tested were found to be active.

### (III) Oral Administration in Dog and Rat

### Dog

The animals (male beagle dogs) were fed a diet of “Certified Canine Diet” #5007, approximately 300-500 grams per day. Water was provided ad libitum. During dosing days, animals were fasted (no morning feeding), and the animals were fed after the 240 minute blood collection time point.

For the first 2 hours of these studies the dogs were placed in restraint slings for dosing and blood collection purposes. They were returned to their cages after the 2 hour time point and individually restrained for all subsequent blood collection time points.

Experimental Procedures

A 4×4 Latin Square Design (4 treatments, 4 experimental days, 1 animals/treatment/day) was followed where treatments were randomly assigned before the first experiment. The entire experiment was completed on 4 separate days following the outline below.

On each of these study days, one animal received either vehicle or compound, so that by the conclusion of the study, all animals were exposed to all treatments.

4 × 4: Latin Square Design

Experiment

| Dog # | 1 | 2 | 3 | 4 |
|---|---|---|---|---|
| 17 | A | D | B | C |
| 22 | B | A | C | D |
| 23 | C | B | D | A |
| 28 | D | C | A | B |

Group Assignments

| Group # | Treatment | dose (mg/kg) |
|---|---|---|
| A | Compound 1 | 10 mg(6 mL)/kg |
| B | Compound 2 | 10 mg(6 mL)/kg |
| C | Compound 3 | 10 mg(6 mL)/kg |
| D | Vehicle | 0 mg/kg |

On each study day 3 dogs received either Compound 1, 2, or 3 at Xmg[6 ml]/kg, and 1 dog received vehicle at a dose volume of 6 mL/kg. Compounds were prepared in 20% Cavitron and 1% DMSO. The formulation was a suspension at all three doses. pH was measured and adjusted if necessary and documented following drug formulation.

All animals were dosed via oral gavage using a 24 french feeding tube attached to a three way stop cock. After introduction of the feeding tube into the stomach, approximately 10 mL of a 20 mL water flush was gavaged to ensure proper placement of the dosing tube. Dose was then administered at Xmg [6 ml]/kg. Following dose administration the remainder of water flush (approximately 10 mL) was gavaged to clear out the dosing tube.

Blood samples (approximately 3 mL) were obtained from either a cephalic or saphenous vein using 20 gauge catheter and injection cap or a 23 gauge needle and syringe. The catheter was locked with a heparin glucose lock (prepared by the LAS department) between samples. Blood samples were obtained just prior to dosing and at 5, 10, 15, 30, 60, 90, 120, 240 and 360 minutes post dose. The whole blood was placed in a sodium heparinized vacutainer tube and slightly vortexed to inhibit clotting and properly mix the sample. From each sample collected, a 100 μL aliquot was used to determine blood ionized calcium using the Medica Easylyte calcium analyzer. Additionally, a 25 μL blood sample was immediately transferred to an appropriately labelled tube. Nanopure water (25 μL) was added to this tube and then vortexed (this was done in duplicate). This sample was allowed to sit for approximately 0.5 min at room temperature to allow for blood cell lysis, and then placed on dry ice. Concentrations of compounds were quantified by HPLC/MS/MS by the DMPK MMPD CEDD department. An aliqout of whole blood (˜200 μL) and approximately 5 mg of compound was quantified. The remainder of whole blood was centrifuged and plasma separated for determination of PTH1-84.

### Rat

1. Experimental Procedures

Compounds were prepared weekly by wetting the appropriate amount of compound in 1% DMSO. Subsequently 40% Cavitron (total volume 20%) was added, followed by sterile water to bring the solution to the appropriate volume. Final pH for each compound and vehicle were measured and adjusted if necessary. Any adjustments to pH were made prior to addition of deionized water and documented. Animals were weighed and dosed.

Volumes were adjusted so each rat received 8 mL/Kg as an oral dose. Blood withdrawals (300 μL) occurred prior to dosing and at 2, 5, 15, 30, 45, 60, 120, 180 and 240 minutes after dosing. A 25 μL aliquot from the whole blood sample collected was vortexed with 25 μL of nanopure water and placed on dry ice for the evaluation of drug levels. This sample was allowed to sit for approximately 0.5 min at room temperature to allow for blood cell lysis, and then placed on dry ice. Concentrations of compound(s) were quantified by HPLC/MS/MS. An aliquot of whole blood (˜200 μL) and approximately 5 mg of compound were quantified. The remaining blood was centrifuged and plasma collected for PTH 1-84 analysis.

Blood was replaced at the end of each experiment (collected from a donor animal on the same day and slightly heparinized, approximately 5 IU/ml). The amount of blood replaced equaled the total amount taken from the animal during the study.

4 × 4 × 2: Latin Square Design

Experiment

| Rat # | 1 | 2 | 3 | 4 |
|---|---|---|---|---|
| 1-2 | A | D | B | C |
| 3-4 | B | A | C | D |
| 5-6 | C | B | D | A |
| 7-8 | D | C | A | B |

Group Assignments

| Group # | Treatment | dose (mg/kg) |
|---|---|---|
| A | Compound 1 | 3 mg/kg |
| B | Compound 1 | 10 mg/kg |
| C | Compound 1 | 30 mg/kg |
| D | Vehicle | 0 mg/kg |

* Only reported data from 3 mg/Kg dose

All compounds disclosed in Examples 249 through 253 and 255 through 269, according to formula (II) of the present invention, were tested and found to be active for the above assays. A compound is deemed active if there is significant PTH release elicited relative to the vehicle. In the rat test, a compound is deemed active at >50 pg/mL. In the dog test, a compound is deemed active at >15 pg/mL.

### EXAMPLES

Nuclear magnetic resonance spectra were recorded at either 300 or 400 MHz using, respectively, a Bruker ARX 300 or Bruker AVANCE 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (Δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Fourier transform infrared (FTIR) spectra were recorded on a Nicolet 510 infrared spectrometer. FTIR spectra were recorded in transmission mode, and band positions are reported in inverse wavenumbers (cm⁻¹). Mass spectra were taken on either a SCIEX5 or Micromass instruments, using electrospray (ES) ionization techniques. Elemental analyses were obtained using a Perkin-Elmer 240C elemental analyzer. Melting points were taken on a Thomas-Hoover melting point apparatus and are uncorrected All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5 μApex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) Celite® is a filter aid composed of acid-washed diatomaceous silica, and is a registered trademark of Manville Corps, Denver, Colo.

The following examples are intended to be illustrative only and not limiting in any way:

### Example 1

### Preparation of 2-(2-Fluoro-3-hydroxyphenyl)-6-methyl-5-(2-methylpropyl)-3-(2-phenylethyl)-4(3H)-pyrimidinone

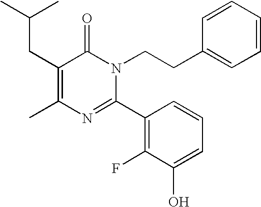

### a. Ethyl 2-acetyl-4-methyl-4-pentenoate

To a solution of ethyl 3-oxobutanoate (5.80 g, 0.05 mol) in dry acetonitrile was added 3-bromo-2-methyl-1-propene (6.75 g, 0.05 mmol) and K₂CO₃. The reaction mixture was stirred at RT for 62 h. The solid was filtered off and the filtrate was concentrated. The crude residue was purified by flash column chromatography using 10% EtOAc in hexanes to give 4.29 g of the desired product 52% yield.

### b. 2-Acetyl-4-methyl-N-(2-phenylethyl)-4-pentenamide

To a solution of ethyl 2-acetyl-4-methyl-4-pentenoate (0.25 g, 1.35 mmol) in DME (2.7 mL) was added phenethylamine (0.057 mL, 0.45 mmol) in a microwave reaction vessel. This mixture was irradiated to 180° C. for 800 s. The reaction mixture was diluted with EtOAc and washed with 1N HCl. The organic layer was separated and dried over Na₂SO₄, filtered, concentrated and purified by chromatography on silica gel (Biotage, 0-40% ethyl acetate/hexane) to afford pure amide (0.21 g) in 60% yield.

### c. 2-Acetyl-4-methyl-N-(2-phenylethyl)pentanamide

To a solution of 2-acetyl-4-methyl-N-(2-phenylethyl)-4-pentenamide (2.0 g, 7.69 mmol) in a solvent mixture of equal volumes of EtOH and EtOAc (100 mL) was added 10% Pd/C (0.1 g). This mixture was placed under Hydrogen atmosphere and stirred for 12 h. The reaction mixture was filtered through a bed of celite, and the concentrated filtrate was used in the next step without purification.

### d. 2-[2-Fluoro-3-(methyloxy)phenyl]-6-methyl-5-(2-methylpropyl)-3-(2-phenylethyl)-4(3H)-pyrimidinone

2-Acetyl-4-methyl-N-(2-phenylethyl)pentanamide (1.00 g, 3.82 mmol) was taken up in dry xylene (38 mL). To this was added 2-fluoro-3-methoxybenzamide (0.65 g, 3.82 mmol) and titanium isopropoxide (4.47 mL, 0.015 mol) sequentially. The reaction was heated to reflux until all the starting material was consumed. The reaction mixture was concentrated and diluted with dichloromethane and washed with 3N HCl. The organic layer was separated and dried over Na₂SO₄, filtered, concentrated and purified by chromatography on silica gel (Biotage, 0-40% ethyl acetate/hexane) to afford pure product (0.57 g) in 38% yield.

### e. 2-(2-Fluoro-3-hydroxyphenyl)-6-methyl-5-(2-methylpropyl)-3-(2-phenylethyl)-4(3H)-pyrimidinone

2-[2-fluoro-3-(methyloxy)phenyl]-6-methyl-5-(2-methylpropyl)-3-(2-phenylethyl)-4(3H)-pyrimidinone (0.10 g, 0.25 mmol) in 1.0 mL of dichloromethane was cooled to 0° C. BBr₃ was then added and the reaction mixture warmed to RT and stirred for 12 h. The reaction mixture was diluted with dichloromethane and aqueous NaHCO₃ was then added. The organic layer was separated and washed with H₂O, brine and dried over Na₂SO₄, filtered, concentrated and purified by chromatography on silica gel (Biotage, 0-60% ethyl acetate/hexane) to afford pure compound (0.043 g) in 45% yield. MS (m/z): 371.2 [M+H]⁺.

### Example 2

### Preparation of 2-(3-Hydroxyphenyl)-6-methyl-5-(2-methylpropyl)-3-(2-phenylethyl)-4(3H)-pyrimidinone

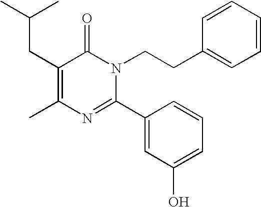

The title compound was prepared by substituting-3-methoxybenzamide for 2-methoxy-3-fluorobenzamide in Example 1d: MS (m/z): 363.4 [M+H]⁺.

### Example 3

### Preparation of 2-(2,3-Dihydroxyphenyl)-6-methyl-5-(2-methylpropyl)-3-(2-phenylethyl)-4(3H)-pyrimidinone

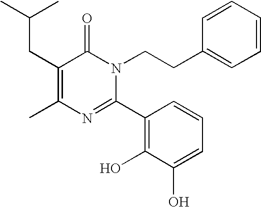

The title compound was prepared by substituting 2,3-dimethoxybenzamide for 2-methoxy-3-fluorobenzamide in Example 1d: MS (m/z): 379.2 [M+H]⁺.

### Example 4

### Preparation of 6-Methyl-5-(2-methylpropyl)-3-(2-phenylethyl)-2-(1H-pyrrol-2-yl)-4(3H)-pyrimidinone

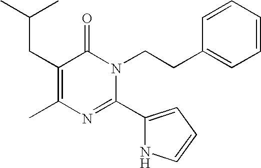

2-Acetyl-4-methyl-N-(2-phenylethyl) pentanamide of Example 1c was taken up in titanium isopropoxide (3.96 mmol, 11.74 mL). To this was added 1H-pyrrole-2-carboxamide (0.5 g, 4.58 mmol) and the reaction was heated to reflux for 48 h. Upon completion, the reaction was diluted with dichloromethane and washed with 3N HCl. The organic layer was separated and dried over Na₂SO₄, filtered, concentrated and purified by chromatography on silica gel (Biotage, 0-50% ethyl acetate/hexane) to afford the title compound (0.42 g) in 42% yield. MS (m/z): 336.2 [M+H]⁺.

### Example 5

### Preparation of 6-Methyl-5-(2-methylpropyl)-3-(2-phenylethyl)-2-(2-thienyl)-4(3H)-pyrimidinone

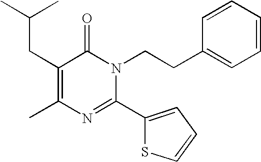

### a. 6-Methyl-5-(2-methyl-2-propen-1-yl)-3-(2-phenylethyl)-2-(2-thienyl)-4(3H)-pyrimidinone

2-Acetyl-4-methyl-N-(2-phenylethyl) pentenamide of Example 1b (0.52 g, 1.98 mmol) was taken up in titanium isopropoxide (2.57 mmol, 7.56 mL). To this was added thiophene-2-carboxamide (0.38 g, 2.97 mmol) and the reaction was heated to reflux for 48 h. Upon completion, the reaction was diluted with dichloromethane and washed with 3N HCl. The organic layer was separated and dried over Na₂SO₄. The crude compound was taken into the next step without purification.

### b. 6-Methyl-5-(2-methylpropyl)-3-(2-phenylethyl)-2-(2-thienyl)-4(3H)-pyrimidinone

6-Methyl-5-(2-methyl-2-propen-1-yl)-3-(2-phenylethyl)-2-(2-thienyl)-4(3H)-pyrimidinone was taken up in ethanol (4.0 mL). To this was added 0.1 g of 10% Pd/C and placed under Hydrogen atmosphere for 16 h. The reaction mixture was filtered through a bed of celite, and the concentrated filtrate was purified by reverse phase HPLC to afford (0.17 g) of the final compound in 25% yield. MS (m/z): 453.2 [M+H]⁺.

### Example 6

### Preparation of 6-Methyl-5-(2-methylpropyl)-3-(2-phenylethyl)-2-(2-pyridinyl)-4(3H)-pyrimidinone

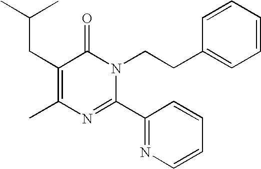

The title compound was prepared by substituting 2-pyridinecarboxamide for thiophene-2-carboxamide of Example 5a: MS (m/z): 348.2 [M+H]⁺.

### Example 7

### Preparation of 2-(2-Furanyl)-6-methyl-5-(2-methylpropyl)-3-(2-phenylethyl)-4(3H)-pyrimidinone

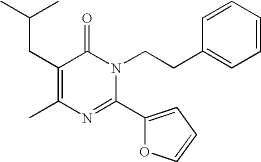

The title compound was prepared by substituting furan-2-carboxamide for thiophene-2-carboxamide of Example 5a: MS (m/z): 337.2 [M+H]⁺.

### Example 8

### Preparation of 2-(1H-imidazol-2-yl)-6-methyl-5-(2-methylpropyl)-3-(2-phenylethyl)-4(3H)-pyrimidinone

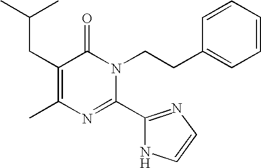

2-Acetyl-4-methyl-N-(2-phenylethyl)-4-pentanamide of Example 1c (0.26 g, 0.99 mmol) was taken up in titanium isopropoxide (12.89 mmol, 3.8 mL). To this was added 1H-imidazole-2-carboxamide (0.17 g, 1.49 mmol) and the mixture was heated to reflux for 48 h. The reaction mixture was concentrated and diluted with dichloromethane and washed with 3N HCl. The organic layer was separated and dried over Na₂SO₄, filtered, concentrated and purified by reverse phase HPLC to afford the pure title compound in 15% yield (0.051 g). MS (m/z): 337.2 [M+H]⁺.

### Example 9

### Preparation of 5-Ethyl-2-(2-fluoro-3-hydroxyphenyl)-3-[2-(3-fluorophenyl)ethyl]-6-methyl-4(3H)-pyrimidinone

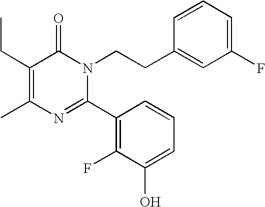

### a. Ethyl 2-(2-methyl-1,3-dioxolan-2-yl)butanoate

A mixture of commercially available 2-ethyl-3-oxo-butyric acid ethyl ester (54 g, 0.34 mol), ethylene glycol (23.3 g, 0.375 mol), and p-toluenesulfonic acid (0.2 g) in toluene (500 mL) was heated to 120° C. for 4 h under a Dean-Stark apparatus. The reaction mixture was cooled to RT, the solvent was removed, and the residue was partitioned between ethyl acetate and saturated NaHCO₃. The layers were separated, and the aqueous portion was extracted 3 times with ethyl acetate. The organic portions were pooled, dried (MgSO₄) and concentrated to give the cyclic ketal product as a colorless oil in 91% yield (63 g).

### b. 2-(2-Methyl-1,3-dioxolan-2-yl)butanoic acid

To a solution of ethyl 2-(2-methyl-1,3-dioxolan-2-yl)butanoate (60 g, 0.297 mol) provided above in EtOH (750 mL) was added 85% KOH solution in water (30 mL), and the mixture stirred at reflux overnight. The reaction mixture was cooled to RT, the solvent evaporated, and the residue was partitioned between CH₂Cl₂ and 2N HCl. After separating the layers, the aqueous portion was extracted 3 times with CH₂Cl₂. The organic portions were pooled, dried (Na₂SO₄), and concentrated to give the acid product as a light yellow oil (27 g, 52% yield).

### c. N-[2-(3-Fluorophenyl)ethyl]-2-(2-methyl-1,3-dioxolan-2-yl)butanamide

To a cold (0° C.) solution of 2-(2-Methyl-1,3-dioxolan-2-yl)butanoic acid (32.89 g, 0.19 mol) in CH₂Cl₂ (30 mL) was added oxalyl chloride (60.0 mL) in a dropwise fashion. After 15 min at 0° C., the mixture was allowed to stir at RT for 2 h. The solvent and excess oxalylchloride were removed to give an oil, which was brought up in fresh CH₂Cl₂ and cooled to 0° C. A pyridine solution (20 mL) of [2-(3-fluorophenyl)ethyl]amine (44 mL, 0.34 mol) was added dropwise, and the resulting solution was allowed to warm to RT while stirring overnight. The reaction mixture was partitioned between CH₂Cl₂ and 1N HCl. After separating the layers, the organic portion was washed with water and aq. NaHCO₃. The organic portion was pooled, dried (Na₂SO₄), and concentrated in vacuo to give the crude amide product (35.0 g) which was used in the next reaction without further purification.

### d. 2-Ethyl-N-[2-(3-fluorophenyl)ethyl]-3-oxobutanamide

To a solution of N-[2-(3-fluorophenyl)ethyl]-2-(2-methyl-1,3-dioxolan-2-yl)butanamide (35.0 g, 0.12 mol) in acetone and water (250 mL n5 mL) was added p-toluenesulfonic acid (36.1 g, 0.19 mol). This mixture was stirred and heated to 95° C. for 4 h. After cooling to RT, the solvent was removed and the residue was partitioned between CH₂Cl₂ and aq. Na₂CO₃. After separating the layers, the aqueous layer was extracted 2 times with fresh CH₂Cl₂, and the combined organic portions were dried (NaSO₄), filtered and concentrated to provide a white solid. The solid was triturated with 1:1 hexanes/diethyl ether to give 24.5 g (85%).

### e. 5-Ethyl-2-(2-fluoro-3-hydroxyphenyl)-3-[2-(3-fluorophenyl)ethyl]-6-methyl-4(3H)-pyrimidinone

The title compound was prepared by the general procedure outlined in Example 1 and substituting 2-ethyl-N-[2-(3-fluorophenyl)ethyl]-3-oxobutanamide for 2-acetyl-4-methyl-N-(2-phenylethyl)pentanamide in step 1d: MS (m/z): 371.2 [M+H]⁺.

### Example 10

### Preparation of 5-Ethyl-3-[2-(3-fluorophenyl)ethyl]-6-methyl-2-(1H-pyrrol-2-yl)-4(3H)-pyrimidinone

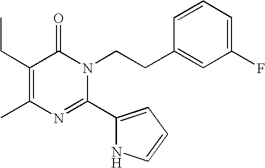

Ethyl-N-[2-(3-fluorophenyl)ethyl]-3-oxobutanamide (0.5 g, 2.14 mmol) of Example 9d was taken up in titanium isopropoxide (2.78 mmol, 8.5 mL). To this was added 1H-pyrrole-2-carboxamide (0.35 g, 3.21 mmol), and reaction was heated to reflux for 48 h. Upon completion, the reaction was diluted with dichloromethane and washed with 3N HCl. The organic layer was separated and dried over Na₂SO₄, filtered, concentrated and purified by chromatography on silica gel (Biotage, 0-50% ethyl acetate/hexane) to afford the title compound (0.42 g) in 32% yield. MS (m/z): 326.2 [M+H]⁺.

### Example 11

### Preparation of 5-Bromo-2-{3-fluoro-2-[(phenylmethyl)oxy]phenyl}-6-methyl-3-(2-phenylethyl)-4(3H)-pyrimidinone

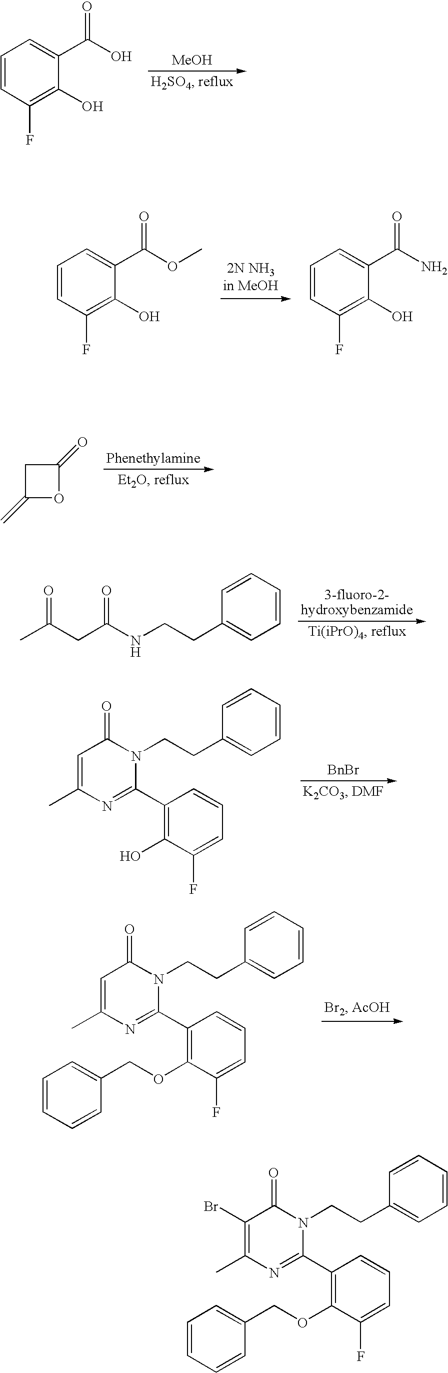

### a. Methyl 3-fluoro-2-hydroxybenzoate

The hydroxy acid (10 g, 0.064 moles) was taken up in anhydrous methanol (215 mL). To this was added catalytic amount of sulfuric acid and the reaction was reflux for 16 h. The reaction was concentrated and the crude product was taken into the next step without purification.

### b. 3-Fluoro-2-hydroxybenzamide

The methyl ester was placed in pressure reaction vessel. To this was added 2N ammonia in methanol (125 mL) and the reaction was heated to 110° C. for 16 h. The reaction was concentrated and taken up in dichloromethane. The undissolved material is filtered off. The reaction is concentrated and dissolved in large amount of methanol and was decolorized. The methanol solution was partly concentrated upon which crystalline solid (pale brown) was crashed out. The solid is filtered and used in the next step.

### c. 3-Oxo-N-(2-phenylethyl)butanamide

Diketene (10.0 g, 0.12 moles) was taken up in anhydrous ether (240 mL). To this was added phenethylamine (14.93 mL, 0.12 moles) dropwise by a dropping funnel. Upon addition of amine was complete the reaction was heated to reflux for 3 h. The crude mixture was taken in separatory funnel and washed with 5% HCl and organic layer was separated, dried over sodium sulfate and concentrated to give 22.78 grams in 93% yield.

### d. 2-(3-Fluoro-2-hydroxyphenyl)-6-methyl-3-(2-phenylethyl)-4(3H)-pyrimidinone

The 3-oxo-N-(2-phenylethyl)butanamide (10 g, 0.049 moles) was placed in 500 mL round bottom flask. To this was added titanium isopropoxide (214 mL, 0.73 moles). While the reaction is stirring 3-fluoro-2-hydroxybenzamide (11.42 g, 0.098 moles) was added, a condenser was placed and the reaction was heated to reflux (oil bath temperature=150° C.). The 2-hydroxy-3-fluorobenzamide dissolved slowly and gave brown homogenous solution upon some time at elevated temperatures. Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude reaction mixture was taken in EtOAc and hexanes were added to crash out the product. The solid (6.79 g, 43%) was filtered and taken into the next step without purification.

### e. 2-{3-Fluoro-2-[(phenylmethyl)oxy]phenyl}-6-methyl-3-(2-phenylethyl)-4(3H)-pyrimidinone

2-(3-fluoro-2-hydroxyphenyl)-6-methyl-3-(2-phenylethyl)-4(3H)-pyrimidinone (6.0 g, 0.019 moles) was dissolved in dry DMF (92 mL). To this was added potassium carbonate (3.83 g, 0.028 moles) and benzyl bromide (2.64 mL, 0.028 moles) sequentially. Reaction was warmed to 60° C. and stirred for 16 h. Reaction mixture was cooled to ambient temperature, filtered and diluted with EtOAc. This was washed successively with 5% HCl and saturated sodium chloride solution. Organic layer was dried over sodium sulfate and concentrated to give the product (7.12 g) in 93% yield.

### f. 5-Bromo-2-{3-fluoro-2-[(phenylmethyl)oxy]phenyl}-6-methyl-3-(2-phenylethyl)-4(3H)-pyrimidinone

2-{3-fluoro-2-[(phenylmethyl)oxy]phenyl}-6-methyl-3-(2-phenylethyl)-4(3H)-pyrimidinone (6.0 g, 0.0145 moles) was taken up in glacial acetic acid (100 mL). To this was added bromine (0.74 mL, 0.0145 moles) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was purified by chromatography on silica gel (Biotage) using ethylacetate and hexane mixtures (10-50%) to obtain the desired product (7.06 g) in 98% yield. MS (m/z): 495.2 [M+H]⁺.

### Example 12

### Preparation of 5-Bromo-2-(3-fluoro-2-hydroxyphenyl)-6-methyl-3-(2-phenylethyl)-4(3H)-pyrimidinone

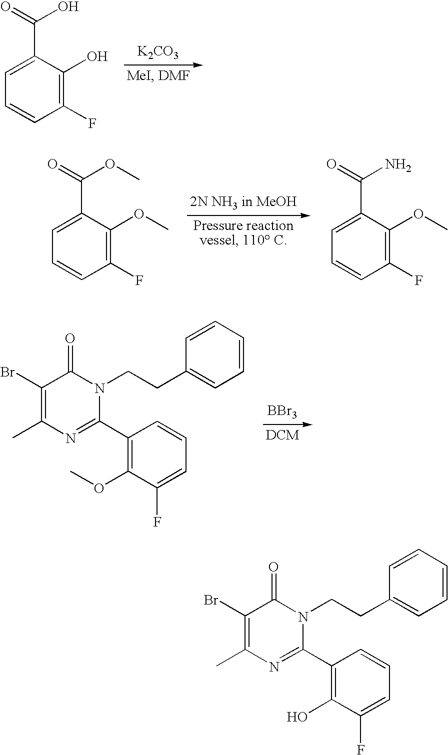

### a. 3-Fluoro-2-(methyloxy)benzamide

To a solution of 2-hydroxy-3-fluorobenzoic acid (30.0 g, 0.19 mol) in dry DMF (320 mL) was added K₂CO₃ (66.4 g, 0.48 mol) and iodomethane (30.0 mL, 0.48 mol) sequentially. Reaction was warmed to 60° C. and stirred for 16 h. Upon cooling, the reaction mixture was diluted with EtOAc and washed with 1N HCl, 5% NaHCO₃ and brine. The organic layer was dried over Na₂SO₄, filtered and concentrated. The resulted methyl ester was placed in a pressure vessel. To this was added 2N NH₃ in methanol and the reaction was heated to 110° C. for 16 h. Upon cooling, the reaction mixture was filtered and concentrated to give the desired amide (26.3 g) in 81% overall yield.

### b. 3-Oxo-2-phenyl-N-[2-(2-thienyl)ethyl]butanamide

To a solution of ethyl 3-oxo-2-phenylbutanoate (5.0 g, 0.024 moles) in DME (21 mL) was added [2-(2-thienyl)ethyl]amine (0.057 mL, 0.45 mmoles) in a microwave reaction vessel. Few drops of ethanol was added to the reaction mixture was irradiated to 180° C. for 800 s. The reaction mixture was diluted with EtOAc and washed with 1N HCl. Organic layer was separated and dried over Na₂SO₄. Filtered, concentrated and purified by chromatography on silica gel (Biotage, 0-40% ethyl acetate/hexane) to afford pure amide (3.42 g) in 49% yield.

### c. 5-Bromo-2-[3-fluoro-2-(methyloxy)phenyl]-6-methyl-3-(2-phenylethyl)-4(3H)-pyrimidinone

The title compound was prepared by the general procedure outlined in Example 1 by substituting 3-fluoro-2-methoxybenzamide for 3-fluoro-2-hydroxybenzamide in step 1d.

### d. 5-Bromo-2-(3-fluoro-2-hydroxyphenyl)-6-methyl-3-(2-phenylethyl)-4(3H)-pyrimidinone

Bromo-2-[3-fluoro-2-(methyloxy)phenyl]-6-methyl-3-(2-phenylethyl)-4(3H)-pyrimidinone (0.065 g, 0.16 mmol) in 2 mL of dichloromethane was cooled to 0° C. 1M DCM solution of BBr₃ (0.8 mL, 0.78 mmol) was then added and the reaction mixture warmed to RT and stirred for 16 h. The reaction mixture was diluted with dichloromethane and aqueous NaHCO₃ was then added. The organic layer was separated and washed with H₂O, brine and dried over Na₂SO₄, filtered, concentrated and purified by chromatography on silica gel (Biotage, 0-60% ethyl acetate/hexane) to afford pure compound (0.022 g) in 35% yield. MS (m/z): 405.0 [M+2H]⁺.

### Example 13

### Preparation of 2-(3-Fluoro-2-hydroxyphenyl)-6-methyl-3-(2-phenylethyl)-5-(6-quinolinyl)-4(3H)-pyrimidinone

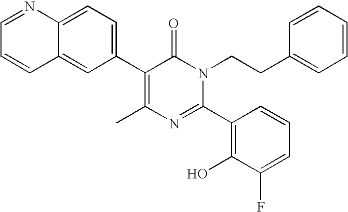

To a solution of 5-bromo-2-{3-fluoro-2-[(phenylmethyl)oxy]phenyl}-6-methyl-3-(2-phenylethyl)-4(3H)-pyrimidinone (0.20 g, 0.4 mmoles) in dioxane (3 mL) was added 6-quinolinylboronic acid (0.14 g, 0.8 mmoles) dissolved in solvent mixture of 0.5 mL ethanol and 0.5 mL of dioxane, and 0.5 mL of aqueous sodium carbonate (0.09 g, 0.8 mmoles) in a microwave reaction vessel and irradiated to 150° C. for 2400 seconds. The reaction mixture was filtered through syringe filter (Acrodisc CR25 mm with 0.20 □m PTFE membrane). The filtrate was diluted with EtOAc and washed with brine, separated, dried over sodium sulfate, filtered, concentrated in vacuo and the residue was purified by chromatography on silica gel (Biotage, 0-60% ethyl acetate/hexane) to afford the desired product (0.12 g) in 66% yield. MS (m/z): 452.4 [M+H]⁺.

### Example 14

### Preparation of 2-(3-Fluoro-2-hydroxyphenyl)-6-meth-yl-3-(2-phenylethyl)-5-(1,2,3,4-tetrahydro-6-quinolinyl)-4(3H)-pyrimidinone

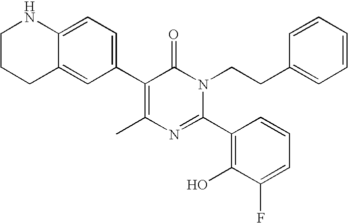

To a solution of 2-(3-Fluoro-2-hydroxyphenyl)-6-methyl-3-(2-phenylethyl)-5-(6-quinolinyl)-4(3H)-pyrimidinone of Example 13 was (0.33 g, 0.073 mmoles) in ethanol was added 10% Pd/C (0.01 g). This mixture was placed under hydrogen atmosphere and stirred for 12 h. The reaction mixture was filtered through a bed of celite and concentrated and purified by chromatography on silica gel (Biotage, 0-60% ethyl acetate/hexane) to afford the desired product (0.020 g) in 60% yield. MS (m/z): 456.2 [M+H]⁺.

### Example 15

### Preparation of 2-(3-Fluoro-2-hydroxyphenyl)-6-methyl-5-(1-methyl-1,2,3,4-tetrahydro-6-quinolinyl)-3-(2-phenylethyl)-4(3H)-pyrimidinone

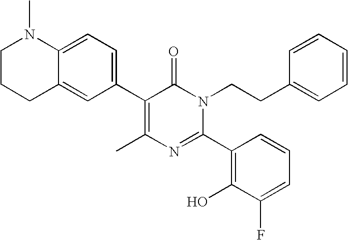

To a solution of Example 14 (0.02 g, 0.044 mol) in methanol was added formaldehyde (0.018 mL, 0.66 mmol) and NaCNBH₃ (8.15 mg, 0.13 mmole) sequentially. Reaction was stirred for 48 h at ambient temperature. The reaction mixture was concentrated and diluted with dichloromethane and washed with water and brine. The organic layer was separated dried over sodium sulphate, filtered and concentrated. The residue was purified by reverse phase HPLC to afford the desired product (7 mg) in 34% yield. MS (m/z): 470.2 [M+H]⁺.

### Example 16

### Preparation of 2-(3-Fluoro-2-hydroxyphenyl)-5-(2-furanyl)-6-methyl-3-(2-phenylethyl)-4(3H)-pyrimidinone

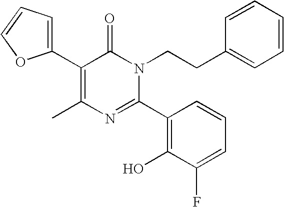

### a. 2-(3-Fluoro-2-hydroxyphenyl)-6-methyl-3-(2-phenylethyl)-5-(2-thienyl)-4(3H)-pyrimidinone

To a solution containing 5-bromo-2-{3-fluoro-2-[(phenylmethyl)oxy]phenyl}-6-methyl-3-(2-phenylethyl)-4(3H)-pyrimidinone (1.0 g, 2.02 mol) of Example 11 in deoxygenated dioxane was added Pd(tBu₃P)₂ (0.10 g, 0.20 mol), cesium fluoride (0.67 g, 4.5 mol) and tributyl(2-furanyl)stannane (0.6 mL, 2.22 mot) was added sequentially. The reaction was heated to 90° C. for 16 h and concentrated. The crude residue is diluted with dichloromethane and washed with saturated aqueous potassium fluoride, water and brine. The organic layer was separated, dried over Na₂SO₄, filtered and concentrated. The crude material was purified by chromatography on silica gel (Biotage, 0-50% ethyl acetate/hexane) to afford the desired product (0.81 g) in 81% yield.

### b. 2-(3-Fluoro-2-hydroxyphenyl)-6-methyl-3-(2-phenylethyl)-5-(2-thienyl)-4(3H)-pyrimidinone

2-(3-fluoro-2-hydroxyphenyl)-6-methyl-3-(2-phenylethyl)-5-(2-thienyl)-4(3H)-pyrimidinone (0.81 g, 1.63 mol) was placed in a round bottom flask equipped with a stir bar and a condenser. To this was added HBr in acetic acid (10 mL), water (1.0 mL) and stirred for 5 h. The reaction was quenched with saturated NaHCO₃ and extracted with dichloromethane. The combined organic layers were dried over Na₂SO₄, filtered and concentrated. The crude residue was purified by chromatography on silica gel (Biotage, 0-50% ethyl acetate/hexane) to afford the desired product (0.61 g) in 91% yield. MS (m/z): 391.2 [M+H]⁺.

### Example 17

### Preparation of 2-(3-Fluoro-2-hydroxyphenyl)-6-methyl-5-phenyl-3-[2-(2-thienyl)ethyl]-4(3H)-pyrimidinone

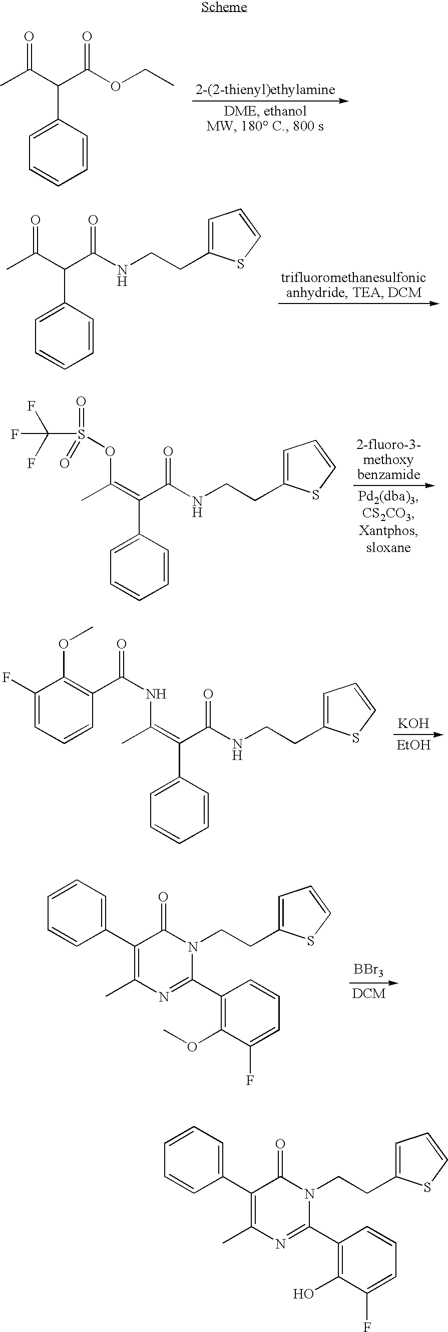

### a. 3-Oxo-2-phenyl-N-[2-(2-thienyl)ethyl]butan amide

To a solution of ethyl 3-oxo-2-phenylbutanoate (5 g, 0.24 moles) in DME (21 mL) was added 2-(2-thienyl)ethylamine (2.92 g, 0.023 mol) in a microwave reaction vessel. Few drops of ethanol was added to the reaction mixture and irradiated to 180° C. for 1200 s. The reaction mixture was diluted with EtOAc and washed with 1N HCl. Organic layer was separated and dried over Na₂SO₄. Filtered, concentrated and purified by chromatography on silica gel to afford pure amide (3.42 g) in 49% yield.

### b. (1Z)-1-Methyl-3-oxo-2-phenyl-3-{[2-(2-thienyl)ethyl]amino}-1-propen-1-yl trifluoromethanesulfonate

To a solution of 3-oxo-2-phenyl-N-[2-(2-thienyl)ethyl]butanamide (3.42 g, 0.012 mol) in dry dichloromethane (50 mL) was cooled to −78° C. To this was added trifluoromethanesulfonic anhydride (2.2 mL, 0.013 mol) and triethyl amine (2.49 mL, 0.018 mol) sequentially and stirred while reaction warmed to 0° C. The reaction was concentrated and purified by chromatography on silica gel (Biotage, 0-40% ethyl acetate/hexane) to afford trfilate (3.55 g) in 71% yield.

### c. 3-Fluoro-N-((1Z)-1-methyl-3-oxo-2-phenyl-3-{[2-(2-thienyl)ethyl]amino}-1-propen-1-yl)-2-(methyloxy)benzamide

To a solution of (1Z)-1-methyl-3-oxo-2-phenyl-3-{[2-(2-thienyl)ethyl]amino}-1-propen-1-yl trifluoromethanesulfonate in dry deoxygenated dioxane was added 3-fluoro-2-methoxybenzamide (0.48 g, 2.82 mmol), cesium carbonate (1.19 g, 3.67 mol), Pd₂(dba)₃ (0.06 g, 0.065 mmol) and xantophos (0.113 g, 0.2 mmol). The reaction was heated to reflux for 16 h. The cooled reaction mixture was filtered through a bed of celite and concentrated. Purification was purified by chromatography on silica gel (Biotage) to provide enamide in 62% yield.

### d. 2-[3-Fluoro-2-(methyloxy)phenyl]-6-methyl-5-phenyl-3-[2-(2-thienyl)ethyl]-4(3H)-pyrimidinone

3-fluoro-N-((1Z)-1-methyl-3-oxo-2-phenyl-3-{[2-(2-thienyl)ethyl]amino}-1-propen-1-yl)-2-(methyloxy)benzamide (0.74 g, 1.69 mol) was dissolved in ethanol (10 mL). To this was added 10 mL of 25% (w/v) aqueous potassium hydroxide and refluxed for 16 h. The crude reaction mixture was acidified by 6N HCl to pH ˜1 and extracted with dichloromethane. The combined organic layers were washed with brine and concentrated. The crude residue was purified by chromatography on silica gel (Biotage) to give the desired product (0.33 g) in 46% yield.

### e. 2-(3-Fluoro-2-hydroxyphenyl)-6-methyl-5-phenyl-3-[2-(2-thienyl)ethyl]-4(3H)-pyrimidinone

2-[3-fluoro-2-(methyloxy)phenyl]-6-methyl-5-phenyl-3-[2-(2-thienyl)ethyl]-4(3H)-pyrimidinone (0.33 g, 0.81 mmol) in 3 mL of dichloromethane was cooled to 0° C. BBr₃ in dichloromethane (1.62 mL) was then added and let the reaction mixture warmed to RT. Upon completion the reaction mixture was diluted with dichloromethane and aq. NaHCO₃ was then added. Organic layer was separated and washed with H₂O, brine and dried over Na₂SO₄. After filtration the reaction mixture was concentrated and purified by chromatography on silica gel (Biotage, 0-60% ethyl acetate/hexane) to afford pure compound (0.186 g) in 46% yield. MS (m/z): 407.2 [M+H]⁺.

### Example 18

### Preparation of 2-(3-Fluoro-2-hydroxyphenyl)-6-methyl-3-(2-phenylethyl)-5-(1-pyrrolidinyl)-4(3H)-pyrimidinone

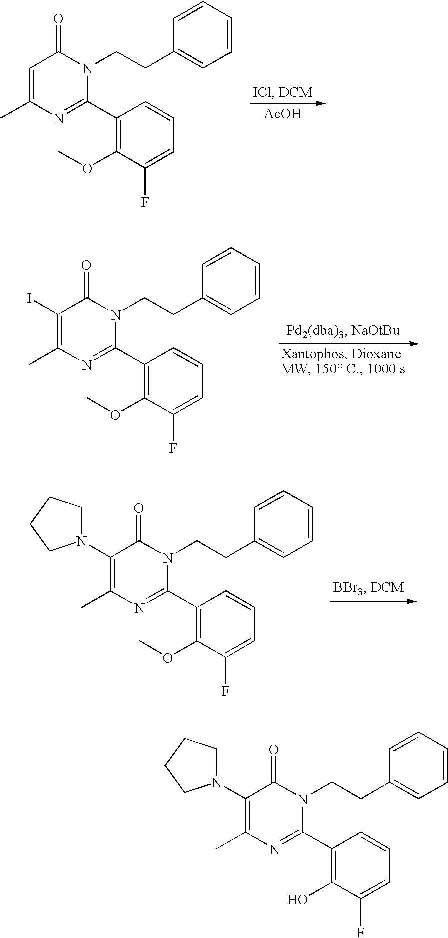

### a. 2-[3-fluoro-2-(methyloxy)phenyl]-5-iodo-6-methyl-3-(2-phenylethyl)-4(3H-pyrimidinone

2-[3-fluoro-2-(methyloxy)phenyl]-6-methyl-3-(2-phenylethyl)-4(3H)-pyrimidinone (4.78 g, 0.014 moles) from Example 12c was taken up in glacial acetic acid (283 mL). To this was added 1M dichloromethane solution of iodine monochloride (71 mL, 0.071 mmoles) and the reaction was stirred for 16 h. Ethyl acetate was added and acetic acid was washed with saturated sodium carbonate. The organic layer dried over sodium sulfate. Sodium sulfate was filtered off and organic layer was concentrated. The crude product was purified by chromatography on silica gel (Biotage) using ethylacetate and hexane mixtures (20-50%) to obtain the desired product (2.1 g) in 32% yield.

### b. 2-(3-Fluoro-2-hydroxyphenyl)-6-methyl-3-(2-phenylethyl)-5-(1-pyrrolidinyl)-4(3H)-pyrimidinone

To a solution of 2-[3-fluoro-2-(methyloxy)phenyl]-5-iodo-6-methyl-3-(2-phenylethyl)-4(3H)-pyrimidinone (0.3 g, 0.65 moles) in deoxygenated toluene (3.2 mL) was added xantophos (0.06 g, 0.096 mmol), Pd₂(dba)₃ (0.59 g, 0.032 mmol) and NaOtBu (0.09 g, 0.91 mmols) in a microwave vessel. The reaction stirred for 5 min and pyrrolidine (0.064 mL, 0.08 mmol) was added, reaction vessel was capped and irradiated in Smith Synthesizer at 150° C. for 1000 s. The reaction mixture was concentrated and purified by chromatography on silica gel (Biotage) using EtOAc and hexane mixtures (5-30%) to obtain the desired product (0.32 g) which contained small amount of impurity. Subsequent deprotection was accomplished using BBr₃ as described in Example 1e produced the title compound: MS (m/z): 394.4 [M+H]⁺.

### Example 19

### Preparation of 5-(5-Chloro-2-thienyl)-2-(3-fluoro-2-hydroxyphenyl)-6-methyl-3-(2-phenylethyl)-4(3H)-pyrimidinone

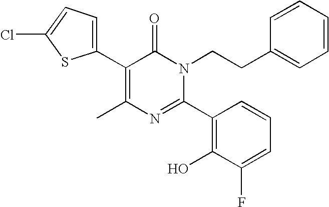

### a. 5-(5-Chloro-2-thienyl)-2-{3-fluoro-2-[(phenylmethyl)oxy]phenyl}-6-methyl-3-(2-phenylethyl)-4(3H)-pyrimidinone

To a solution of 5-bromo-2-[3-fluoro-2-[(phenylmethyl)oxy]phenyl]-6-methyl-3-(2-phenylethyl)-4(3H)-pyrimidinone (0.5 g, 1.01 mmoles) 11 in deoxygenated dioxane was added 2-chloro-5-bromothiophene (0.2 g, 1.01 mmoles), tetrakistriphenylphosphine (0.18 g, 0.1 mmoles) and hexamethylditin (0.21 mL, 1.01 mmoles). The reaction was refluxed for 48 h and concentrated. The crude residue was purified by chromatography on silica gel (Biotage) using ethylacetate and hexane mixtures (0-60%) to obtain the desired product (0.031 g) in 6% yield.

### b. 5-(5-Chloro-2-thienyl)-2-(3-fluoro-2-hydroxyphenyl)-6-methyl-3-(2-phenylethyl)-4(3H)-pyrimidinone

Into a round bottom flask equipped with a stirring bar and a condenser was placed 5-(5-chloro-2-thienyl)-2-{3-fluoro-2-[(phenylmethyl)oxy]phenyl}-6-methyl-3-(2-phenylethyl)-4(3H)-pyrimidinone (0.031 g, 0.06 mmoles). To this was added 2 mL of 45% HBr in acetic acid and the reaction was stirred at RT for 3 h. The crude residue was diluted with dichloromethane and extracted with saturated sodium carbonate and brine. The organic layer was concentrated and purified by reverse phase HPLC to give pure product (11 mg) in 42% yield: MS (m/z): 441.2 [M+H]⁺.

### Example 20

### Preparation of 5-bromo-6-methyl-3-(2-phenylethyl)-2-{2-[(phenylmethyl)oxy]phenyl}-4(3H)-pyrimidinone

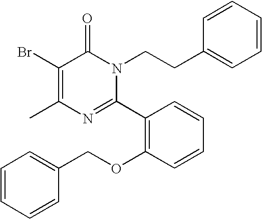

The title compound was prepared following the methods described for Example 11 except substituting 2-hydroxybenzamide for 3-fluoro-2-hydroxybenzamide in step 1d: MS (m/z): 477.2 [M+H]⁺.

### Example 21

### Preparation of 2-(2-Hydroxyphenyl)-3-(2-phenylethyl)-6-(1-piperidinylmethyl)-4(3H)-pyrimidinone

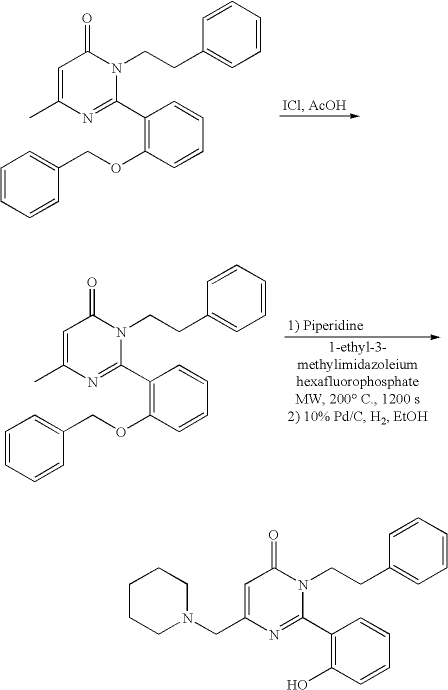

### a. 6-Methyl-3-(2-phenylethyl)-2-{2-[(phenylmethyl)oxy]phenyl}-4(3H)-pyrimidinone 6

Methyl-3-(2-phenylethyl)-2-{2-[(phenylmethyl)oxy]phenyl}-4(3H)-pyrimidinone was prepared according to the procedures described in Example 11d except 3-fluoro-2-hydroxybenzamide was replaced with 2-hydroxybenzamide.

### b. 5-Iodo-6-methyl-3-(2-phenylethyl)-2-{2-[(phenylmethyl)oxy]phenyl}-4(3H)-pyrimidinone

Methyl-3-(2-phenylethyl)-2-{2-[(phenylmethyl)oxy]phenyl}-4(3H)-pyrimidinone (4.23 g, 10.7 mmoles) was taken up in glacial acetic acid (107 mL). To this was added 1M dichloromethane solution of iodine monochloride (31 mL, 32.1 mmoles) and the reaction was stirred for 16 h. Ethyl acetate was added and acetic acid was washed with saturated sodium carbonate. The organic layer dried over sodium sulfate. Sodium sulfate was filtered off and organic layer was concentrated. The crude product was purified by chromatography on silica gel (Biotage) using ethyl acetate and hexane mixtures (20-50%) to obtain the desired product (2.5 g) in 45% yield.

### c. 3-(2-Phenylethyl)-2-{2-[(phenylmethyl)oxy]phenyl}-6=(1-piperidinylmethyl)-4(3H)-pyrimidinone

To a solution of 5-iodo-6-methyl-3-(2-phenylethyl)-2-{2-[(phenylmethyl)oxy]phenyl}-4(3H)-pyrimidinone (0.1 g, 0.19 mmoles) in 3 mL of piperidine was added 1-ethyl-3-methylimidazolium hexafluorophosphate. The reaction was irradiated in Smith synthesizer at 200° C. for 1200 s. The reaction mixture was concentrated and purified by chromatography on silica gel (Biotage) using ethylacetate and hexane mixtures (0-50%) followed by MeOH and dichloromethane (0-10%) to obtain the desired product (0.07 g) in 77% yield.

### d. 2-(2-Hydroxyphenyl)-3-(2-phenylethyl)-6-(1-piperidinylmethyl)-4(3H)-pyrimidinone

To a solution of 3-(2-phenylethyl)-2-{2-[(phenylmethyl)oxy]phenyl}-6-(1-piperidinylmethyl)-4(3H)-pyrimidinone (0.12 g, 0.25 mmoles) in a ethanol (2 mL) was added 10% Pd/C (0.03 g). This mixture was placed under hydrogen atmosphere and stirred for 12 h. The reaction mixture was filtered through a bed of celite and concentrated and purified by chromatography on silica gel (Biotage) using (0-50%) ethylacetate and hexane mixtures (0-50%) followed by (0-10%) MeOH and dichloromethane to obtain the desired product (0.7 g) in 69% yield. MS (m/z): 390.4 [M+H]⁺.

### Example 22

### Preparation of 2-(2-Hydroxyphenyl)-6-{[methyl(2-methylpropyl)amino]methyl}-3-(2-phenylethyl)-4(3H)-pyrimidinone

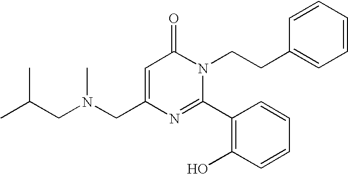

The title compound was prepared by substituting methyl(2-methylpropyl)amine for piperidine of Example 21: MS (m/z): 392.4 [M+H]⁺.

### Example 23

### Preparation of 2-(2-Hydroxyphenyl)-6-methyl-5-[(1-methylethyl)oxy]-3-(2-phenylethyl)-4(3H)-pyrimidinone

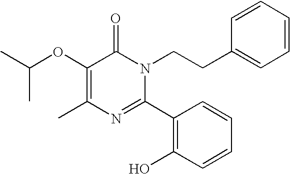

To a solution of 5-iodo-6-methyl-3-(2-phenylethyl)-2-{2-[(2-phenylmethyl)oxy]phenyl}-4(3H)-pyrimidinone (0.13 g, 0.25 mmoles) from Example 21 in 3 mL of toluene was added copper iodide (23 mg, 0.13 mmoles), phenanthroline (0.044 g, 0.25 mmoles) and cesium carbonate (0.16 g, 0.50 mmoles). The reaction mixture stirred for 5 min and isopropyl alcohol was added and heated to refluxed for 16 h. The reaction was concentrated and purified by chromatography on silica gel (Biotage) using ethyl acetate and hexane mixtures (0-30%) to obtain the desired product (0.094 g) in 84% yield. Catalytic hydrogenolysis as described previously provided the title compound: MS (m/z): 365 [M+H]⁺.

### Example 24

### Preparation of 5-(2-Furanyl)-2-(2-hydroxyphenyl)-6-methyl-3-(2-phenylethyl)-4(3H)-pyrimidinone

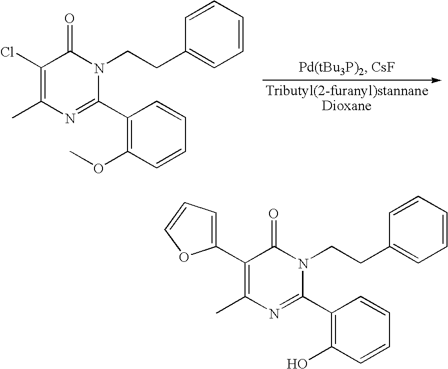

### a. 5-(2-Furanyl)-6-methyl-2-[2-(methyloxy)phenyl]-3-(2-phenylethyl)-4(3H)-pyrimidinone

To a solution containing 5-chloro-6-methyl-2-[2-(methyloxy)phenyl]-3-(2-phenylethyl)-4(3H)-pyrimidinone (0.06 g, 0.000176 mol) in deoxygenated dioxane was added Pd(tBu₃P)₂ (6.3 mg, 0.01 mmol), cesium fluoride (0.06 g, 0.00039 mol) and tributyl(2-furanyl)stannane (0.06 mL, 0.000176 mol) was added sequentially. The reaction was heated to 90° C. for 16 h and concentrated. The crude residue is diluted with dichloromethane and washed with saturated aqueous potassium fluoride, water and brine. The organic layer was separated, dried over Na₂SO₄, filtered and concentrated. The crude material was purified by chromatography on silica gel (0-50% ethyl acetate/hexane) to afford the desired product (6.6 mg) in 10% yield. MS (m/z): 373.4 [M+H]⁺.

### Example 25

### Preparation of 2-(2-Hydroxyphenyl)-6-methyl-3-(2-phenylethyl)-5-(2-thienyl)-4(3H)-pyrimidinone

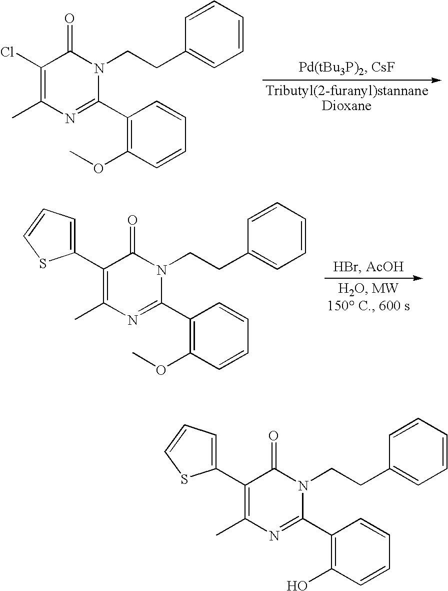

### a. 6-Methyl-2-[2-(methyloxy)phenyl]-3-(2-phenylethyl)-5-(2-thienyl)-4(3H)-pyrimidinone

To a solution containing 5-chloro-6-methyl-2-[2-(methyloxy)phenyl]-3-(2-phenylethyl)-4(3H)-pyrimidinone (0.32 g, 0.000903 mol) of Example 26 in deoxygenated dioxane was added Pd(tBu₃P)₂ (0.028 mg, 0.054 mmol), cesium fluoride (0.30 g, 0.00198 mol) and tributyl(2-thienyl)stannane (0.32 mL, 0.00099 mol) was added sequentially. The reaction was heated to 90° C. for 16 h and concentrated. The crude residue is diluted with dichloromethane and washed with saturated aqueous potassium fluoride, water and brine. The organic layer was separated, dried over Na₂SO₄, filtered and concentrated. The crude material was purified by chromatography on silica gel (0-50% ethyl acetate/hexane) to afford the desired product (0.2 g) in 54% yield. MS (m/z): 403 [M+H]⁺.

### b. 2-(2-Hydroxyphenyl)-6-methyl-3-(2-phenylethyl)-5-(2-thienyl)-4(3H)-pyrimidinone

In a 5 mL microwave vessel was charged with 6-methyl-2-[2-(methyloxy)phenyl]-3-(2-phenylethyl)-5-(2-thienyl)-4(3H)-pyrimidinone (0.1 g, 0.25 mmoles), 1.0 mL of AcOH and 2 mL of HBr. The reaction mixture was sealed and irradiated in the Smith synthesizer for 600 s at 150° C. The reaction mixture was diluted with dichloromethane and washed with NaHCO₃, brine and dried over Na₂SO₄. Sodium sulfate was filtered and concentrated. The crude product is purified by flash column chromatography (0-50% ethyl acetate/hexane) to give product (0.027 g) in 28% yield MS (m/z): 389.2 [M+H]⁺.

### Example 26

### Preparation of 2-(2-Hydroxyphenyl)-6-methyl-5-(4-morpholinyl)-3-(2-phenylethyl)-4(3H)-pyrimidinone

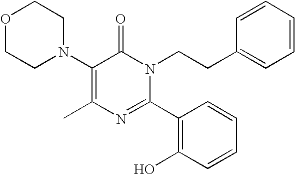

### a. 2-(Methyloxy)benzenecarboximidamide

To anhydrous ether at 0° C. was introduced to flask under argon, LiHMDS (94 ml, 93.9 mmol) was introduced and stirred for 5 min. 2-methoxy-benzonitrile (5 g, 37.6 mmol) was then added and the mixture was stirred at room temperature for 3 days. When all the starting material is consumed the reaction mixture was concentrated and 200 mL of cold 1N HCl was added and stirred for 0.5 h. The aqueous layer was extracted with diethyl ether then adjusted the pH of the aqueous layer to 13 by addition of 6N NaOH. The 2-(methyloxy)benzenecarboximidamide free base was extracted with dichloromethane (×3). The combined organic layers were dried over Na₂SO₄ and concentrated to give pure product in 91% yield.

### b. 5-Chloro-6-methyl-2-[2-(methyloxy)phenyl]-4(1H)-pyrimidinone

To a solution of 2-(methyloxy)benzenecarboximidamide (4.76 g, 0.032 mol) in 150 mL of solvent mixture of MeOH/Dioxane (1/5) was added NaOMe (2.56 g) and stirred for 15 mins. Ethyl 2-chloro-3-oxobutanoate (7.82 g, 0.048 mol) was introduced and the reaction mixture was heated to reflux for 16 h. Upon completion the reaction mixture was concentrated, diluted with dichloromethane and added dilute HCl. The dichloromethane layer was separated and washed with brine, dried over Na₂SO₄. Upon filteration and concentration the crude product was purified by flash column chromatography (30% ethylacetate/hexane) to give product (3.09 g) in 39% yield

### c. 5-Chloro-6-methyl-2-[2-(methyloxy)phenyl]-3-(2-phenylethyl)-4(3H)-pyrimidinone

To a solution of 5-chloro-6-methyl-2-[2-(methyloxy)phenyl]-4(1H)-pyrimidinone (3.2 g, 0.013 mol) in dry DMF was added LiH (0.122 g, 0.015 mol) and stirred for 10 min at room temperature. Then (2-bromoethyl)benzene was added and stirred overnight. The reaction mixture was quenched by addition of ice and 6N HCl. This mixture was extracted with EtOAc and the organic layer was washed with aqueous NaHCO₃, brine and dried over Na₂SO₄. The sodium sulfate was filtered and concentrated. The crude product is purified by flash column chromatography (30% ethyl acetate/hexane) to give product (2.13 g) in 47% yield.

### d. 2-(2-Hydroxyphenyl)-6-methyl-5-(4-morpholinyl)-3-(2-phenylethyl)-4(3H)-pyrimidinone

To a solution of 5-chloro-6-methyl-2-[2-(methyloxy)phenyl]-3-(2-phenylethyl)-4(3H)-pyrimidinone (0.07 g, 0.2 mmoles) in 3 mL of dioxane was added Pd₂(dba)₃ (0.009 mg, 0.001 mmoles), dicyclohexylphosphino-2′(n,N-dimethylaminobiphenyl) (0.008 g, 0.02 mmoles), NaOtBu (0.26 g, 0.27 mmoles) and morpholine (0.024 mL). The reaction mixture was irradiated at 180° C. for 2400 s. The reaction was concentrated and diluted with dichloromethane and washed with 5% HCl and brine. The reaction mixture was dried over sodium sulfate, filtered, concentrated and purified by flash column chromatography using MeOH/dichloromethane (0-5%) to obtain the desired product (0.020 g) in 37% yield. The resulting product was deprotected as described in Example 1e to furnish the title compound: MS (m/z): 392.4 [M+H]⁺,

### Example 27

### Preparation of 5-Ethyl-2-(3-fluoro-2-hydroxyphenyl)-3-[2-(3-fluorophenyl)ethyl]-6-(1-Piperidinyl)-4(3H)-pyrimidinone

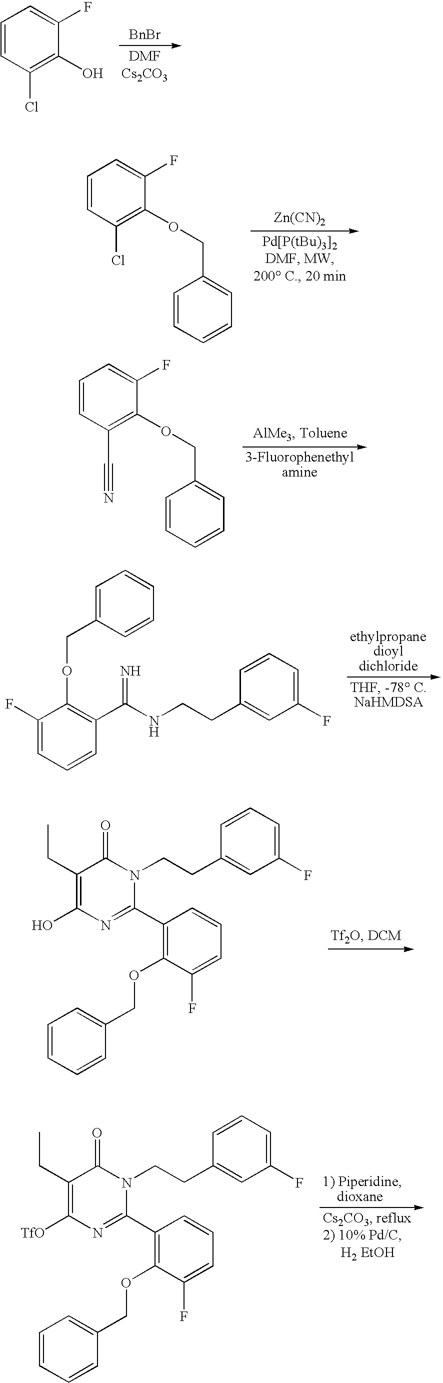

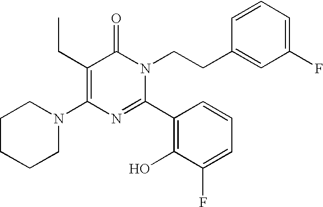

### a. 2-Chloro-6-fluorophenyl phenylmethyl ether

2-Chloro-6-fluoro phenol (2.0 g, 13.6 mmol) was dissolved in 68 ml DMF. To this solution was added Cs₂CO₃ (6.67 g, 20.5 mmol) and benzyl bromide (1.78 ml, 15 mmol) sequentially and stirred for 12 hr. The reaction mixture was diluted with EtOAc and washed with brine (3×100 mL). The organic layer was dried over Na₂SO₄, filtered and concentrated to give 2.97 g of product in 92% yield.

### b. 3-Fluoro-2-[(phenylmethyl)oxy]benzonitrile

To the solution of 1-Benzyloxy-2-chloro-6-fluoro-benzene (200 mg, 0.42 mmol) in 8 ml dry DMF was added Zn(CN)₂ (110 mg, 0.93 mmol) and Pd(t-Bu₃P)₂ (86 mg, 0.08 mmol) and the mixture was placed in microwave reactor (150° C., 20 min). The reaction mixture was diluted with EtOAc and washed with brine. Organic layer was dried over Na₂SO₄, filtered and concentrated. The crude product was purified by flash column chromatography (0 to 20% EtOAc/Hexane) to produce the desired product (0.8 g) in 83% in yield.

### c. 3-Fluoro-N-[2-(3-fluorophenyl)ethyl]-2-[(phenylmethyl)oxy]benzenecarboximidamide

A round bottom flask was charged with [2-(3-fluorophenyl)ethyl]amine (0.8 g, 0.59 mmol) and 5 ml of toluene and cooled to 0° C. Me₃Al (2.0M in Hexane, 0.88 ml, 0.18 mmol) was added dropwise over 30 min and the resulted mixture was stirred at 0° C. for 0.5 hr and warmed to RT for 4 hr. 3-fluoro-2-[(phenylmethyl)oxy]benzonitrile (200 mg, 0.88 mmol) was then added at RT in portions and heated to 80° C. under Argon overnight. After cooling to RT, the mixture was poured slowly to a slurry of silica gel in CHCl₃ and stirred for 30 min. The mixture was filtered and rinsed with 20% MeOH in chloroform (×3). The filtrate was concentrated and purified by flash column chromatography (50% EtOAc/hexane to 10% MeOH/dichloromethane and 0.1% NH₃) to afford the desired product (0.054 mg) in 25% yield.

### d. 5-Ethyl-3-[2-(3-fluorophenyl)ethyl]-2-{3-fluoro-2-[(phenylmethyl)oxy]phenyl}-6-hydroxy-4(3H)-pyrimidinone

To a cold solution (−78° C.) of 3-fluoro-N-[2-(3-fluorophenyl)ethyl]-2-[(phenylmethyl)oxy]benzenecarboximidamide in THF (0.28 g, 0.00077 mol) was added NaHMDSA (0.768 mL, 0.00077 mol) and stirred for 10 minutes. Ethyl malonyl chloride was added (0.143 mL, 0.00084 mol) dropwise via a cannula. After stirring overnight while warmed to RT, the reaction mixture was diluted with EtOAc and washed with brine. The organic layer was separated, dried and concentrated. The crude product was purified by flash column chromatography (30% EtOAc/hexane) to afford the desired product (0.12 g) in 42% yield.

### e. 5-Ethyl-1-[2-(3-fluorophenyl)ethyl]-6-oxo-2-{2-[(phenylmethyl)oxy]phenyl}-1,6-dihydro-4-pyrimidinyl trifluoromethanesulfonate

5-Ethyl-3-[2-(3-fluorophenyl)ethyl]-2-{3-fluoro-2-[(phenylmethyl)oxy]phenyl}-6-hydroxy-4(3H)-pyrimidinone (0.14 g, 2.94 mmol) was taken up in DCM (5 mL) and cooled to −78° C. To this was added collidine (0.057 mL, 4.31 mmol) and reaction stirred for 5 minutes. At this time trifluoromethanesulfonic anhydride (0.066 mL, 3.96 mmol) was added and the reaction warmed to 0° C. and stirred overnight. The reaction mixture was diluted with EtOAc and washed with H₂O, brine and dried (Na₂SO₄) and concentrated. The crude product was purified by flash column chromatography (30% EtOAc/hexane) to afford the desired product (0.081 g) in 47% yield.

### f. 5-Ethyl-2-(3-fluoro-2-hydroxyphenyl)-3-[2-(3-fluorophenyl)ethyl]-6-(1-piperidinyl)-4(3H)-pyrimidinone

To the solution of 5-ethyl-1-[2-(3-fluorophenyl)ethyl]-6-oxo-2-{2-[(phenylmethyl)oxy]phenyl}-1,6-dihydro-4-pyrimidinyl trifluoromethanesulfonate (30 mg, 0.07 mmol) in dry dioxane was added piperidine (7.7 ul, 0.08 mmol) and Cs₂CO₃ (31 mg, 0.1 mmol). The reaction mixture was heated at 105° C. overnight. The reaction mixture was concentrated and purified by flash column chromatography (0 to 50% EtOAc/Hexane) to give desired product (25.5 mg) in 74% yield. The title compound was prepared by carrying out the deprotection using catalytic hydrogenolysis protocol. MS (m/z): 440.4 [M+H]⁺.

### Example 28

### Preparation of 5-Ethyl-1-[2-(3-fluorophenyl)ethyl]-2-[2-(methyloxy)phenyl]-6-oxo-1,6-dihydro-4-pyrimidinecarboxylic acid

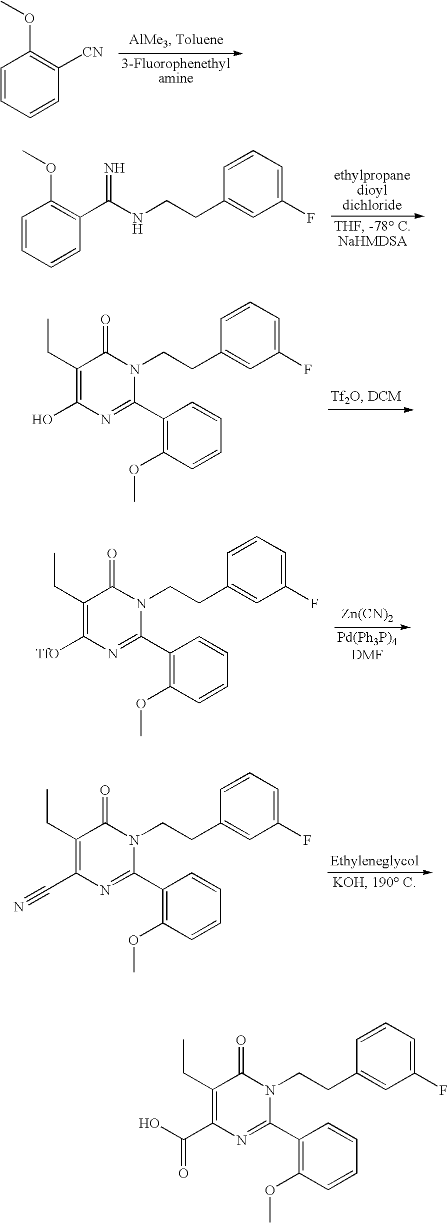

### a. 5-Ethyl-1-[2-(3-fluorophenyl)ethyl]-6-oxo-2-{2-[(phenylmethyl)oxy]phenyl}-1,6-dihydro-4-pyrimidinyl trifluoromethanesulfonate

The title compound was prepared following the general procedure outlined in Example 27 except substituting 2-methoxybenzonitrile for 3-fluoro-2-[(phenylmethyl)oxy]benzonitrile in steps 27c.

### b. 5-Ethyl-1-[2-(3-fluorophenyl)ethyl]-2-[2-(methyloxy)phenyl]-6-oxo-1,6-dihydro-4-pyrimidinecarbonitrile

To the solution of 5-ethyl-1-[2-(3-fluorophenyl)ethyl]-6-oxo-2-{2-[(phenyl methyl)oxy]phenyl}-1,6-dihydro-4-pyrimidinyl trifluoromethanesulfonate

(0.1 g, 0.20 mmol) in 2 mL dry DMF was added Zn(CN)₂ (0.026 g, 0.22 mmol) and Pd(Ph₃P)₄ (0.023 g, 0.02 mmol) and the mixture was placed in microwave reactor (150° C., 2500 s). The reaction mixture was diluted with EtOAc and washed with brine. Organic layer was dried over Na₂SO₄, filtered and concentrated. The crude product was purified by flash column chromatography (0 to 20% EtOAc/Hexane) to produce the desired product (0.06 g) in 83% in yield.

### c. 5-Ethyl-1-[2-(3-fluorophenyl)ethyl]-2-[2-(methyloxy)phenyl]-6-oxo-1,6-dihydro-4-pyrimidinecarboxylic acid

To a solution of 5-ethyl-1-[2-(3-fluorophenyl)ethyl]-2-[2-(methyloxy)phenyl]-6-oxo-1,6-dihydro-4-pyrimidinecarbonitrile (0.29 g, 0.77 mmoles) in ethylene glycol (7 mL) was added KOH (0.22 g, 3.84 mmoles) and the reaction heated to 190° C. for 16 h. Some solvent was removed under reduced pressure at elevated temperatures. The remaining reaction mixture was diluted with dichloromethane and acidified to pH ˜5 with 1N HCl. The organic layer was separated, dried over Na₂SO₄, filtered and concentrated. The crude product was purified by flash column chromatography using (0 to 20%) MeOH in dichloromethane (saturated with ammonia) to produce the desired product (0.15 g) in 52% in yield. MS (m/z): 397.2 [M+H]⁺.

### Example 29

### Preparation of 5-Ethyl-2-(2-hydroxyphenyl)-6-methyl-3-[(E)-2-phenylethenyl]-4 (3H)-pyrimidinone

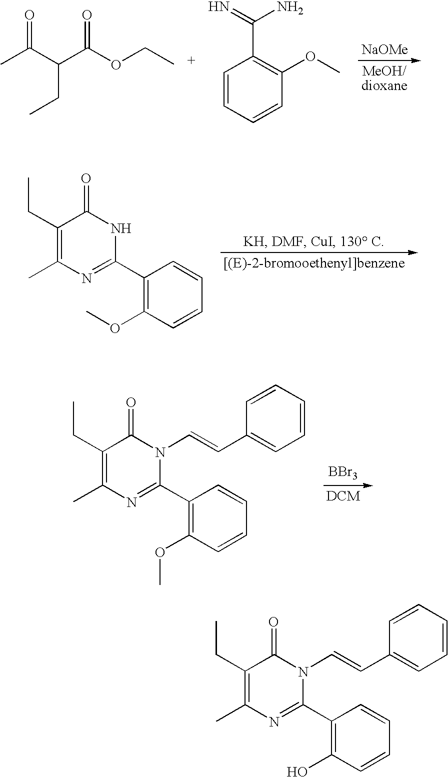

### a. 5-Ethyl-6-methyl-2-[2-(methyloxy)phenyl]-4(1H)-pyrimidinone

25% NaOMe solution in methanol (58.6 mL) was added to a 0° C. solution of 2-(methoxy)benzenecarboxamidine (2.0 g, 0.013 mol) and ethyl 2-ethyl-3-oxobutanoate (3.16 g, 0.02 mol) in methanol (126 mL) and 1,4-dioxane (25 mL). The resulting mixture was refluxed overnight. The solvents were removed and the residue was diluted with H₂O and pH was adjusted to 8 with acetic acid. The layers were separated and the aqueous layer was extracted with dichlormethane 3 times. The combined organic portions were dried over Na₂SO₄ and purified by flash column chromatography (0-100% EtOAc/hexanes) to give pure of product.

### b. 5-Ethyl-6-methyl-2-[2-(methyloxy)phenyl]-3-[(E)-2-phenylethenyl]4(3H)-pyrimidinone

To a solution of intermediate 5-ethyl-6-methyl-2-[2-(methyloxy)phenyl]-4(1H)-pyrimidinone (0.1 g, 0.41 mmoles) in dry DMF (1 mL) was added KH (0.016 g, 0.41 mmoles) and stirred for 5 min. [(E)-2-bromoethenyl]benzene (0.053 mL, 0.41 mmoles) and CuI (0.078 g, 0.41 mmoles) were added to the reaction sequentially and heated to 130° C. for 16 h. The reaction was cooled, diluted with EtOAc and washed with brine. Organic layer was dried over Na₂SO₄, filtered and concentrated. The crude product was purified by flash column chromatography (0 to 20% EtOAc/Hexane) to produce the desired product (0.05 g) in 36% in yield.

### c. 5-Ethyl-2-(2-hydroxyphenyl)-6-methyl-3-[(E)-2-phenylethenyl]-4(3H)-pyrimidinone

The deprotection of 5-ethyl-6-methyl-2-[2-(methyloxy)phenyl]-3-[(E)-2-phenylethenyl]-4(3H)-pyrimidinone was accomplished using BBr₃ as detailed in Example 1e to provide the title compound: MS (m/z): 333.4 [M+H]⁺.

### Example 30

### Preparation of 2-(3,6-Difluoro-2-hydroxyphenyl)-5-ethyl-3-[2-(3-fluorophenyl)ethyl]-6-methyl-4(3H)-pyrimidinone

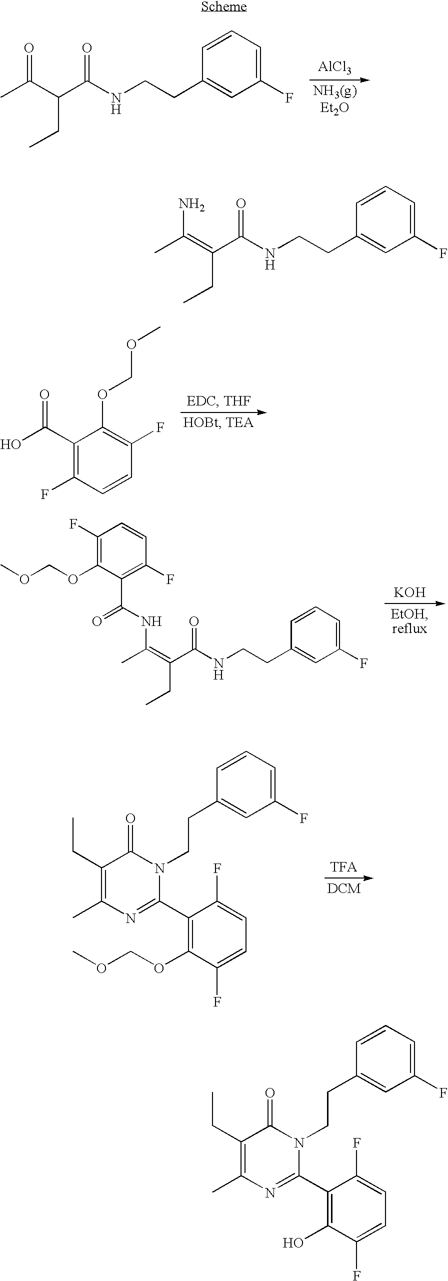

### a. (2Z)-3-Amino-2-ethyl-N-[2-(3-fluorophenyl)ethyl]-2-butenamide

A solution of 2-ethyl-N-[2-(3-fluorophenyl)ethyl]-3 oxobutanamide (3.1 g, 0.012 moles) of Example 9 in dry diethyl ether (350 mL) at 0° C. was saturated with gaseous ammonia for 3 h. AlCl₃ (2.0 g) was added, and the mixture was addowed to warm to RT while stirring overnight. The resulting suspension was filtered, and the filtrate was concentrated to provide product as a colorless oil (2.1 g) in 68% yield.

### b. 3,6-Difluoro-2-{[(methyloxy)methyl]oxy}benzoic acid

This compound was prepared according to the procedure reported in the literature (Eur. J. Org. Chem. 2001, 15, 2911-2915).

### c. 3,6-Difluoro-N-[(1Z)-2-({[2-(3-fluorophenyl)ethyl]amino}carbonyl)-1-methyl-1-buten-1-yl]-2-{[(methyloxy)methyl]oxy}benzamide

To a solution of 3,6-difluoro-2-{[(methyloxy)methyl]oxy}benzoic acid (0.2 g, 0.91 mmoles) and (2Z)-3-amino-2-ethyl-N-[2-(3-fluorophenyl)ethyl]-2-butenamide (0.22 g, 0.87 mmoles) in dry THF was added EDC (0.21 g, 1.09 mmoles), HOBt (0.15 g, 1.09 mmoles) and TEA (0.51 mL, 3.65 mmoles) sequentially. The reaction was stirred at ambient temperature for 48 h. The reaction was diluted with EtOAc and washed with dilute HCl, 5% NaHCO₃ and brine. The organic layer was separated dried over Na₂SO₄, filtered and concentrated. The crude product was purified by flash column chromatography (30% EtOAc/Hexane) to produce the desired product 28c (0.081 g).

### d. 2-(3,6-Difluoro-2-{[(methyloxy)methyl]oxy}phenyl)-5-ethyl-6-methyl-3-(2-phenylethyl)-4(3H)-pyrimidinone

3,6-Difluoro-N-[(1Z)-2-({[2-(3-fluorophenyl)ethyl]amino}carbonyl)-1-methyl-1-buten-1-yl]-2-{[(methyloxy)methyl]oxy}benzamide (0.39 g, 0.87 mmoles) was taken up in ethanol (7 mL) and 5 mL of 25% KOH was added and the reaction refluxed overnight. After reaction was cooled to RT the pH is adjusted to ˜1 with 3N HCl and extracted with dichloromethane. The combined organic layers were dried over Na₂SO₄, filtered and concentrated. The crude product was purified by flash column chromatography (30% EtOAc/Hexane) to produce the desired product (0.32 g) along with some impurity.

### e. 2-(3,6-Difluoro-2-hydroxyphenyl)-5-ethyl-3-[2-(3-fluorophenyl)ethyl]-6-methyl-4(3H)-pyrimidinone

The product mixture obtained from the previous step was dissolved in dry dichloromethane and to this was added TFA (2 mL) and the reaction stirred for 3 h. Upon completion of the reaction the reaction was concentrated diluted with dichloromethane and washed with 5% NaHCO₃, brine and dried over Na₂SO₄. The crude product was purified by flash column chromatography (30% EtOAc/Hexane) to produce the desired product (0.048 g) in 15% overall yield. MS (m/z): 389.2 [M+H]⁺.

### Example 31

### Preparation of 2-(3-Fluoro-2-hydroxyphenyl)-6-methyl-5-propyl-3-[2-(2-thienyl)ethyl]-4(3H)-pyrimidinone

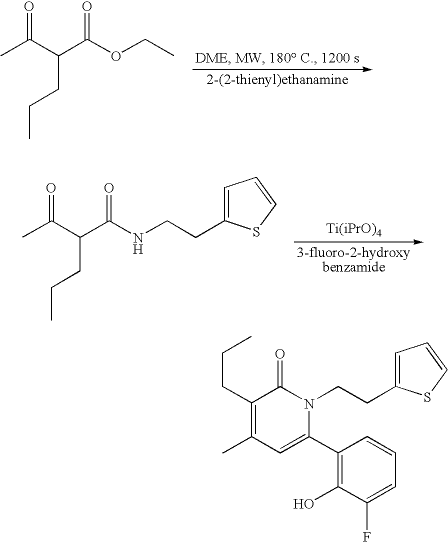

The title compound was prepared according to the general procedures outlined in Example 1 except by substituting allylbromide for 3-bromo-2-methyl-1-propene in step 1a, 2-thienlylenthylamine for phenethylamine in step 1 b and 3-fluoro-2-hydroxybenzamide for 2-fluoro-3-methoxybenzamide in step 1d. MS (m/z): 373.2 [M+H]⁺.

### Example 32

### Preparation of 2-(2-Hydroxyphenyl)-5,5-dimethyl-3-[2-(2-thienyl)ethyl]-5,6,7,8-tetrahydro-4(3H)-quinazolinone

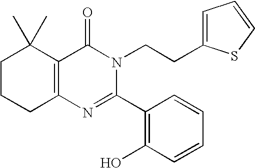

The title compound was prepared according to the procedures described in Example 26 except substituting methyl 2,2-dimethyl-6-oxocyclohexanecarboxylate for ethyl 2-chloro-3-oxobutanoate and 2-(2-bromoethyl)thiophene for (2-bromethyl)benzene: ¹H NMR (400 MHz, CHCl₃-d) δ ppm 1.42-1.46 (m, 6H), 1.66-1.72 (m, 2H), 1.82-1.90 (m, 2H), 2.75 (t, J=6.06 Hz, 2H), 3.15 (t, J=7.07 Hz, 2H), 4.18-4.25 (m, 2H), 6.54 (d, J=3.28 Hz, 1H), 6.75 (d, J=8.34 Hz, 1H), 6.82-6.92 (m, 3H), 7.12 (d, J=5.05 Hz, 1H), 7.32-7.39 (m, 1H).

### Example 33

### Preparation of 3-[2-(2-Fluorophenyl)ethyl]-2-(2-hydroxyphenyl)-5,5-dimethyl-5,6,78-tetrahydro-4(3H)-quinazolinone

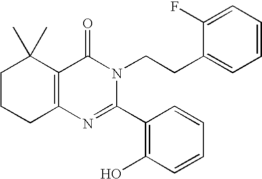

The title compound was prepared according to the procedures of Example 26 except substituting methyl 2,2-dimethyl-6-oxocyclohexanecarboxylate for ethyl 2-chloro-3-oxobutanoate and 2-fluorophenethyl bromide for (2-bromethyl)benzene: ¹H NMR (400 MHz, CHCl₃-d) δ ppm 1.25-1.37 (m, 2H), 1.38-1.47 (m, 6H), 1.59-1.71 (m, 2H), 1.77-1.85 (m, 2H), 2.61 (t, J=6.32 Hz, 2H), 3.06 (t, J=7.33 Hz, 2H), 4.32-4.41 (m, 2H,) 6.92-7.04 (m, 4H), 7.14-7.24 (m, 1H) 7.27-7.36 (m, 3H).

### Example 34

### Preparation of 2-(2-Hydroxyphenyl)-3-(2-phenylethyl)-3,5,6,7,8,9-hexahydro-4H-cyclohepta[d]pyrimidin-4-one

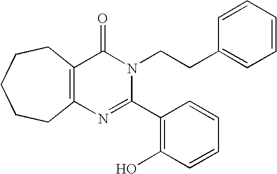

The title compound was prepared according to the procedures of Example 26 except substituting methyl 2-oxocycloheptanecarboxylate for ethyl 2-chloro-3-oxobutanoate: MS (m/z): 361.2 [M+H]⁺.

### Example 35

### Preparation of 2-(3-Fluoro-2-hydroxyphenyl)-3-(2-phenylethyl)-5,6,7,8-tetrahydro-4(3H)-quinazolinone

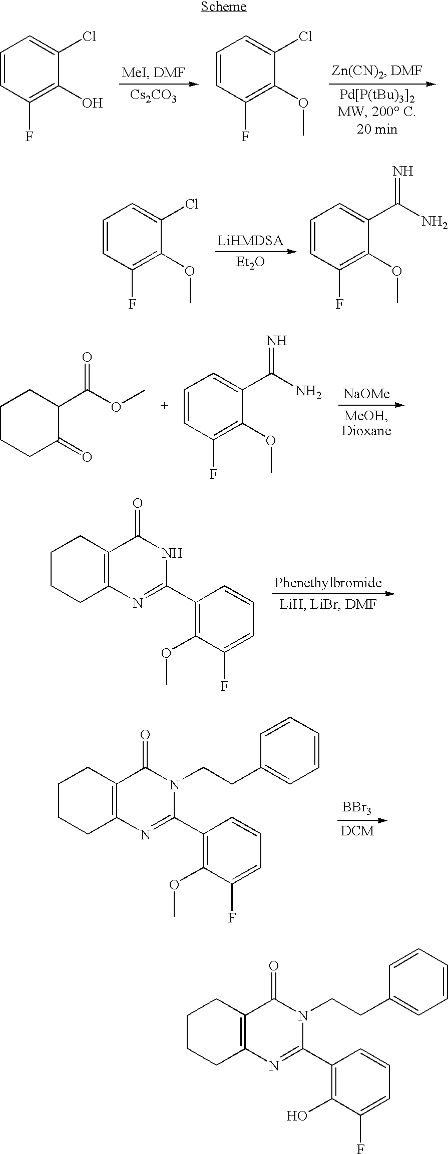

### a. 3-Fluoro-2-(methyloxy)benzonitrile

This compound was prepared by following the general procedures outlined in Example 27 and substituting methyl iodide in place of benzyl bromide in step 1a.

### b. 3-Fluoro-2-(methyloxy)benzenecarboximidamide

3-Fluoro-2-methoxybenzonitrile (4-9 g, 0.032 mol) was added to a 0° C. solution of LiHMDS (81 mL, 1M in hexanes. 0081 mol) in anhydrous Et₂O (65 mL, 0.5 M) under N₂. After warming to room temperature, the mixture stirred for three days. The resulting reaction mixture was quenched by the addition of 1N HCl. The layers were separated and the aqueous phase was extracted 2 times with Et₂O. The aqueous layer was cooled in an ice-bath, adjusted to pH 12, and extracted 3 times with dichlormethane. The organic portions were pooled, dried over Na₂SO₄, and concentrated to a brown oil which solidified to a brown solid under vacuum (5.0 g, 93% yield): Check with YL

### c. 2-[3-Fluoro-2-(methyloxy)phenyl]-5,6,7,8-tetrahydro-4(1H)-quinazolinone

25% (w/v) solution of NaOMe (3.68 mL, 0.0257 mol) was added to a 0° C. solution of 3-fluoro-2-(methyloxy)benzenecarboximidamide (1.32 g, 0.0117 mol) and methyl 2-oxocyclohexanecarboxylate (2.0 g, 0.0117 mol) in methanol (70 mL) and 1,4-dioxane (20 mL). The resulting mixture was refluxed overnight. The solvents were removed and the residue was brought up in ethyl acetate and 1N HCl. The layers were separated and the aqueous layer was extracted with dichlormethane 3 times. The combined organic portions were dried over Na₂SO₄ and purified by flash column chromatography to give 2.05 g of product (75% yield).

### d. 2-[3-Fluoro-2-(methyloxy)phenyl]-3-(2-phenylethyl)-5,6,7,8-tetrahydro-4(3H)-quinazolinone

LiH (0.032 g, 4.0 mmol) and LiBr (0.52, 6.0 mmol) was added to a 0° C. solution of 2-[3-fluoro-2-(methyloxy)phenyl]-5,6,7,8-tetrahydro-4(1H)-quinazolinone (0.55 g, 2.0 mmol) in DMF (10 mL) and stirred at 0° C. for 30 minutes. Bromoethyl benzene (1.36 mL, 10 mmol) was added and the resulting mixture stirred at room temperature for 40 hours. The reaction was quenched by the addition of ethyl acetate (15 mL) and water (15 mL). The layers were separated and the organic portion was washed 3 times with water, dried over NaSO₄, filtered, and concentrated. Flash column chromatography (30% ethyl acetate/hexanes) provided pure product.

### e. 2-(3-Fluoro-2-hydroxyphenyl)-3-(2-phenylethyl)-5,6,7,8-tetrahydro-4(3H)-quinazolinone

To a 0° C. dichlormethane solution of the 2-[3-fluoro-2-(methyloxy)phenyl]-3-(2-phenylethyl)-5,6,7,8-tetrahydro-4(3H)-quinazolinone (0.12 g, 0.32 mmol) was added BBr₃ (1.6 mL, 1M in dichlormethane) dropwise. The resulting solution was allowed to warm to room temperature while stirring overnight. The reaction was quenched by the addition of saturated Na₂CO₃ and dichlormethane. The layers were separated and the organic portion was dried over MgSO₄, filtered and concentrated. The crude residue was purified by flash column chromatography to give the title compound. MS (m/z): 365.2 [M+H]⁺.

### Example 36

### Preparation of 5-Cyclopentyl-2-(3-fluoro-2-hydroxyphenyl)-6-methyl-3-(2-phenylethyl)-4(3H-pyrimidinone

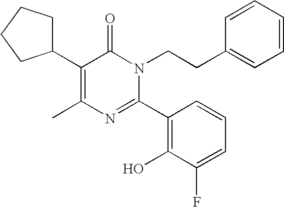

The title compound was prepared following the general procedure outlined in Example 35 except substituting ethyl □-acetylcyclopentaneacetate for methyl 2-oxocyclohexanecarboxylate. MS (m/z): 393.2 [M+H]⁺.

### Example 37

### Preparation of 5-(2,3-Dihydro-1,4-benzodioxin-6-yl)-6-methyl-3-(2-phenylethyl)-2-(2-thienyl)-4(3H)-pyrimidinone

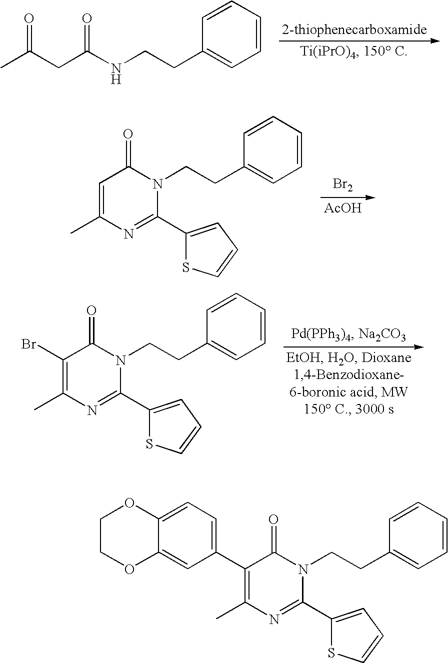

### a. 5-Bromo-6-methyl-3-(2-phenylethyl)-2-(2-thienyl)-4(3H)-pyrimidinone

The title compound was prepared according to the procedure of Example 11 except substituting 2-thiophenecarboxamide for 3-fluoro-2-hydroxybenzamide in step 11d.

### b. 5-(2,3-Dihydro-1,4-benzodioxin-6-yl)-6-methyl-3-(2-phenylethyl)-2-(2-thienyl)-4(3H)-pyrimidinone

To a solution of 5-bromo-6-methyl-3-(2-phenylethyl)-2-(2-thienyl)-4(3H)-pyrimidinone (0.20 g, 0.53 mmoles) in dioxane was added 1,4-benzodioxane-6-boronic acid (0.19 g, 1.06 mmoles) dissolved in solvent mixture of 0.5 mL ethanol and 0.5 mL of dioxane, and 0.5 mL of aqueous sodium carbonate (0.09 g, 0.8 mmoles) in a microwave reaction vessel. To this was added Pd(PPh₃)₄ (0.12 g, 0.11 mmol) and irradiated to 150° C. for 3000 seconds. The reaction mixture was filtered through syringe filter (Acrodisc CR25 mm with 0.2 □m PTFE membrane). The filtrate was diluted with EtOAc and washed with brine, separated, dried over sodium sulfate, filtered, concentrated in vacuo and the residue was purified reverse phase HPLC to afford the desired product. MS (m/z): 431.2 [M+H]⁺.

### Example 38

### Preparation of 2-(2-Hydroxyphenyl)-6-[(methyloxy)methyl]-3-(2-phenylethyl)-4(3H) pyrimidinone

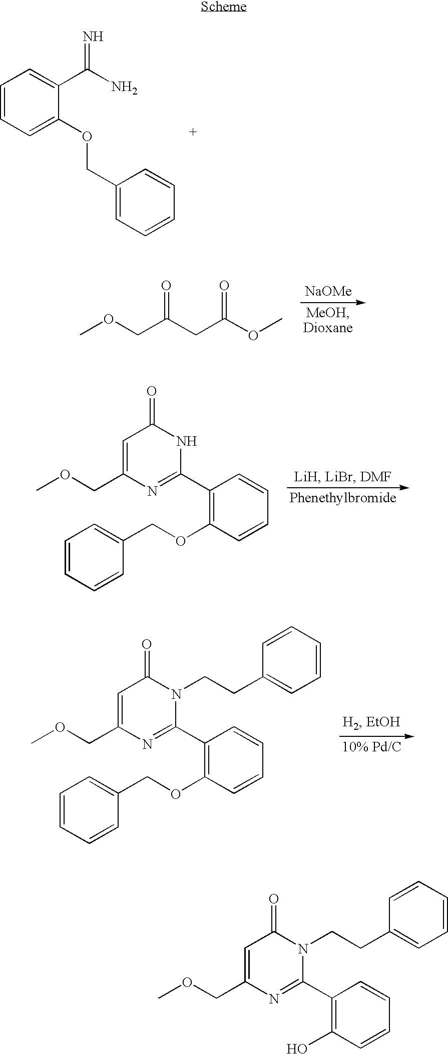

### a. 6-[(Methyloxy)methyl]-2-{2-[(phenylmethyl)oxy]phenyl}-4(1H)-pyrimidinone

The title compound was prepared following the general procedure outlined in Example 35 except substituting 2-[(phenylmethyl)oxy]benzenecarboximidamide for 3-fluoro-2-(methyloxy)benzenecarboximidamide and methyl 4-(methyloxy)-3-oxobutanoate for 2-oxocyclohexanecarboxylate in step 35c.

### b. 2-(2-Hydroxyphenyl)-6-[(methyloxy)methyl]-3-(2-phenylethyl)-4(3H)-pyrimidinone

6-[(Methyloxy)methyl]-2-{2-[(phenylmethyl)oxy]phenyl}-4(1H)pyrimidinone (0.05 g, 0.11 mmoles) dissolved in ethanol was added 10% Pd/C (0.01 g). This mixture was placed under hydrogen atmosphere and stirred for 12 h. The reaction mixture was filtered through a bed of celite and concentrated afford the desired product (0.021 g) in 56% yield. MS (m/z): 337.0 [M+H]⁺.

### Example 39

### Preparation of 2-(2-Hydroxyphenyl)-6-[(methyloxy)methyl]-5-(2-methylpropyl)-3-(2-phenylethyl)-4(3H)-pyrimidinone

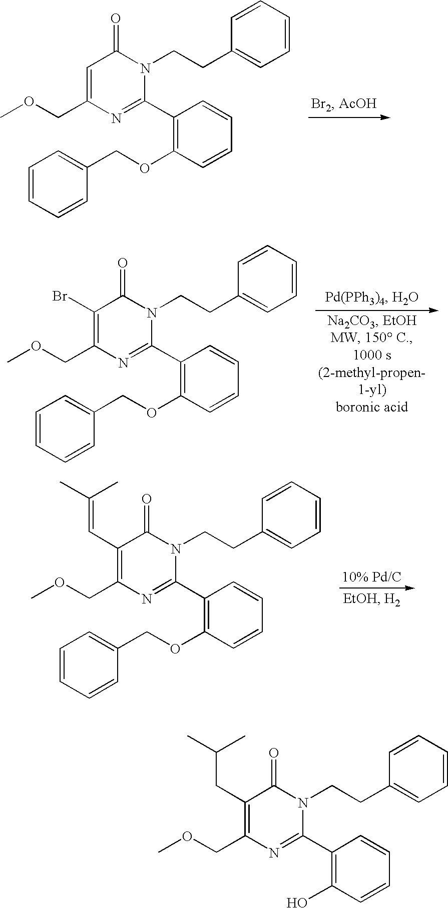

### a. 5-Bromo-6-[(methyloxy)methyl]-3-(2-phenylethyl)-2-{2-[(phenylmethyl)oxy]phenyl}-4(3H)-pyrimidinone

6-[(Methyloxy)methyl]-3-(2-phenylethyl)-2-[2-[(phenylmethyl)oxy]phenyl]-4(3H)-pyrimidinone (0.8 g, 1.9 mmol) of Example 38 was taken up in glacial acetic acid. To this was added bromine (0.144 mL, 2.8 mmol) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was purified by chromatography on silica gel (Biotage) to obtain the desired product.

### b. 6-[(Methyloxy)methyl]-5-(2-methyl-1-propen-1-yl)-3-(2-phenylethyl)-2-{2-[(phenylmethyl)oxy]phenyl}-4(3H)-pyrimidinone

To a solution of 5-bromo-6-[(methyloxy)methyl]-3-(2-phenylethyl)-2-(2-[(phenylmethyl)oxy]phenyl)-4(3H)-pyrimidinone (0.50 g, 0.99 mmol) in dioxane was added 2,2-dimethylenylboronic acid (0.20 g, 1.98 mmol) dissolved in solvent mixture of 0.5 mL ethanol and 0.5 mL of dioxane, and 0.5 mL of aqueous sodium carbonate (0.09 g, 0.8 mmoles) in a microwave reaction vessel. To this was added Pd(PPh₃)₄ (0.172 g, 0.15 mmol) and irradiated to 150° C. for 1000 seconds. The reaction mixture was filtered through syringe filter (Acrodisc CR25 mm with 0.2 δm PTFE membrane). The filtrate was diluted with EtOAc and washed with brine, separated, dried over sodium sulfate, filtered, concentrated in vacuo and the residue was purified by chromatography on silica gel (Biotage) to afford the desired product.

### c. 2-(2-Hydroxyphenyl)-6-[(methyloxy)methyl]-5(2-methylpropyl)-3-(2-phenylethyl)-4(3H)-pyrimidinone

To a solution of 6-[(methyloxy)methyl]-5-(2-methyl-1-propen-1-yl)-3-(2-phenylethyl)-2-{2-[(phenylmethyl)oxy]phenyl}-4(3H)-pyrimidinone (0.409 g, 0.81 mmol) in acetic acid (30 mL) was added 10% Pd/C (0.10 g). This mixture was placed under hydrogen atmosphere (50 psi) for 72 h. The reaction mixture was filtered through a bed of celite and concentrated to afford the desired product. MS (m/z): 393.2 [M+H]⁺.

### Example 40

### Preparation of 2-(3-Fluoro-2-hydroxyphenyl)-6-methyl-5-[2-(methyloxy)ethyl]-3-(2-phenylethyl)-4(3H)-pyrimidinone

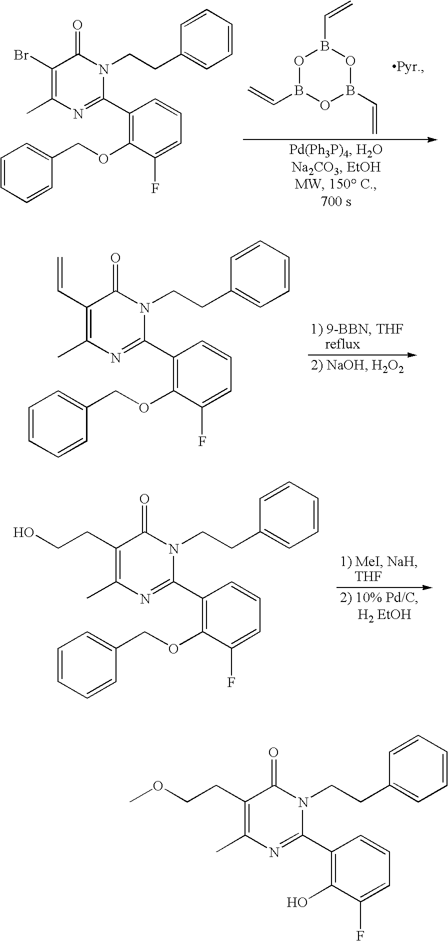

### a. 5-Ethenyl-2-{3-fluoro-2-[(phenylmethyl)oxy]phenyl}-6-methyl-3-(2-phenylethyl)-4(3H)-pyrimidinone

To a solution of 5-bromo-2-{3-fluoro-2-[(phenylmethyl)oxy]phenyl}-6-methyl-3-(2-phenylethyl)-4(3H)-pyrimidinone (1.5 g, 0.003 moles) in dioxane (10 mL) was added 2,4,6-trivinylcycloboroxane pyridine complex (0.88 g, 0.0036 mmoles) dissolved in solvent mixture of 0.5 mL ethanol and 0.5 mL of dioxane, and 0.5 mL of aqueous sodium carbonate (0.64 g, 0.0061 moles) in a microwave reaction vessel. This mixture was irradiated to 150° C. for 700 seconds. The reaction mixture was filtered through syringe filter (Acrodisc CR25 mm-with-0.2 δm PTFE membrane). The filtrate was diluted with EtOAc and washed with brine, separated, dried over sodium sulfate, filtered, concentrated in vacuo and the residue was purified by chromatography on silica gel (Biotage, 0-60% ethyl acetate/hexane) to afford the desired product (0.86 g) in 64% yield.

### b. 2-(3-Fluoro-2-hydroxyphenyl)-6-methyl-5-[2-(methyloxy)ethyl]-3-(2-phenylethyl)-4(3H)-pyrimidinone

To a solution of 5-ethenyl-2-{3-fluoro-2-[(phenylmethyl)oxy]phenyl}-6-methyl-3-(2-phenylethyl)-4(3H)-pyrimidinone (0.19 g, 0.45 mmoles) in dry THF was added 0.5 M solution of 9-BBN (1.07 mL, 0.54 mmoles) and the reaction refluxed for 1 h. An additional 1 mL of 9-BBN was added and reaction continued to reflux for another 2 h. The reaction mixture was cooled and added 14 mL of 3N NaOH and 2 mL of 30% H₂O₂ and stirred for 6 h. The crude reaction mixture was extracted with EtOAc dried over Na₂SO₄. The crude product was purified by flash column chromatography (40% EtOAc/Hexane) to produce the desired product (0.10 g) in 57% yield.

### c. 2-(3-Fluoro-2-hydroxyphenyl)-6-methyl-5-[2-(methyloxy)ethyl]-3-(2-phenylethyl)-4(3H)-pyrimidinone

To a solution of 2-(3-Fluoro-2-hydroxyphenyl)-6-methyl-5-[2-(methyloxy)ethyl]-3-(2-phenylethyl)-4(3H)-pyrimidinone (0.22 g, 0.48 mmoles) in dry THF was added NaH (0.029 g, 0.71 moles) and stirred for 2 min. Iodomethane (0.059 mL, 0.95 mmoles) was added and the reaction was warmed to 50° C. and stirred for 6 h. The reaction was quenched with 1N HCl and extracted with EtOAc. The organic layer was separated dried over Na₂SO₄. The crude product was purified by flash column chromatography (40% EtOAc/Hexane) to produce the desired product (0.17 g) in 77% yield. Removal of the benzyl protecting group via catalytic hydrogenolysis as previously described provided the title compound: MS (m/z): 383.2 [M+H]⁺.

### Example 41

### Preparation of 2-(3-Fluoro-2-hydroxyphenyl)-6-methyl-3-(2-phenylethyl)-5-propyl-4(3H)-pyrimidinethione

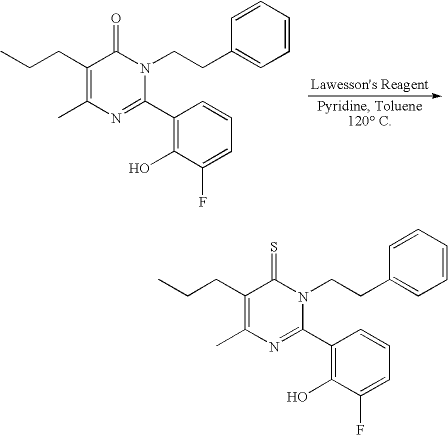

### a. 2-Acetyl-N-(2-phenylethyl)pentanamide

To a solution of ethyl 2-acetylpentanoate (1.0 g, 5.81 mmol) from example 31 in DMIE (21 mL) was added phenethylamine (0.7 g, 5.23 mmol) in a microwave reaction vessel. Few drops of ethanol was added to the reaction mixture was irradiated to 180° C. for 1200 s. The reaction mixture was diluted with EtOAc and washed with 1N HCl. Organic layer was separated and dried over Na₂SO₄. Filtered, concentrated and purified by chromatography on silica gel (Biotage, 0-40% ethyl acetate/hexane) to afford pure amide (0.6 g) in 42% yield. MS (m/z): 248.2 [M+H]⁺.

### b. 2-(3-Fluoro-2-hydroxyphenyl)-6-methyl-3-(2-phenylethyl)-5-propyl-4(3H)-pyrimidinone

The 3-oxo-N-(2-phenylethyl)butanamide (6.2 g, 0.025 moles) was placed in 500 mL round bottom flask and added 251 mL of m-xylene followed by titanium isopropoxide (74 mL, 0.25 moles). While the reaction is stirring 3-fluoro-2-hydroxybenzamide (3.92 g, 0.025 moles) was added, a condenser was placed and the reaction was heated to reflux (oil bath temperature=150° C.). The 2-hydroxy-3-fluorobenzamide dissolved slowly and gave brown homogenous solution upon some time at elevated temperatures. Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude reaction mixture was purified by EtOAc/hexanes and followed by MeOH in dichloromethane to give the pure product in 46% (4.21 g) yield. ¹H NMR (400 MHz, CDCl₃) δ ppm 1.04 (t, J=7.4 Hz, 2H), 1.55-1.61 (m, 2H), 2.27 (s, 3H), 2.52-2.56 (m, 2H), 2.88 (t, J=7.4 Hz, 2H), 4.17 (t, J=7.4 Hz, 2H), 6.85-6.89 (m, 5H), 7.04-7.19 (m, 3H), 9.98 (brs, 1H). MS (m/z): 367.2 [M+H]⁺.

### c. 2-(3-Fluoro-2-hydroxyphenyl)-6-methyl-3-(2-phenylethyl)-5-propyl-4(3H)-pyrimidinethione

To sealed tube containing 2-(3-fluoro-2-hydroxyphenyl)-6-methyl-3-(2-phenylethyl)-5-propyl-4(3H)-pyrimidinone (0.1 g, 0.27 mmoles) in dry toluene (2.0 mL) was added Lawesson's reagent (0.32 g, 0.82 mmoles) and pyridine (0.065 mL, 0.82 moles). The sealed tube was closed and heated to 120° C. for 16 h whereupon it was allowed to cool to room temperature. The resulting solid was filtered and crude product was purified by chromatography on silica gel (Biotage) using (0-50%) EtOAc/hexane to provide the title compound (0.037 g) in 36%. MS (m/z): 383.2 [M+H]⁺.

### Example 42

### Preparation of 2-(3-Fluoro-2-hydroxyphenyl)-6-methyl-5-phenyl-3-(2-phenylethyl)-4(3H)-pyrimidinethione

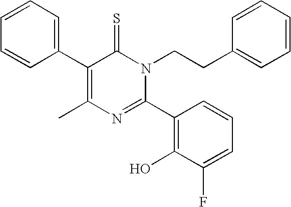

### a. 3-Oxo-2-phenyl-N-(2-phenylethyl)butanamide

To a solution of ethyl 3-oxo-2-phenylbutanoate (5 g, 0.24 moles) in DME (21 mL) was added 2-thiophenethylamine (2.92 g, 0.023 mol) in a microwave reaction vessel. Few drops of ethanol was added to the reaction mixture and irradiated to 180° C. for 1200 s. The reaction mixture was diluted with EtOAc and washed with 1N HCl. Organic layer was separated and dried over Na₂SO₄. Filtered, concentrated and purified by chromatography on silica gel to afford pure amide (3.42 g) in 49% yield.

### b. (1Z)-1-Methyl-3-oxo-2-phenyl-3-[(2-phenylethyl)amino]-1-propen-1-yl trifluoromethanesulfonate

To a solution of 3-oxo-2-phenyl-N-(2-phenylethyl)butanamide (17.26 g, 0.061 mol) in dry dichloromethane was cooled to −78° C. To this was added trifluoromethanesulfonic anhydride (12.36 mL, 0.073 mol) and triethyl amine (12.80 mL, 0.092 mol) sequentially and stirred while reaction warmed to 0° C. The reaction was concentrated and purified by chromatography on silica gel (Biotage, 0-40% ethyl acetate/hexane) to afford the trfilate (14.3 g) in 56% yield.

### c. 3-Fluoro-N-{(1Z)-1-methyl-3-oxo-2-phenyl-3-[(2-phenylethyl)amino]-1-propen-1-yl}-2-(methyloxy)benzamide

To a solution of (1Z)-1-methyl-3-oxo-2-phenyl-3-[(2-phenylethyl)amino]-1-propen-1-yl trifluoromethanesulfonate (13.2 g, 32 mmol) in dry deoxygenated dioxane was added 3-fluoro-2-hydroxybenzamide (5.49 g, 35 mmol), cesium carbonate (14.7 g, 45 mol), Pd₂(dba)₃ (0.74 g, 0.081 mmol) and xantophos (1.40 g, 2.4 mmol). The reaction was heated to reflux for 16 h. The cooled reaction mixture was filtered through a bed of celite and concentrated. Purification was purified by chromatography on silica gel (Biotage) to provide enamide (7.56 g) in 56% yield.

### d. 2-(3-Fluoro-2-hydroxyphenyl)-6-methyl-5-phenyl-3-(2-phenylethyl)-4(3H)-pyrimidinone

The 3-fluoro-N-((1Z)-1-methyl-3-oxo-2-phenyl-3-{[2-(2-thienyl)ethyl]amino}-1-propen-1-yl)-2-(methyloxy)benzamide (7.56 g, 0.018 mol) was dissolved in ethanol (100 mL). To this was added 20 mL of 25% (w/v) aqueous potassium hydroxide and refluxed for 16 h. The crude reaction mixture was acidified by 6N HCl to pH ˜1 and extracted with dichloromethane. The combined organic layers were washed with brine and concentrated. The crude residue was purified by chromatography on silica gel (Biotage) followed by recrystallization from EtOAc provided the desired product (6.3 g) in 88% yield. MS (m/z): 401.2 [M+H]⁺. ¹H NMR (400 MHz, CDCl₃) δ ppm 2.29 (s, 3H), 3.01 (t, J=7.8 Hz, 2H), 4.28 (t, J=7.8 Hz, 2H), 6.94-7.09 (m, 4H), 7.11-7.39 (m, 4H), 7.41-7.51 (m, 5H).

### e. 2-(3-Fluoro-2-hydroxyphenyl)-6-methyl-5-phenyl-3-(2-phenylethyl)-4(3H)-pyrimidinethione

The title compound is prepared as according to the procedure outlined in Example 47 except substituting 2-(3-fluoro-2-hydroxyphenyl)-6-methyl-5-phenyl-3-(2-phenylethyl)-4(3H)-pyrimidinone for 2-(3-fluoro-2-hydroxyphenyl)-6-methyl-3-(2-phenylethyl)-5-propyl-4(3H)-pyrimidinone. MS (m/z): 417.2 [M+H]⁺.

### Example 43

### Preparation of 2-(3-Fluoro-2-hydroxyphenyl)-6-methyl-5-(2-methylpropyl)-3-(2-phenylethyl)-4(3H)-pyrimidinethione

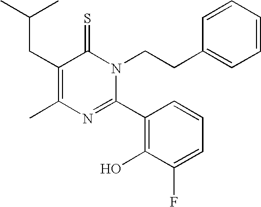

### a. 2-Acetyl-4-methylpentanoate

To a suspension of NaOMe (12.78 g, 0.24 mol) in dry methanol (430 mL) was added methyl acetoacetate (25 g, 0.22 mol) and stirred for 15 minutes and heated to gentle reflux. 1-bromo-2-methylpropane (29.5 g, 0.22 mol) added in portions within two hours and heated continued overnight. The reaction was concentrated and dilute with NH₄Cl and extracted with diethylether. The ether layer was dried and concentrated. The residue was purified by flash column chromatography (10% EtOAc/hexanes) to provide 2 g (5%) of the title compound.

### b. 2-{3-Fluoro-2-[(phenylmethyl)oxy]phenyl}-6-methyl-5-(2-methylpropyl)-4(1H)-pyrimidinone

To a solution of sodium methoxide (3.08 g) was added 3-fluoro-2-[(phenylmethyl)oxy]benzenecarboximidamide (3.95 g, 1.6 mmol). This mixture was maintained at room temperature for 15 minutes whereupon methyl 2-acetyl-4-methylpentanoate (2.23 g, 13 mmol) was added. This mixture was refluxed overnight whereupon it was cooled to room temperature and quenched with NH4Cl. The residue was diluted with EtOAc and washed with brine. The aqueous layer was reextracted with EtOAc and the combined organic layers were dried, filtered and concentrated. The residue was purified by flash column chromatography (30% EtOAc/hexanes) to provide 0.9 g (19%) of the title compound.

### c. 2-{3-fluoro-2-[(phenylmethyl)oxy]phenyl}-6-methyl-5-(2-methylpropyl)-3-(2-phenylethyl)-4(3H)-pyrimidinone

To a solution of 2-{3-fluoro-2-[(phenylmethyl)oxy]phenyl}-6-methyl-5-(2-methylpropyl)-4(1H)-pyrimidinone (0.9 g, 2.46 mmol) in DMF (25 mL) was added lithium hydride (0.039 g, 4.91 mmol) and lithium bromide (0.64 g, 7.37 mmol). This mixture was stirred at room temperature for 15 minutes whereupon phenethylbromide (2.27 g, 12.3 mmol) was added. This mixture was maintained at room temperature for 12 hours whereupon it was diluted with EtOAc, washed with brine (3×'s) and concentrated. Column chromatography of the residue (25% EtOAc/hexanes) provided 0.323 g (28%) of the title compound.

### d. 2-(3-Fluoro-2-hydroxyphenyl)-6-methyl-5-(2-methylpropyl)-3-(2-phenylethyl)-4(3H)-pyrimidinone

To a 0° C. solution of 2-{3-fluoro-2-[(phenylmethyl)oxy]phenyl}-6-methyl-5-(2-methylpropyl)-3-(2-phenylethyl)-4(3H)-pyrimidinone (0.323 g, 0.68 mmol) in was added BBr₃ (2.0 mL of 1 M DCM solution, 2.06 mmol). This mixture was allowed to warm to room temperature overnight whereupon methanol was added and the mixture concentrated. Column chromatography of the residue (0-30% EtOAc/hexanes) provided 0.22 g (85%) of the title compound. MS (EI) 381.2 (M+H)⁺.

### e. 2-(3-Fluoro-2-hydroxyphenyl)-6-methyl-5-(2-methylpropyl)-3-(2-phenylethyl)-4(3H)-pyrimidinethione

The title compound was prepared as described according to the procedure outlined in Example 45 except substituting 2-(3-fluoro-2-hydroxyphenyl)-6-methyl-5-(2-methylpropyl)-3-(2-phenylethyl)-4(3H)-pyrimidinone for 2-(3-fluoro-2-hydroxyphenyl)-6-methyl-3-(2-phenylethyl)-5-propyl-4(3H)-pyrimidinone: MS (m/z): 397.2 [M+H]⁺.

### Example 44

### Preparation of 3-(2,3-Dihydro-1H-inden-2-yl)-2-(2-hydroxyphenyl)-6-methyl-5-(1-methylethyl)-4(3H)-pyrimidinone

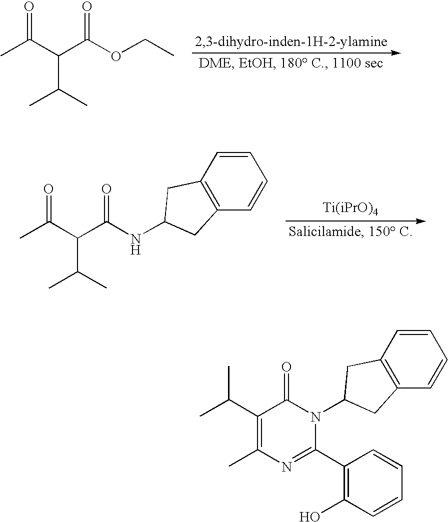

### a. 2-Acetyl-N-(2,3-dihydro-1H-inden-2-yl)-3-methylbutanamide

The title compound was prepared using procedures outlined in Example 1 except substituting ethyl 2-acetyl-3-methylbutanoate for ethyl 2-acetyl-4-methyl-4-pentenoate and phenethylamine for 2,3-dihydro-inden-1H-2-ylamine in step 11b.

### b. 3-(2,3-Dihydro-1H-inden-2-yl)-2-(2-hydroxyphenyl)-6-methyl-5-(1-methylethyl)-4(3H)-pyrimidinone

The 2-acetyl-N-(2,3-dihydro-1H-inden-2-yl)-3-methylbutanamide (1.2 g, 0.0046 moles) was placed in 100 mL round bottom flask. To this was added titanium isopropoxide (13.62 mL). While the reaction is stirring salicylamide (0.956 g, 0.069 moles) was added, a condenser was placed and the reaction was heated to reflux (oil bath temperature=150° C.). Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude solid was purified by reverse phase HPLC to afford the pure product. MS (m/z) 361.2 [M+H]⁺.

### Example 45

### Preparation of 5,6-Diethyl-2-(3-fluoro-2-hydroxyphenyl)-3-[2-(2-fluorophenyl)ethyl]-4(3H)-pyrimidinone

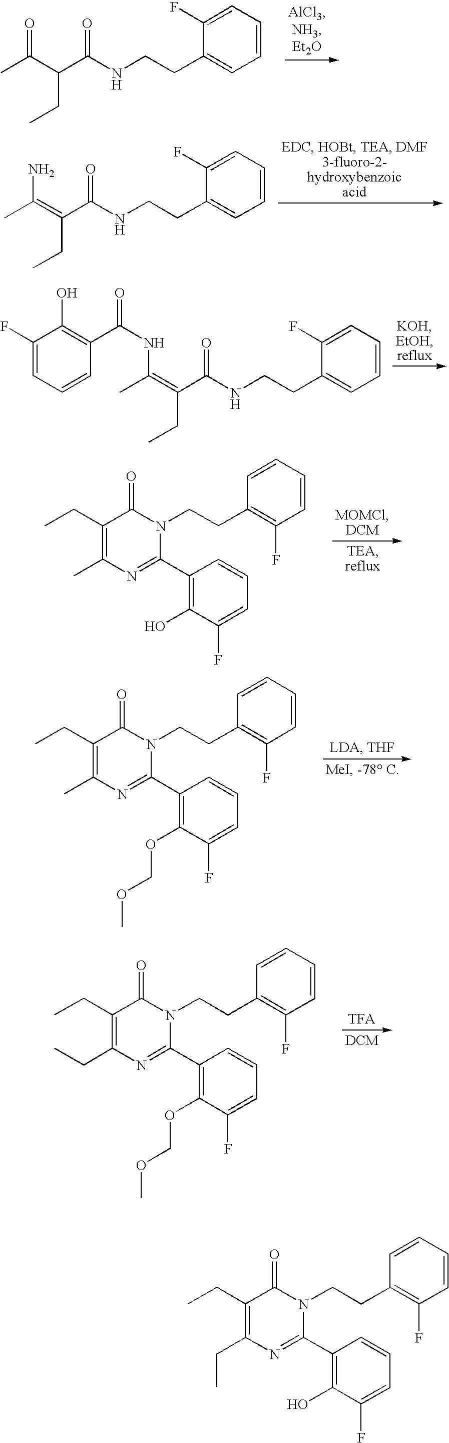

### a. (2Z)-3-Amino-2-ethyl-N-[2-(2-fluorophenyl)ethyl]-2-butenamide

The title compound was prepared following the procedure outlined in Example 30 except substituting 2-fluorophenethylamine for 3-fluorophenethylamine in step 30a

### b. 3-Fluoro-N-[(1Z)-2-({[2-(2-fluorophenyl)ethyl]amino}carbonyl)-1-methyl-1-buten-1-yl]-2-hydroxybenzamide

To a solution of (2Z)-3-amino-2-ethyl-N-[2-(2-fluorophenyl)ethyl]-2-butenamide (4.48 g, 0.0179 mol) and 3-fluoro-2-hydroxybenzoic acid (5.60 g, 0.038 mol) in dry THF was added EDC (4.13 g, 0.022 mol), HOBt (2.91 g, 0.022 mol) and TEA (0.8 mL) sequentially. The reaction was stirred at ambient temperature for 48 h. The reaction was diluted with EtOAc and washed with dilute HCl, 5% NaHCO₃ and brine. The organic layer was separated dried over Na₂SO₄, filtered and concentrated. The crude product was purified by flash column chromatography (30% EtOAc/Hexane) to produce the desired product (4.0 g) in 57% yield.

### c. 5-Ethyl-2-(3-fluoro-2-hydroxyphenyl)-3-[2-(2-fluorophenyl)ethyl]-6-methyl-4(3H)-pyrimidinone

3-Fluoro-N-[(1Z)-2-({[2-(2-fluorophenyl)ethyl]amino}carbonyl)-1-methyl-1-buten-1-yl]-2-hydroxybenzamide (4.00 g, 0.01 moles) was taken up in ethanol (60 mL) and 50 mL of 25% KOH was added and the reaction refluxed overnight. After reaction was cooled to RT the pH is adjusted to ˜1 with 3N HCl and extracted with dichloromethane. The combined organic layers were dried over Na₂SO₄, filtered and concentrated. The crude product was purified by flash column chromatography (30% EtOAc/Hexane) to produce the desired product (1.37 g) in 36% yield.

### d. 5-Ethyl-2-(3-fluoro-2-{[(methyloxy)methyl]oxy}phenyl)-3-[2-(2-fluorophenyl)ethyl]-6-methyl-4(3H)-pyrimidinone

To a solution of 5-ethyl-2-(3-fluoro-2-hydroxyphenyl)-3-[2-(2-fluorophenyl)ethyl]-6-methyl-4(3H)-pyrimidinone (1.37 g, 3.7 mmoles) in dry dichloromethane was added MOMCl (0.28 mL, 4.1 mmoles) and TEA (0.57 mL, 4.1 mmoles) and refluxed overnight. The reaction mixture was diluted with EtOAc and washed with dilute HCl and brine. The organic layer was dried over Na₂SO₄, filtered and concentrated. The residue was purified by flash column chromatography using 30% EtOAc in hexanes to provide the product (1.28 g) in 84% yield.

### e. 5,6-Diethyl-2-(3-fluoro-2-{[(methyloxy)methyl]oxy}phenyl)-3-[2-(2-fluorophenyl)ethyl]-4(3H)-pyrimidinone

To a −78° C. solution of 5-ethyl-2-(3-fluoro-2-{[(methyloxy)methyl]oxy}phenyl)-3-[2-(2-fluorophenyl)ethyl]-6-methyl-4(3H)-pyrimidinone (0.2 g, 0.48 mmoles) in THF was added 2M LDA (0.25 mL) in THF, hexane and ethyl benzene solvent mixture and the reaction stirred for 1 h. Iodomethane (0.03 mL) was added and the reaction stirred until starting material is all consumed. The reaction was quenched by NH₄Cl, extracted with EtOAc. The organic layer was washed with brine, dried over Na₂SO₄, filtered and concentrated. The residue was purified by flash column chromatography (20% EtOAc/hexane) to provide the product (0.08 g) in 43% yield.

### f. 5,6-Diethyl-2-(3-fluoro-2-hydroxyphenyl)-3-[2-(2-fluorophenyl)ethyl]-4(3H)-pyrimidinone

To a solution of 5,6-diethyl-2-(3-fluoro-2-{[(methyloxy)methyl]oxy}phenyl)-3-[2-(2-fluorophenyl)ethyl]-4(3H)-pyrimidinone (0.08 g, 0.18 mmoles) in dichloromethane at 0° C. was added TFA (0.3 mL, 9.3 mmoles) and reaction stirred for 1 h. The reaction mixture was diluted with EtOAc and washed with NaHCO₃ and brine. The EtOAc layer was dried over Na₂SO₄, filtered and concentrated. The residue was purified by flash column chromatography (30% EtOAc/hexane) to give product (0.05 g) in 73% yield. MS (m/z: 385.0 [M+H]⁺.

### Example 46

### Preparation of 6-(2-Cyclohexylethyl)-5-ethyl-2-(3-fluoro-2-hydroxyphenyl)-3-[2-(2-fluorophenyl ethyl]-4(3H)-pyrimidinone

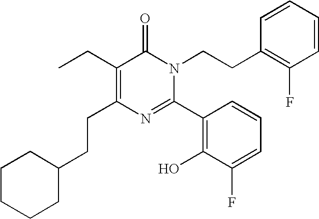

The title compound was prepared according to the procedures of Example 45 except substituting cyclohexylmethyl bromide for iodomethane in step 45e: MS (m/z): 467.4 [M+H]⁺.

### Example 47

### Preparation of 6-[2-(3,4-Dichlorophenyl)ethyl]-5-ethyl-2-(3-fluoro-2-hydroxyphenyl)-3-[2-(2-fluorophenyl)ethyl]-4(3H)-pyrimidinone

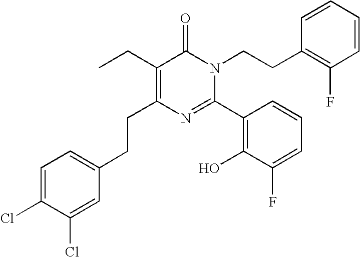

The title compound was prepared according to the procedures of Example 45 except substituting 3,4-dichlorobenzylbromide for iodomethane step 45e: MS (m/z): 529.4 [M+H]⁺.

### Example 48

### Preparation of 2-(3-Fluoro-2-hydroxyphenyl)-3-[2-(2-fluorophenyl ethyl]-6-methyl-5-(2-methylpropyl)-4(3H)-pyrimidinone

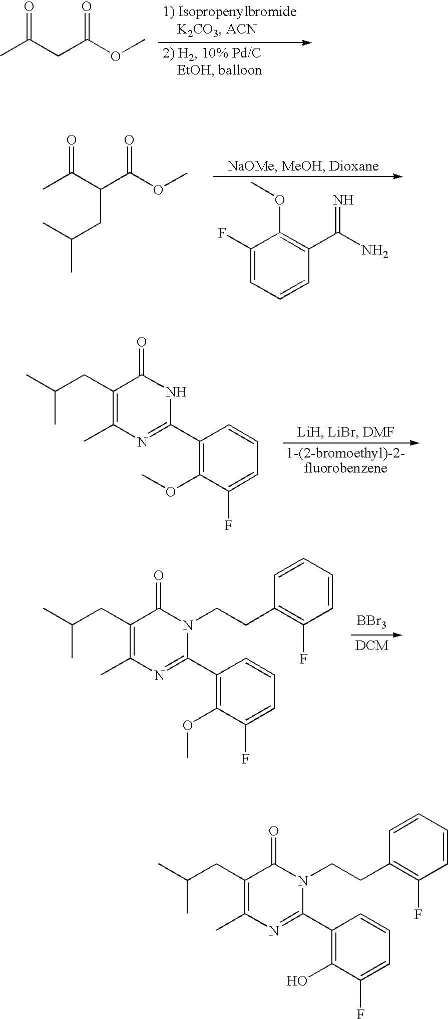

### a. Methyl 2-acetyl-4-methylpentanoate

3-Bromo-2-methyl-1-propene (6.75 g, 0-05 moles) and potassium carbonate (4.84 g, 0.035 mol) were added to a stirred solution of methyl acetoacetate in ACN (500 mL). The resulted heterogeneous mixture was stirred for 4 days and the solid was removed by filteration. Et₂O was added and washed with H₂O and brine. Organic layer was dried (Na₂SO₄), filtered and concentrated. The crude residue was purified by flash chromatography (10% EtOAc/hexanes) to produce the product (4.29 g). Subsequent catalytic hydrogenolysis produced the product.

### b. 2-[3-Fluoro-2-(methyloxy)phenyl]-6-methyl-5-(2-methylpropyl)-4(1H)-pyrimidinone

NaOMe (3.58 g, 0.066 mol) was added to a 0° C. solution of 3-fluoro-2-(methyloxy)benzenecarboximidamide (5.07 g, 0.03 mol) and methyl 2-acetyl-4-methylpentanoate (6.23 g, 0.036 mol) in methanol (75 mL) and 1,4-dioxane (15 mL). The resulting mixture was refluxed overnight. The solvents were removed and the residue was quenched with NH₄Cl and EtOAc. The layers were separated and the aqueous layer was extracted with dichlormethane 3 times. The combined organic portions were dried over Na₂SO₄ and purified by flash column chromatography (30% EtOAc/hexanes to give 3.46 g of product in 40% yield.

### c. 2-[3-Fluoro-2-(methyloxy)phenyl]-3-[2-(2-fluorophenyl)ethyl]-6-methyl-5-(2 methylpropyl)-4(3H)-pyrimidinone

To a solution of 2-[3-fluoro-2-(methyloxy)phenyl]-6-methyl-5-(2-methylpropyl)-4(1H)-pyrimidinone (0.80 g, 0.0027 mol) in dry DMF was added LiH (0.044 g, 0.0055 mol), LiBr (0.72 g, 0.0083 mol) and stirred for 10 min at room temperature. Then 2-fluorophenethylbromide (1.68 g, 0.0083 mol) was added and stirred overnight. The reaction mixture was quenched by addition of ice and 6N HCl. This mixture was extracted with EtOAc and the organic layer was washed with aqueous NaHCO₃, brine and dried over Na₂SO₄. The sodium sulfate was filtered and concentrated. The crude product is purified by flash column chromatography (25% ethyl acetate/hexane) to give product (0.207 g) in 18% yield.

### d. 2-(3-Fluoro-2-hydroxyphenyl)-3-[2-(2-fluorophenyl)ethyl]-6-methyl-5-(2-methylpropyl)-4(3H)-pyrimidinone

2-[3-Fluoro-2-(methyloxy)phenyl]-3-[2-(2-fluorophenyl)ethyl]-6-methyl-5-(2-methylpropyl)-4(3H)-pyrimidinone (0.274 g, 0.67 mmol) in 2.0 mL of dichloromethane was cooled to 0° C. 1M DCM solution of BBr₃ (3.0 mL, 0.33 mmol) was then added and the reaction mixture warmed to RT and stirred for 12 h. The reaction mixture was diluted with dichloromethane and aqueous NaHCO₃ was then added. The organic layer was separated and washed with H₂O, brine and dried over Na₂SO₄, filtered, concentrated and purified by chromatography on silica gel (Biotage, 25% ethyl acetate/hexane) to afford pure compound (0.221 g) in 82% yield. MS (m/z): 399.2 [M+H]⁺.

### Example 49

### Preparation of 2-(3-Fluoro-2-hydroxyphenyl)-6-methyl-5-(2-methylpropyl)-3-[2-(2-thienyl)ethyl]-4(3H)-pyrimidinone

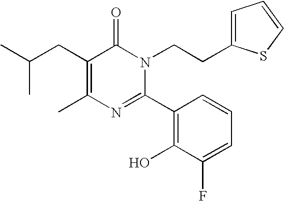

The title compound was prepared according to the procedures of Example 48 except substituting 2-(2-bromoethyl)thiophene for 1-(2-bromoethyl)-2-fluorobenzene in step 48c. MS (m/z); 387.4 [M+H]⁺.

### Example 50

### Preparation of 2-(3-Fluoro-2-hydroxyphenyl)-3-[2-(4-fluorophenyl)ethyl]-6-methyl-5-(2-methylpropyl)-4(3H)-pyrimidinone

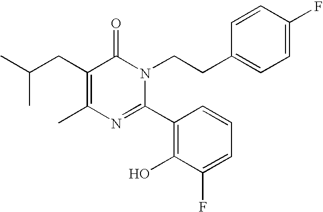

The title compound was prepared according to the procedures of Example 48 except substituting by substituting 1-(2-bromoethyl)-4-fluorobenzene for 1-(2-bromoethyl)-2-fluorobenzene in step 48c. MS (m/z): 399.2 [M+H]⁺.

### Example 51

### Preparation of 2-(3-Fluoro-2-hydroxyphenyl)-3-[2-(3-fluorophenyl)ethyl]-6-methyl-5-(2-methylpropyl)-4(3H)-pyrimidinone

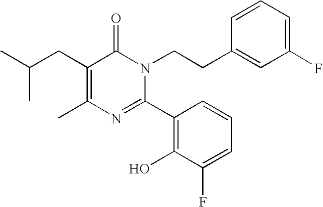

The title compound was prepared according to the procedures of Example 48 except substituting by substituting 1-(2-bromoethyl)-3-fluorobenzene for 1-(2-bromoethyl)-2-fluorobenzene in step 48c. MS (m/z): 399.2 [M+H]⁺.

### Example 52

### Preparation of 2-(2-Hydroxyphenyl)-7-methyl-3-(2-phenylethyl)-3,5,6,7,8,9-hexahydro-4H-pyrimido[4,5-d]azepin-4-one

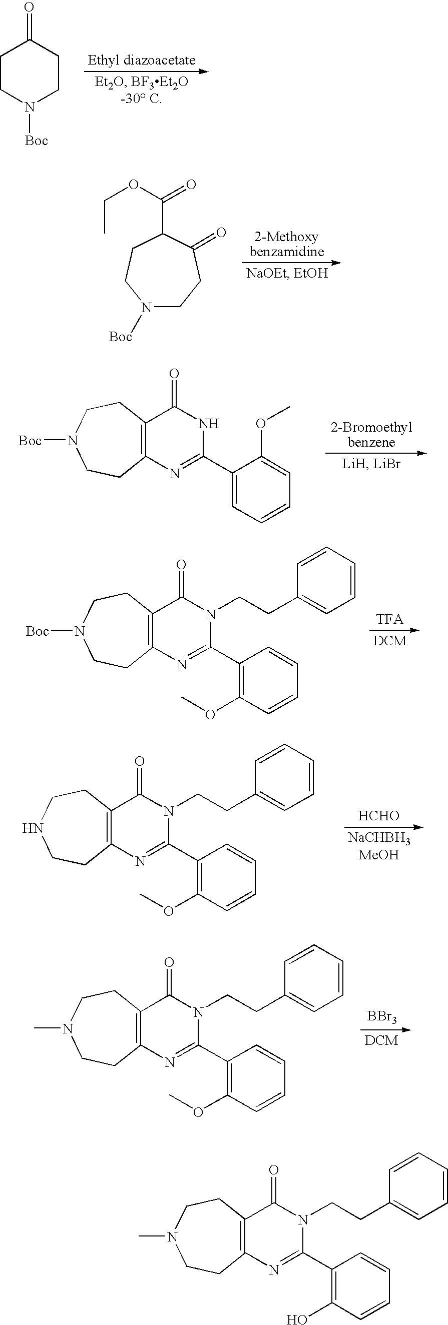

### a. 1-(1,1-Dimethylethyl) 4-ethyl 5-oxohexahydro-1H-azepine-1,4-dicarboxylate

Synthesis was accomplished as reported in the literature (Synth. Commun., 1992, 22(9), 1249-1258).

### b. 1,1-Dimethylethyl 2-[2-(methyloxy)phenyl]-4-oxo-1,4,5,6,8,9-hexahydro-7H-pyrimido[4,5-d]azepine-7-carboxylate

NaOMe (0.98 g, 0.018 mol) was added to a 0° C. solution of 2-(methoxy)benzenecarboxamidine (1.36 g, 0.0091 mol) and 1-(1,1-dimethylethyl) 4-ethyl 5-oxohexahydro-1H-azepine-1,4-dicarboxylate (2.58 g, 0.0091 mol) in methanol (45 mL) and 1,4-dioxane (45 mL). The resulting mixture was refluxed overnight. The solvents were removed and the residue was quenched with NH₄Cl and EtOAc. The layers were separated and the aqueous layer was extracted with dichlormethane 3 times. The combined organic portions were dried over Na₂SO₄ and purified by flash column chromatography to give 2.75 g of product in 81% yield.

### c. 1,1-Dimethylethyl 2-[2-(methyloxy)phenyl]-4-oxo-3-(2-phenylethyl)-3,4,5,6,8,9-hexahydro-7H-pyrimido[4,5-d]azepine-7-carboxylate

To a solution of 1,1-Dimethylethyl 2-[2-(methyloxy)phenyl]-4-oxo-1,4,5,6,8,9-hexahydro-7H-pyrimido[4,5-d]azepine-7-carboxylate (2.75 g, 0.0074 mol) in dry DMF was added LiH (0.118 g, 0.015 mol), LiBr (1.93 g, 0.022 mol) and stirred for 10 min at room temperature. Then (2-bromoethyl)benzene (6.85 g, 0.037 mol) was added and stirred overnight. The reaction mixture was quenched by addition of ice and 6N HCl. This mixture was extracted with EtOAc and the organic layer was washed with aqueous NaHCO₃, brine and dried over Na₂SO₄. The sodium sulfate was filtered and concentrated. The crude product is purified by flash column chromatography (30% ethyl acetate/hexane) to give product (2.15 g) in 61% yield.

### d. 2-[2-(Methyloxy)phenyl]-3-(2-phenylethyl)-3,5,6,7,8,9-hexa hydro-4H-pyrimido[4,5-d]azepin-4-one

1,1-Dimethylethyl 2-[2-(methyloxy)phenyl]-4-oxo-3-(2-phenylethyl)-3,4,5,6,8,9-hexahydro-7H-pyrimido[4,5-d]azepine-7-carboxylate (2.33 g, 4.9 mmoles) was taken in dichloromethane and to this was added trifluoroacetic acid (5.58 g, 49 mmoles). The reaction was neutralized by aqueous NaOH and extracted with dichloromethane. The dichloromethane layer was washed with brine and dried over sodium sulfate. The reaction mixture was filtered and concentrated in vacuo to produce free amine (1.79 g, 97%).

### e. 2-(2-Hydroxyphenyl)-7-methyl-3-(2-phenylethyl)-3,5,6,7,8,9-hexahydro-4H-pyrimido[4,5-d]azepin-4-one

To a solution of 2-[2-(methyloxy)phenyl]-3-(2-phenylethyl)-3,5,6,7,8,9-hexahydro-4H-pyrimido[4,5-d]azepin-4-one (1.0 g, 2 mmoles) in methanol at 0° C. was added formaldehyde (2.3 mL, 30 mmoles) and sodium cyanoborohydride (0.39 g, 6 mmoles). The reaction mixture was stirred overnight. The reaction was quenched with water, extracted with dichloromethane and the dichloromethane was dried over sodium sulfate. Filtered, concentrated in vacuo and the residue was purified by flash chromatography (0-30% ethyl acetate/hexane) to afford the product (0.4 g, 50%). Subsequent demethylation using BBr₃ described previously produced the title compound: MS (m/z): 376.4 [M+H]⁺.

### Example 53

### Preparation of 7-acetyl-2-(2-hydroxyphenyl)-3-(2-phenylethyl)-3,5,6,7,8,9-hexahydro-4H-pyrimido[4,5-d]azepin-4-one

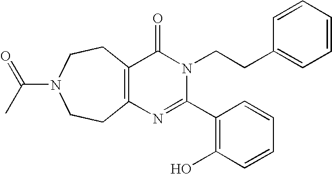

To a solution of 2-[2-(methyloxy)phenyl]-3-(2-phenylethyl)-3,5,6,7,8,9-hexahydro-4H-pyrimido[4,5-d]azepin-4-one (0.94 g, 1.9 mmoles) of Example 52 in dichloromethane were added acetyl chloride (0.45 g, 5.8 mmoles) and triethylamine (0.58 g, 5.8 mmoles). The reaction mixture stirred until all the starting material is consumed. The reaction was quenched with saturated sodium carbonate. dichloromethane layer was washed with brine and dried over sodium sulfate. Filtered, concentrated in vacuo and the residue was purified by flash chromatography (0-30% ethyl acetate/hexane) to afford the product (0.27, 34%). Subsequent demethylation using BBr₃ described previously produced the title compound (0.18 g, 69%). MS (m/z): 404.2 [M+H]⁺.

### Example 54

### Preparation of 2-(2-Hydroxyphenyl)-7-(methylsulfonyl)-3-(2-phenylethyl)-3,5,6,7,8,9-hexahydro-4H-pyrimido[4,5-d]azepin-4-one

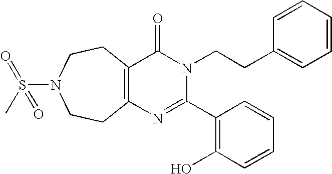

The title compound was prepared according to the procedure of Example 53 except substituting methylsulfonyl chloride for acetyl chloride: MS (m/z): 440.2 [M+H]⁺.

### Example 55

### Preparation of 5-Bromo-2-(2-hydroxyphenyl)-6-methyl-3-(2-phenylethyl)-4(3H)-pyrimidinone

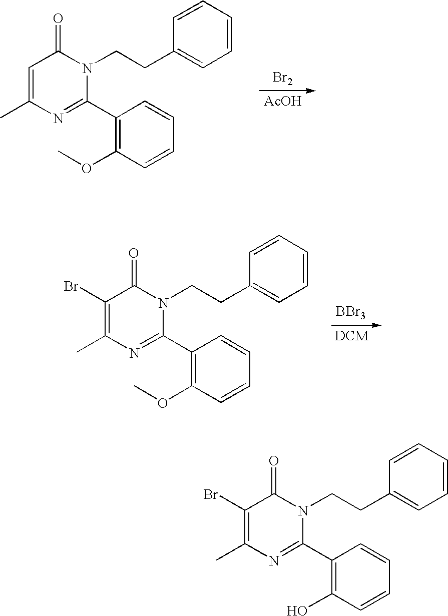

To a solution of 6-methyl-2-[2-(methyloxy)phenyl]-3-(2-phenylethyl)-4(3H)-pyrimidinone (0.16 g, 0.5 mmoles) in 50 mL acetic acid was added bromine (0.08 g, 0.5 mmoles) dropwise. The reaction was quenched with saturated sodium carbonate and adjusted pH to ˜8 at 0° C. The reaction mixture was extracted with dichloromethane and combined organic layers were washed with brine and dried over sodium sulfate. The organic layer was filtered, concentrated in vacuo and the residue was purified by flash chromatography (0-20% ethyl acetate/hexane) to afford the product (0.2 g) in 98% yield. Subsequent demethylation using BBr₃ as described previously produced the title compound (0.15 g) in 79% yield. MS (m/z): 384.8 [M+H]⁺.

### Example 56

### Preparation of 2-(2-Hydroxyphenyl-5-iodo-6-methyl-3-(2-phenylethyl)-4(3H)-pyrimidinone

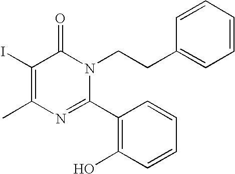

6-Methyl-2-[2-(methyloxy)phenyl]-3-(2-phenylethyl)-4(3H)-pyrimidinone (0.21 g, 0.66 mmoles) was taken up in glacial acetic acid (13 mL). To this was added 1M dichloromethane solution of iodine monochloride (0.72 mL, 0.72 mmoles) and the reaction was stirred for 16 h. Ethyl acetate was added and acetic acid was washed with saturated sodium carbonate. The organic layer dried over sodium sulfate. Sodium sulfate was filtered off and organic layer was concentrated. The crude product was purified by chromatography on silica gel (Biotage) using ethyl acetate and hexane mixtures (20-50%) to obtain the desired product (0.058 g) in 20% yield. Deprotection using BBr₃ as detailed previously produced the title compound: MS (m/z): 433.0 [M+H]⁺.

### Example 57

### Preparation of 5-Chloro-3-(2-cyclohexylethyl)-2-(2-hydroxyphenyl)-6-methyl-4(3H)-pyrimidinone

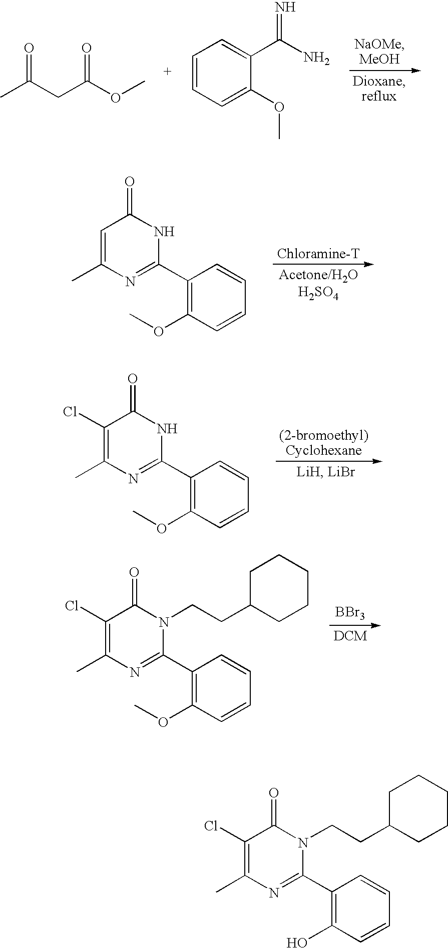

### a. 6-Methyl-2-[2-(methyloxy)phenyl]-4(1H)-pyrimidinone

NaOMe (3.95 g, 0.073 mol) was added to a 0° C. solution of 2-(methoxy)benzenecarboxamidine (5.49 g, 0.0366 mol) and methylacetoacetate (4.24 g, 0.0366 mol) in methanol (45 mL) and 1,4-dioxane (15 mL). The resulting mixture was refluxed overnight. The solvents were removed and the residue was diluted with H₂O and pH was adjusted to 8 with acetic acid. The layers were separated and the aqueous layer was extracted with dichlormethane 3 times. The combined organic portions were dried over Na₂SO₄ and purified by flash column chromatography to give 2.98 g of product.

### b. 5-Chloro-6-methyl-2-[2-(methyloxy)phenyl]-4(1H)-pyrimidinone

To a solution of 6-methyl-2-[2-(methyloxy)phenyl]-4(1H)-pyrimidinone (0.043 g, 0.2 mmoles) in 1:1 of acetone/water was added chloramine-T (0.045 g, 0.2 mmoles) and sulfuric acid (0.020 g, 0.2 mmoles). The reaction mixture was refluxed overnight. Upon cooling, the reaction mixture was diluted with ethyl acetate, washed with saturated sodium carbonate, brine, dried over sodium sulfate. Filtered, concentrated in vacuo and the residue purified by flash chromatography (0-50% ethyl acetate/hexane) to afford 5-chloro-6-methyl-2-[2-(methyloxy)phenyl]-4(1H)-pyrimidinone (0.017 g) in 34% yield.

### c. 5-Chloro-3-(2-cyclohexylethyl)-2-(2-hydroxyphenyl)-6-methyl-4(3H)-pyrimidinone

To a solution of chloro-6-methyl-2-[2-(methyloxy)phenyl]-4(1H)-pyrimidinone (0.42 g, 1.7 mmoles) in DMF were added lithium hydride (0.027 g, 3.4 mmoles), lithium bromide (0.436 g, 5.0 mmoles), and 2-cyclohexylethyl bromide (1.6 g, 8.4 mmoles). Upon stirring overnight at room temperature, the reaction was quenched with saturated ammonium chloride, extracted with ethyl acetate. The combined organic layers were washed with brine, dried over sodium sulfate, filtered, concentrated in vacuo and the residue purified by flash chromatography (0-30% ethyl acetate/hexane) to afford the desired product (0.23 g, 38%). Subsequent deprotection using BBr₃ was accomplished to produce the title compound (0.2 g, 90%). MS (m/z): 347.2 [M+H]⁺.

### Example 58

### Preparation of 5-Chloro-2-(2-hydroxyphenyl)-6-methyl-3-[2-(2-thienyl)ethyl]-4(3H)-pyrimidinone

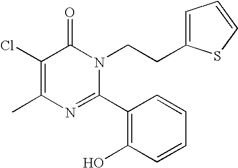

The title compound was prepared according to the procedure outlined in Example 57 except substituting 2-thiophenethyl bromide for 2-cyclohexylethyl bromide. MS (m/z): 347.2

### Example 59

### Preparation of 5-Chloro-2-(2-hydroxyphenyl)-6-methyl-3-(2-Phenylethyl)-4(3H)-pyrimidinethione

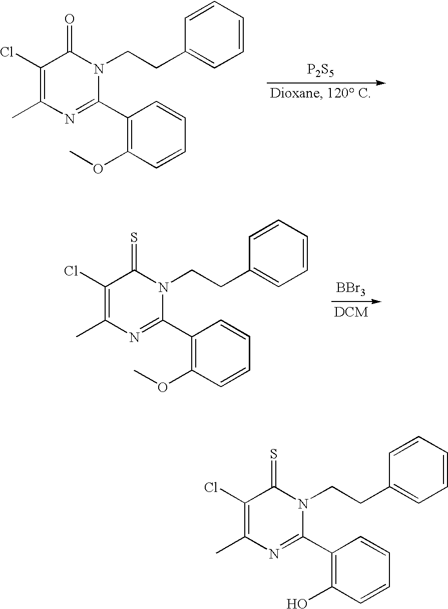

A mixture of 5-chloro-6-methyl-2-[2-(methyloxy)phenyl]-3-(2-phenylethyl)-4(3H)-pyrimidinone of Example 26c (0.36 g, 1.0 mmole) and phosphorus pentasulfide (0.73 g, 5 mmoles) in dioxane were heated in a sealed tube at 120° C. overnight. The mixture was concentrated in vacuo and the residue purified by flash chromatography (ethyl acetate/hexane=10-25%) to yield 0.16 g. Demethylation using BBr₃ (3 eq.) in dichloromethane as previously detailed yielded the title compound (0.083 g, 54%). MS (m/z): 357.2 [M+H]⁺.

### Example 60

### Preparation of 5-Bromo-2-(3-hydroxyphenyl)-6-methyl-3-(2-phenylethyl)-4(3H)-pyrimidinone

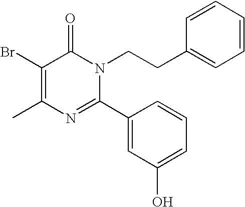

The title compound was prepared by the general procedures outlined in Example 11 and substituting 3-methoxybenzamide for 2-hydroxy-3-fluoro benzamide in step 11d. Subsequent deprotection with BBr₃ as previously described provided the product. MS (m/z): 386.0 [M+H]⁺.

### Example 61

### Preparation of 2-(3-Hydroxyphenyl)-6-methyl-5-phenyl-3-(2-phenylethyl)-4(3H)-pyrimidinone

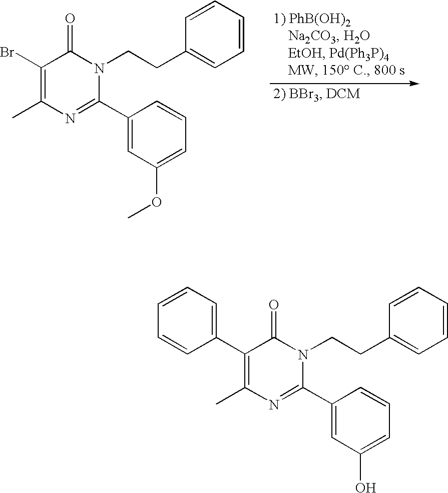

The title compound was prepared according to the procedure outlined in Example 13 except substituting phenylboronic acid for 6-quinolinylboronic acid and 5-bromo-6-methyl-2-[3-(methyloxy)phenyl]-3-(2-phenylethyl)-4(3H)-pyrimidinone for 5-bromo-2-(3-fluoro-2-[(phenylmethyl)oxy]phenyl)-6-methyl-3-(2-phenylethyl)-4(3H)-pyrimidinone. Subsequent deprotection with BBr₃ as previously described provided the product. MS (m/z): 383.2 [M+H]⁺.

### Example 62

### Preparation of 2-(2-hydroxyphenyl)-6-methyl-5-(phenylamino)-3-(2-phenylethyl)-4(3H)-pyrimidinone

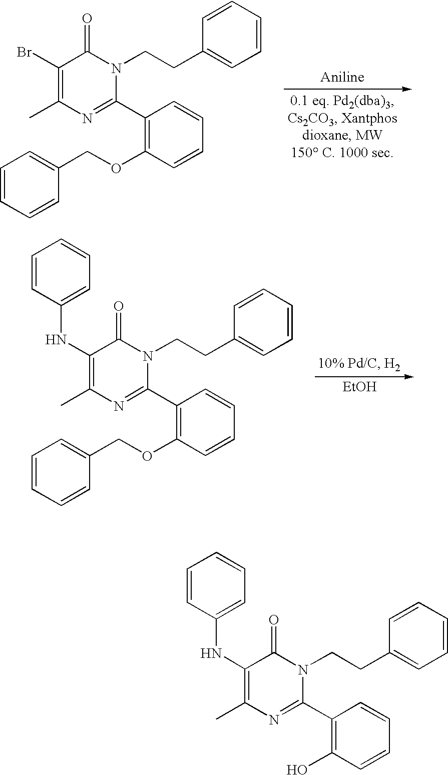

To a solution of 5-bromo-6-methyl-3-(2-phenylethyl)-2-{2-[(phenylmethyl)oxy]phenyl}-4(3H)-pyrimidinone (0.10 g, 0.21 mmoles) of Example 20 in dioxane (5 mL) was added aniline (0.027 g, 0.30 mmoles), xantphos (0.037 g, 0.06 mmoles) and cesium carbonate (0.096 g, 0.30 mmoles) in a microwave reaction vessel. After bubbling nitrogen for 10 min. tris(dibenzylideneacetone)dipalladium (0.019 g, 0.02 mmoles) was added. The mixture in the sealed vessel was irradiated to 150° C. for 1000 seconds. The reaction mixture was filtered through syringe filter (Acrodisc CR25 mm with 0.2 □m PTFE membrane). The vessel and filter were washed with ethyl acetate. And the combined organic layers were combined with filtrate and washed with brine, dried over sodium sulfate, filtered, concentrated in vacuo and the residue purified by flash chromatography (0-50% ethyl acetate/hexane) to afford the desired product (0.087 g, 85%). Debenzylation using palladium on active carbon as previously described provided the title compound (0.056 g, 79%): MS (m/z): 398.2 [M+H]⁺.

### Example 63

### Preparation of 5-(1-Azetidinyl)-2-(3-fluoro-2-hydroxyphenyl)-6-methyl-3-(2-phenylethyl)-4(3H)-pyrimidinone

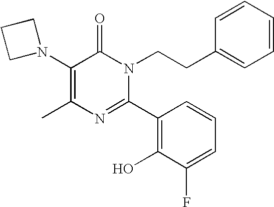

The title compound was prepared according to the procedure of Example 62 except substituting azetidine (10 eq.) for aniline. During the course of the reaction partial debenzylation occurred eliminating the subsequent hydrogenolysis step and afforded the title compound directly (0.1 g, 53%). MS (m/z): 380.2 (M+H).

### Example 64

### Preparation of 2-(3-Fluoro-2-hydroxyphenyl)-6-methyl-3-(2-phenylethyl)-5-(propylamino)-4(3H)-pyrimidinone

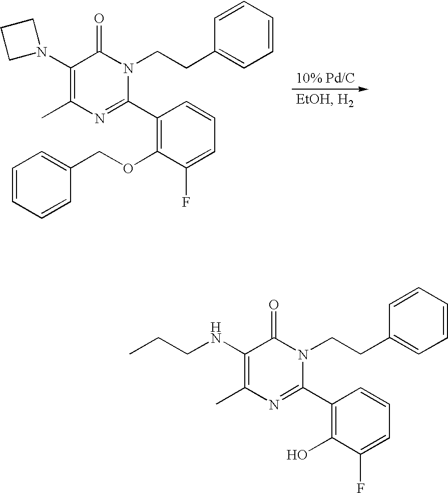

To a solution of 5-(1-azetidinyl)-2-{3-fluoro-2-[(phenylmethyl)oxy]phenyl}-6-methyl-3-(2-phenylethyl)-4(3H)-pyrimidinone (0.15 g, 0.32 mmoles) in ethanol was added 10% Pd/C (0.02 g). This mixture was placed under hydrogen atmosphere and stirred for 16 h. The reaction mixture was filtered through a bed of celite and concentrated and purified by chromatography on silica gel (Biotage) to afford the desired product. MS (m/z): 382.0 [M+H]

### Example 65

### Preparation of 2-(2-Fluoro-3-hydroxyphenyl)-6-methyl-5-phenyl-3-(2-phenylethyl)-4(3H)-pyrimidinone

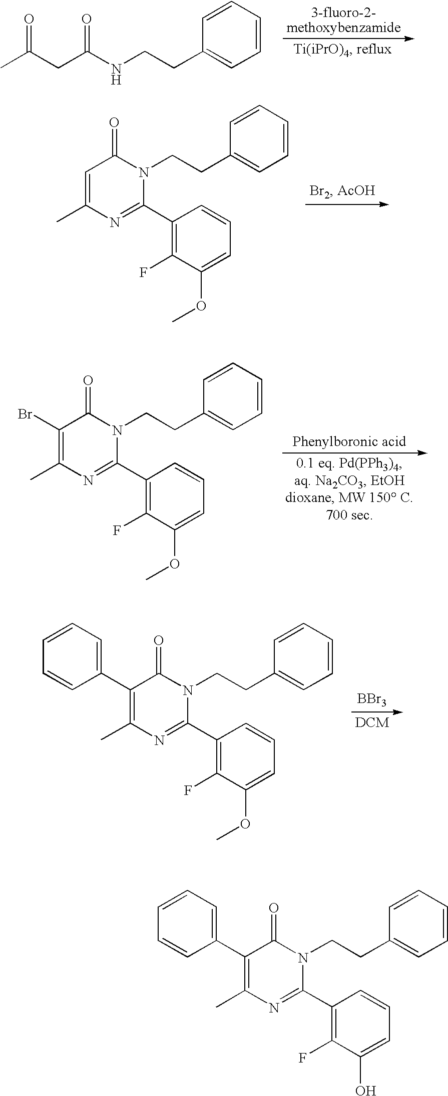

### a. 2-[2-Fluoro-3-(Methyloxy)phenyl]-6-methyl-3-(2-phenylethyl)-4(3H)-pyrimidinone

3-Oxo-N-(2-phenylethyl)butanamide (2.09 g, 0.01 mmol) was taken up in dry xylene (38 mL). To this was added 2-fluoro-3-(methyloxy)benzamide (2.58 g, 0.015 mmol) and titanium isopropoxide (0.15 mol) sequentially. The reaction was heated to reflux until all the starting material was consumed. The reaction mixture was concentrated and diluted with dichloromethane and washed with 3N HCl. The organic layer was separated and dried over Na₂SO₄ filtered, concentrated and purified by chromatography on silica gel (Biotage, 0-40% ethyl acetate/hexane) to afford 1.5 g of pure product.

### b. 5-Bromo-2-[2-fluoro-3-(methyloxy)phenyl]-6-methyl-3-(2-phenylethyl)-4(3H)-pyrimidinone

2-[2-Fluoro-3-(methyloxy)phenyl]-6-methyl-3-(2-phenylethyl)-4(3H)-pyrimidinone (1.5 g, 0.0044 moles) was taken up in glacial acetic acid. To this was added bromine (0.34 mL, 0.0066 moles) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was purified by chromatography on silica gel (Biotage) to obtain the desired product.

### c. 2-[2-fluoro-3-(methyloxy)phenyl]-6-methyl-5-phenyl-3-(2-phenylethyl)-4(3H)-pyrimidinone

To a solution of 5-bromo-2-[2-fluoro-3-(methyloxy)phenyl]-6-methyl-3-(2-phenylethyl)-4(3H)-pyrimidinone (0.75 g, 1.8 mmoles) in dioxane was added phenylboronic acid (0.44 g, 3.6 mmoles), 2 mL ethanol, and 2 mL aqueous sodium carbonate (0.38 g, 3.6 mmoles) in a microwave reaction vessel. After bubbling nitrogen for 10 min. tetrakis(triphenylphosphine)palladium (0.21 g, 0.18 mmoles) was added and the reaction was capped and irradiated to 150° C. for 700 s. The reaction mixture was filtered through syringe filter (Acrodisc CR25 mm with 0.2 □m PTFE membrane). The vessel and filter were washed with ethyl acetate. EtOAc combined with filtrate were washed with brine, separated, dried over sodium sulfate. Filtered, concentrated in vacuo and the residue was purified by flash chromatography (0-40% ethyl acetate/hexane) to afford the desired product (0.61 g, 82%).

### d. 2-(2-Fluoro-3-hydroxyphenyl)-6-methyl-5-phenyl-3-(2-phenylethyl)-4(3H)-pyrimidinone

2-[2-Fluoro-3-(methyloxy)phenyl]-6-methyl-5-phenyl-3-(2-phenylethyl)-4(3H)-pyrimidinone (0.81 g, 1.94 mmol) in dichloromethane was cooled to 0° C. BBr₃ (9.71 mmol) was then added and the reaction mixture warmed to RT and stirred for 12 h. The reaction mixture was diluted with dichloromethane and aqueous NaHCO₃ was then added. The organic layer was separated and washed with H₂O, brine and dried over Na₂SO₄, filtered, concentrated and purified by reverse phase HPLC (ACN/H₂O; 0.1% TFA) to afford pure product (0.64 g) in 89% yield. MS (m/z): 400.8 [M+H]⁺.

### Example 66

### Preparation of 2-(2-Hydroxyphenyl)-6-methyl-3-(2-phenylethyl)-5-(3-thienyl)-4(3H)-pyrimidinone

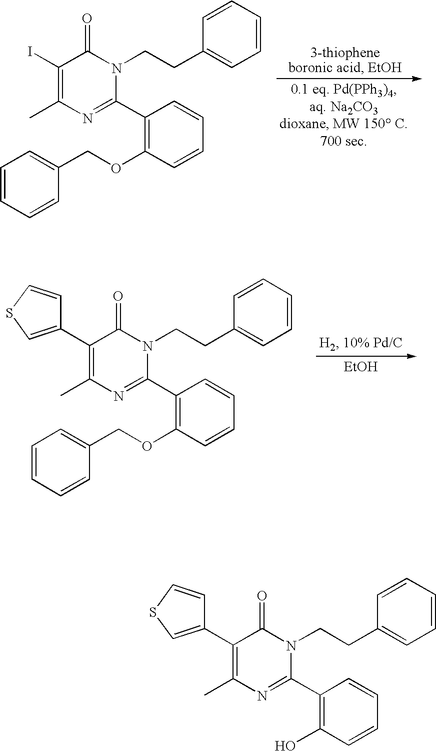

To a solution of 5-iodo-6-methyl-3-(2-phenylethyl)-2-{2-[(phenylmethyl)oxy]phenyl}-4(3H)-pyrimidinone (0.20 g, 0.38 mmoles) of Example 21 in dioxane (5 mL) was added thiophene-3-boronic acid (0.098 g, 0.76 mmoles), 0.5 mL ethanol, and 0.5 mL aqueous sodium carbonate (0.081 g, 0.76 mmoles) in a microwave reaction vessel. After 10 min. of deoxygenation, tetrakis(triphenylphosphine)palladium (0.044 g, 0.04 mmoles) was added. The mixture in sealed vessel was irradiated to 150° C. for 700 s. The reaction mixture was filtered through syringe filter (Acrodisc CR25 mm with 0.2 □m PTFE membrane). The vessel and filter were washed with ethyl acetate. The ethyl acetate layers were combined with filtrate and washed with brine, separated, dried over sodium sulfate, filtered, concentrated in vacuo and the residue purified by flash chromatography (0-40% ethyl acetate/hexane) to afford 6-methyl-3-(2-phenylethyl)-2-{2-[(phenylmethyl)oxy]phenyl}-5-(3-thienyl)-4(3H)-pyrimidinone (0.17 g, 93%). 6-methyl-3-(2-phenylethyl)-2-{2-[(phenylmethyl)oxy]phenyl}-5-(3-thienyl)-4(3H)-pyrimidinone was debenzylated using palladium on activated carbon under hydrogen atmosphere overnight to provide the title compound. MS (m/z): 389.4 [M+H]⁺.

### Example 67

### Preparation of 5-(3-Furanyl)-2-(2-hydroxyphenyl)-6-methyl-3-(2-phenylethyl)-4(3H)-pyrimidinone

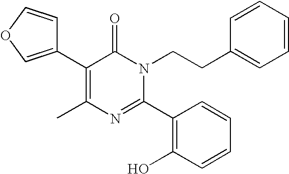

The title compound was prepared according to the procedures outlined in Example 66 except substituting 3-furanboronic acid for thiophene-3-boronic acid: MS (m/z): 373.0 [M+H]⁺.

### Example 68

### Preparation of 5-(4-Biphenyl)-2-(2-hydroxyphenyl)-6-methyl-3-(2-phenylethyl)-4(3H)-pyrimidinone

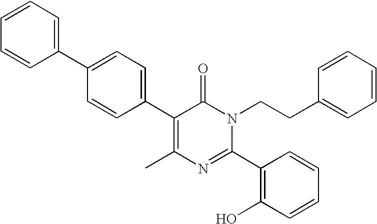

The title compound was prepared according to the procedures outlined in Example 66 except substituting 4-biphenylboronic acid for thiophene-3-boronic acid: MS (m/z): 459.2 [M+H]⁺.

### Example 69

### Preparation of 5-(1,3-Benzodioxol-5-yl)-2-(2-hydroxyphenyl)-6-methyl-3-(2-phenylethyl)-4(3H)-pyrimidinone

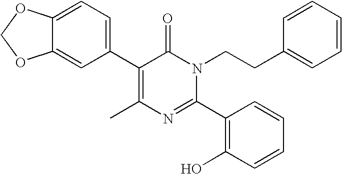

The title compound was prepared according to the procedures outlined in Example 66 except substituting 3,4-methylenedioxyphenylboronic acid for thiophene-3-boronic acid: MS (m/z): 427.2 [M+H]⁺.

### Example 70

### Preparation of 5-(2-fluorophenyl)-2-(2-hydroxyphenyl)-6-methyl-3-(2-phenylethyl)-4(3H)-pyrimidinone

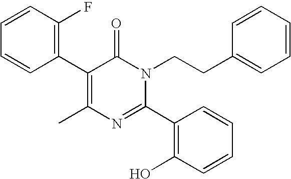

The title compound was prepared according to the procedures outlined in Example 66 except substituting 2-fluorophenylboronic acid for thiophene-3-boronic acid. MS (m/z): 401.2 [M+H]⁺.

### Example 71

### Preparation of 2-(2-Hydroxyphenyl)-6-methyl-3-(2-phenylethyl)-5-[4-(trifluoromethyl)phenyl]-4(3H)-pyrimidinone

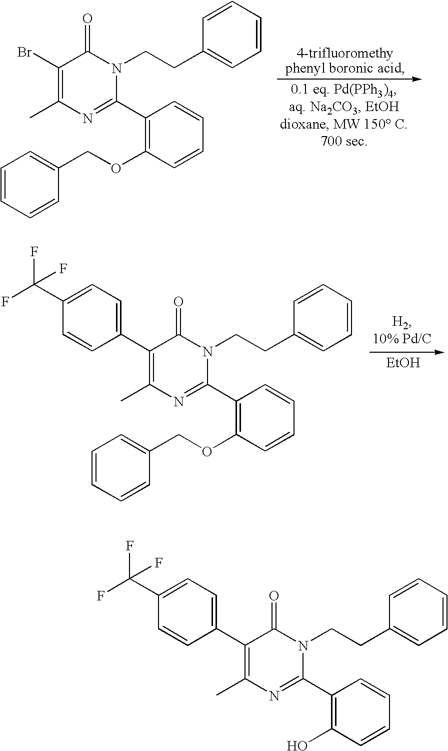

The title compound was prepared according to the procedures of Example 66 except substituting 5-bromo-6-methyl-3-(2-phenylethyl)-2-{2-[(phenylmethyl)oxy]phenyl}-4(3H)-pyrimidinone (example 20) for 5-iodo-6-methyl-3-(2-phenylethyl)-2-{2-[(phenylmethyl)oxy]phenyl}-4(3H)-pyrimidinone and 4-trifluoromethylbenzeneboronic acid for thiophene-3-boronic acid provided the title compound: MS (m/z): 451.2 [M+H]⁺.

### Example 72

### Preparation of 5-(3-Fluorophenyl)-2-(2-hydroxyphenyl)-6-methyl-3-(2-phenylethyl)-4(3H)-pyrimidinone

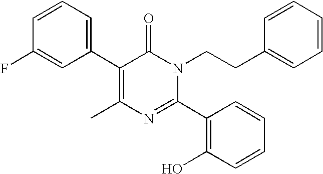

The title compound was prepared according to the procedures of Example 66 except substituting 5-bromo-6-methyl-3-(2-phenylethyl)-2-{2-[(phenylmethyl)oxy]phenyl}-4(3H)-pyrimidinone (example 20) for 5-iodo-6-methyl-3-(2-phenylethyl)-2-{2-[(phenylmethyl)oxy]phenyl}-4(3H)-pyrimidinone and 3-fluorophenylboronic acid for thiophene-3-boronic acid: MS (m/z): 401.2 [M+H]⁺.

### Example 73

### Preparation of 5-(2,4-Difluorophenyl)-2-(2-hydroxyphenyl)-6-methyl-3-(2-phenylethyl)-4(3H)-pyrimidinone

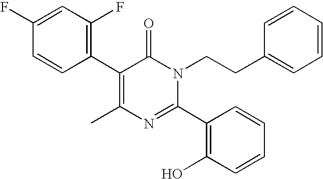

The title compound was prepared according to the procedures of Example 66 except substituting 5-bromo-6-methyl-3-(2-phenylethyl)-2-{2-[(phenylmethyl)oxy]phenyl}-4(3H)-pyrimidinone (example 20) for 5-iodo-6-methyl-3-(2-phenylethyl)-2-{2-[(phenylmethyl)oxy]phenyl}-4(3H)-pyrimidinone and 2,4-difluorophenylboronic acid for thiophene-3-boronic acid: MS (m/z): 419.2 [M+H]⁺.

### Example 74

### Preparation of 5-[4-(Dimethylamino)phenyl]-2-(3-fluoro-2-hydroxyphenyl)-6-methyl-3-(2-phenylethyl)-4(3H)-pyrimidinone

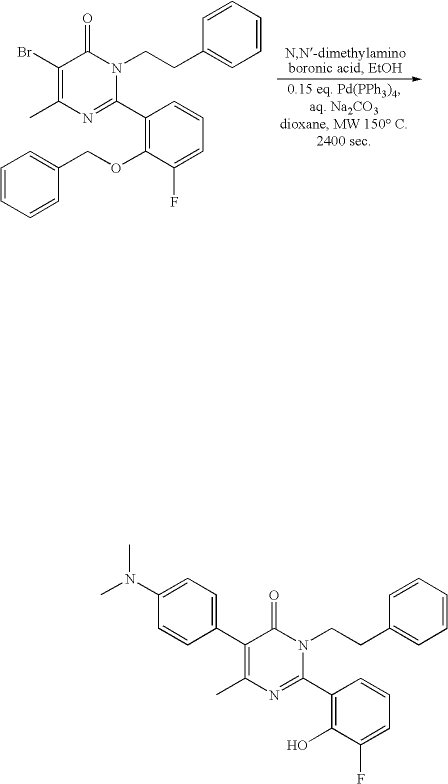

To a solution of 5-bromo-2-{3-fluoro-2-[(phenylmethyl)oxy]phenyl}-6-methyl-3-(2-phenylethyl)-4(3H)-pyrimidinone (0.20 g, 0.41 mmoles) of Example 11 in dioxane (5 mL) was added 4-(N,N-dimethylamino)phenylboronic acid (0.134 g, 0.81 mmoles), 0.5 mL ethanol, and 0.5 mL aqueous sodium carbonate (0.089 g, 0.81 mmoles) in a microwave reaction vessel. After 10 min. of deoxygenation, tetrakis(triphenylphosphine)palladium (0.070 g, 0.06 mmoles) was added. The mixture in a sealed vessel was irradiated to 150° C. for 2400 seconds. The reaction mixture was filtered through syringe filter (Acrodisc CR25 mm with 0.2 □m PTFE membrane). The vessel and filter were washed with ethyl acetate. EtOAc combined with filtrate were washed with brine, separated, dried over sodium sulfate. Filtered, concentrated in vacuo and the residue was purified by flash chromatography (0-40% ethyl acetate/hexane) to afford the title compound (0.13 g, 72%). MS (m/z): 444.2 [M+H].

### Example 75

### Preparation of 5-[4-(Ethyloxy)phenyl]-2-(3-fluoro-2-hydroxyphenyl)-6-methyl-3-(2-phenylethyl)-4(3H)-pyrimidinone

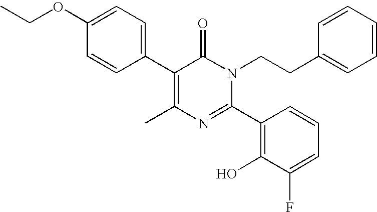

The title compound was prepared according to the procedures outlined in Example 74 except substituting 4-ethoxyphenylboronic acid for 4-(N,N-Dimethylamino)phenylboronic acid: MS (m/z): 445.4 [M+H]⁺.

### Example 76

### Preparation of 5-(1-Benzothien-3-yl)-2-(3-fluoro-2-hydroxyphenyl)-6-methyl-3-(2-phenylethyl)-4(3H)-pyrimidinone

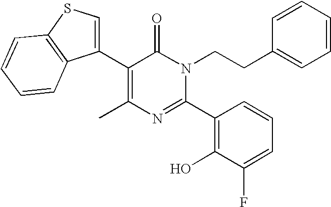

The title compound was prepared according to the procedures outlined in Example 74 except substituting thianaphthene-3-boronic acid for 4-(N,N-dimethylamino)phenylboronic acid. MS (m/z): 457.2 [M+H]⁺.

### Example 77

### Preparation of 5-(1-Benzothien-4-yl)-2-(3-fluoro-2-hydroxyphenyl)-6-methyl-3-(2-phenylethyl)-4(3H)-pyrimidinone

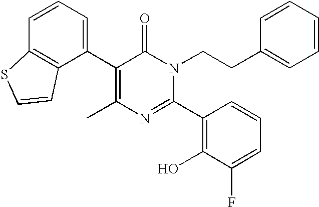

The title compound was prepared according to the procedures outlined in Example 74 except substituting thianaphthene-4-boronic acid for 4-(N,N-dimethylamino)phenylboronic acid: MS (m/z): 457.2 [M+H]⁻.

### Example 78

### Preparation of 2-[2-(3-Fluoro-2-hydroxyphenyl)-4-methyl-6-oxo-1-(2-phenylethyl)-1,6-dihydro-5-pyrimidinyl]benzonitrile

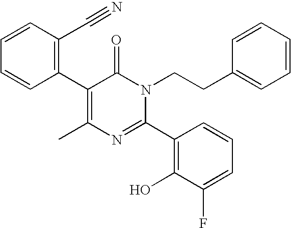

The title compound was prepared according to the procedures outlined in Example 74 except substituting 2-cyanophenylboronic acid for 4-(N,N-dimethylamino)phenylboronic acid. MS (m/z): 426.2 [M+H]⁺.

### Example 79

### Preparation of 4-[2-(3-Fluoro-2-hydroxyphenyl)-4-methyl-6-oxo-1-(2-phenylethyl)-1,6-dihydro-5-pyrimidinyl]benzonitrile

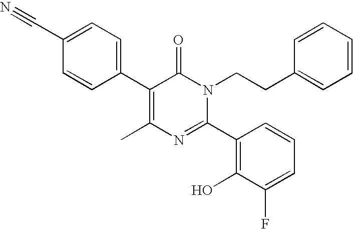

The title compound was prepared according to the procedures outlined in Example 74 except substituting 3-cyanophenylboronic acid for 4-(N,N-dimethylamino)phenylboronic acid. MS (m/z): 426.2 [M+H]⁺.

### Example 80

### Preparation of 5-[2-(Ethyloxy)Phenyl]-2-(3-fluoro-2-hydroxyphenyl)-6-methyl-3-(2-phenylethyl)-4(3H)-pyrimidinone

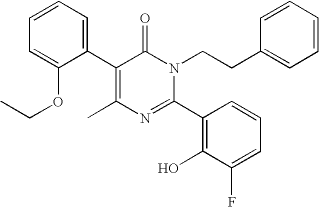

The title compound was prepared according to the procedures outlined in Example 74 except substituting 2-ethoxyphenylboronic acid for 4-(N,N-dimethylamino)phenylboronic acid: MS (m/z): 445.4 [M+H]⁺.

### Example 81

### Preparation of 5-[3-(Ethyloxy)phenyl]-2-(3-fluoro-2-hydroxyphenyl)-6-methyl-3-(2-phenylethyl)-4(3H)-pyrimidinone

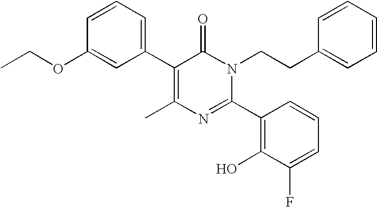

The title compound was prepared according to the procedures outlined in Example 74 except substituting 3-ethoxyphenylboronic acid for 4-(N,N-dimethylamino)phenylboronic acid: MS (m/z): 445.4 [M+H]⁺.

### Example 82

### Preparation of 5-(1-Benzofuran-2-yl)-2-(3-fluoro-2-hydroxyphenyl)-6-methyl-3-(2-phenylethyl)-4(3H)-pyrimidinone

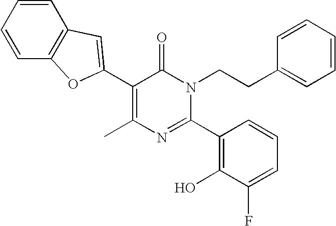

The title compound was prepared according to the procedures outlined in Example 74 except substituting 2-benzofuranboronic acid for 4-(N,N-dimethylamino)phenylboronic acid. MS (m/z): 441.2 [M+H]⁺.

### Example 83

### Preparation of 2-(3-Fluoro-2-hydroxyphenyl)-6-methyl-3-(2-phenylethyl)-5-(1H-pyrrol-2-yl)-4(3H)-pyrimidinone

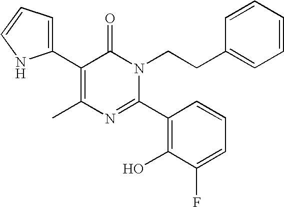

The title compound was prepared according to the procedures outlined in Example 74 except substituting N-Boc-pyrrole-2-boronic acid for 4-(N,N-dimethylamino)phenylboronic acid: MS (m/z): 390.2 [M+H]⁺.

### Example 84

### Preparation of 2-(3-Fluoro-2-hydroxyphenyl)-5-[3-(hydroxymethyl)phenyl]-6-methyl-3-(2-phenylethyl)-4(3H)-pyrimidinone

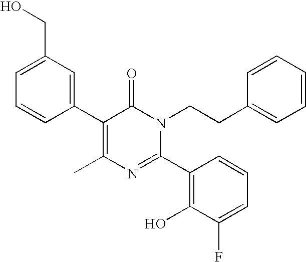

The title compound was prepared according to the procedures outlined in Example 74 except substituting [3-(hydroxymethyl)phenyl]boronic acid for 4-(N,N-dimethylamino)phenylboronic acid. MS (m/z): 431.2 [M+H]⁺.

### Example 85

### Preparation of 2-(3-Fluoro-2-hydroxyphenyl)-6-methyl-5-[3-(methylsulfonyl)phenyl]-3-(2-phenylethyl)-4(3H)-pyrimidinone

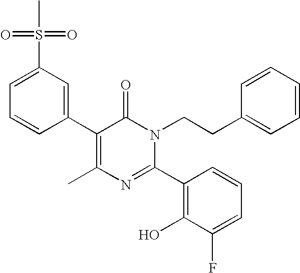

The title compound was prepared according to the procedures outlined in Example 74 except substituting 3-methanesulfonylphenylboronic acid for 4-(N,N-Dimethylamino)phenylboronic acid: MS (m/z): 479.2 [M+H]⁺.

### Example 86

### Preparation of 2-(3-Fluoro-2-hydroxyphenyl)-6-methyl-3-(2-phenylethyl)-5-[3-(trifluoromethyl)phenyl]-4(3H)-pyrimidinone

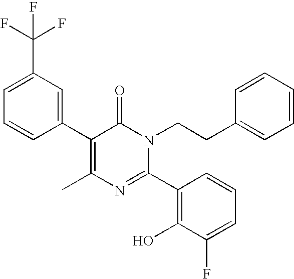

The title compound was prepared according to the procedures outlined in Example 74 except substituting 3-trifluoromethylphenylboronic acid for 4-(N,N-Dimethylamino)phenylboronic acid: MS (m/z): 469.2 [M+H]⁺.

### Example 87

### Preparation of 5-(3,4-Difluorophenyl)-2-(3-fluoro-2-hydroxyphenyl)-6-methyl-3-(2-phenylethyl) 4(3H)-pyrimidinone

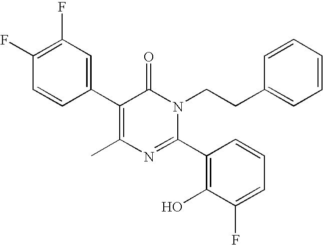

The title compound was prepared according to the procedures outlined in Example 74 except substituting 3,4-difluorophenylboronic acid for 4-(N,N-dimethylamino)phenylboronic acid: MS (m/z): 437.2 [M+H]⁺.

### Example 88

### Preparation of 5-[4-(1,1-Dimethylethyl)phenyl]-2-(3-fluoro-2-hydroxyphenyl)-6-methyl-3-(2-phenylethyl)-4(3H)-pyrimidinone

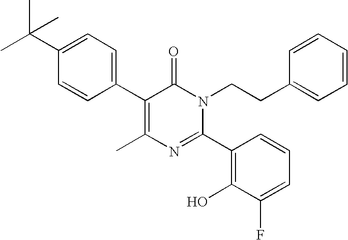

The title compound was prepared according to the procedures outlined in Example 74 except substituting 4-t-butylphenylboronic acid for 4-(N,N-dimethylamino)phenylboronic acid: MS (m/z): 457.2 [M+H]⁺.

### Example 89

### Preparation of 5-(5-Acetyl-2-thienyl)-2-(3-fluoro-2-hydroxyphenyl)-6-methyl-3-(2-phenylethyl)-4(3H)-pyrimidinone

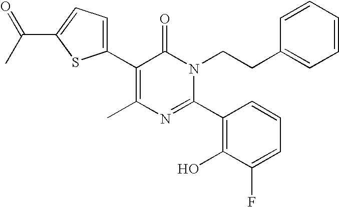

The title compound was prepared according to the procedures outlined in Example 74 except substituting 5-acetylthiophene-2-boronic acid for 4-(N,N-Dimethylamino)phenylboronic acid. MS (m/z): 449.2 [M+H]⁺.

### Example 90

### Preparation of 2-(3-Fluoro-2-hydroxyphenyl)-6-methyl-3-(2-phenylethyl)-5-{3-[(trifluoromethyl)oxy]phenyl}-4(3H)-pyrimidinone

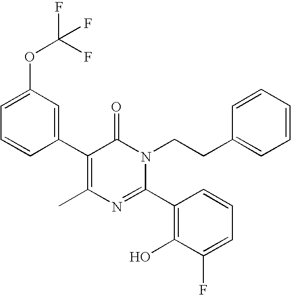

The title compound was prepared according to the procedures outlined in Example 74 except substituting 4-(trifluoromethoxy)benzeneboronic acid for 4-(N,N-dimethylamino)phenylboronic acid: MS (m/z): 485.2 [M+H]⁺.

### Example 91

### Preparation of 5-{3-[(Dimethylamino)methyl]phenyl}-2-(3-fluoro-2-hydroxyphenyl)-6-methyl-3-(2-phenylethyl)-4(3H)-pyrimidinone

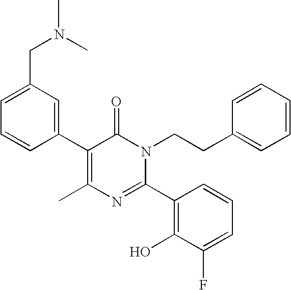

The title compound was prepared according to the procedures outlined in Example 74 except substituting N,N-dimethylaminomethylphenyl-3-boronic acid pinacol for 4-(N,N-dimethylamino)phenylboronic acid: MS (m/z): 458.2 [M+H]⁺.

### Example 92

### Preparation of 3-[2-(3-Fluoro-2-hydroxyphenyl)-4-methyl-6-oxo-1-(2-phenylethyl)-1,6-dihydro-5-pyrimidinyl]-N,N-dimethylbenzamide

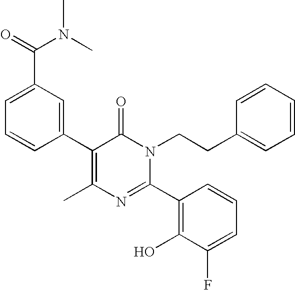

The title compound was prepared according to the procedures outlined in Example 74 except substituting 3-(dimethylcarbamoyl)phenylboronic acid for 4-(N,N-dimethylamino)phenylboronic acid: MS (m/z): 472.2 [M+H]⁺.

### Example 93

### Preparation of 5-(4,5-Dimethyl-2-thienyl)-2-(3-fluoro-2-hydroxyphenyl)-6-methyl-3-(2-phenylethyl)-4(3H)-pyrimidinone

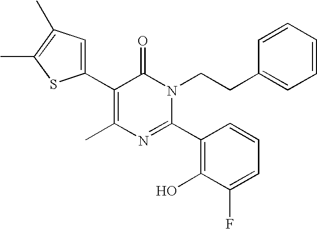

The title compound was prepared according to the procedures outlined in Example 74 except substituting ethyl (4,5-dimethyl-2-thienyl)borinate for 4-(N,N-dimethylamino)phenylboronic acid: MS (m/z): 435.2 [M+H]⁺.

### Example 94

### Preparation of 5-[2-(3-Fluoro-2-hydroxyphenyl)-4-methyl-6-oxo-1-(2-phenylethyl)-1,6-dihydro-5-pyrimidinyl]-2-thiophenecarbonitrile

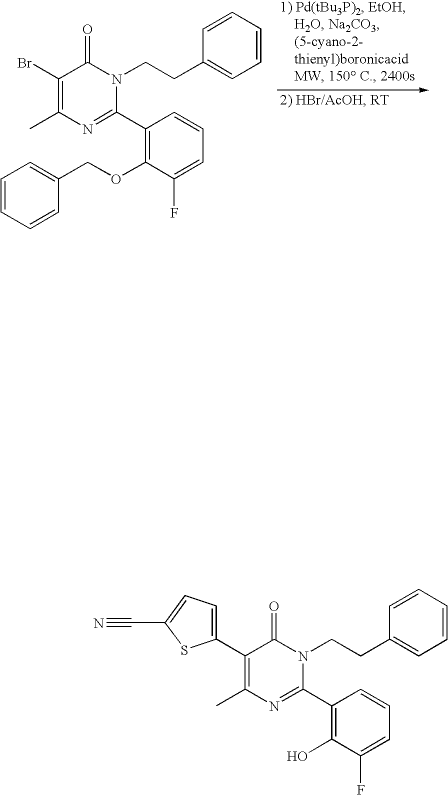

The title compound was prepared according to the procedures of Example 74 except substituting 5-cyanothiophene-2-boronic acid for 4-(N,N-dimethylamino)phenylboronic acid and bis-(tri-t-butylphosphine)palladium for tetrakis(triphenylphosphine)palladium. Microwave irradiation at 150° C. for 2400 seconds produced the desired compound. Debenzylation using hydrobromic acid in acetic acid as previously detailed produced the title compound: MS (m/z): 432.2 [M+H]⁺.

### Example 95

### Preparation of 2-(3-Fluoro-2-hydroxyphenyl)-6-methyl-5-(1-methyl-1H-pyrrol-2-yl)-3-(2-Phenylethyl)-4(3H)-pyrimidinone

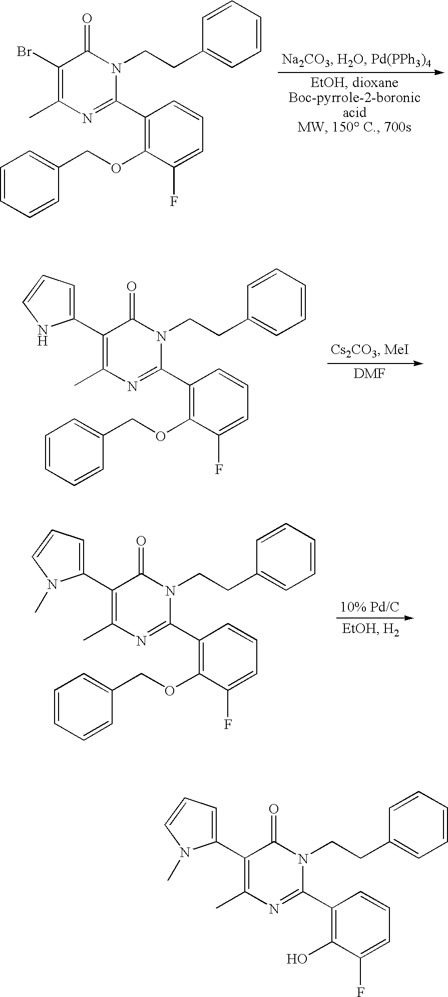

### a. 2-{3-Fluoro-2-[(phenylmethyl)oxy]phenyl}-6-methyl-3-(2-phenylethyl)-5-(1H-pyrrol-2-yl)-4(3H)-pyrimidinone

To a solution of 5-bromo-2-{3-fluoro-2-[(phenylmethyl)oxy]phenyl}-6-methyl-3-(2-phenylethyl)-4(3H)-pyrimidinone (0.60 g, 1.2 mmoles) of Example 11 in dioxane was added N-Boc-pyrrole-2-boronic acid (0.51 g, 2.4 mmoles), 0.5 mL ethanol, and 0.5 mL aqueous sodium carbonate (0.26 g, 2.4 mmoles) in a microwave reaction vessel. After 10 min. of deoxygenation, tetrakis(triphenylphosphine)palladium (0.14 g, 0.12 mmoles) was added. The mixture in a sealed vessel was irradiated to 150° C. for 700 seconds. The reaction mixture was filtered through syringe filter (Acrodisc CR25 mm with 0.20 □m PTFE membrane). The vessel and filter were washed with ethyl acetate. EtOAc combined with filtrate were washed with brine, separated, dried over sodium sulfate. Filtered, concentrated in vacuo and the residue was purified by flash chromatography to afford the title compound (0.285 g).

### b. 2-(3-Fluoro-2-hydroxyphenyl)-6-methyl-5-(1-methyl-1H-pyrrol-2-yl)-3-(2-phenylethyl)-4(3H)-pyrimidinone

To 2-{3-fluoro-2-[(phenylmethyl)oxy]phenyl}-6-methyl-3-(2-phenylethyl)-5-(1H-pyrrol-2-yl)-4(3H)-pyrimidinone (0.29 g, 0.59 mmoles) in DMF were added cesium carbonate (0.39 g, 1.2 mmoles) and methyl iodide (0.17 g, 1.2 mmoles). The reaction was quenched with water and diluted with ethyl acetate. The ethyl acetate layer was separated, washed with brine, dried over sodium sulfate, filtered, concentrated in vacuo and the residue was purified by flash chromatography (0-40% ethyl acetate/hexane) to afford 0.19 g (47%) yield of 2-{3-fluoro-2-[(phenylmethyl)oxy]phenyl}-6-methyl-5-(1-methyl-1H-pyrrol-2-yl)-3-(2-phenylethyl)-4(3H)-pyrimidinone. Catalytic hydrogenolysis yielded the title compound. MS (m/z): 404.2 [M+H]⁺.

### Example 96

### Preparation of 2-(3-fluoro-2-hydroxyphenyl)-6-methyl-5-(1-methyl-1H-indol-2-yl)-3-(2-phenylethyl)-4(3H)-pyrimidinone

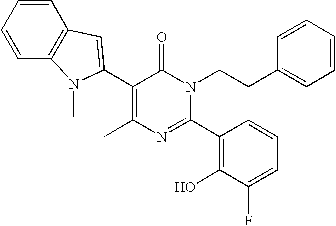

The title compound was prepared according to the procedure of Example 95 except substituting N-Boc-indole-2-boronic acid for 4-(N,N-dimethylamino)phenylboronic acid: MS (m/z): 454.0 [M+H]⁺.

### Example 97

### Preparation of 2-(3-Fluoro-2-hydroxyphenyl)-6-methyl-3-(2-phenylethyl)-5-(1,3-thiazol-2-yl)-4(3H)-pyrimidinone

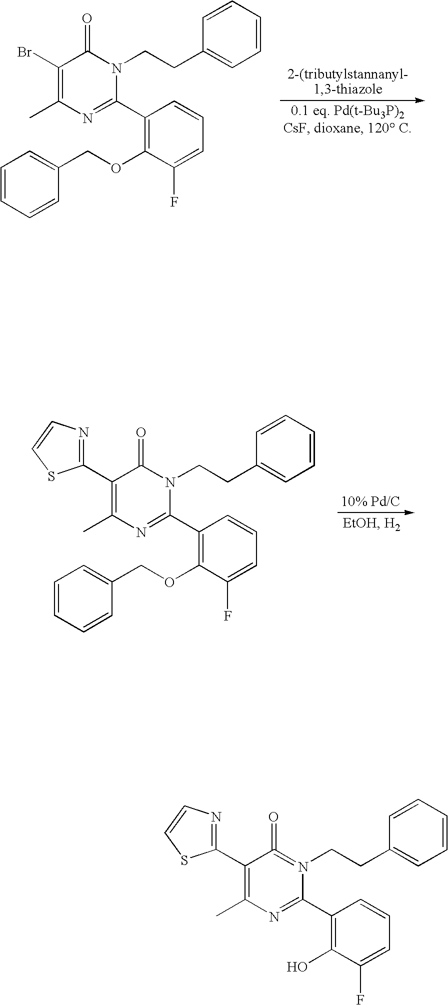

To a solution of 5-bromo-2-{3-fluoro-2-[(phenylmethyl)oxy]phenyl}-6-methyl-3-(2-phenylethyl)-4(3H)-pyrimidinone (0.20 g, 0.41 mmoles) of Example 11 in dioxane (5 mL) was added cesium fluoride (0.154 g, 1 mmoles). After 10 min. of deoxygenation, bis(tri-t-phosphine)palladium (0.021 g, 0.04 mmoles) and 2-(tributylstannanyl)thiazole were added. The mixture in sealed vessel was heated to 120° C. overnight. The reaction mixture was filtered through celite and diluted with ethyl acetate. The filtrate was washed with 10% w/v potassium fluoride, separated, dried over sodium sulfate, filtered, concentrated in vacuo and the residue purified by flash chromatography (0-40% ethyl acetate/hexane) to afford 0.16 g in 79% yield. Catalytic hydrogenolysis followed by reverse phase HPLC separation yielded the title compound (0.095 g, 72%): MS (m/z): 408.2 [M+H]⁺.

### Example 98

### Preparation of 2-(2-Hydroxyphenyl)-6-methyl-3-(2-phenylethyl)-5-(3-pyridinyl)-4(3H)-pyrimidinone

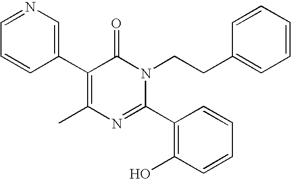

To the solution of 5-chloro-6-methyl-2-[2-(methyloxy)phenyl]-3-(2-phenylethyl)-4(3H)-pyrimidinone of Example 26 (0.15 g, 0.42 mmoles) in 10 mL of dioxane was added Pd(t-BU₃P)₂ (0.022 g, 0.04 mmoles), 3-pyridinylboronic acid (0.05 g, 0.47 mmoles) and Cs₂CO₃ (0.17 g, 0.51 mmoles). The reaction was degassed for 10 min and then heated at 90° C. for 12 h. The reaction mixture was cooled to room temperature, concentrated and crude product was chromatographed on flash silica gel column using 30% EtOAc/hexanes to provide 6-methyl-2-[2-(methyloxy)phenyl]-3-(2-phenylethyl)-5-(3-pyridinyl)-4(3H)-pyrimidinone: MS (ES) m/e 398[M+H]⁺. Subsequent deprotection with BBr₃ as previously described provided the title compound: MS (ES) m/e 384[M+H]⁺.

### Example 99

### Preparation of 2-(2-Hydroxyphenyl)-6-methyl-3-(2-phenylethyl)-5-(2-pyrazinyl)-4(3H)-pyrimidinone

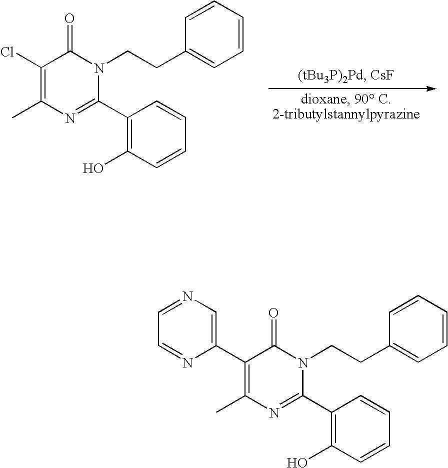

### a. 5-Chloro-2-(2-hydroxyphenyl)-6-methyl-3-(2-phenylethyl)-4(3H)-pyrimidinone

5-Chloro-6-methyl-2-[2-(methyloxy)phenyl]-3-(2-phenylethyl)-4(3H)pyrimidinone (0.23 g, 0.65 mmol) of Example 26 in dichloromethane was cooled to 0° C. 1M BBr₃ in dichloromethane (0.8 mL) was then added and let the reaction mixture stirred at this temperature until all the starting material is consumed. Upon completion the reaction mixture was diluted with dichloromethane and aq. NaHCO₃ was then added. Organic layer was separated and washed with H₂O, brine and dried over Na₂SO₄. After filtration the reaction mixture was concentrated and purified by chromatography on silica gel (40% ethyl acetate/hexane) to afford pure compound (0.10 g) in 45% yield

### b. 2-(2-Hydroxyphenyl)-6-methyl-3-(2-phenylethyl)-5-(2-pyrazinyl)-4(3H)-pyrimidinone

To a solution of 5-chloro-2-(2-hydroxyphenyl)-6-methyl-3-(2-phenylethyl)-4(3H)-pyrimidinone (0.17 g, 0.50 mmol) in dioxane under argon atmosphere was added Pd(tBu₃P)₂ (0.015 g, 0.029 mmol) and CsF (0.167 g, 0.0011 mol) then degassed for 10 minutes. 2-tributylstannylpyrazine (0.406 g, 0.0011 mol) was then added and the reaction was heated for 16 h. The reaction was the filtered through a pad of silica gel and concentrated. The crude product was purified by prep. TLC to give the desired product. MS (ES) m/e 385[M+H]⁺.

### Example 100

### Preparation of 6-Methyl-2-[2-(methyloxy)phenyl]-3-(2-phenylethyl)-5-(2-thienyl)-4(3H)-pyrimidinone

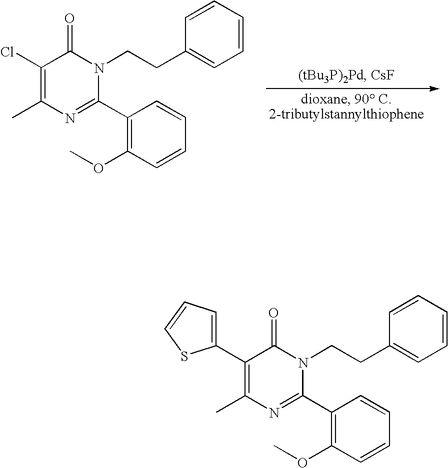

To a solution of 5-chloro-6-methyl-2-[2-(methyloxy)phenyl]-3-(2-phenylethyl)-4(3H)-pyrimidinone (0.32 g, 0.0.903 mmol) of Example 26 in dioxane under argon atmosphere was added Pd(tBu₃P)₂ (0.028 g, 0.054 mmol) and CsF (0.3 g, 1.98 mmol) then degassed for 10 minutes. 2-tributylstannylthiophene (0.32 mL, 0.99 mmol) was then added and the reaction was heated for 16 h. The reaction was the filtered through a pad of silica gel and concentrated. The crude product was purified by flash chromatography using 20% EtOAc/hexanes to give the product (0.2 g) in 54% yield. MS (ES) m/e 403[M+H]⁺.

### Example 101

### Preparation of 2-(2-Hydroxyphenyl)-6-methyl-5-phenyl-3-(2-phenylethyl)-4(3H)-pyrimidinone

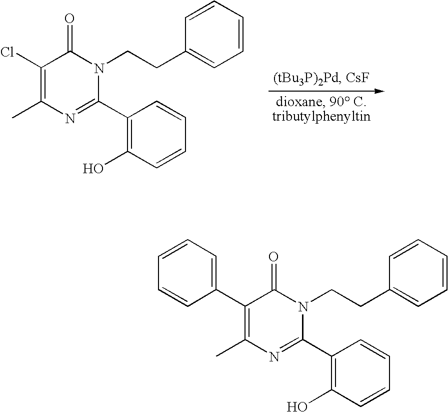

The title compound was prepared following the general procedure outlined in Example 99 except substituting tributylphenyltin for 2-tributylstannylthiophene. MS (ES) m/e 383[M+H]⁺.

### Example 102

### Preparation of 5-(4-Fluorophenyl)-2-(2-hydroxyphenyl)-6-methyl-3-(2-phenylethyl)-4(3H)-pyrimidinone

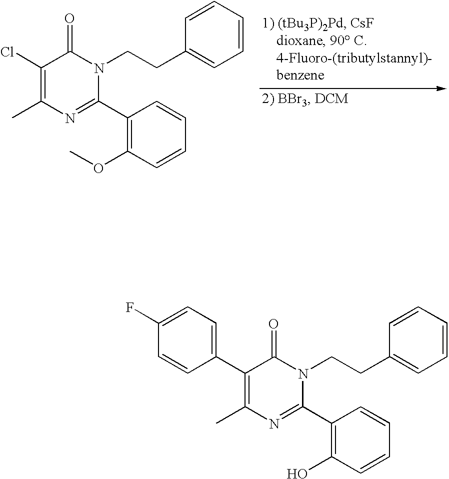

The title compound was prepared following the general procedure outlined in Example 99 except substituting 5-chloro-2-(2-methoxyphenyl)-6-methyl-3-(2-phenylethyl)-4(3H)-pyrimidinone for 5-chloro-2-(2-hydroxyphenyl)-6-methyl-3-(2-phenylethyl)-4(3H)-pyrimidinone and 4-fluoro-(tributylstannyl)-benzene for 2-tributylstannylpyrazine. Subsequent deprotection using BBr₃ as previously detailed produced the final compound. MS (ES) m/e 401 [M+H]⁺.

### Example 103

### Preparation of 2-(2-Hydroxyphenyl)-6-methyl-5-(3-methylphenyl)-3-(2-Phenylethyl)-4(3H)-pyrimidinone

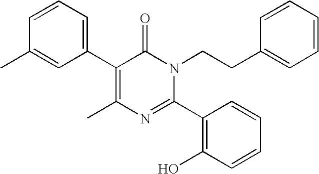

The title compound was prepared following the general procedure outlined in Example 99 except substituting 5-chloro-2-(2-methoxyphenyl)-6-methyl-3-(2-phenylethyl)-4(3H)-pyrimidinone for 5-chloro-2-(2-hydroxyphenyl)-6-methyl-3-(2-phenylethyl)-4(3H)-pyrimidinone and 3-methyl-(tributylstannyl)-benzene for 2-tributylstannylpyrazine. Subsequent deprotection using BBr₃ as previously detailed produced the final compound. MS (ES) m/e 396 [M+H]⁺.

### Example 104

### Preparation of 2-(3-Fluoro-2-hydroxyphenyl)-6-methyl-5-(1-methyl-1H-indol-5-yl)-3-(2-phenylethyl)-4(3H)-pyrimidinone

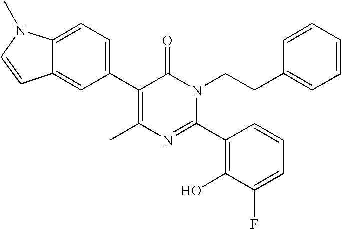

The title compound was prepared following the general procedure outlined in Example 13 except substituting N-methylindole-5-boronic acid for quinoline-6-boronic acid. MS (ES) m/e 454[M+H]⁺.

### Example 105

### Preparation of 2-(3-Fluoro-2-hydroxyphenyl)-6-methyl-3-(2-phenylethyl)-5-{4-[(trifluoromethyl)oxy]phenyl}-4(3H)-pyrimidinone

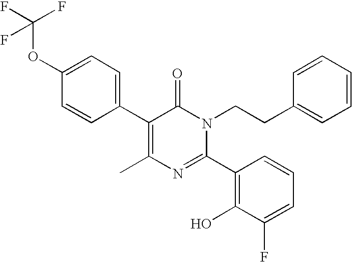

The title compound was prepared following the general procedure outlined in Example 13 except substituting 4-(trifluoromethoxy)benzene boronic acid for quinoline-6-boronic acid. MS (ES) m/e 485[M+H]⁺.

### Example 106

### Preparation of 2-(3-Fluoro-2-hydroxyphenyl)-6-methyl-5-{4-[(1-methylethyl)oxy]phenyl}-3-(2-phenylethyl)-4(3H)-pyrimidinone

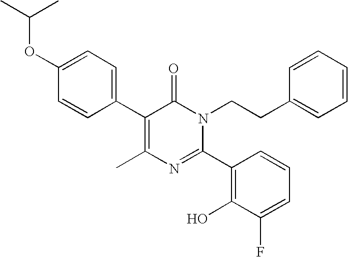

The title compound was prepared following the general procedure outlined in Example 13 except substituting 4-isopropoxyphenyl boronic acid for quinoline-6-boronic acid MS (ES) m/e 459[M+H]⁺.

### Example 107

### Preparation of 2-(3-Fluoro-2-hydroxyphenyl)-6-methyl-3-(2-phenylethyl)-5-(6-quinolinyl)-4(3H)-pyrimidinone

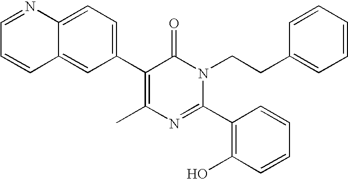

The title compound was prepared following the general procedure outlined in Example 13 except substituting 5-bromo-6-methyl-3-(2-phenylethyl)-2-{2-[(phenylmethyl)oxy]phenyl}-4(3H)-pyrimidinone of Example 20 for 5-bromo-2-{3-fluoro-2-[(phenylmethyl)oxy]phenyl}-6-methyl-3-(2-phenylethyl)-4(3H)-pyrimidinone. Subsequent deprotection using BBr₃ as previously detailed produced the final compound. MS (ES) m/e 434[M+H]⁺.

### Example 108

### Preparation of 5-(2,3-Dihydro-1,4-benzodioxin-6-yl)-2-(2-hydroxyphenyl)-6-methyl-3-(2-phenylethyl)-4(3H)-pyrimidin one

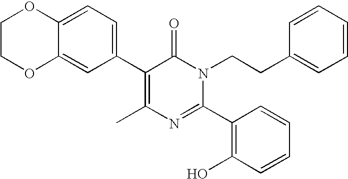

The title compound was prepared following the general procedure outlined in Example 13 except substituting 5-bromo-6-methyl-3-(2-phenylethyl)-2-{2-[(phenylmethyl)oxy]phenyl}4(3H)-pyrimidinone of Example 20 for 5-bromo-2-{3-fluoro-2-[(phenylmethyl)oxy]phenyl}-6-methyl-3-(2-phenylethyl)-4(3H)-pyrimidinone and 1,4-benzodioxane-6-boronic acid for quinoline-6-boronic acid. Subsequent deprotection using catalytic hydrogenolysis as previously detailed produced the final compound. MS (ES) m/e 441[M+H]⁺.

### Example 109

### Preparation of 5-(5-Chloro-3-methyl-1-benzothien-2-yl)-2-(3-fluoro-2-hydroxyphenyl)-6-methyl-3-(2-Phenylethyl)-4(3H)-pyrimidinone

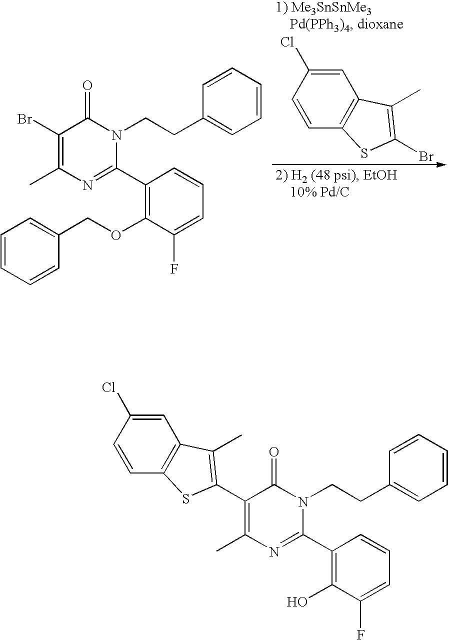

### a. 5-(5-Chloro-3-methyl-1-benzothien-2-yl)-2-{3-fluoro-2-[(phenylmethyl)oxy]phenyl}-6-methyl-3-(2-phenylethyl)-4(3H)-pyrimidinone

A solution of 5-bromo-2-{3-fluoro-2-[(phenylmethyl)oxy]phenyl}-6-methyl-3-(2-phenylethyl-4(3H)-pyrimidinone (0.3 g, 0.61 mmol) of Example 11, 2-bromo-5-chloro-3-methyl-1-benzothiophene (0.16 g, 0.61 mmol), hexamethyldistannane (0.13 mL, 0.61 mmol), Pd(PPh₃)₄ (0.070 g, 0.061 mmol) in 10 mL dioxane was degassed for 10 min and then heated at 90° C. for 16 h. The reaction mixture was concentrated, diluted with dichloromethane, filtered through Celite, and concentrated. Crude product was purified on flash Silica gel column and eluted with hexane/EtOAc (7:3) to give product (0.2 g) in yield 55%. MS (ES) m/e 594[M+H]⁺.

### b. 5-(5-Chloro-3-methyl-1-benzothien-2-yl)-2-(3-fluoro-2-hydroxyphenyl)-6-methyl-3-(2-phenylethyl)-4(3H)-pyrimidinone

5-(5-Chloro-3-methyl-1-benzothien-2-yl)-2-{3-fluoro-2-[(phenylmethyl)oxy]phenyl}-6-methyl-3-(2-phenylethyl)-4(3H)-pyrimidinone (0.1 g, 0.168 mmol) was taken up in glacial acetic acid. To this was added 10% Pd/C (0.02 g). This mixture was placed under hydrogen atmosphere at 48 psi and shaken for 16 h. The reaction mixture was filtered through a bed of celite and concentrated. The crude residue was taken up in dichloromethane and washed with NaHCO₃ and brine. The organic layer was dried over Na₂SO₄, filtered and concentrated. The crude residue was purified by chromatography on silica gel (15% ethyl acetate/hexane) to afford the desired product (0.030 g). MS (ES) m/e 505 [M+H]⁺.

### Example 110

### Preparation of 2-(3-Fluoro-2-hydroxyphenyl)-6-methyl-5-[5-(1,3-oxazol-5-yl)-2-thienyl]-3-(2-phenylethyl)-4(3H)-pyrimidinone

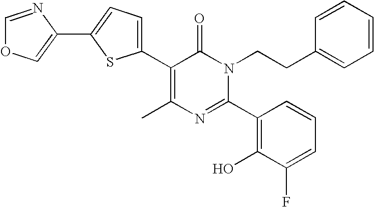

### a. 4-[5-(Trimethylstannanyl)-2-thienyl]-1,3-oxazole

5-(5-Bromo-2-thienyl)-1,3-oxazole (0.53 g, 2.30 mmol), hexamethyldistannane (3.0 g, 9.2 mmol), Pd(PPh₃)₄ (0.40 g, 0.35 mmol) in 20 mL toluene was degassed for 10 min and then heated at 90° C. for 16 h. The reaction mixture was concentrated, diluted with dichloromethane, filtered off the solid material and reconcentrated. The crude product was purified on flash Silica gel column and eluted with hexane/EtOAc (7:3) to give 0.2 g, (28%) of the title compound: MS (ES) m/e 314[M+H]⁺.

### b. 2-(3-Fluoro-2-hydroxyphenyl)-6-methyl-5-[5-(1,3-oxazol-5-yl)-2-thienyl]-3-(2-phenylethyl)-4(3H)-pyrimidinone

A solution of 5-bromo-2-{3-fluoro-2-[(phenylmethyl)oxy]phenyl}-6-methyl-3-(2-phenylethyl)-4(3H)-pyrimidinone of example 11f (0.315 g, 0.64 mmoles), 5-[5-(trimethylstannanyl)-2-thienyl]-1,3-oxazole (0.2 g, 0.64 mmoles), Pd(tBu₃P)₂ (0.020 g, 0.038 mmoles), CsF 0.21 g (1.41 mmoles) in 20 mL dioxane was degassed for 10 min and heated at 90° C. for 48 h. The reaction mixture was concentrated in vacuum, diluted with dichloromethane, filtered washed with 10% KF solution, dried (MgSO₄) and purified on flash Silica gel column to give 0.12 g, yield 33% of 2-{3-fluoro-2-[(phenylmethyl)oxy]phenyl}-6-methyl-5-[5-(1,3-oxazol-5-yl)-2-thienyl]-3-(2-phenylethyl)-4(3H)-pyrimidinone: MS (ES) m/e 564[M+H]⁺. Subsequent-catalytic hydrogenolysis provided the title compound. MS (ES) m/e 474[M+H]⁺.

### Example 111

### Preparation of 5-Fluoro-2-(2-hydroxyphenyl)-6-methyl-3-(2-phenylethyl)-4(3H)-pyrimidinone

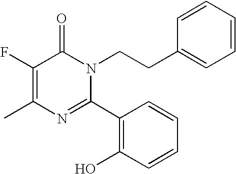

The title compound was prepared according to the procedure of Example 26 except substituting ethyl 2-fluoro-3-oxobutanoate for ethyl 2-chloro-3-oxobutanoate and subsequent deprotection using BBr₃ method: MS (ES) m/e 325[M+]⁺.

### Example 112

### Preparation of 2-(2-Hydroxyphenyl)-6-methyl-5-(2-methylpropyl)-3-(2-phenylethyl)-4(3H)-pyrimidinone

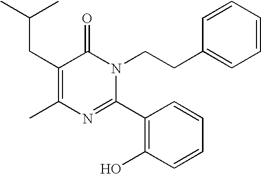

The title compound was prepared according to the procedure of Example 26 except substituting ethyl 2-acetyl-4-methylpentanoate for ethyl 2-chloro-3-oxobutanoate and subsequent deprotection using BBr₃ method: MS (ES) m/e 363[M+H]⁺.

### Example 113

### Preparation of 2-(2-Hydroxyphenyl)-6-methyl-5-(2-methyl-2-propen-1-yl)-3-(2-phenylethyl)-4(3H)-pyrimidinone

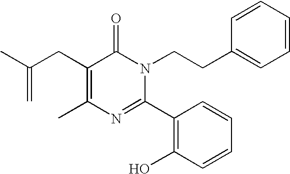

### a. Methyl 2-acetyl-4-methyl-4-pentenoate

3-Bromo-2-methyl-1-propene (6.75 g, 0.05 mol) and potassium carbonate (4.84 g, 0.035 mol) were added to a stirred solution of methyl acetoacetate in ACN (500 mL). The resulting heterogeneous mixture was stirred for 4 days and the solid was removed by filteration. Et₂O was added and washed with H₂O and brine. Organic layer was dried (Na₂SO₄), filtered and concentrated. The crude residue was purified by flash chromatography (10% EtOAc/hexanes) to produce the product (4.29 g).

### b. 2-(2-Hydroxyphenyl)-6-methyl-5-(2-methyl-2-propen-1-yl)-3-(2-phenylethyl)-4(3H)-pyrimidinone

The title compound was prepared according to the procedure of Example 26 except substituting methyl 2-acetyl-4-methyl-4-pentenoate for ethyl 2-chloro-3-oxobutanoate and subsequent deprotection using BBr₃ method. MS (ES) m/e 361 [M+H]⁺.

### Example 114

### Preparation of 5-(Cyclobutylmethyl)-6-methyl-2-[2-(methyloxy)phenyl]-3-(2-phenylethyl)-4(3H)-pyrimidinone

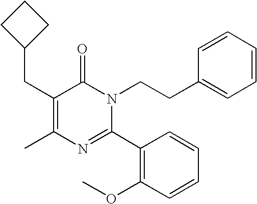

The title compound was prepared according to the procedure of Example 26 except substituting 2-cyclobutylmethyl-3-oxo-butyric acid ethyl ester for ethyl 2-chloro-3-oxobutanoate. MS (ES) m/e 389 [M+H]⁺.

### Example 115

### Preparation of 5-(Cyclobutylmethyl)-2-(2-hydroxyphenyl)-6-methyl-3-(2-phenylethyl)-4(3H)-pyrimidinone

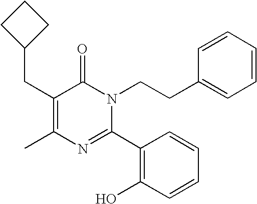

The title compound was prepared upon deprotection of Example 114 using BBr₃ as previously described: MS (ES) m/e 375[M+H]⁺.

### Example 116

### Preparation of 2-(2-Hydroxyphenyl)-6,6-dimethyl-3-(2-phenylethyl)-4a,5,6,7,8,8a-hexahydro-4(3H)-quinazolinone

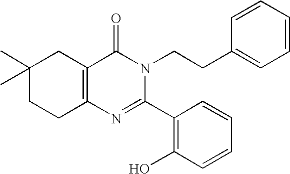

The title compound was prepared according to the procedure of Example 26 except substituting methyl 5,5-dimethyl-2-oxocyclohexanecarboxylate (synthesized according to procedure reported in Can. J. Chem., 66(9), 2345-2347, 1988) for ethyl 2-chloro-3-oxobutanoate and subsequent deprotection using BBr₃ method: MS (ES) m/e 375[M+H]⁺.

### Example 117

### Preparation of 5-(Cyclopropylmethyl)-2-(3-fluoro-2-hydroxyphenyl)-6-methyl-3-(2-phenylethyl)-4(3H)-pyrimidinone

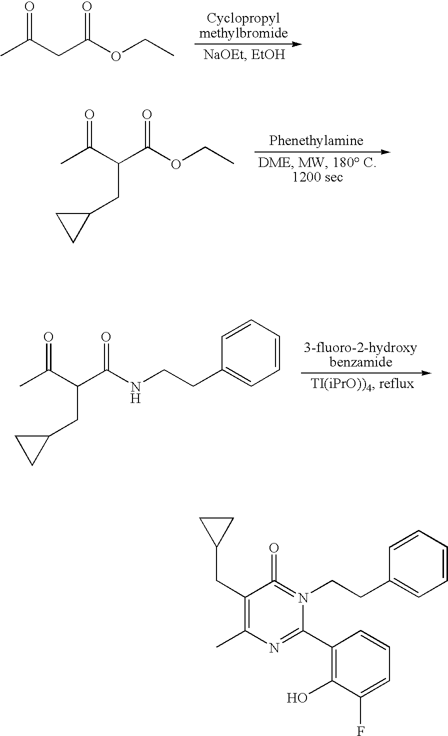

### a. Ethyl 2-(cyclopropylmethyl)-3-oxobutanoate

Ethyl 3-oxobutanoate (4.0 g, 31 mmol) was added to a suspension of NaOEt (4.9 g 36.2 mmol) in 40 mL of absolute EtOH. The reaction temperature was raised to 50° C. and stirred for 15 min whereupon bromomethyl)cyclopropane (4.9 g, 36.2 mmol) was added at 90° C. in 2 portions in 0.5 h and heated under mild reflux for 12 h. The reaction was concentrated in vacuum and residue was treated with saturated NH₄Cl solution and extracted with ether. The combined organic layers were dried (MgSO₄) filtered and concentrated to give ethyl 2-(cyclopropylmethyl)-3-oxobutanoate (4.8 g) in 84% yield.

### b. 2-(Cyclopropylmethyl)-3-oxo-N-(2-phenylethyl)butanamide

A DME (5 mL) solution of phenylmethyl 2-cyclopropyl-3-oxobutanoate (4.8 g, 0.026 mol), phenethylamine (3.1 mL, 0.024 mol) and ethanol (0.5 mL) was subjected to microwave irradiation at 180° C. for 1200 seconds. The reaction mixture was purified by flash column chromatography (30% ethyl acetate/hexanes) to give the desired product as a white solid in 30% yield (2.0 g).

### c. 5-(Cyclopropylmethyl)-2-(3-fluoro-2-hydroxyphenyl)-6-methyl-3-(2-phenylethyl)-4(3H)-pyrimidinone

The title compound was prepared following the procedure outlined in Example 11, step d except substituting 2-(cyclopropylmethyl)-3-oxo-N-(2-phenylethyl)butanamide for 3-oxo-N-(2-phenylethyl)butanamide. MS (ES) m/e 379[M+H]⁺.

### Example 118

### Preparation of 5-Cyclopropyl-2-(3-fluoro-2-hydroxyphenyl)-6-methyl-3-(2-phenylethyl)-4(3H)-pyrimidinone

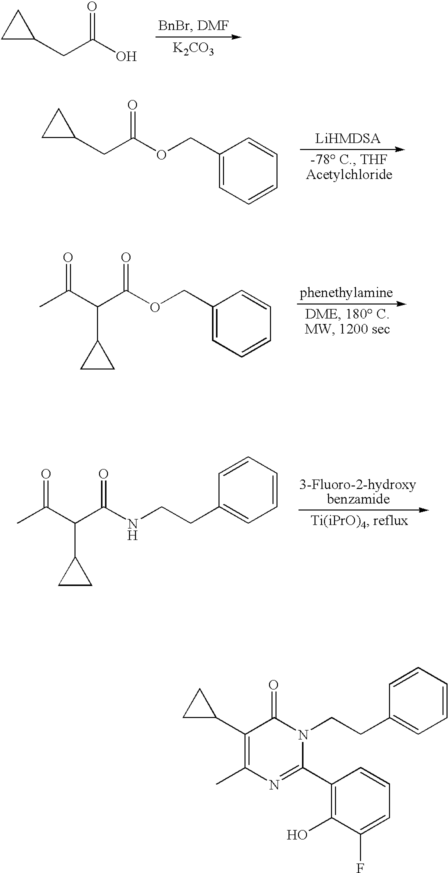

### a. Phenylmethyl Cyclopropylacetate

To a solution of cyclopropylacetic acid (5.0 g, 0.05 moles) in DMF (100 mL) was added K₂CO₃ (6.9 g, 0.05 mol) and benzyl bromide (5.95 mL, 0.05 mol). The reaction was stirred at RT overnight. The reaction mixture was diluted with EtOAc and washed with dilute HCl and brine. Dried over Na₂SO₄, filtered and concentrated. The crude residue was purified by chromatography on silica gel (Biotage) using EtOAc/hexane (0-60%) to provide the product (8.4 g) in 89% yield.

### b. Phenylmethyl 2-cyclopropyl-3-oxobutanoate

To a solution of phenylmethyl cyclopropylacetate (0.6 μg, 3.15 mmol) in THF at −78° C. was added 1M solution of LiHMDSA (3.75 mL, 3.78 mmol). The reaction stirred for 10 minutes then acetyl chloride (0.27 mL, 3.78 mmol) was added and continued stirring for additional 1 h. The reaction was quenched with saturated NH₄Cl and diethyl ether was added. This mixture was poured into H₂O followed by separation of organic layer. Organic layer was dried over Na₂SO₄, filtered and concentrated. The crude residue was purified by flash column chromatography purification system using EtOAc/hexane (0-60%) to provide the product (0.4 g) in 55% yield.

### c. 2-Cyclopropyl-3-oxo-N-(2-phenylethyl)butanamide

A DME (10 mL) solution of phenylmethyl 2-cyclopropyl-3-oxobutanoate (1.6 g, 6.88 mmol), phenethylamine (0.8 g, 6.54 mmol) and ethanol (0.5 mL) was subjected to microwave irradiation at 180° C. for 1200 seconds. The reaction mixture was purified by flash column chromatography (30% ethyl acetate/hexanes) to give the desired product as a white solid in 38% yield (0.64 g). MS (m/z): 246 (M+H)

### d. 5-Cyclopropyl-2-(3-fluoro-2-hydroxyphenyl)-6-methyl-3-(2-phenylethyl)-4(3H)-pyrimidinone

The title compound was prepared following the procedure outlined in Example 11, step d except substituting 2-cyclopropyl-3-oxo-N-(2-phenylethyl)butanamide for 3-oxo-N-(2-phenylethyl)butanamide. MS (ES) m/e 365[M+H]⁺.

### Example 119

### Preparation of 2-(3-Fluoro-2-hydroxyphenyl)-6-methyl-5-(3-methylbutyl)-3-(2-phenylethyl)-4(3H)-pyrimidinone

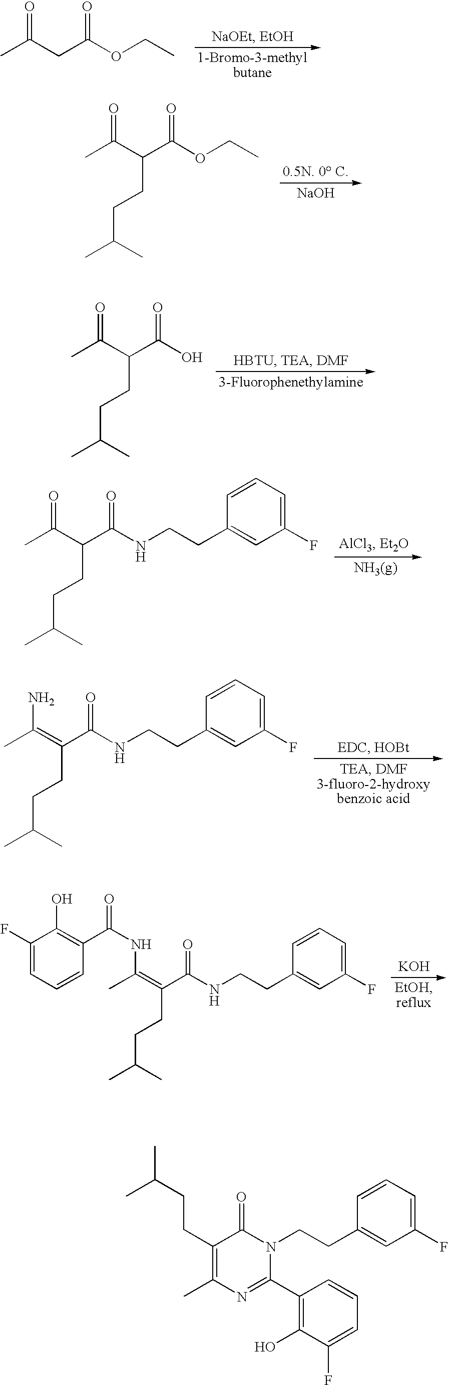

### a. Ethyl 2-acetyl-5-methylhexanoate

To the suspension of NaOEt (2.5 g, 0.037 mol) in 40 mL of dry ETOH was added ethyl 3-oxobutanoate (4.0 g, 0.031 mol) from a syringe and stirred for 15 minutes. Then the reaction was heated to gentle reflux and the 1-bromo-3-methyl butane (4.4 mL, 0.037 mol) was added in portions for two hours. The reflux was continued for 16 h. Upon cooling it was concentrated and diluted with mixture of Et₂O and ammonium chloride. The aqueous layer was reextrated with EtOAc, dried (Na₂SO₄), filtered and concentrated. The crude residue was purified by 10% EtOAc in hexanes to give the clean product (4.18 g) in 67% yield.

### b. 2-Acetyl-5-methylhexanoic acid

A round bottom flask was charged with ethyl 2-acetyl-5-methylhexanoate (4.18 g, 0.021 moles). To this was added a cold solution of 41.5 mL of 0.5N NaOH. The reaction mixture stirred at this temperature until starting material is all consumed. The reaction mixture was extracted with diethyl ether and the aqueous layer was acidified by 5% HCl and extracted with dichloromethane (×3). The combined organic layers were dried (Na₂SO₄), filtered and concentrated and taken directly into the next step.

### c. 2-Acetyl-N-[2-(3-fluorophenyl)ethyl]-5-methylhexanamide

To a solution of 2-acetyl-5-methylhexanoic acid (2.4 g, 0.014 moles) in DMF was added 3-fluorophenethylamine (1.64 mL, 0.0126 moles), HBTU (5.49 g, 0.0145 moles) and TEA (2 mL, 1.15 moles) were added sequentially and the reaction stirred at RT. The reaction mixture was concentrated, diluted with H₂O and extracted with dichloromethane. Extracts were washed with 1N HCl, followed by 5% NaHCO₃ solution and brine. The combined organic layers were dried (Na₂SO₄), filtered and concentrated and purified by flash column chromatography (5% MeOH/dichloromethane) to give 0.73 g of product.

### d. (2Z)-3-Amino-N-[2-(3-fluorophenyl)ethyl]-2-(3-methylbutyl)-2-butenamide

A solution of 2-acetyl-N-[2-(3-fluorophenyl)ethyl]-5-methylhexanamide (0.73 g, 0.00249 moles) in dry diethyl ether (100 mL) and THF (10 mL) at 0° C. was saturated with ammonia gas for 3 h. AlCl₃ (0.5 g) was added, and the mixture was allowed to warm to RT while stirring overnight. The resulting suspension was filtered, and the filtrate was concentrated to provide product (0.35 g).

### e. 3-Fluoro-N-[(1Z)-2-({[2-(3-fluorophenyl)ethyl]amino}carbonyl)-1,5-dimethyl-1-hexen-1-yl]-2-hydroxybenzamide

To a solution of 3-fluoro-2-hydroxybenzoic acid (0.21 g, 1.32 mmoles) and (2Z)-3-Amino-N-[2-(3-fluorophenyl)ethyl]-2-(3-methylbutyl)-2-butenamide (0.35 g, 1.2 mmoles) in dry THF was added EDC (0.25 g, 1.32 mmoles), HOBt (0.178 g, 1.32 mmoles) and TEA (0.20 mL, 1.32 mmoles) sequentially. The reaction was stirred at ambient temperature for 16 h. The reaction was diluted with EtOAc and washed with dilute HCl, 5% NaHCO₃ and brine. The organic layer was separated dried over Na₂SO₄, filtered and concentrated. The crude product was purified by flash column chromatography (5% MeOH/DCM) to produce the desired product (0.08 g). MS (ES) m/e 431 [M+H]⁺.

### f. 2-(3-Fluoro-2-hydroxyphenyl)-6-methyl-5-(3-methylbutyl)-3-(2-phenylethyl)-4(3H)-pyrimidinone

3-Fluoro-N-[(1Z)-2-({[2-(3-fluorophenyl)ethyl]amino}carbonyl)-1,5-dimethyl-1-hexen-1-yl]-2-hydroxybenzamide (0.08 g) was taken up in ethanol (5 mL) and 5 mL of 25% NaOH was added and the reaction refluxed overnight. After reaction was cooled to RT the pH is adjusted to ˜1 with 3N HCl and extracted with dichloromethane. The combined organic layers were dried over Na₂SO₄, filtered and concentrated. The crude product was purified by flash column chromatography (5% MeOH/DCM) followed by prep. TLC (50% EtOAc/hexanes) to produce the desired product. MS (ES) m/e 413[M+H]⁺.

### Example 120

### Preparation of 5-(2-Cyclohexylethyl)-2-(3-fluoro-2-hydroxyphenyl)-6-methyl-3-(2-phenylethyl)-4(3H)-pyrimidinone

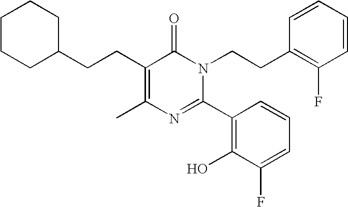

Preparation of the title compound followed the methods described in Example 119 except substituting 1-bromo-3-methyl butane for bromoethyl cyclohexane and 3-fluorophenethylamine for 2-fluorophenethylamine. MS (ES) m/e 453[M+H]⁺.

### Example 121

### Preparation of 5-(Cyclohexylmethyl)-2-(2-hydroxyphenyl)-6-methyl-3-(2-phenylethyl)-4(3H)-pyrimidinone

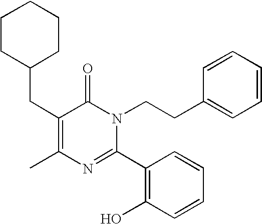

The title compound was prepared following the general procedures of Example 119 except substituting 2-(2-cyclohexylethyl)-3-oxobutanoic acid for 2-acetyl-5-methylhexanoic acid and phenethylamine for 3-fluorophenethylamine in step 119c and salicylic acid for 3-fluoro-2-hydroxybenzoic acid in step 119d. MS (ES) m/e 403[M+H]⁺.

### Example 122

### Preparation of 2-(3-Fluoro-2-hydroxyphenyl)-6-methyl-3-(2-phenylethyl)-5-(phenylmethyl-4(3H)-pyrimidinone

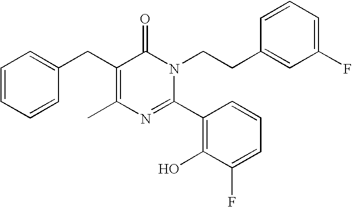

The title compound was prepared following the general procedures of Example 119 except substituting benzyl bromide for 1-bromo-3-methyl butane. MS (ES) m/e 433[M+H]⁺.

### Example 123

### Preparation of 5-Amino-2-(2-hydroxyphenyl)-6-methyl-3-(2-phenylethyl)-4(3H)-pyrimidinone

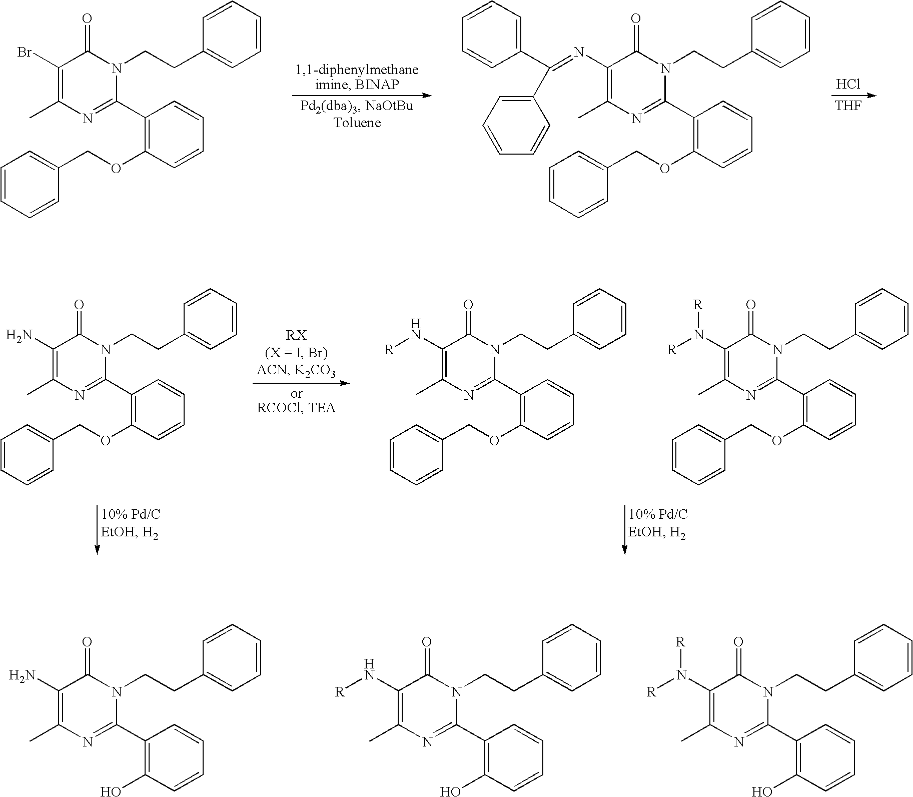

### a. 5-[(Diphenylmethylidene)amino]-6-methyl-3-(2-phenylethyl)-2-{2-[(phenylmethyl)oxy]phenyl}-4(3H)-pyrimidinone

To a solution of 5-bromo-6-methyl-3-(2-phenylethyl)-2-{2-[(phenylmethyl)oxy]phenyl}-4(3H)-pyrimidinone (2.74 g, 5.76 mmoles) and 1,1 diphenylmethaneimine (1.25 g, 6.92 mmoles) in 45 mL of toluene were degassed for 5 min; then Pd₂(dba)₃ (0.264 g, 0.283 mmoles), and BINAP (0.538 g, 0.864 mmoles) was added and degassed again for 10 min followed by NaOtBu (0.775 g, 0.864 mmoles) and heated for 12 h at 80° C. The reaction mixture was concentrated in vacuum and chromatographed on flash Silica gel column and eluted with hexane/EtOAc provided 3.2 g of the title compound (79%): MS (ES) m/e 576M+H]⁺.

### b. 5-Amino-6-methyl-3-(2-phenylethyl)-2-{2-[(phenylmethyl)oxy]phenyl}-4(3H)-pyrimidinone

5-[(diphenylmethylidene)amino]-6-methyl-3-(2-phenylethyl)-2-{2-[(phenylmethyl)oxy]phenyl}-4(3H)-pyrimidinone] was treated with 3 mL of 3N HCl in 20 ml of THF at RT for 12 h. The reaction was concentrated and triturated with ether. The resulting white solid was filtered off, dissolved in water and the pH adjusted to 13. The aqueous solution was extracted with dichloromethane washed with brine, dried (MgSO₄), filtered and concentrated to give 2 g of the title compound (87%): MS (ES) m/e 412 [M+H]⁻.

### c. 5-Amino-2-(2-hydroxyphenyl)-6-methyl-3-(2-phenylethyl)-4(3H)-pyrimidinone

The title compound was prepared by catalytic hydrogenolysis of 5-Amino-6-methyl-3-(2-phenylethyl)-2-{2-[(phenylmethyl)oxy]phenyl}-4(3H)-pyrimidinone

as previously described: MS (ES) m/e 322 [M+H]⁺.

### Example 124

### Preparation of 2-(2-Hydroxyphenyl)-6-methyl-3-(2-phenylethyl)-5-(1-piperidinyl)-4(3H)-pyrimidinone

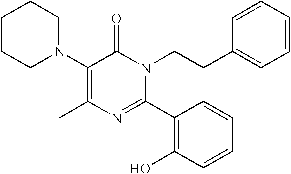

To a solution of 5-amino-6-methyl-3-(2-phenylethyl)-2-{2-[(phenylmethyl)oxy]phenyl}-4(3H)-pyrimidinone (0.1 g, 0.24 mmoles) of Example 123b in 2.5 mL of acetonitrile were added 1,5-dibromopentane (0.033 mL, 0.24 mmoles) and K₂CO₃ (0.084 g, 6.1 mmoles). The reaction was heated to reflux overnight. LCMS showed a very small amount of product. Additional amount of 1,5-dibromopentane (0.066, 0.48 mmoles) was added and the reaction was continued for 48 h. Upon cooling, the reaction mixture was diluted with EtOAc and washed with 1N HCl. Organic layer was separated and dried over Na₂SO₄. Filtered, concentrated and purified by Biotage (0-50% ethyl acetate/hexane) to afford pure amide (0.05 g) in 43% yield. Subsequent debenzylation provided the title compound. MS (m/z): 390.2 [M+H)]⁺.

### Example 125

### Preparation of 5-(Dimethylamino)-2-(2-hydroxyphenyl)-6-methyl-3-(2-phenylethyl)-4(3H)-pyrimidinone

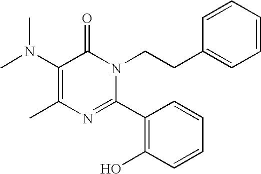

To a solution of 5-amino-6-methyl-3-(2-phenylethyl)-2-{2-[(phenylmethyl)oxy]phenyl}-4(3H)-pyrimidinone of Example 123 (0.1 g, 0.243 mmoles), iodomethane (1 mL) and K₂CO₃ (1.0 g) in acetone stirred at RT for 12 h. The reaction mixture was concentrated in vacuum re-dissolved in DME, washed with water, dried (MgSO₄), filtered and concentrated to give 0.1 g of product in 90% yield: MS (ES) m/e 440M+H]⁺. Subsequent deprotection via BBr₃ as previously described provided the target: MS (ES) m/e 350 [M+H]⁺.

### Example 126

### Preparation of N-[2-(2-Hydroxyphenyl)-4-methyl-6-oxo-1-(2-phenylethyl)-1,6-dihydro-5-pyrimidinyl]-2,2-dimethylpropanamide

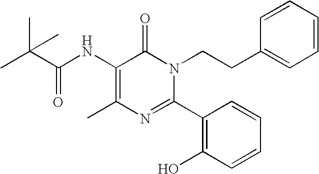

To a solution of 5-amino-6-methyl-3-(2-phenylethyl)-2-{2-[(phenylmethyl)oxy]phenyl}-4(3H)-pyrimidinone Example 123b (0.2 g, 0.486 mmoles) and 2,2-dimethylpropanoylchloride (0.072 g, 0.584 mmoles), TEA (0.14 mL, 0.973 mmoles) in dichloromethane at RT and stirred for 12 h. The reaction mixture was concentrated in vacuum, re-dissolved in dichloromethane and washed with 1N HCl, brine, dried (MgSO₄), filtered and concentrated to provide 2,2-dimethyl-N-(4-methyl-6-oxo-1-(2-phenylethyl)-2-{2-[(phenylmethyl)oxy]phenyl}-1,6-dihydro-5-pyrimidinyl)propanamide as an amber oil (0.2 g) in 83% yield: MS (ES) m/e 496M+H]⁺. Subsequent deprotection via BBr₃ as previously described produced the title compound: MS (ES) m/e 406 [M+H]⁺.

### Example 127

### Preparation of N-[2-(2-Hydroxyphenyl)-4-methyl-6-oxo-1-(2-phenylethyl)-1,6-dihydro-5-pyrimidinyl]-2-methylpropanamide

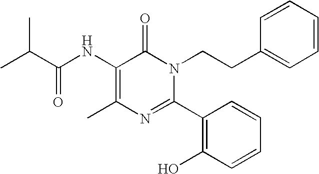

The title compound was synthesized according to procedure of Example 126 except substituting 2-methylpropanoyl chloride for 2,2-dimethylpropanoylchloride: MS (ES) m/e 392 [M+H]⁺.

### Example 128

### Preparation of N-[2-(2-hydroxyphenyl)-4-methyl-6-oxo-1-(2-phenylethyl)-1,6-dihydro-5-pyrimidinyl]-N,2-dimethylpropanamide

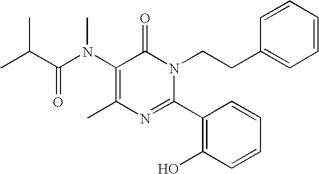

To a solution of 2,2-dimethyl-N-(4-methyl-6-oxo-1-(2-phenylethyl)-2-{2-[(phenylmethyl)oxy]phenyl}-1,6-dihydro-5-pyrimidinyl)propanamide of Example 127 (0.2 g, 0.415 mmoles), iodomethane (0.12 mL, 1.2 mmoles) and 60% NaH (0.031 g, 0.72 mmoles) in 10 mL DMF stirred at RT for 12 h, concentrated in vacuum and diluted with water and extracted with DME. Extracts were washed with brine, dried and concentrated. Crude product was chromatographed on flash Silica gel column and eluted with hexane/EtOAc (7:3) to give 0.11 g, in 53% yield: MS (ES) m/e 496M+H]⁺. The final target was prepared via BBr₃ deprotection as previously described. MS (ES) m/e 406 [M+H]⁺.

### Example 129

### Preparation of 5-(Dipropylamino)-2-(2-hydroxyphenyl-6-methyl-3-(2-phenylethyl)-4 (3H)-pyrimidinone

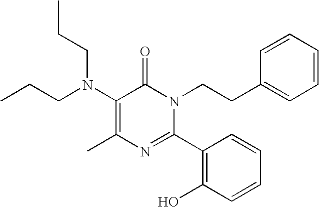

A solution of 5-amino-6-methyl-3-(2-phenylethyl)-2-{2-[(phenylmethyl)oxy]phenyl}-4(3H)-pyrimidinone (0.15 g, 0.365 mmol), vinylbromide (0.132 g, 1.09 mmoles) and K₂CO₃ (0.151 g, 1.09 mmoles) in acetonitrile was heated under reflux for 18 h. The reaction mixture was concentrated in vacuum diluted with water and extracted with dichloromethane; extracts were washed with brine, dried (MgSO₄), filtered and concentrated to give mixture of mono and dialkylated products. Bis-alkylated product: MS (ES) m/e 492M+H]⁺; monoalkylated product: MS (ES) m/e 452M+H]⁺. The above mixture 0.13 g was hydrogenated under atmospheric pressure in 10 mL of EtOH and 10 mg 10%/Pd/C for 18 h. Crude product was chromatographed on flash silica gel column and eluted with hexane/EtoAc (6:4) to give 0.023 g of the title compound: MS (ES) m/e 406 [M+H]⁺.

### Example 130

### Preparation of 5-(Diethylamino)-2-(2-hydroxyphenyl)-6-methyl-3-(2-Phenylethyl)-4(3H)-pyrimidinone

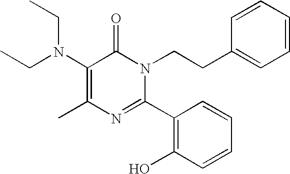

A solution of 5-amino-6-methyl-3-(2-phenylethyl)-2-{2-[(phenylmethyl)oxy]phenyl}-4(3H)-pyrimidinone (0.16 g, 0.389 mmol), ethylbromide (0.212 g, 1.95 mmoles) and Cs₂CO₃ (1.9 g, 5.85 mmoles) in DMF was stirred at 40° C. for 18 h. The reaction mixture was concentrated in vacuum diluted with water and extracted with dichloromethane; extracts were washed with brine, dried (MgSO₄), filtered and concentrated to give mixture of mono and dialkylated products. Bis-alkylated product: MS (ES) m/e 468 M+H]⁺; monoalkylated product: MS (ES) m/e 440 M+H]⁺. The above mixture was hydrogenated under atmospheric pressure as previously detailed to afford the desired product. MS (ES) m/e 378 [M+H]⁺

### Example 131

### Preparation of 5-(Ethylamino)-2-(2-hydroxyphenyl)-6-methyl-3-(2-phenylethyl)-4(3H)-pyrimidinone

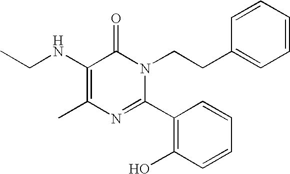

The title compound was produced as a by-product of Example 130. MS (ES) m/e 350 [M+H]⁺.

### Example 132

### Preparation of 2-(2-Hydroxyphenyl)-5-(2-methylpropyl)-3-(2-phenylethyl)-6-propyl-4(3H)-pyrimidinone

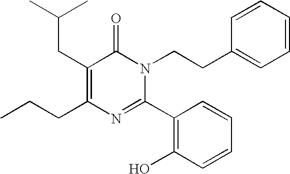

The title compound was prepared according to the procedures outlined in Example 45 except substituting 6-methyl-2-(2-{[(methyloxy)methyl]oxy}phenyl)-5-(2-methylpropyl)-3-(2-phenylethyl)-4(3H)-pyrimidinone of Example 112 for 5-ethyl-2-(3-fluoro-2{[(methyloxy)methyl]oxy}phenyl)-3-[2-(2-fluorophenyl)ethyl]-6-methyl-4(3H)-pyrimidinone and iodoethane for iodomethane in step 45e. MS (ES) m/e 391[M+H]⁺.

### Example 133

### Preparation of 6-Ethyl-2-(2-hydroxyphenyl)-5-(2-methylpropyl)-3-(2-phenylethyl)-4(3H)-pyrimidinone

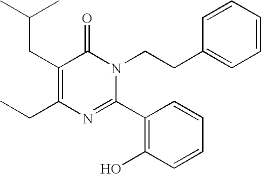

The title compound was prepared according to the procedures outlined in Example 45 except substituting 6-methyl-2-(2-{[(methyloxy)methyl]oxy}phenyl)-5-(2-methylpropyl)-3-(2-phenylethyl)-4(3H)-pyrimidinone of Example 112 for 5-ethyl-2-(3-fluoro-2-{[(methyloxy)methyl]oxy}phenyl)-3-[2-(2-fluorophenyl)ethyl]-6-methyl-4(3H)-pyrimidinone in step 45e. MS (ES) m/e 377[M+H]⁺.

### Example 134

### Preparation of 6-Butyl-2-(2-hydroxyphenyl)-5-(2-methylpropyl)-3-(2-phenylethyl)-4(3H)-pyrimidinone

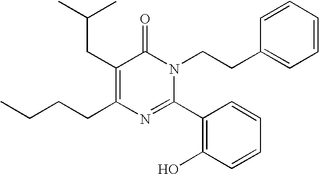

The title compound was prepared according to the procedures outlined in Example 45 except substituting 6-methyl-2-(2-{[(methyloxy)methyl]oxy}phenyl)-5-(2-methylpropyl)-3-(2-phenylethyl)-4(3H)-pyrimidinone of Example 112 for 5-ethyl-2-(3-fluoro-2-{[(methyloxy)methyl]oxy}phenyl)-3-[2-(2-fluorophenyl)ethyl]-6-methyl-4(3H)-pyrimidinone and iodopropane for iodomethane in step 45e: MS (ES) m/e 405[M+H]⁺.

### Example 135

### Preparation of 2-(2-Hydroxyphenyl)-5-(2-methylpropyl)-3-(2-phenylethyl)-6-{2-[(phenylmethyl)oxy]ethyl}-4(3H)-pyrimidinone

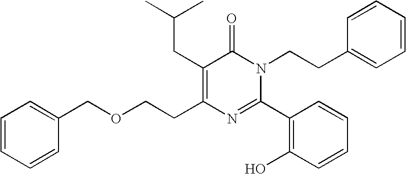

The title compound was prepared according to the procedures outlined in Example 45 except substituting 6-methyl-2-(2-{[(methyloxy)methyl]oxy}phenyl)-5-(2-methylpropyl)-3-(2-phenylethyl)-4(3H)-pyrimidinone of Example 112 for 5-ethyl-2-(3-fluoro-2-{[(methyloxy)methyl]oxy}phenyl)-3-[2-(2-fluorophenyl)ethyl]-6-methyl-4(3H)-pyrimidinone and chloromethylphenylmethyl ether for iodomethane in steps 45e. MS (ES) m/e 483[M+H]⁺.

### Example 136

### Preparation of 6-(2-Hydroxyethyl)-2-(2-hydroxyphenyl)-5-(2-methylpropyl)-3-(2-phenylethyl)-4(3H)-pyrimidinone

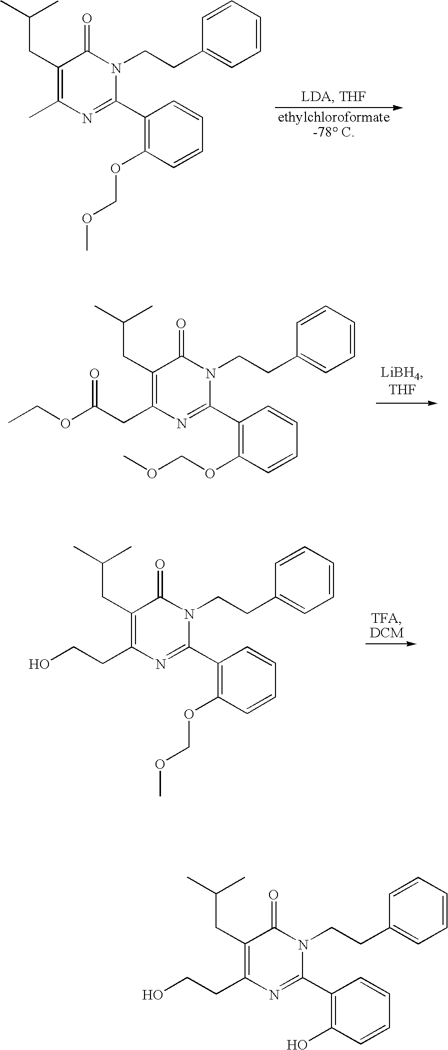

### a. Methyl [2-(2-hydroxyphenyl)-5-(2-methylpropyl)-6-oxo-1-(2-phenylethyl)-1,6-dihydro-4-pyrimidinyl]acetate

The title compound was prepared according to the procedures outlined in Example 45 except substituting 6-methyl-2-(2-{[(methyloxy)methyl]oxy}phenyl)-5-(2-methylpropyl)-3-(2-phenylethyl)-4(3H)-pyrimidinone of Example 112 for 5-ethyl-2-(3-fluoro-2-{[(methyloxy)methyl]oxy}phenyl)-3-[2-(2-fluorophenyl)ethyl]-6-methyl-4(3H)-pyrimidinone and ethyl chloroformate for iodomethane in steps 45e. MS (ES) m/e 479[M+H]⁺.

### b. 6-(2-Hydroxyethyl)-2-(2-{[(methyloxy)methyl]oxy}phenyl)-5-(2-methylpropyl)-3-(2-phenylethyl)-4(3H)-pyrimidinone

The crude product 0.2 g (0.419 mmoles) in THF 10 ml and at RT was treated with 0.1 g (4.8 mmoles) of LiBH₄ and stirred for 18 h. The reaction mixture was concentrated in vacuum, solid residue was treated with excess of 1N HCl solution and extracted with dichloromethane, dried (MgSO₄), filtered and concentrated to give 0.2 g of crude product. MS (ES) m/e 437[M+H]⁺.

### c. 6-(2-Hydroxyethyl)-2-(2-hydroxyphenyl)-5-(2-methylpropyl)-3-(2-phenylethyl)-4(3H)-pyrimidinone

The above crude product was treated with 5 mL TFA in 10 mL dichloromethane and stirred at RT for 16 h. Concentrated in vacuum and treated with 5% Na₂CO₃ solution and extracted with dichloromethane, dried (MgSO₄), filtered and concentrated to give crude product which was purified on flash Silica gel column and eluted with hexane/EtOAc to give the pure product (0.030 g). MS (ES) m/e 393[M+H]⁺.

### Example 137

### Preparation of 6-[2-(methyloxy)ethyl]-5-(2-methyl-1-propen-1-yl)-3-(2-phenylethyl)-4(3H)-pyrimidinone

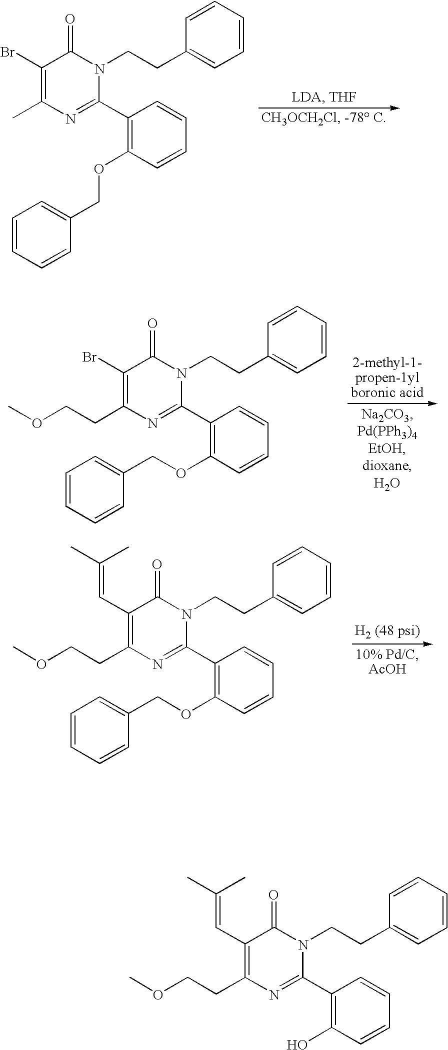

### a. 5-Bromo-6-[2-(methyloxy)ethyl]-3-(2-phenylethyl)-2-{2-[(phenylmethyl)oxy]phenyl}-4(3H)-pyrimidinone

To a −78° C. solution of 5-bromo-6-methyl-3-(2-phenylethyl)-2-{2-[(phenylmethyl)oxy]phenyl}-4(3H)-pyrimidinone (0.475 g, 0.001 moles) in THF was added LDA (0.0015 moles; prepared from n-BuLi and diisopropylamine) in THF and the reaction stirred for 1 h. MOMCl (0.12 mL, 0.0015 mmol) was added and the reaction stirred until starting material is all consumed. The reaction was quenched by NH₄Cl, extracted with EtOAc. The organic layer was washed with brine, dried over Na₂SO₄, filtered and concentrated. The residue was purified by flash column chromatography (30% EtOAc/hexane) to provide the product (0.2 g) in 39% yield.

### b. 6-[2-(Methyloxy)ethyl]-5-(2-methyl-1-propen-1-yl)-3-(2-phenylethyl)-2-{2-[(phenylmethyl)oxy]phenyl}-4(3H)-pyrimidinone

To a solution of 5-bromo-6-[2-(methyloxy)ethyl]-3-(2-phenylethyl)-2-{2-[(phenylmethyl)oxy]phenyl}-4(3H)-pyrimidinone (0.2 g, 0.39 mmoles) in dioxane (5 mL) was added 2-methyl-1-propen-1-yl-boronic acid (0.077 g) dissolved in solvent mixture of 0.5 mL ethanol and 0.5 mL of aqueous sodium carbonate (0.19 g, 0.39 mmoles) in a microwave reaction vessel. This mixture was irradiated to 150° C. for 1000 seconds. The reaction mixture was filtered through syringe filter (Acrodisc CR25 mm with 0.2 □m PTFE membrane). The filtrate was diluted with EtOAc and washed with brine and the organic layer was separated, dried over sodium sulfate, filtered and concentrated. The residue was purified by chromatography on silica gel (30% ethyl acetate/hexane) to afford the desired product (0.20 g) in 40% yield. Catalytic hydrogenolysis provided the title compound: MS (ES) m/e 405[M+H]⁺.

### Example 138

### Preparation of 2-(2-hydroxyphenyl)-6-[2-(methyloxy)ethyl]-5-(2-methylpropyl)-3-(2-phenylethyl)-4(3H)-pyrimidinone

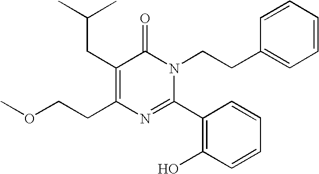

The title compound was a byproduct of the deprotection step 137c in Example 137: MS (ES) m/e 407[M+H]⁺.

### Example 139

### Preparation of 5-(dimethylamino)-2-(3-fluoro-2-hydroxyphenyl)-6-methyl-3-(2-phenylethyl)-4(3H)-pyrimidinone

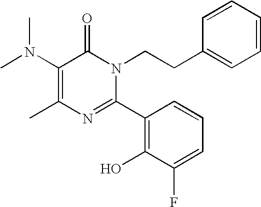

### a. 5-Amino-2-{3-fluoro-2-[(phenylmethyl)oxy]phenyl}-6-methyl-3-(2-phenylethyl)-4(3H)-pyrimidinone

The title compound was prepared following the methods described for Example 123 except using 5-bromo-2-{3-fluoro-2-[(phenylmethyl)oxy]phenyl}-6-methyl-3-(2-phenylethyl)-4(3H)-pyrimidinone of Example 11 in place of 5-bromo-2-{2-[(phenylmethyl)oxy]phenyl}-6-methyl-3-(2-phenylethyl)-4(3H)-pyrimidinone.

### b. 5-(Dimethylamino)-2-(3-fluoro-2-hydroxyphenyl)-6-methyl-3-(2-phenylethyl)-4(3H)-pyrimidinone

The title compound was prepared following the methods described for Example 125 except using 5-amino-2-{3-fluoro-2-[(phenylmethyl)oxy]phenyl}-6-methyl-3-(2-phenylethyl)-4(3H)-pyrimidinone in place of 5-amino-6-methyl-3-(2-phenylethyl)-2-{2-[(phenylmethyl)oxy]phenyl}-4(3H)-pyrimidinone. MS (ES) m/e 368[M+H]⁺.

### Example 140

### Preparation of 5-(Dimethylamino-2-(2-fluoro-3-hydroxyphenyl)-6-methyl-3-(2-phenylethyl)-4(3H)-pyrimidinone

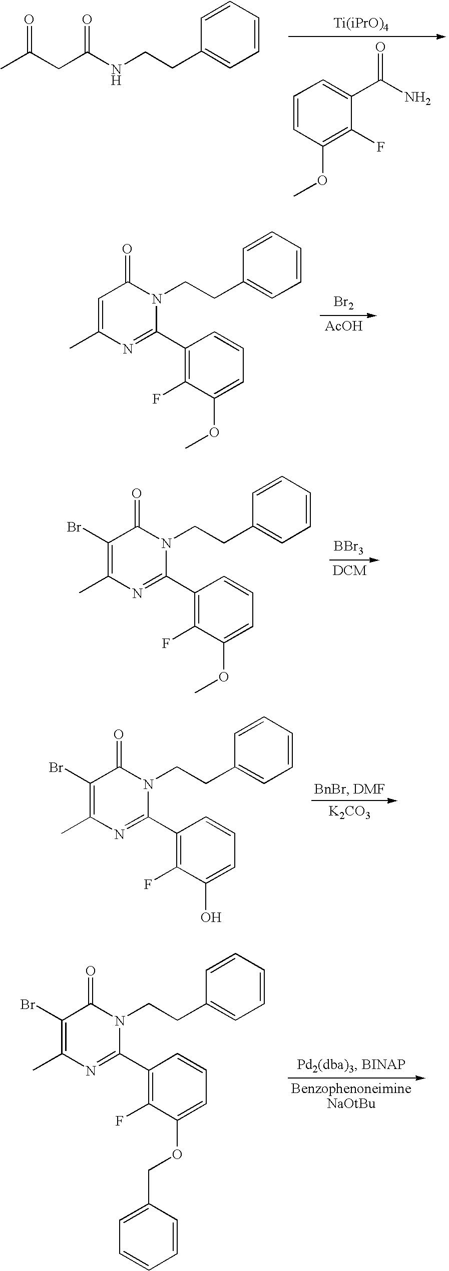

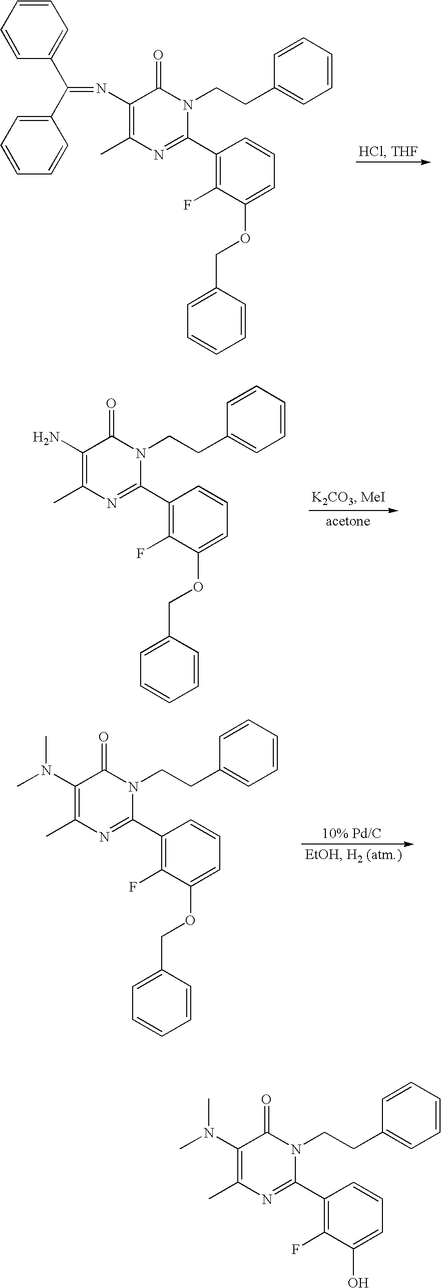

### a. 2-[2-Fluoro-3-(methyloxy)phenyl]-6-methyl-3-(2-phenylethyl)-4(3H)-pyrimidinone

The 3-oxo-N-(2-phenylethyl)butanamide (2.09 g, 0.01 mol) was placed in 44 mL round bottom flask. To this was added titanium isopropoxide ( ). While the reaction is stirring 2-fluoro-3-(methyloxy)benzamide (2.58, 0.015 mol) was added, a condenser was placed and the reaction was heated to reflux (oil bath temperature=150° C.). Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude solid was triturated with Et₂O. The solid (1.5 g) was filtered and taken into the next step without purification.

### b. 5-Bromo-2-[2-fluoro-3-(methyloxy)phenyl]-6-methyl-3-(2-phenylethyl)-4(3H)-pyrimidinone

2-[2-Fluoro-3-(methyloxy)phenyl]-6-methyl-3-(2-phenylethyl)-4(3H)-pyrimidinone (1.5 g, 4.44 mmol) was taken up in glacial acetic acid. To this was added bromine (0.34 mL, 6.66 mmol) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was triturated with Et₂O to obtain the desired product.

### c. 5-Bromo-2-(2-fluoro-3-hydroxyphenyl)-6-methyl-3-(2-phenylethyl)-4(3H)-pyrimidinone

5-Bromo-2-[2-fluoro-3-(methyloxy)phenyl]-6-methyl-3-(2-phenylethyl)-4(3H)-pyrimidinone (1.0 g, 0.0024 mol) was dissolved in DCM (15 mL) and cooled to 0° C. To this was added 12 mL of 1M BBr₃ in DCM and stirred overnight while warm to RT. The reaction was diluted with DCM and washed with Na₂CO₃ and organic layers were dried (Na₂SO₄), filtered and concentrated to produce the product (0.9 g) in 93% yield. MS (ES) m/e 405[M+H]⁺.

### d. 5-Bromo-2-{2-fluoro-3-[(phenylmethyl)oxy]phenyl}-6-methyl-3-(2-phenylethyl)-4(3H)-pyrimidinone

5-Bromo-2-(2-fluoro-3-hydroxyphenyl)-6-methyl-3-(2-phenylethyl)-4(3H)-pyrimidinone (0.9 g, 0.00223 moles) was dissolved in dry DMF (10 mL). To this was added potassium carbonate (0.463 g, 0.00335 moles) and benzyl bromide (0.4 mL, 0.0035 moles) sequentially. Reaction was warmed to 60° C. and stirred for 16 h. Reaction mixture was cooled to ambient temperature, filtered and diluted with EtOAc. This was washed successively with 5% HCl and saturated sodium chloride solution. Organic layer was dried over sodium sulfate and concentrated to give 1.0 gram of the desired compound. MS (ES) m/e 493[M+H]⁺.

### e. 5-[(Diphenylmethylidene)amino]-2-{2-fluoro-3-[(phenylmethyl)oxy]phenyl}-6-methyl-3-(2-phenylethyl)-4(3H)-pyrimidinone

To a solution of 5-bromo-2-{2-fluoro-3-[(phenylmethyl)oxy]phenyl}-6-methyl-3-(2-phenylethyl)-4(3H)-pyrimidinone (1.0 g, 0.00203 moles) and 1,1 diphenylmethaneimine (0.41 mL, 0.00243 moles) in 10 mL of toluene were degassed for 5 min; then Pd₂(dba)₃ (0.093 g, 0.0001 moles) and BINAP (0.189 g, 0.000304 moles) was added and degassed again for 10 min followed by NaOtBu (0.273 g, 0.00283 moles) and heated for 12 h at 80° C. The reaction mixture was concentrated in vacuum and chromatographed on flash Silica gel column and eluted with hexane/EtOAc provided 0.3 g of the title compound (25%): MS (ES) m/e 594 M+H]⁺.

### f. 5-Amino-2-{2-fluoro-3-[(phenylmethyl)oxy]phenyl}-6-methyl-3-(2-phenylethyl)-4(3H)-pyrimidinone

5-[(Diphenylmethylidene)amino]-2-{2-fluoro-3-[(phenylmethyl)oxy]phenyl}-6-methyl-3-(2-phenylethyl)-4(3H)-pyrimidinone (0.3 g, 0.000506 mol) was treated with 3 mL of 3N HCl in 20 mL of THF at RT for 12 h. The reaction was concentrated and triturated with ether. The resulting white solid was filtered off, dissolved in water and the pH adjusted to 13. The aqueous solution was extracted with dichloromethane washed with brine, dried (MgSO₄), filtered and concentrated to give 0.2 g of the title compound (92%).

### g. 5-(Dimethylamino)-2-{2-fluoro-3-[(phenylmethyl)oxy]phenyl}-6-methyl-3-(2-phenylethyl)-4(3H)-pyrimidinone

The amine (0.2 g, 0.465 mmol) was taken up in dry acetone (5 mL). To this was added potassium carbonate (0.128 g, 0.93 mmol) and methyl iodide (0.2 mL, 0.00233 mol) sequentially. The reaction was stirred overnight and concentrated. The crude mixture was diluted with H₂O and extracted with DCM. The combined organic layers were combined, dried (Na₂SO₄) and concentrated. The residue was purified by flash chromatography using 30% EtOAc/hexanes to afford product (0.1 g) in 47% yield. Catalytic hydrogenolysis as previously described provided the product: MS (ES) m/e 368[M+H]⁺.

### Example 141

### Preparation of 6-Methyl-2,5-diphenyl-3-(2-phenylethyl)-4(3H)-pyrimidinone

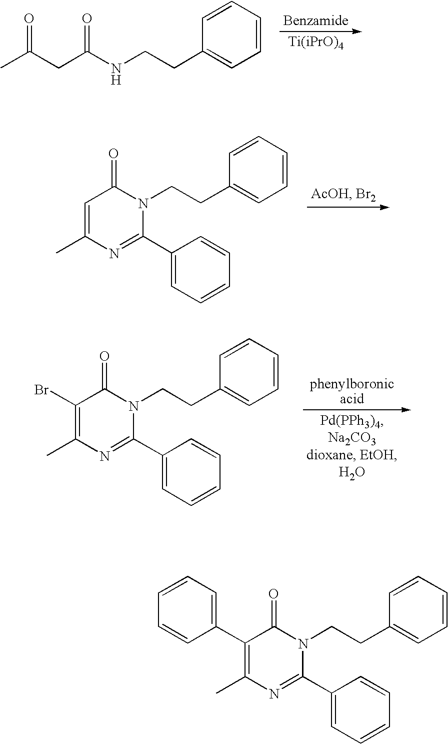

### a. 6-Methyl-2-phenyl-3-(2-phenylethyl)-4(3H)-pyrimidinone

The 3-oxo-N-(2-phenylethyl)butanamide (2 g, 0.0097 mol) of Example 11 was placed in 500 mL round bottom flask. To this was added titanium isopropoxide (37 mL, 0.13 mol). While the reaction is stirring benzamide (1.8 g, 0.0146 mol) was added, a condenser was placed and the reaction was heated to reflux (oil bath temperature=150° C.). Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude solid was triturated with Et₂O. The solid (2.1 g, 50%) was filtered and taken into the next step without purification.

### b. 5-Bromo-6-methyl-2-phenyl-3-(2-phenylethyl)-4(3M-pyrimidinone

6-Methyl-2-phenyl-3-(2-phenylethyl)-4(3H)-pyrimidinone (2.1 g, 0.0074 mol) was taken up in glacial acetic acid (29 mL). To this was added bromine (1.2 mL, 0.0074 mol) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was triturated with Et₂O to obtain the desired product (2 g) in 75% yield.

### c. 6-Methyl-2,5-diphenyl-3-(2-phenylethyl)-4(3H)-pyrimidinone

To a solution of 5-bromo-6-methyl-2-phenyl-3-(2-phenylethyl)-4(3H)-pyrimidinone (0.25 g, 0.68 mmol) in dioxane (6 mL) was added phenylboronic acid (0.165 g, 0.0014 mol) dissolved in solvent mixture of 0.5 mL ethanol and 0.5 mL of aqueous sodium carbonate (0.09 g, 0.8 mmol) in a microwave reaction vessel. This mixture was irradiated to 150° C. for 2400 seconds. The reaction mixture was filtered through syringe filter (Acrodisc CR25 mm with 0.2 □m PTFE membrane). The filtrate was diluted with EtOAc and washed with brine, separated, dried over sodium sulfate, filtered, concentrated in vacuo and the residue was purified by chromatography on silica gel (30% ethyl acetate/hexane) to afford the desired product (0.07 g). MS (ES) m/e 366[M+H]⁺.

### Example 142

### Preparation of 2-(2-Fluorophenyl)-6-methyl-5-phenyl-3-(2-phenylethyl)-4(3H)-pyrimidinone

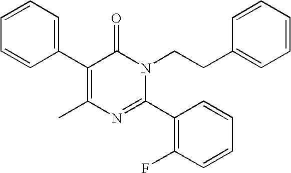

The title compound was prepared according to the procedures of Example 141 except substituting 2-fluorobenzamide for benzamide. MS (ES) m/e 385[M+H]⁺.

### Example 143

### Preparation of 3-[2-(2-chlorophenyl)ethyl]-2-(2-hydroxyphenyl)-5,6,7,8-tetrahydro-4(3H)-quinazolinone

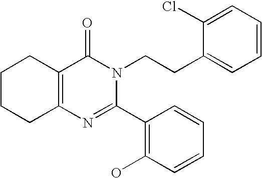

### a. 2-Methoxy-benzamidine

At 0° C. anhydrous ether was introduced to flask under Ar, LiHMDS (94 ml, 93.9 mmol) was then introduced and stirred for 5 mins. 2-methoxy-benzonitrile (5 g, 37.6 mmol) was added and the mixture was stirred at room temperature for 2-3 days. Upon completion of the reaction solvent was removed and 200 mL cold 1N HCl was added and stirred. The aqueous layer was extracted with Et₂O, then adjust the pH was adjusted by 6N NaOH to 13. Extraction with CH₂Cl₂, dried over Na₂SO₄ and filtered. Upon concentration the above benzamidine compound was obtained in 91% yield.

### b. 2-[2-(methyloxy)phenyl]-5,6,7,8-tetrahydro-4(1H)-quinazolinone

Dissolved 2-methoxy-benzamidine (150 mg, 1.0 mmol) in MeOH/dioxane (15 ml/5 ml) and cooled to 0° C. 25% NaOCH₃ in MeOH (0.44 ml) was then added and stirred for 15 mins. 2-oxo-cyclohexanecarboxylic acid ethyl ester (260 mg, 1.5 mmol) was introduced and the reaction mixture was heated to reflux for 1 h. The reaction was concentrated and the residue was taken up in 10 mL H₂O and acetic acid was used to adjust pH to 7-8. Extracted with CH₂Cl₂ (3×100 ml). The combined organic layers were dried over Na₂SO₄. Purified by flash column chromatography, (70% ethylacetate/hexane) to produce the product 220 mg in 86% yield.

### c. 3-[2-(2-chlorophenyl)ethyl]-2-(2-methoxyphenyl)-5,6,7,8-tetrahydro-4(3H)-quinazolinone

2-(2-Methoxy-phenyl)-5,6,7,8-tetrahydro-3H-quinazolin-4-one (150 mg, 0.6 mmol) was dissolved in dry DMF (3 mL). NaH (29 mg, 1.2 mmol) was added and stirred for 10 mins at room temperature. Then 2-chlorophenethyl bromide (655 mg, 3.00 mmol) was added and stirred at RT overnight. The mixture was poured the reaction mixture into ice and 6N HCl mixture. Extracted with EtOAc and the organic layer was washed with aqueous NaHCO₃, brine and dried over Na₂SO₄. Filtered and concentrated and purified by flash column chromatography to obtain desired product in (85 mg) yield 38%.

### d. 3-[2-(2-Chlorophenyl)ethyl]-2-(2-hydroxyphenyl)-5,6,7,8-tetrahydro-4(3H)-quinazolinone

3-[2-(2-Chlorophenyl)ethyl]-2-(2-methoxyphenyl)-5,6,7,8-tetrahydro-4(3H)-quinazolinone (161 mg, 0.41 mmol) in 5 mL CH₂Cl₂ was cooled to −60° C. 2.46 ml BBr₃ (1M in CH₂Cl₂) was then added and the reaction mixture was allowed to warmed to room temperature. Upon completion the reaction mixture was diluted with CH₂Cl₂ and aq. NaHCO₃ was then added. Organic layer was separated. The aqueous layer was neutralized by 1N HCl until pH is 4 and extracted with CH₂Cl₂. Organic layers were combined and washed with H₂O and brine. The organic layer was dried over Na₂SO₄, filtered and concentrated. The crude residue was purified by flash column chromatography (3%-5% methanol/methylenechloride) to product (0.11 g) in 69% yield. ¹H NMR (400 MHz, CDCl₃): [symbol] 8.59(s, 1H), 7.28-7.07 (m, 6H), 6.83 (t, 1H), 6.81 (d, 1H), 4.33 (t, 2H), 3.05 (t, 2H), 2.58 (m, 4H), 1.80 (m, 4H). MS (m/z): 381/383(M+H).

### Example 144

### Preparation of 3-[2-(3-fluorophenyl)ethyl]-2-(2-hydroxyphenyl)-5,5-dimethyl-5,6,7,8-tetrahydro-4(3H)-quinazolinone

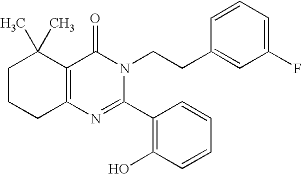

The title compound was prepared by substituting 3-fluorophenethyl bromide for 2-chlorophenethyl bromide and 3,3-dimethyl-2-oxo-cyclohexanecarboxylic acid methyl ester (synthesized according to J. Org. Chem.; 59(23), 1994; 6922-6927) for 2-oxo-cyclohexanecarboxylic acid ethyl ester in Example 143. ¹H NMR (400 MHz, CDCl₃): δ 9.58(br, 1H), 7.30 (m, 2H), 7.28 (m, 1H), 7.01 (m, 3H), 6.78 (d, 1H), 6.68 (m, 1H), 4.35 (t, 2H), 2.97 (t, 2H), 2.59 (t, 2H), 1.80 (m, 2H), 1.64 (m, 2H), 1.45 (s, 6H). MS (m/z): 393.4 (M+H).

### Example 145

### Preparation of 3-(2-cyclohexylethyl)-2-(2-hydroxyphenyl)-5,5-dimethyl-5,6,7,8-tetrahydro-4(3H)-quinazolinone

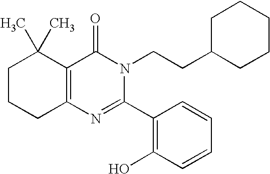

The title compound was prepared by substituting 2-cyclohexylethyl bromide for 2-chlorophenethyl bromide and 3,3-Dimethyl-2-oxo-cyclohexanecarboxylic acid methyl ester (synthesized according to J. Org. Chem.; 59(23), 1994; 6922-6927) for 2-oxo-cyclohexanecarboxylic acid ethyl ester in Example 143. ¹H NMR (400 MHz, CDCl₃): δ9.95(br, 1H), 7.01 (m, 2H), 6.70 (t, 1H), 6.65 (d, 1H), 3.90 (m, 2H), 2.58 (t, 2H), 1.80 (m, 2H), 1.54 (m, 2H), 1.50 (3H), 1.47 (s, 6H), 1.38 (m, 4H), 0.99 (m, 4H), 0.64 (m, 2H). MS (m/z): 381.5 (M+H).

### Example 146

### Preparation of 3-[2-(3-fluorophenyl)ethyl]-2-(2-furanyl)-5,6,7,8-tetrahydro-4(3H)-quinazolinone

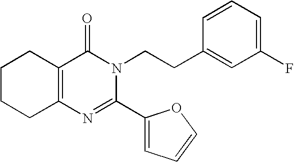

### a. Ethyl 2-[(2-furanylcarbonyl)amino]-1-cyclohexene-1-carboxylate

To a stirring solution of ethyl (2Z)-3-amino-2-propyl-2-pentenoate (500 mg, 2.95 mmol) in dichlormethane was added 2-furancarbonyl chloride (300 uL, 2.95 mmol) at 0° C. and allowed to warm to rt. The reaction was then heated to 50° C. and stirred for 1 hr. The reaction mixture was washed with aq NaHCO₃, the organic layer was dried over Na₂SO₄ and purified on a silica gel column to give the product (630 mg, 81%). MS (m/z) (M+H) 264.2

### b. 2-(2-Furanyl)-5,6,7,8-tetrahydro-4H-3,1-benzoxazin-4-one

To a stirring solution of ethyl 2-[(2-furanylcarbonyl)amino]-1-cyclohexene-1-carboxylate (700 mg, 2.95 mmol) in THF:H₂O (10 mL) was added LiOH.H₂O (280 mg, 7.35 mmol) and refluxed at 50° C. for 4 hr. The solvent was removed in vacuo and diluted with dichlormethane followed by the addition of 1N HCl. The organic layer was dried over Na₂SO₄, filtered, concentrated and used directly in the next step. (500 mg, 80%). To a stirring solution of 2-[(2-furanylcarbonyl)amino]-1-cyclohexene-1-carboxylic acid (500 mg, 2.12 mmol) in dichlormethane (10 mL) was added EDC.HCl (410 mg, 2.13 mmol) and HOBT (60 mg, 0.84 mmol) and stirred for 16 hr. The reaction mixture was washed with water, followed by brine, and the organic layer was dried over Na₂SO₄, filtered, concentrated and purified on a silica gel column to give (280 mg, 60%) the product. MS (m/z) 218.2 (M+H).

### c. 3-[2-(3-Fluorophenyl)ethyl]-2-(2-furanyl)-5,6,7,8-tetrahydro-4(3H)-quinazolinone

To a solution of 2-(2-furanyl)-5,6,7,8-tetrahydro-4H-3,1-benzoxazin-4-one (40 mg, 0.184 mmol) dissolved in AcOH (1 ml) was added 2-(3-fluorophenyl)ethanamine (51 mg, 0.368 mmol) and refluxed for 16 hr. AcOH was quenched with 6N NaOH and the product extracted into dichlormethane. The organic layer was separated, dried over Na₂SO₄, filtered, concentrated and purified by flash chromatography to give the title product (25 mg, 40%). MS (m/z) 339.4 (M+H).

### Example 147

### Preparation of 3-[2-(3-fluorophenyl)ethyl]-2-(2-thienyl)-5,6,7,8-tetrahydro-4(3H)-quinazolinone

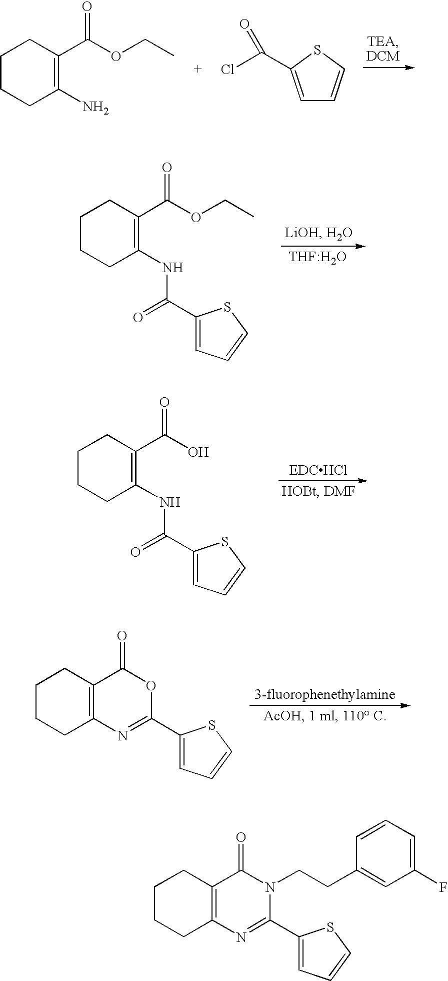

### a. Ethyl 2-[(2-thienylcarbonyl)amino]-1-cyclohexene-1-carboxylate

To a stirring solution of ethyl (2Z)-3-amino-2-propyl-2-pentenoate (1.0 g, 5.91 mmol) in dichlormethane was added 2-thiophenecarbonyl chloride (0.87 g, 5.93 mmol) at 0° C. and allowed to warm to room temperature. The reaction was then heated to 50° C. and stirred for 1 hr. The reaction mixture was washed with aq NaHCO₃, the organic layer was dried over Na₂SO₄ and purified on a silica gel column to give the product (1.25 g, 76%).

### b. 2-(2-Thienyl)-5,6,7,8-tetrahydro-4H-3,1-benzoxazin-4-one

To a stirring solution of ethyl 2-[(2-thienylcarbonyl)amino]-1-cyclohexene-1-carboxylate (1.25 g, 4.48 mmol) in THF:H₂O (20 mL) was added LiOH.H₂O (600 mg, 14.28 mmol) and refluxed at 50° C. for 4 h. The solvent was removed in vacuo and diluted with dichlormethane followed by the addition of 1N HCl. The organic layer was dried over Na₂SO₄, filtered, concentrated and used directly in the next step (89 mg, 81%). To a stirring solution of 2-[(2-thienylcarbonyl)amino]-1-cyclohexene-1-carboxylic acid (200 mg, 0.796 mmol) in dichlormethane (10 mL) was added EDC.HCl (170 mg, 0.582 mmol) and HOBT (22 mg, 0.162 mmol) and stirred for 16 h. The reaction mixture was washed with water, followed by brine, and the organic layer was dried over Na₂SO₄, filtered, concentrated and purified on a silica gel column to give (110 mg, 59%) of the title compound. MS (ESI) 234.2 (M+H).

### c. 3-[2-(3-Fluorophenyl)ethyl]-2-(2-thienyl)-5,6,7,8-tetrahydro-4(3H)-quinazolinone

To 2-(2-thienyl)-5,6,7,8-tetrahydro-4H-3,1-benzoxazin-4-one (50 mg, 0.214 mmol) dissolved in AcOH (1 ml) was added 2-(3-fluorophenyl)ethylamine (59 mg, 0.428 mmol) and refluxed for 16 hr. AcOH was quenched with 6N NaOH and the product extracted into dichlormethane. The organic layer was separated, dried over Na₂SO₄, filtered, concentrated and purified by flash chromatography to give the title product (30 mg, 40%). MS (ESI) 355.2 (M+H).

### Example 148

### Preparation of ethyl 2-(2-hydroxyphenyl)-4-methyl-6-oxo-1-(2-phenylethyl)-1,6-dihydro-5-pyrimidinecarbonitrile

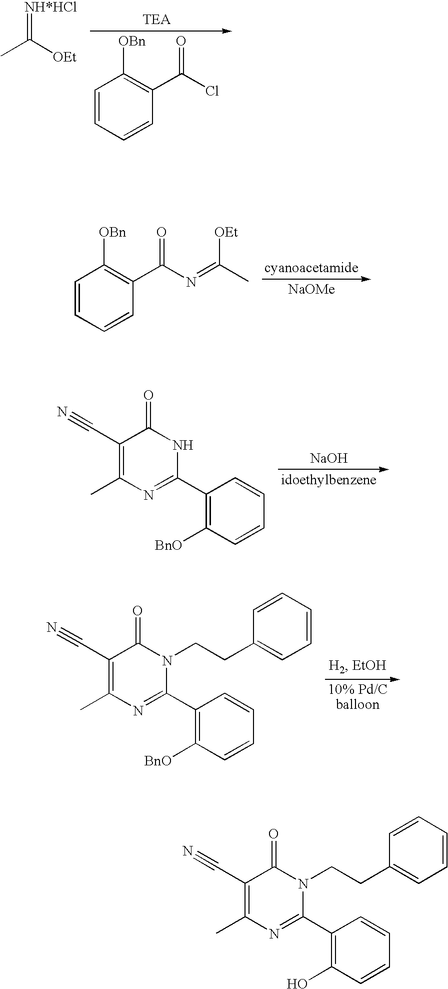

### a. Ethyl (1Z)-N-({2-[(phenylmethyl)oxy]phenyl}carbonyl)ethanimidoate

Ethyl acetamidate hydrochloride (1.08 g, 8.74 mmol) was dissolved in toluene (24 mL) and placed under argon. Triethylamine (2.75 mL, 19.7 mmol) was added and the reaction stirred at room temperature for 10 min. 2-[(phenylmethyl)oxy]benzoyl chloride (2.16 g, 8.76 mmol) in toluene (8 mL) was added dropwise via addition funnel over 15 min. The resulting reaction mixture was stirred for 5 days. The precipitated white solid was filtered away and rinsed with an excess of toluene. The filtrate was concentrated in vacuo and the crude product (2.45 g) was carried to the next step without purification.

### b. 4-Methyl-6-oxo-2-{2-[(phenylmethyl)oxy]phenyl}-1,6-dihydro-5-pyrimidinecarbonitrile

A flask was charged with ethanol (27 mL) and argon. Sodium ethoxide (95%, 0.731 g, 10.2 mmol) was added and reaction stirred for 3-5 min. Cyanoacetamide (0.695 g, 8.27 mmol) was added in one portion and the reaction stirred for 5 min. Ethyl (1Z)-N-({2[(phenylmethyl)oxy]phenyl}carbonyl)ethanimidoate (2.45 g, 8.24 mmol) in ethanol (6 mL) was added dropwise over 8 min. The reaction mixture was stirred at room temperature for 60 h. The reaction was neutralized with conc. H₂SO₄ (0.31 mL) and a yellow solid formed. The reaction was filtered but upon washing the filtered solid with water, the material dissolved. The resulting filtrate was extracted three times with CH₂Cl₂. The combined organic layers were dried over Na₂SO₄, filtered, and concentrated. Column chromatography (0-1% CH₃OH/CH₂Cl₂) yielded 4-methyl-6-oxo-2-{2-[(phenylmethyl)oxy]phenyl}-1,6-dihydro-5-pyrimidinecarbonitrile (1.85 g, 71% yield): ¹H NMR (400 MHz, CHLOROFORM-d) δ ppm 8.55 (d, 1H), 7.63 (m, 1H), 7.46 (m, 6H), 7.20 (m, 2H), 5.38 (s, 2H), 2.64 (s, 3H); MS (ESI) 318.2 (M+H)⁺.

### c. 4-Methyl-6-oxo-1-(2-phenylethyl)-2-{2-[(phenylmethyl)oxy]phenyl}-1,6-dihydro-5-pyrimidinecarbonitrile

To a solution of 4-methyl-6-oxo-2-{2-[(phenylmethyl)oxy]phenyl}-1,6-dihydro-5-pyrimidinecarbonitrile (0.265 g, 0.836 mmol) in ethanol:H₂0 (95:5, 5.6 mL) was added sodium hydroxide (0.193 g, 4.83 mmol). After the complete dissolution of 4-methyl-6-oxo-2-{2-[(phenylmethyl)oxy]phenyl}-1,6-dihydro-5-pyrimidinecarbonitrile, 2-iodoethylbenzene (2.5 mL, 17.3 mmol) was added. The reaction flask was sealed and heated at reflux for 27 h. The reaction mixture was cooled to room temperature and poured into cold H₂O. The resulting aqueous layer was extracted five times with CH₂Cl₂. The combined organic layers were washed with sat. Na₂S₂O₃, and brine, dried over Na₂SO₄, filtered, and concentrated. Column chromatography (0-2% CH₃OH/CH₂Cl₂) afforded 0.117 g (33%) of the title compound: ¹H NMR (400 MHz, CHLOROFORM-d) δ ppm 7.45 (m, 1H), 7.30 (m, 3H), 7.18 (m, 5H), 7.05 (m, 2H), 6.95 (m, 1H), 6.75 (m, 2H), 5.1 (dd, 2H), 4.40 (m, 1H), 3.72 (m, 1H), 2.84 (m, 1H), 2.76 (m, 1H), 2.58 (s, 3H); MS (ESI) 422.2 (M+H)⁺.

### d. 2-(2-Hydroxyphenyl)-4-methyl-6-oxo-1-(2-phenylethyl)-1,6-dihydro-5-pyrimidinecarbonitrile

Pd/C (10%, 0.017 g) was added to an argon purged solution of 4-methyl-6-oxo-1-(2-phenylethyl)-2-{2-[(phenylmethyl)oxy]phenyl}-1,6-dihydro-5-pyrimidinecarbonitrile (0.156 g, 0.370 mmol) in ethanol (4.0 mL). The reaction was then placed under balloon pressure of H₂ and stirred for 21 h. The reaction mixture was filtered through a Celite-plugged filter frit, rinsed with CH₃OH and CH₂Cl₂, and concentrated. Column chromatography (4% CH₃OH/CH₂Cl₂) yielded the title compound (0.109 g, 89%): ¹H NMR (400 MHz, DMSO-d₆) δ ppm 10.4 (s, 1H), 7.40 (m, 1H), 7.15 (m, 3H), 7.05 (m, 1H), 6.95 (m, 1H), 6.88 (m, 1H), 6.75 (m, 2H), 3.95 (m, 2H), 3.31 (s, 3H), 2.73 (m, 2H); MS (ESI) 332.2 (M+H)⁺.

### Example 149

### Preparation of Ethyl 2-(2-hydroxyphenyl)-4-methyl-6-oxo-1-(2-phenylethyl)-1,6-dihydro-5-pyrimidinecarboxylate

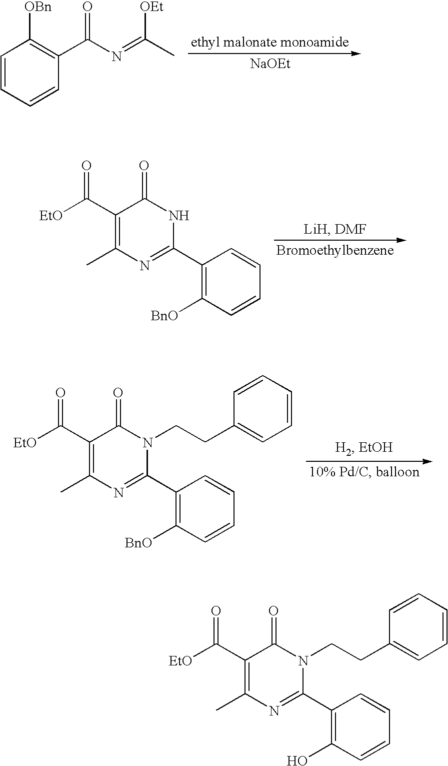

### a. Ethyl 4-methyl-6-oxo-2-{2-[(phenylmethyl)oxy]phenyl}-1,6-dihydro-5-pyrimidinecarboxylate

A flask was charged with ethanol (25 mL) and argon. Sodium ethoxide (95%, 0.827 g, 11.5 mmol) was added and reaction stirred for 3-5 min. Ethyl malonate monoamide (1.25 g, 9.53 mmol) was added in one portion and the reaction stirred for 45 min. Ethyl (1Z)-N-({2-[(phenylmethyl)oxy]phenyl}carbonyl)ethanimidoate (2.83 g, 9.52 mmol) in ethanol (14 mL) was added dropwise over 7 min. The reaction mixture was stirred at room temperature for 67 h. The reaction was neutralized with conc. H₂SO₄ (0.35 mL). The reaction was diluted with H₂O and extracted three times with CH₂Cl₂. The combined organic layers were dried over Na₂SO₄, filtered, and concentrated. Column chromatography (0-1% CH₃OH/CH₂Cl₂) yielded ethyl 4-methyl-6-oxo-2-{2-[(phenylmethyl)oxy]phenyl}-1,6-dihydro-5-pyrimidinecarboxylate (1.68 g crude) that was carried to the next step: MS (ESI) 365.4 (M+H)⁺.

### b. Ethyl 4-methyl-6-oxo-1-(2-phenylethyl)-2-{2-[(phenylmethyl)oxy]phenyl}-1,6-dihydro-5-pyrimidinecarboxylate

To a solution of ethyl 4-methyl-6-oxo-2-{2-[(phenylmethyl)oxy]phenyl}-1,6-dihydro-5-pyrimidinecarboxylate (1.68 g, 4.61 mmol) in DMF (13 mL) under argon was added lithium hydride (95%, 0.063 g, 7.53 mmol) and the reaction was stirred for 5 min. 2-(Bromoethyl)benzene (2.0 mL, 14.6 mmol) was added and reaction stirred for 28 h. The reaction mixture was quenched with H₂O and extracted two times with ethyl acetate. Combined organic layers were washed with brine, dried over MgSO₄, filtered, and concentrated. Purification by column chromatography (25-33% ethyl acetate:hexane) afforded 0.190 g (4% yield over two steps) of ethyl 4-methyl-6-oxo-1-(2-phenylethyl)-2-{2-[(phenylmethyl)oxy]phenyl}-1,6-dihydro-5-pyrimidinecarboxylate: ¹H NMR (400 M Hz, CHLOROFORM-d) δ ppm 7.46 (m, 1H), 7.29 (m, 5H), 7.18 (m, 3H), 7.07 (m, 3H), 6.83 (m, 2H), 5.12 (d, J=2.73 Hz, 2H), 4.47 (m, 2H), 4.35 (m, 1H), 3.72 (m, 1H), 2.95 (m, 1H), 2.74 (m, 1H), 2.44 (s, 3H), 1.46 (t, J=7.14 Hz, 3H); MS (ESI) 469.3 (M+H)⁺.

### c. ethyl 2-(2-hydroxyphenyl)-4-methyl-6-oxo-1-(2-phenylethyl)-1,6-dihydro-5-pyrimidinecarboxylate

Pd/C (10%, 0.010 g) was added to an Ar purged solution of ethyl 4-methyl-6-oxo-1-(2-phenylethyl)-2-{2-[(phenylmethyl)oxy]phenyl}-1,6-dihydro-5-pyrimidinecarboxylate (0.092 g, 0.196 mmol) in ethanol (2.4 mL). The reaction was then placed under balloon pressure of H₂ and stirred for 24 h. The reaction mixture was filtered through a Celite-plugged filter frit, rinsed with CH₃OH and CH₂Cl₂, and concentrated. Column chromatography (1-4% CH₃OH/CH₂Cl₂) yielded ethyl 2-(2-hydroxyphenyl)-4-methyl-6-oxo-1-(2-phenylethyl)-1,6-dihydro-5-pyrimidinecarboxylate (0.053 g, 72%): ¹H NMR (400 MHz, CHLOROFORM-d) δ ppm 7.38 (m, 1H), 7.21 (m, 4H), 6.96 (m, 4H), 4.46 (q, J=7.13 Hz, 2H), 4.30 (t, J=7.69 Hz, 2H), 2.98 (t, J=7.71 Hz, 2H), 2.43 (s, 3H), 1.44 (t, J=7.14 Hz, 3H); MS (ESI) 379.4 (M+H)⁺.

### Example 150

### Preparation of 2-(2-hydroxyphenyl)-6-methyl-5-(1-methylpropyl)-3-[2-(2-thienyl)ethyl]-4(3H)-pyrimidinone

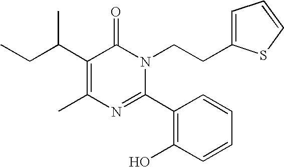

### a. Ethyl 2-acetyl-3-methylpentanoate

A THF (170 mL, 0.2 M) solution of LDA (21 mL, 2M in heptane/THF/ethylbenzene) was cooled to −78° C. in an oven-dried flask under N₂. A THF solution (10 mL) of ethyl 3-methylpentanoate (5.0 g, 34.67 mmol) was added dropwise and the resulting solution stirred at −78° C. for 1 h. Acetyl chloride (7.4 mL, 104.01 mmol) was added neat to the cold solution, and the resulting mixture was allowed to warm to room temperature over several hours. The reaction mixture was quenched by the addition of 1N HCl. The layers were separated, and the organic phase was washed with 5% NaHCO₃ and brine. The combined organic portion was dried over Na₂SO₄ and concentrated to an orange oil for purification. flash column chromatography purification (5-30% ethyl acetate/hexanes) provided pure product as a slightly yellow volatile oil which was visualized by I₂ staining (3.64 g, 56%). ¹H NMR (400 MHz, CHLOROFORM-d) δ ppm 4.13-4.24 (m, J=7.12, 7.12, 7.12, 1.64, 1.52 Hz, 2H) 3.31 (dd, J=12.76, 9.22 Hz, 1H) 2.19-2.28 (m, 4H) 1.37-1.48 (m, 1H) 1.29 (ddd, J=13.83, 6.76, 4.42 Hz, 3H) 1.18 (ddd, J=14.08, 6.76, 2.15 Hz, 1H) 0.87-0.97 (m, 6H)

### b. 2-Acetyl-3-methyl-N-[2-(2-thienyl)ethyl]pentanamide

A DME (3 mL) solution of ester (1.0 g, 5.37 mmol) from example 150a, thienylethylamine (0.57 mL, 4.89 mmol) and ethanol (0.5 mL) was subjected to microwave irradiation at 180° C. for 15 minutes. The reaction mixture was purified by flash column chromatography (10-70% ethyl acetate/hexanes) to give the desired product as a white solid in 41% yield (0.54 g). ¹H NMR (400 MHz, CHLOROFORM-d) δ ppm 7.17 (dd, J=5.18, 1.14 Hz, 1H) 6.95 (dd, J=5.05, 3.54 Hz, 1H) 6.82 (t, J=2.40 Hz, 1H) 6.36 (d, J=20.72 Hz, 1H) 3.47-3.58 (m, J=19.39, 6.63, 6.38, 6.38, 3.92 Hz, 2H) 3.19 (t, J=10.36 Hz, 1H) 3.02 (t, J=6.69 Hz, 2H) 2.23-2.26 (m, 3H) 2.01-2.11 (m, 1H) 1.35-1.47 (m, 1H) 1.05-1.16 (m, 1H) 0.89 (ddd, J=9.09, 7.07, 2.78 Hz, 5H) 0.86 (s, 1H); MS (m/z): 268 (M+H)

### c. 2-(2-Hydroxyphenyl)-6-methyl-5-(1-methylpropyl)-3-[2-(2-thienyl)ethyl]-4(3H)-pyrimidinone

To a suspension of ketoamide (0.23 g, 0.84 mmol) from example 150b in m-xylene (2.1 mL, 0.4 M), salicylamide (0.17 g, 1.26 mmol) and a few drops of isopropanol was added titanium(IV)isopropoxide (1.2 mL, 4.21 mmol). The resulting mixture was stirred at reflux for 3 days. The reaction mixture was quenched by the addition of 6N HCl and ethyl acetate and was allowed to stir overnight. The layers were separated, and the aqueous phase was extracted with 2 portions of dichlormethane. The combined organic portions were dried over Na₂SO₄ and concentrated to a brown oil. flash column chromatography purification (5% methanol/dichlormethane provided the title compound as a white solid (0.03 g, 10%): ¹H NMR (400 MHz, CHLOROFORM-d) δ ppm 9.84 (br. s., 1H) 7.35 (ddd, J=8.53, 7.14, 1.77 Hz, 1H) 7.10 (dd, J=5.18, 1.14 Hz, 1H) 7.02 (dd, J=8.34, 1.01 Hz, 1H) 6.93-6.98 (m, 1H) 6.86 (dd, J=5.18, 3.41 Hz, 1H) 6.62-6.66 (m, 1H) 4.34-4.42 (m, 2H) 3.23-3.29 (m, 2H) 2.81-2.91 (m, 1H) 2.35 (s, 3H) 1.92-2.04 (m, 1H) 1.69-1.80 (m, 1H) 1.31-1.37 (m, 3H) 0.89 (t, J=7.45 Hz, 3H); MS (m/z): 369 (M+H)

### Example 151

### Preparation of 2-(2-hydroxyphenyl)-6-methyl-5-(1-methylpropyl)-3-(2-phenylethyl)-4(3H)-pyrimidinone

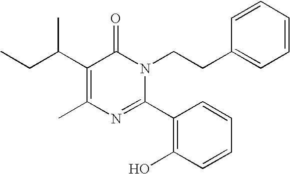

The title compound was prepared following the general procedure outlined in Example 150 except substituting phenethylamine for [2-(2-thienyl)ethyl]amine (0.07 g, 20% yield). ¹H NMR (400 MHz, CHLOROFORM-d) δ ppm 7.36 (td, J=7.20, 1.52 Hz, 2H) 7.17-7.25 (m, 3H) 6.94-7.04 (m, 4H) 4.33-4.41 (m, 2H) 2.94-3.01 (m, 2H) 2.79-2.90 (m, 1H) 2.33 (s, 3H) 1.98 (ddd, J=13.52, 8.08, 7.96 Hz, 1H) 1.75 (dt, J=13.83, 6.85 Hz, 1H) 1.35 (d, J=7.07 Hz, 3H) 0.84-0.92 (t, J=8.0 Hz, 3H); MS (m/z): 363 (M+H)

### Example 152

### Preparation of 2-(3-fluoro-2-hydroxyphenyl)-6-methyl-5-(1-methylpropyl)-3-(2-phenylethyl-4(3H)-pyrimidinone

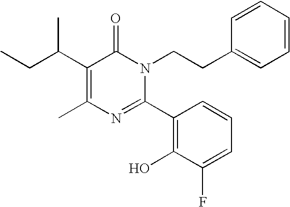

### a. 2-[3-fluoro-2-(methyloxy)phenyl]-6-methyl-(1-methylpropyl)-3-(2-phenylethyl)-4(3M)-pyrimidinone

The title compound was prepared following the general procedure outlined in Example 150 except substituting 3-fluoro-2-methoxybenzamide for salicylamide (0.03 g, 8% yield) in step c; MS (m/z): 395 (M+H).

### b. 2-(3-Fluoro-2-hydroxyphenyl)-6-methyl-5-(1-methylpropyl)-3-(2-phenylethyl)-4(3H)-pyrimidinone

To a 0° C. dichlormethane solution (4 mL, 0.2 M) of methyl ether from Example 3a (0.03 g, 0.076 mmol) was added BBr₃ (0.23 mL, 1M in dichlormethane) dropwise under N₂. The reaction mixture was allowed to warm to room temperature overnight. The reaction was quenched by the addition of methanol and was purified in two stages by flash column chromatography (5% methanol/dichlormethane then 20-50% ethyl acetate/hexanes) to provide pure product as a white solid (0.02 g, 74% yield): ¹H NMR (400 MHz, CHLOROFORM-D) δ ppm 7.18-7.25 (m, 3H) 7.10 (ddd, J=10.11, 8.21, 1.64 Hz, 1H) 6.85-6.96 (m, 4H) 4.19 (dt, J=10.55, 7.61 Hz, 2H) 2.92 (t, J=7.58 Hz, 2H) 2.85 (d, J=7.83 Hz, 1H) 2.31 (s, 3H) 1.90-2.02 (m, 1H) 1.78 (ddd, J=13.77, 7.07, 6.95 Hz, 1H) 1.37 (d, J=7.07 Hz, 3H) 0.85-0.94 (m, 3H); MS (m/z): 381 (M+H).

### Example 153

### Preparation of 5-butyl-2-(2-hydroxyphenyl)-6-methyl-3-(2-phenylethyl)-4(3H)-pyrimidinone

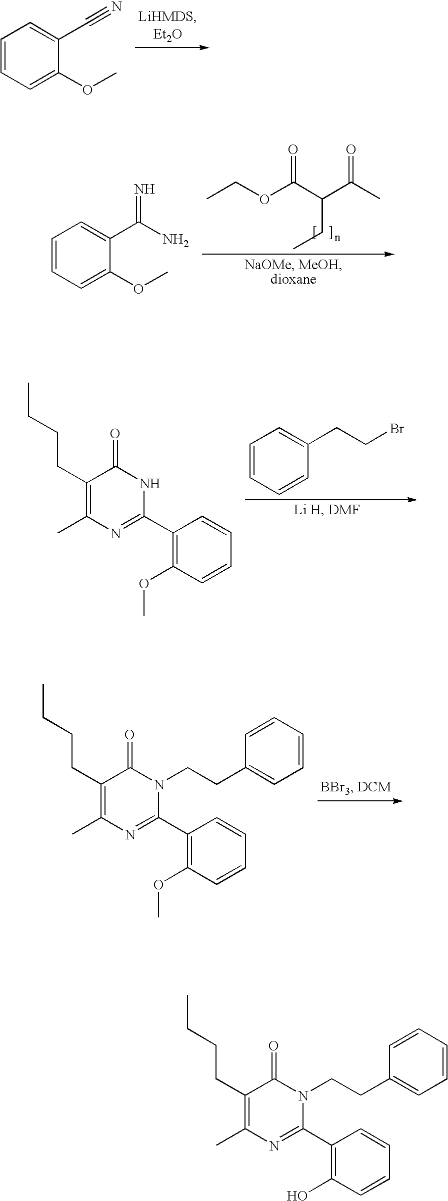

### a. 2-(Methoxy)benzenecarboxamidine

2-Methoxybenzonitrile (5 g, 37.5 mmol) was added to a 0° C. solution of LiHMDS (94 mL, 1M in hexanes) in anhydrous Et₂O (75 mL, 0.5 M) under N₂. After warming to room temperature, the mixture stirred for three days. The resulting reaction mixture was quenched by the addition of 1N HCl. The layers were separated and the aqueous phase was extracted 2 times with Et₂O. The aqueous layer was cooled in an ice-bath, adjusted to pH 12, and extracted 3 times with dichlormethane. The organic portions were pooled, dried over Na₂SO₄, and concentrated to a brown oil which solidified to a brown solid under vacuum (4.5 g, 80% yield): ¹H NMR (400 MHz, CHLOROFORM-d) δ ppm 7.57 (dd, J=7.58, 1.77 Hz, 1H) 7.35-7.41 (m, 1H) 6.94-7.03 (m, 2H) 5.24 (s, 3H) 3.89 (s, 3H).

### b. 5-Butyl-6-methyl-2-[2-(methyloxy)phenyl]-4(1H)-pyrimidinone

NaOMe (1.14 g, 20.0 mmol) was added to a 0° C. solution of 2-(methoxy)benzenecarboxamidine (1.0 g, 6.67 mmol) and ethyl 2-acetylhexanoate (1.49 g, 8.0 mmol) in methanol (70 mL) and 1,4-dioxane (20 mL). The resulting mixture was heated in a 120° C. oil bath in a sealed tube for 6 h. The solvents were removed and the residue was brought up in ethyl acetate and 1N HCl. The layers were separated and the aqueous layer was extracted with dichlormethane 3 times. The combined organic portions were dried over Na₂SO₄ and purified by flash column chromatography (20% dichlormethane/ethyl acetate) to give 0.64 g of product (35% yield): ¹H NMR (400 MHz, CHLOROFORM-d) ␣ ppm 10.97 (s, 1H) 8.41 (dd, J=7.83, 1.77 Hz, 1H) 7.49 (ddd, J=8.53, 7.14, 1.77 Hz, 1H) 7.08-7.15 (m, 1H) 7.04 (d, J=7.83 Hz, 1H) 4.04 (s, 3H) 2.53-2.60 (m, 2H) 2.39 (s, 3H) 1.47-1.55 (m, 2H) 1.39-1.45 (m, 2H) 0.95 (t, J=7.20 Hz, 3H); MS (m/z): 273 (M+H).

### c. Butyl-6-methyl-2-[2-(methyloxy)phenyl]-3-(2-phenylethyl)-4(3H)-pyrimidinone

LiH (0.02 g, 2.57 mmol) was added to a 0° C. solution of 5-butyl-6-methyl-2-[2-(methyloxy)phenyl]-4(1H)-pyrimidinone (0.20 g, 0.74 mmol) in DMF (5 mL, 0.15M) and stirred at 0° C. for 30 minutes. Bromoethyl benzene (0.3 mL, 2.21 mmol) was added and the resulting mixture stirred at room temperature for 40 hours. The reaction was quenched by the addition of ethyl acetate (15 mL) and water (25 mL). The layers were separated and the organic portion was washed 3 times with water, dried over NaSO₄, filtered, and concentrated to a yellow oil. flash column chromatography (10-100% ethyl acetate/hexanes) provided pure product as a white solid (0.14 g, 50%) plus the O-alkylated by-product (0.12 g, 43%); Desired N-alkylation product: ¹H NMR (400 MHz, CHLOROFORM-d) δ ppm 7.44-7.49 (m, 1H) 7.14-7.21 (m, 3H) 7.07-7.12 (m, 1H) 7.01-7.06 (m, 1H) 6.97 (cd, J=8.34 Hz, 1H) 6.85 (dd, J=7.20, 2.15 Hz, 2H) 4.30 (ddd, J=13.14, 10.48, 4.93 Hz, 1H) 3.79 (s, 3H) 3.62 (ddd, J=13.14, 10.36, 6.32 Hz, 1H) 2.89 (ddd, J=12.82, 10.29, 4.93 Hz, 1H) 2.76 (ddd, J=12.69, 10.42, 6.44 Hz, 1H) 2.55-2.66 (m, 2H) 2.36 (s, 3H) 1.53-1.60 (m, 2H) 1.44-1.52 (m, 2H) 1.00 (t, J=7.20 Hz, 3H). Undesired O-alkylation by-product: ¹H NMR (400 MHz, CHLOROFORM-d) δ ppm 7.67 (dd, J=7.58, 1.77 Hz, 1H) 7.38 (td, J=7.83, 1.77 Hz, 1H) 7.31 (td, J=6.25, 1.89 Hz, 4H) 7.25 (dd, J=6.19, 2.40 Hz, 1H) 6.99-7.07 (m, 2H) 4.63 (t, J=6.69 Hz, 2H) 3.86 (s, 3H) 3.13 (t, J=6.82 Hz, 2H) 2.54-2.61 (m, 2H) 2.51 (s, 3H) 1.36-1.47 (m, 4H) 0.96 (t, J=7.07 Hz, 3H)

### d. 5-Butyl-2-(2-hydroxyphenyl)-6-methyl-3-(2-phenylethyl)-4(3H)-pyrimidinone

To a 0° C. dichlormethane solution (1.8 mL, 0.2 M) of the butyl-6-methyl-2-[2-(methyloxy)phenyl]-3-(2-phenylethyl)-4(3H)-pyrimidinone (0.14 g, 0.37 mmol) was added BBr₃ (1.1 mL, 1M in dichlormethane) dropwise. The resulting solution was allowed to warm to room temperature while stirring overnight. The reaction was quenched by the addition of saturated Na₂CO₃ and dichlormethane. The layers were separated and the organic portion was dried over MgSO₄, filtered and concentrated to a yellow oil which was purified by flash column chromatography (15-100% ethyl acetate/hexanes) to give the title compound as a white solid (0.13 g, 98% yield); ¹H NMR (400 MHz, CHLOROFORM-d) δ ppm 7.14-7.25 (m, 5H) 6.87-6.93 (m, 3H) 6.82 (d, J=8.08 Hz, 1H) 4.12-4.23 (m, 2H) 2.85-2.94 (m, 2H) 2.51-2.60 (m, 2H) 2.27 (s, 3H) 1.47-1.57 (m, 2H) 1.39-6

### Example 154

### Preparation of 2-(2-hydroxyphenyl)-6-methyl-5-pentyl-3-(2-phenylethyl)-4(3H)-pyrimidinone

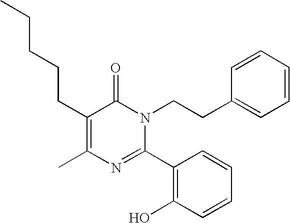

The title compound was prepared following the general procedure described in Example 153 except substituting ethyl 2-acetyl heptanoate for ethyl 2-acetyl hexanoate (0.13 g, quantitative yield) in step 153b. ¹H NMR (400 MHz, CHLOROFORM-d) □ ppm 9.74 (br. s., 1H) 7.24-7.32 (m, 2H) 7.18-7.22 (m, 3H) 6.89-6.98 (m, 4H) 4.24-4.32 (m, 2H) 2.89-2.98 (m, 2H) 2.52-2.61 (m, 2H) 2.29 (s, 3H) 1.54 (d, J=7.83 Hz, 2H) 1.41 (ddd, J=6.82, 3.79, 3.54 Hz, 4H) 0.89-0.98 (m, 3H); MS (m/z): 377 (M+H)

### Example 155

### Preparation of 5-hexyl-2-(2-hydroxyphenyl)-6-methyl-3-(2-phenylethyl)-4(3H)-pyrimidinone

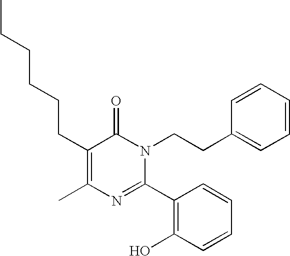

The title compound was prepared following the general procedure described in Example 153 except substituting ethyl 2-acetyl octanoate for ethyl 2-acetyl hexanoate (0.083 g, 81% yield) in step 153b; ¹H NMR (400 MHz, CHLOROFORM-d) δ ppm 9.61 (s, 1H) 7.35 (d, J=7.33 Hz, 2H) 7.17-7.26 (m, 3H) 6.94-7.03 (m, 4H) 4.31-4.39 (m, 2H) 2.96-3.03 (m, 2H) 2.53-2.60 (m, 2H) 2.32 (s, 3H) 1.49-1.60 (m, 2H) 1.33-1.45 (m, 5H) 1.27 (t, J=7.07 Hz, 1H) 0.87-0.96 (m, 3H); MS (m/z): 391 (M+H)

### Example 156

### Preparation of 5-butyl-2-(2-hydroxyphenyl)-6-methyl-3-[2-(2-thienyl)ethyl]-4(3H)-pyrimidinone

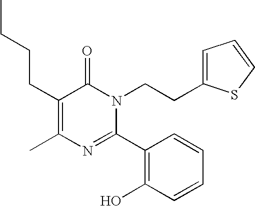

The title compound was prepared following the general procedure described in Example 153 except substituting 2-(2-bromoethyl)thiophene for bromoethyl benzene (0.081 g, 66% yield) in step 153b; ¹H NMR (400 MHz, CHLOROFORM-d) δ ppm 9.78 (br. s., 1H) 7.32 (td, J=7.83, 1.52 Hz, 1H) 7.20-7.26 (m, 1H) 7.10 (dd, J=5.05, 1.26 Hz, 1H) 6.95 (td, J=8.59, 2.02 Hz, 2H) 6.86 (dd, J=5.05, 3.54 Hz, 1H) 6.63 (d, J=2.53 Hz, 1H) 4.30-4.38 (m, 2H) 3.21-3.28 (m, 2H) 2.53-2.60 (m, 2H) 2.31 (s, 3H) 1.49-1.55 (m, 2H) 1.43-1.47 (m, 2H) 0.98 (t, J=7.20 Hz, 3H); MS (m/z): 369 (M+H)

### Example 157

### Preparation of 2-(2-hydroxyphenyl)-6-methyl-5-pentyl-3-[2-(2-thienyl)ethyl]-4(3H)-pyrimidinone

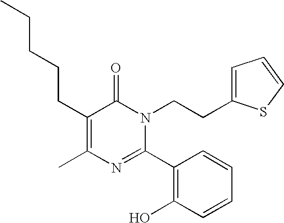

The title compound was prepared following the general procedure described in Example 153 except substituting ethyl 2-acetyl heptanoate for ethyl 2-acetyl hexanoate in step 153b and 2-(2-bromoethyl)thiophene for bromoethyl benzene (0.094 g, 78% yield) in 153c. ¹H NMR (400 MHz, CHLOROFORM-d) δ ppm 9.77 (br. s., J=1.77 Hz, 1H) 7.29-7.36 (m, 2H) 7.10 (dd, J=5.18, 1.14 Hz, 1H) 6.93-7.01 (m, 2H) 6.86 (dd, J=5.05, 3.54 Hz, 1H) 6.63 (d, J=2.53 Hz, 1H) 4.31-4.40 (m, 2H) 3.26 (t, J=7.33 Hz, 2H) 2.53-2.60 (m, 2H) 2.32 (s, 3H) 1.50-1.61 (m, 2H) 1.35-1.44 (m, 4H) 0.88-0.97 (m, 3H); MS (m/z): 383 (M+H)

### Example 158

### Preparation of 5-hexyl-2-(2-hydroxyphenyl)-6-methyl-3-[2-(2-thienyl)ethyl]-4(3H)-pyrimidinone

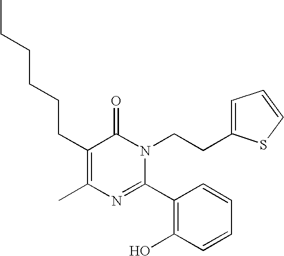

The title compound was prepared following the general procedure described in Example 153 except substituting ethyl 2-acetyl octanoate for ethyl 2-acetyl hexanoate in step 153b and 2-(2-bromoethyl)thiophene for bromoethyl benzene (0.056 g, 64% yield) in step 153c. ¹H NMR (400 MHz, CHLOROFORM-d) 5 ppm 9.75 (br. s., 1H) 7.31-7.38 (m, 1H) 7.11 (dd, J=5.18, 1.14 Hz, 1H) 7.01 (d, J=8.34 Hz, 1H) 6.93-6.99 (m, 1H) 6.87 (dd, J=5.05, 3.28 Hz, 1H) 6.65 (d, J=2.78 Hz, 1H) 4.34-4.41 (m, 2H) 3.27(t, J=0.33 Hz, 2H) 2.53-2.60 (m, 2H) 2.33 (s, 3H) 1.50-1.61 (m, 2H) 1.32-1.44 (m, 6H) 0.86-0.96 (m, 3H); MS (m/z): 397 (M+H)

### Example 159

### Preparation of 2-(3-fluoro-2-hydroxyphenyl)-3-[2-(3-fluorophenyl)ethyl]-5,6,7,8-tetrahydro-4(3H)-quinazolinone

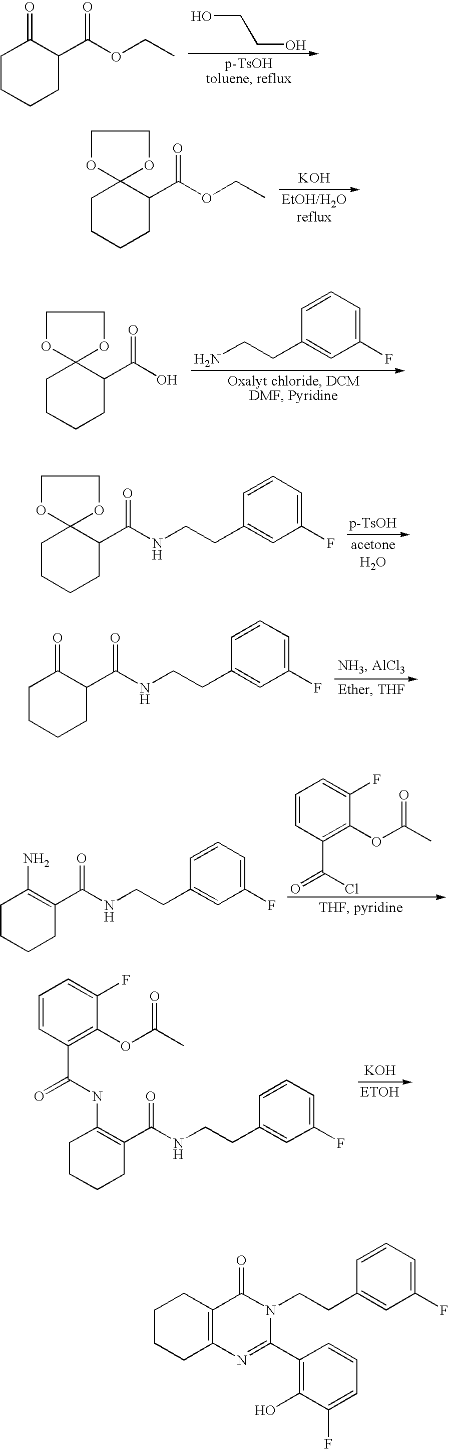

### a. Ethyl 1,4-dioxaspiro[4.5]decane-6-carboxylate

A mixture of commercially available ethyl 2-oxocyclohexanecarboxylate (20 g, 117 mmol), ethylene glycol (8.02 g, 129 mmol), and p-toluenesulfonic acid (1.0 g) in toluene (200 mL) was heated to 120° C. for 4 h under a Dean-Stark apparatus. The reaction mixture was cooled to RT, the solvent was removed, and the residue was partitioned between ethyl acetate and saturated NaHCO₃. The layers were separated, and the aqueous portion was extracted 3 times with ethyl acetate. The organic portions were combined, dried (MgSO₄) and concentrated to give the product as a colorless oil which was carried on to next step without further purification.

### b. 1,4-dioxaspiro[4.5]decane-6-carboxylic acid

To a solution of ethyl 1,4-dioxaspiro[4.5]decane-6-carboxylate in EtOH (150 mL) was added 85% KOH solution in water (15 g/100 mL), and the mixture stirred at reflux overnight. The reaction mixture was cooled to RT, the solvent evaporated, and the residue was partitioned between CH₂Cl₂ and 2N HCl. After separating the layers, the aqueous portion was extracted 3 times with CH₂Cl₂. The organic portions were combined, dried (Na₂SO₄), and concentrated in vacuo to give the acid product as a light yellow oil (14 g, two step yield: 65%).

### c. N-[2-(3-fluorophenyl)ethyl]-1,4-dioxaspiro[4.5]decane-6-carboxamide

To a 0° C. solution of 1,4-dioxaspiro[4.5]decane-6-carboxylic acid (7.0 g, 34.65 mmol) in CH₂Cl₂ (200 mL) was added oxalyl chloride (4.9 mL) in a dropwise fashion. After 15 min stirring at 0° C., the mixture was allowed to stir at RT for 2 h. The solvent and excess oxalyl chloride were removed to give an oil, which was brought up in fresh CH₂Cl₂ and cooled to 0° C. A pyridine solution (20 mL) of 2-(3-fluorophenyl)ethanamine (7.22, 51.98 mmol) was added dropwise, and the resulting solution was allowed to warm to RT while stirring overnight. The reaction mixture was partitioned between CH₂Cl₂ and 1N HCl. After separating the layers, the organic portion was washed with water and aq. NaHCO₃. The organic portion was combined, dried (Na₂SO₄), and concentrated in vacuo to give the product as a white solid (11.0 g, yield=95%) which was used in the next reaction without further purification.

### d. N-[2-(3-fluorophenyl)ethyl]-2-oxocyclohexanecarboxamide

To a solution of N-[2-(3-fluorophenyl)ethyl]-1,4-dioxaspiro[4.5]decane-6-carboxamide (11.0 g, 34.1 mol) in acetone and water (200 mL/100 mL) was added p-toluenesulfonic acid (9.72 g, 51.15 mol). This mixture was stirred and heated to 95° C. for 8 h. After cooling to RT, the solvent was removed and the residue was partitioned between CH₂Cl₂ and aq. Na₂CO₃. After separating the layers, the aqueous layer was extracted 2 times with fresh CH₂Cl₂, and the combined organic portions were dried (Na₂SO₄), filtered and concentrated to provide crude as a white solid. Purification by silica gel column chromatography (50% ethyl acetate/hexanes) gave the product as a white solid in 82% yield (7.3 g).

### e. 2-amino-N-[2-(3-fluorophenyl)ethyl]-1-cyclohexene-1-carboxamide

A 0° C. solution of N-[2-(3-fluorophenyl)ethyl]-2-oxocyclohexanecarboxamide (2.08 g, 7.91 mmol) in diethyl ether (250 mL) and THF (10 mL) was saturated with gaseous ammonia for 3 h. AlCl₃ (2 g) was added, and the mixture was allowed to warm to RT while stirring overnight. The resulting suspension was filtered, and the filtrate was concentrated to provide product as a colorless oil in 97% yield (2.0 g); MS (m/z): 263 (M+H).

### f. 2-fluoro-6-{N-[2-({[2-(3-fluorophenyl)ethyl]amino}carbonyl)-1-cyclohexen-1-yl]glycyl}phenyl acetate

To a solution of 2-amino-N-[2-(3-fluorophenyl)ethyl]-1-cyclohexene-1-carboxamide (1.0 g, 3.82 mmol) in THF (100 mL) and pyridine (7 mL) was added 2-(chlorocarbonyl)-6-fluorophenyl acetate (1.46 g, 6.10 mmol). The mixture was heated to reflux overnight. After cooling to RT, diethyl ether (200 mL) was added, and the precipitated salts were removed by filtration. The filtrate was concentrated, diluted with diethyl ether (250 mL), and washed three times with 2N HCl (50 mL portions). The organic layer was washed successively with water and brine, and dried over Na₂SO₄, filtered, and concentrated for purification. Purified by silica gel column chromatography (30-50% ethyl acetate/hexanes) to provide the pure product as a white solid in 33% yield (0.51 g). MS (m/z) 442 (M+H).

### g. 2-(3-fluoro-2-hydroxyphenyl)-3-[2-(3-fluorophenyl)ethyl]-5,6,7,8-tetrahydro-4(3H)-quinazolinone

A solution of 2-fluoro-6-{N-[2-({[2-(3-fluorophenyl)ethyl]amino}carbonyl)-1-cyclohexen-1-yl]glycyl}phenyl acetate (0.510 g, 1.15 mmol) in EtOH (20 mL) and 85% KOH (20 mL) was heated to reflux overnight. After cooling to RT, the pH was adjusted to about 1 with 2N HCl and extracted three times with CH₂Cl₂. The organic portions were combined, dried (Na₂SO₄), filtered, and concentrated. Purification by silica gel column chromatography (2-3% CH₃OH/CH₂Cl₂) provided the pure product as a white solid in 59% yield (260 mg). MS (m/z): 383.2 (M+H).

### Example 160

### Preparation of 2-(3-fluoro-2-hydroxyphenyl)-3-[2-(3-fluorophenyl)ethyl]-3,5,6,7,8,9-hexahydro-4H-cyclohepta[d]pyrimidin-4-one

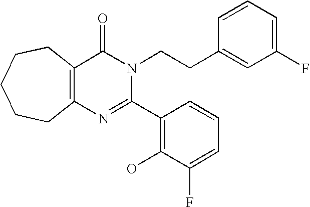

The title compound was prepared following the general procedure of Example 159 except substituting ethyl 2-oxocyclohexanecarboxylate with ethyl 2-oxocycloheptanecarboxylate; MS (m/z): 397.4 (M+H)

### Example 161

### Preparation of Ethyl 2-(2-hydroxyphenyl)-4-oxo-3-(2-phenylethyl)-3,5, 7,8-tetrahydropyrido[4,3-d]pyrimidine-6(4H)-carboxylate

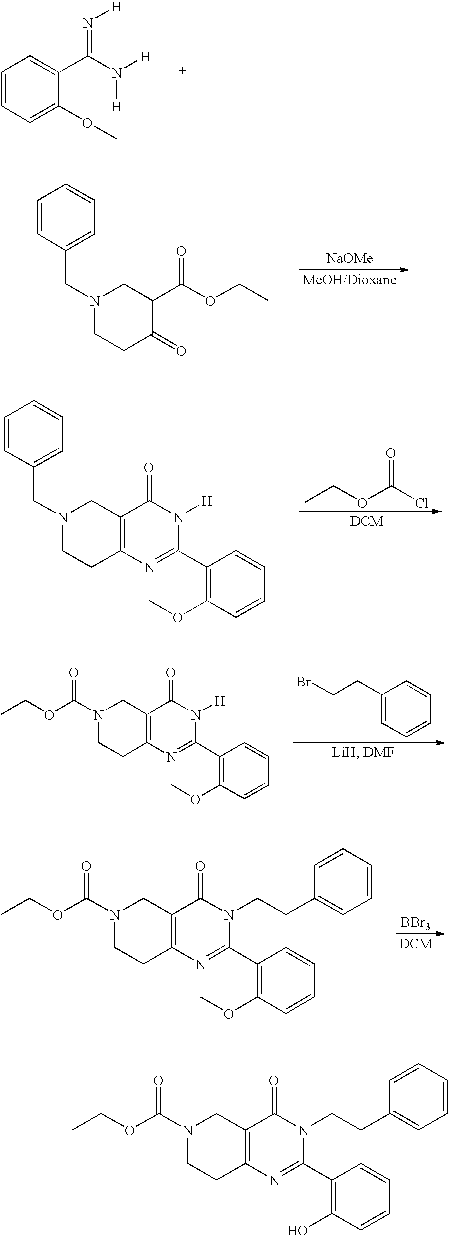

### a. 6-Benzyl-2-(2-methoxy-phenyl)-5,6,7,8-tetrahydro-3H-pyrido[4,3-d]pyrimidin-4-one

To a 0° C. solution of 2-(methyloxy)benzenecarboximidamide (300 mg, 2.0 mmol) in MeOH/1,4-dioxane (20 mL/7 mL) was added 25% NaOCH₃ in MeOH (1.39 mL) and then stirred for 15 mins. 1-Benzyl-4-oxo-piperidine-3-carboxylic acid ethyl ester hydrochloride salt (893 mg, 3.0 mmol) was introduced and the reaction mixture was heated to reflux for 2 h. The solvent was removed, the residue was diluted with 10 mL H₂O, the pH=7-8 was adjusted by adding acetic acid and the aqueous layer was extracted by CH₂Cl₂ (3×100 mL). The organic layers were combined, dried over Na₂SO₄ and concentrated. Purified by silica gel column chromatography (10% to 95% ethylacetate/hexane, with 1% MeOH) to give product 1.21 g as a white solid in 87% yield. MS (m/z): 348 (M+H)

### b. Ethyl 2-[2-(methyloxy)phenyl]-4-oxo-3,5,7,8-tetrahydropyrido[4,3-d]pyrimidine-6(4H)-carboxylate

To a solution of 6-benzyl-2-(2-methoxy-phenyl)-5,6,7,8-tetrahydro-3H-pyrido[4,3-d]pyrimidin-4-one (300 mg, 0.865 mmol) in dichlormethane (8 mL) was added ethyl chloroformate. The mixture was heated to reflux for 2.5 h. The reaction mixture was concentrated and purified by silica gel column chromatography (30% to 90% ethylacetate/hexane) to give product as white solid in 89% yield (230 mg). MS (mfz): 330 (M+H)

### c. Ethyl 2-[2-(methyloxy)phenyl]-4-oxo-3-(2-phenylethyl)-3,5,7,8-tetrahydropyrido[4,3-d]pyrimidine-6(4H)-carboxylate

To a 0° C. solution of ethyl 2-[2-(methyloxy)phenyl]-4-oxo-3,5,7,8-tetrahydropyrido[4,3-d]pyrimidine-6(4H)-carboxylate (220 mg, 0.699 mmol) in dry DMF was added lithium hydride (10.57 mg, 1.34 mmol) and the mixture was stirred for 5 min at this temperature and 1-(2-bromo-ethyl)-benzene (0.55 ml, 0.01 mmol) was added and stirred at RT overnight. The mixture was concentrated and diluted with Et₂O, dried over MgSO₄, filtered and concentrated. The crude residue was purified by silica gel column chromatography (30% to 90% ethylacetate/hexane) to give desired product in 59% yield (170 mg). MS (m/z): 434 (M+H).

### d. Ethyl 2-(2-hydroxyphenyl)-4-oxo-3-(2-phenylethyl)-3,5,7,8-tetrahydropyrido[4,3-d]pyrimidine-6(4H)-carboxylate

To a solution of ethyl 2-[2-(methyloxy)phenyl]-4-oxo-3-(2-phenylethyl)-3,5,7,8-tetrahydropyrido[4,3-d]pyrimidine-6(4H)-carboxylate (150 mg, 0.346 mmol) in dichlormethane at −60° C. was added boron tribromide (1.0 M solution in dichloromethane) (2.0 mL, 2.0 mmol) and stirred at this temperature for 1 h and then stirred at RT overnight. The reaction was quenched by water (15 mL). The aqueous layer was separated, was extracted with dichlormethane twice after the pH was adjusted to 7 with 2 N NaOH. The organic layers were combined and washed with brine, dried (MgSO₄), filtered and concentrated. Purification by silica gel column chromatography (30%-90% ethyl acetate/hexane) gave the product as a white solid in 93% yield (135 mg). MS (m/z): 420(M+H).

### Example 162

### Preparation of (2-hydroxyphenyl)-6-(3-methylbutanoyl)-3-(2-phenylethyl)-5,6,7,8-tetrahydropyrido[4,3-o]pyrimidin-4(3H)-one

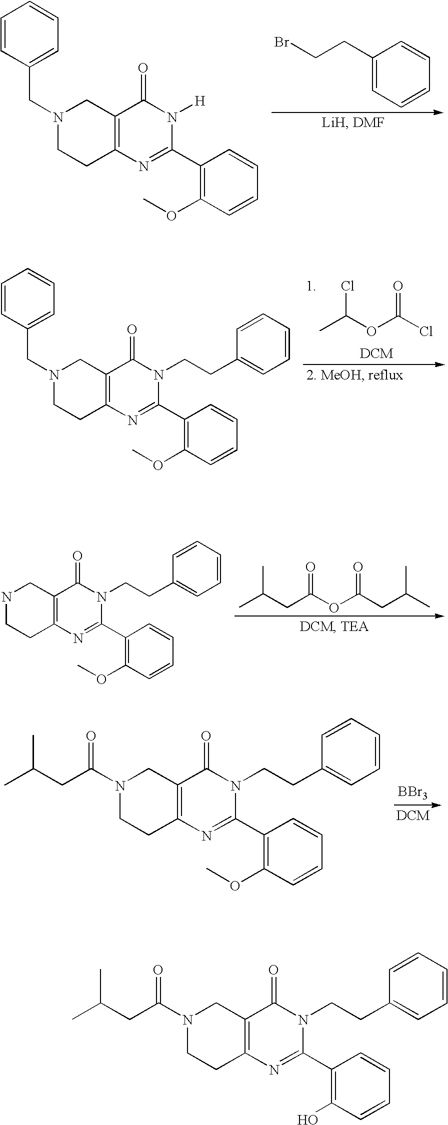

### a. 2-[2-(Methyloxy)phenyl]-3-(2-phenylethyl)-6-(phenyl methyl)-5,6,7,8-tetrahydropyrido[4,3-d]pyrimidin-4(3H)-one

The title compound was prepared from alkylation of 6-benzyl-2-(2-methoxy-phenyl)-5,6,7,8-tetrahydro-3H-pyrido[4,3-d]pyrimidin-4-one of Example 161 using phenethylbromide as alkylating agent as previously outlined. MS (m/z): 452 (M+H)

### b. 2-[2-(methyloxy)phenyl]-3-(2-phenylethyl)-5,6,7,8-tetrahydropyrido[4,3-a]pyrimidin-4(3H)-one

To a solution of 2-[2-(methyloxy)phenyl]-3-(2-phenylethyl)-6-(phenylmethyl)-5,6,7,8-tetrahydropyrido[4,3-d]pyrimidin-4(3H)-one (300 mg, 0.665 mmol) in dichlormethane (5 mL) was added 1-chloroethyl chloridocarbonate (0.29 mL, 2.66 mmol). The mixture was heated to reflux for an hour. The solvent was removed and refilled with methanol (6 mL) and heated to reflux for 1 h. After concentrated down, the crude was purified by silica gel column chromatography (10% methanol/dichlormethane) to give product as a white solid in 90% yield (216 mg). MS (m/z): 362(M+H).

### c. 6-(3-methylbutanoyl)-2-[2-(methyloxy)phenyl]-3-(2-phenylethyl)-5,6,7,8-tetrahydropyrido[4,3-d]pyrimidin-4(3H)-one

To a solution of 2-[2-(methyloxy)phenyl]-3-(2-phenylethyl)-5,6,7,8-tetrahydropyrido[4,3-d]pyrimidin-4(3H)-one (100 mg, 0.277 mmol) in dichlormethane was added 3-methylbutanoic anhydride (0.065 mL, 0.332 mmol). The mixture was stirred at rt for 4 h. The reaction was concentrated and purified by silica gel column chromatography (40-90% ethyl acetate/hexanes) which gave product as a white solid in 92% yield (113 mg). MS (m/z): 445(M+H). Subsequent deprotection using BBr3 as previously outlined resulted in the title compound. MS (m/z): 432 (M+H)

### Example 163

### Preparation of 5-ethyl-2-(2-hydroxyphenyl)-6-methyl-3-[2-(2-thienyl)ethyl]-4(3H)-pyrimidinone

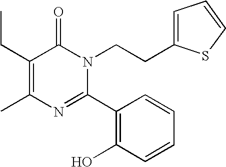

### a. 2-(2-Methyl-[1,3]dioxolan-2-yl)-butyric acid

A mixture of commercially available 2-ethyl-3-oxo-butyric acid ethyl ester (54 g, 0.34 mol), ethylene glycol (23.3 g, 0.375 mol), and p-toluenesulfonic acid (0.2 g) in toluene (500 mL) was heated to 120° C. for 4 h under a Dean-Stark apparatus. The reaction mixture was cooled to RT, the solvent was removed, and the residue was partitioned between ethyl acetate and saturated NaHCO₃. The layers were separated, and the aqueous portion was extracted 3 times with ethyl acetate. The organic portions were pooled, dried (MgSO₄) and concentrated to give the ethyl 2-(2-methyl-1,3-dioxolan-2-yl)butanoate product as a colorless oil in 91% yield (63 g). To a solution of the ester (60 g, 0.297 mol) provided above in EtOH (750 mL) was added 85% KOH solution in water (30 mL), and the mixture stirred at reflux overnight. The reaction mixture was cooled to RT, the solvent evaporated, and the residue was partitioned between CH₂Cl₂ and 2N HCl. After separating the layers, the aqueous portion was extracted 3 times with CH₂Cl₂. The organic portions were pooled, dried (Na₂SO₄), and concentrated in vacuo to give the acid product as a light yellow oil (27 g, 52% yield).

### b. 2-Ethyl-3-oxo-N-(2-thiophen-2-yl-ethyl)butyramide

To a 0° C. solution of 2-(2-methyl-[1,3]dioxolan-2-yl)-butyric acid (4 g, 0.023 mol) in CH₂Cl₂ (30 mL) was added oxalyl chloride (7.2 mL) in a dropwise fashion. After 15 min at 0° C., the mixture was allowed to stir at RT for 2 h. The solvent and excess oxalyl chloride were removed to give an oil, which was brought up in fresh CH₂Cl₂ and cooled to 0° C. A pyridine solution (4 mL) of 2-thiophen-2-yl ethylamine (5.3 g, 0.041 mol) was added dropwise, and the resulting solution was allowed to warm to RT while stirring overnight. The reaction mixture was partitioned between CH₂Cl₂ and 1N HCl. After separating the layers, the organic portion was washed with water and aq. NaHCO₃. The organic portion was pooled, dried (Na₂SO₄), and concentrated in vacuo to give 2-(2-methyl-1,3-dioxolan-2-yl)-N-[2-(2-thienyl)ethyl]butanamide product (4.4 g, 68%) which was used in the next reaction without further purification. To a solution of ketal (4.4 g, 0.016 mol) in acetone and water (50 mL/1 mL) was added p-toluenesulfonic acid (4.7 g, 0.025 mol). This mixture was stirred and heated to 95° C. for 4 h. After cooling to RT, the solvent was removed and the residue was partitioned between CH₂Cl₂ and aq. Na₂CO₃. After separating the layers, the aqueous layer was extracted 2 times with fresh CH₂Cl₂, and the combined organic portions were dried (NaSO₄), filtered and concentrated to provide a white solid. The solid was triturated with 1:1 hexanes/diethyl ether to give 3.2 g (86%) the product.

### c. (2Z)-3-amino-2-ethyl-N-[2-(2-thienyl)ethyl]-2-butenamide

A 0° C. solution of 2-ethyl-3-oxo-N-[2-(2-thienyl)ethyl]butanamide (3.2 g, 11.3 mmol) in diethyl ether (250 mL) and THF (10 mL) was saturated with gaseous ammonia for 3 h. AlCl₃ (2 g) was added, and the mixture was allowed to warm to RT while stirring overnight. The resulting suspension was filtered, and the filtrate was concentrated to provide product as a colorless oil (0.8 g, 25%).

### d. 5-Ethyl-2-(2-hydroxyphenyl)-6-methyl-3-[2-(2-thienyl)ethyl]-4(3H)-pyrimidinone

To a solution of (Z)-3-amino-2-ethyl-but-2-enoic acid [2-(thiophene-2yl)-ethyl]-amide (1.9 g, 8.15 mmol) in THF (50 mL) and pyridine (3 mL) was added acetic acid 2-chlorocarbonyl-phenyl ester (2.6 g, 13 mmol). The mixture was heated to reflux overnight. After cooling to RT, diethyl ether (200 mL) was added, and the precipitated salts were removed by filtration. The filtrate was concentrated, diluted with diethyl ether (250 mL), and washed three times with 2N HCl (50 mL portions). The organic layer was washed successively with water and brine, and dried over Na₂SO₄, filtered, and concentrated for purification. Flash column chromatography (15% ethyl acetate/hexanes) provided the pure product 1.6 g. A solution of amide (0.4 g, 0.1 mmol) in EtOH (40 mL) and 85% KOH (40 mL) was heated to reflux overnight. After cooling to RT, the reaction mixture was adjusted to pH 1 with 2N HCl and extracted three times with CH₂Cl₂. The organic portions were combined, dried (Na₂SO₄), filtered, and concentrated for purification. Flash column chromatography (2-3% CH₃OH/CH₂Cl₂) provided the pure pyrimidinone product 0.15 g. yield 45%. ¹H NMR (400 MHz, CDCl₃): δ 9.66(br, 1H), 7.14 (m, 2H), 6.95 (m, 3H), 6.55 (d, 1H), 4.28 (t, 2H), 3.30 (t, 2H), 2.46 (q, 2H), 2.33 (s, 3H), 1.08 (t, 3H). MS (m/z): 341.2 (M+H).

### Example 164

### Preparation of 5-Isopropyl-2-(2-hydroxy-phenyl)-6-methyl-3-(2-thiophen-2-yl-ethyl)-3H-pyrimidin-4-one

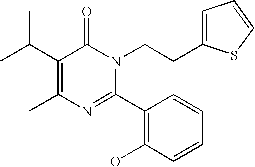

The title compound was prepared by substituting 2-isopropyl-3-oxo-butyric acid ethyl ester for 5-chloro-3-oxobutanoate in Example 26b followed by deprotection led to the product: ¹H NMR (400 MHz, CDCl₃): [symbol] 9.84 (br, 1H, OH), 7.40-7.26 (m, 2H), 7.10 (d, 1H), 7.05 (d, 1H), 6.96 (t, 1H), 6.87 (t, 1H), 6.68 (d, 1H), 4.39 (t, 2H), 3.30 (t, 2H), 3.15 (m, 1H), 2.38 (s, 3H), 1.40 (d, 6H). MS (m/z): 355.4 (M+H).

### Example 165

### Preparation of 5-isopropyl-2-(2-hydroxy-phenyl)-6-methyl-3-(2-cyclohexyl-ethyl)-3H-pyrimidin-4-one

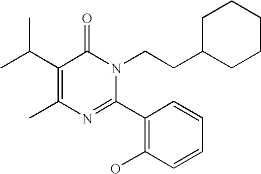

The title compound was prepared by substituting 2-isopropyl-3-oxo-butyric acid ethyl ester for 5-chloro-3-oxobutanoate in 26b and substituting 2-cyclohexyl ethyl bromide for phenethylbromide in Example 26c followed by deprotection led to the product: ¹H NMR (400 MHz, CDCl₃): 9.60 (br, 1H, OH), 7.33-7.27 (m, 2H), 6.97-6.91 (m, 2H), 4.02 (t, 2H), 3.13-3.09 (m, 1H), 2.32 (s, 3H), 1.61-1.52 (m, 7H), 1.36 (d, 6H), 1.16-1.09 (m, 4H), 0.81-0.78 (m, 2H). MS (m/z): 355.2 (M+H).

### Example 166

### Preparation of 5-Ethyl-2-(2-hydroxy-3-fluorophenyl)-6-methyl-3-(2-fluorophenylethyl)-3H-pyrimidin-4-one

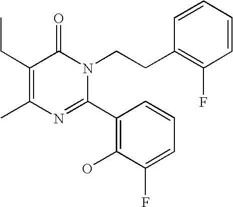

The title compound was a synthetic intermediate in preparation of Example 45. ¹H NMR (400 MHz, CDCl₃): [symbol] 9.41 (br, 1H, OH), 7.19-7.11 (m, 1H), 7.16-7.05 (m, 1H), 6.98-6.90 (m, 2H), 6.88-6.81 (m, 3H), 4.21 (t, 2H), 2.97 (t, 2H), 2.57 (q, 2H), 2.26 (s, 3H), 1.14 (t, 3H). MS (m/z): 371.2 (M+H).

### Example 167

### Preparation of 5-propenyl-2-(2-hydroxy-3-fluorophenyl)-6-methyl-3-(3-fluorophenylethyl)-3H-pyrimidin-4-one

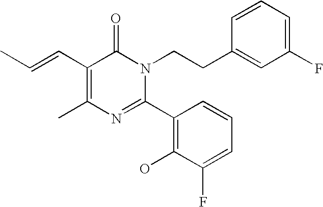

The title compound was prepared by substituting 2-acetyl-pent-4-enoic acid for 2-ethyl 2-oxocyclohexanecarboxylate in Example 159. ¹H NMR (400 MHz, CDCl₃): [symbol] 9.55 (br, 1H, OH), 7.10-6.99 (m, 2H), 6.90-6.72 (m, 4H), 6.58 (d, 1H), 6.47 (d, 1H), 6.21 (d, 1H), 4.16 (t, 2H), 2.80 (t, 2H), 2.25 (s, 3H), 1.90 (d, 3H). MS (m/z): 383.2 (M+H).

### Example 168

### Preparation of 3-(2-cyclohexylethyl)-2-(2-hydroxyphenyl)-5, 6,7,8-tetrahydro-4(3H)-quinazolinone

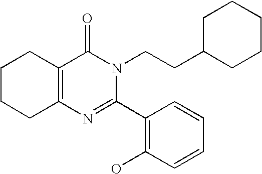

### a. 2-Methoxy-benzamidine

At 0° C. anhydrous ether was introduced to flask under Argon, LiHMDS (94 ml, 93.9 mmol) was then introduced and stirred for 5 mins. 2-Methoxy-benzonitrile (5 g, 37.6 mmol) was added and the mixture was stirred at room temperature for 2-3 days. When all the starting material was consumed, solvent was removed and 200 mL cold 1N HCl was added and stirred to make HCl salt. Extracted with Et₂O (3×300 mL) and then pH was adjusted to 13 by 6N NaOH. Extraction with CH₂Cl₂ and dried over Na₂SO₄. Filtered and the filtrate was concentrated to obtain the above benzamidine compound in 91% yield.

### b. 2-[2-(methyloxy)phenyl]-5,6,7,8-tetrahydro-4(1H)-quinazolinone

25% NaOMe in methanol (0.44 mL) was added to a 0° C. solution of 2-(methoxy)benzenecarboxamidine (0.15 g, 1.0 mmol) and 2-oxo-cyclohexanecarboxylic acid ethyl ester (0.26 g, 1.5 mmol) in methanol (15 mL) and 1,4-dioxane (5 mL). The resulting mixture was heated in a 120° C. oil bath in a sealed tube for 1 h. The solvents were removed and the residue was diluted with H₂O and pH was adjusted to 8 with acetic acid. The layers were separated and the aqueous layer was extracted with dichlormethane 3 times. The combined organic portions were dried over Na₂SO₄ and purified by flash column chromatography (70% ethyl acetate/hexanes) to give 0.22 g of product (86% yield).

### c. 3-(2-cyclohexylethyl)-2-[2-(methyloxy)phenyl]-5,6,7,8-tetrahydro-4(3H)-quinazolinone

NaH (0.029 g, 1.2 mmol) was added to a 0° C. solution of 2-[2-(methyloxy)phenyl]-5,6,7,8-tetrahydro-4(1H)-quinazolinone (0.15 g, 0.60 mmol) in DMF (3 mL) and stirred at RT for 10 minutes. Bromoethyl cyclohexane (0.3 mL, 2.21 mmol) was added and the resulting mixture stirred at room temperature overnight. The reaction was quenched with cold 6N HCl and extracted with ethyl acetate. The layers were separated and the organic portion was washed 3 times with water, dried over NaSO₄, filtered, and concentrated. Crude product was puririded by flash column chromatography to afford pure product (0.081 mg) in 38% yield.

### d. 3-(2-cyclohexylethyl)-2-(2-hydroxyphenyl)-5,6,7,8-tetrahydro-4(3H)-quinazolinone

To a −60° C. dichlormethane solution (5 mL) of the 3-(2-cyclohexylethyl)-2-[2-(methyloxy)phenyl]-5,6,7,8-tetrahydro-4(3H)-quinazolinone (0.15 g, 0.41 mmol) was added BBr₃ dropwise. The resulting solution was allowed to warm to room temperature while stirring overnight. The reaction was quenched by the addition of saturated NaHCO₃ and dichlormethane. The layers were separated and the organic portion was dried over Na₂SO₄, filtered and concentrated to a yellow oil which was purified by flash column chromatography (3-5% MeOH/DCM) to give the title compound (0.10 g, 69% yield). ¹H NMR (400 MHz, CDCl₃): [symbol] 9.97(br, 1H), 7.03 (m, 2H), 6.72 (t, 1H), 6.68 (d, 1H), 3.90 (m, 2H), 2.32 (m, 4H), 1.70 (m, 4H), 1.50 (m, 3H), 1.32 (m, 4H), 0.99 (m, 4H), 0.59 (m, 2H). MS (m/z): 353.4 (M+H).

### Example 169

### Preparation of 3-(2-thiophen-2-yl-ethyl)-2-(2-hydroxy-phenyl)-5,6,7,8-tetrahydro-3H-quinazolin-4-one

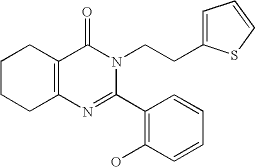

The title compound was prepared by substituting 2-thiophene-2-yl-ethyl bromide for (2-Bromo-ethyl)-cyclohexane in Example 168: ¹H NMR (400 MHz, CDCl₃): 6.62(br, 1H), 7.22 (m, 2H), 7.18 (d, 1H), 7.00 (m, 2H), 6.89 (t, 1H), 6.63 (d, 1H), 4.27 (t, 2H), 3.29 (t, 2H), 2.68 (m, 4H), 1.90 (m, 4H). MS (m/z): 353.4 (M+H).

### Example 170

### Preparation of 3-(2-thiophen-2-yl-ethyl)-2-(2-hydroxy-3-fluorophenyl)-5,6,7,8-tetrahydro-3H-quinazolin-4-one

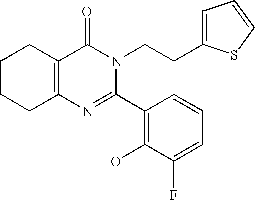

The title compound was prepared by substituting 2-thiophene-2-yl-ethyl bromide for (2-Bromo-ethyl)-cyclohexane in Example 159c: ¹H NMR (400 MHz, CDCl₃): δ 9.22(br, 1H), 7.13(m, 2H), 6.92 (m, 3H), 6.60 (d, 1H), 4.28 (t, 2H), 3.20 (t, 2H), 2.70 (m, 4H), 1.84 (m, 4H). MS (m/z): 371.2 (M+H).

### Example 171

### Preparation of 3-(2-thiophen-3-yl-ethyl)-2-(2-hydroxy-phenyl)-5,6,7,8-tetrahydro-3H-quinazolin-4-one

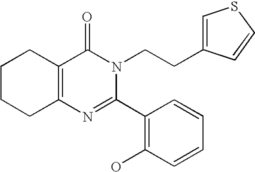

The title compound was prepared by substituting 3-(2-bromoethyl)thiophene for (2-Bromo-ethyl)-cyclohexane in Example 168: ¹H NMR (400 MHz, CDCl₃): [symbol] 9.61(br, 1H), 7.34-7.13 (m, 4H), 7.11-6.82 (m, 3H), 4.32 (t, 2H), 2.93 (t, 2H), 2.56 (m, 4H), 1.84 (m, 4H). MS (m/z): 353.4 (M+H).

### Example 172

### Preparation of 3-(3-chlorophenethyl)-2-(2-hydroxy-phenyl)-5,6,7,8-tetrahydro-3H-quinazolin-4-one

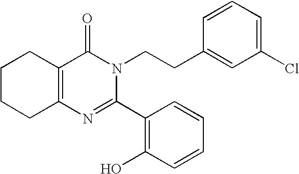

The title compound was prepared by substituting 3-chlorophenethyl bromide for (2-Bromo-ethyl)-cyclohexane in Example 168. ¹H NMR (400 MHz, CDCl₃): [symbol] 8.59(s, 1H), 7.28-7.07(m, 6H), 6.83 (t, 1H), 6.81 (d, 1H), 4.33 (t, 2H), 3.05 (t, 2H), 2.58 (m, 4H), 1.80 (m, 4H). MS (m/z): 381/383(M+H).

### Example 173

### Preparation of 3-(2-cyclopentylethyl)-2-(2-hydroxy-phenyl)-5,6,7,8-tetrahydro-3H-quinazolin-4-one

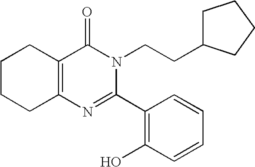

The title compound was prepared by substituting 2-cyclopentylethyl bromide for (2-Bromo-ethyl)-cyclohexane in Example 168: ¹H NMR (400 MHz, CDCl₃): [symbol] 9.94(br, 1H), 7.33 (m, 2H), 6.96 (m, 2H), 4.08 (t, 2H), 2.55 (m, 4H), 1.78 (m, 4H), 1.63 (M, 4H), 1.51 (m, 5H), 0.95 (m, 2H). MS (m/z): 339.4 (M+H).

### Example 174

### Preparation of 3-(3-trifluoromethylphenethyl)-2-(2-hydroxy-phenyl)-5,6,7,8-tetrahydro-3H-quinazolin-4-one

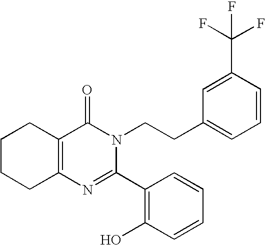

The title compound was prepared by substituting 3-trifluoromethylphenethyl bromide for (2-Bromo-ethyl)-cyclohexane in Example 168: ¹H NMR (400 MHz, CDCl₃): [symbol] 9.66(br, 1H), 7.44 (d, 1H), 7.28 (m, 2H), 7.13 (m, 3H), 6.85 (t, 1H), 6.83 (d, 1H), 4.31 (t, 2H), 2.96 (t, 2H), 257(m, 4H), 1.79 (m, 4H). MS (m/z): 415.2 (M+H).

### Example 175

### Preparation of 2-(2-Hydroxyphenyl)-3-(2-phenylethyl)-5,6,8,9-tetrahydrooxepino[4,5-d]pyrimidin-4(3H)-one

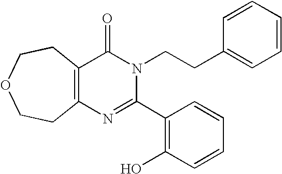

### a. Ethyl 5-oxo-4-oxepanecarboxylate

A solution of tetrahydro-4H-pyran-4-one (4.89 g, 0.049 moles) in dry diethyl ether was cooled to −30° C. BF₃.Et₂O (7.36 mL) was added dropwise keeping the temperature the same. Diethyl ether solution of ethyl diazoacetate (6.08 mL, 0.058 moles) was added slowly for 15 min and the reaction stirred for additional 3 h while the reaction warmed to −15° C. The reaction mixture was poured onto ice and saturated NaHCO₃ and organic layer was separated dried over Na₂SO₄. The crude product was purified on FCC (40% EtOAc/Hexane) to produce the desired product (6.5 g) in 72% yield.

### b. 2-(2-Hydroxyphenyl)-3-(2-phenylethyl)-5,6,8,9-tetrahydrooxepino[4,5-d]pyrimidin-4(3H)-one

To a solution of 2-(methyloxy)benzenecarboximidamide (0.81 g, 5.38 mmol) in 54 mL of solvent mixture of MeOH/Dioxane (1/1) was added NaOMe (0.58 g, 10.8 mmol) and stirred for 15 min. Ethyl 5-oxo-4-oxepanecarboxylate (1.0 g, 5.38 mmol) was introduced and the reaction mixture was heated to reflux for 16 h. Upon completion the reaction mixture was concentrated, diluted with dichloromethane and added dilute HCl. The dichloromethane layer was separated and washed with brine, dried over Na₂SO₄. Upon filteration and concentration the crude product was purified by FCC (0-10% MeOH/dichloromethane) to give product (0.36 g) in 25% yield.

### c. 2-[2-(Methyloxy)phenyl]-3-(2-phenylethyl)-5,6,8,9-tetrahydrooxepino[4,5-d]pyrimidin-4(3H)-one

To a solution of 2-(2-Hydroxyphenyl)-3-(2-phenylethyl)-5,6,8,9-tetrahydrooxepino[4,5-d]pyrimidin-4(3H)-one (1.18 g, 4.33 mmol) in dry DMF (43 mL) was added lithium hydride (0.069 g, 8.66 mmol) and lithium bromide (1.13 g, 13.0 mmol) and stirred for 10 min at room temperature. Then (2-bromoethyl)benzene (4.01 g, 21.7 mmol) was added and stirred overnight. The reaction mixture was quenched by addition of ice and 6N HCl. This mixture was extracted with EtOAc and the organic layer was washed with aqueous NaHCO₃, brine and dried over Na₂SO₄. The sodium sulfate was filtered and concentrated. The crude product is purified by FCC (30% ethyl acetate/hexane) to give product (0.86 g) in 53% yield.

### d. 2-(2-Hydroxyphenyl)-3-(2-phenylethyl)-5,6,8,9-tetrahydrooxepino[4,5-d]pyrimidin-4(3H)-one

2-[2-(Methyloxy)phenyl]-3-(2-phenylethyl)-5,6,8,9-tetrahydrooxepino[4,5-d]pyrimidin-4(3H)-one (0.18 g, 0.48 mmol) was placed in a sealed tube. To this was added large excess (8.3 g) of pyridine hydrochloride salt and reaction vessel was placed in an oil bath. The reaction was heated to 150° C. for 16 h. The crude reaction mixture was diluted with dichloromethane and washed with water and brine. Upon drying over Na₂SO₄, it was concentrated and purified on Biotage purification system using EtOAc/hexane mixture (0-60%) to produce the desired product (0.034 g) in 20% yield. MS (m/z): 363.2 [M+H]⁺.

### Example 176

### Preparation of 3-(2-Cyclohexyl-ethyl)-2-(2-hydroxy-phenyl)-3,5,6,7, 8,9-hexahydro-cycloheptapyrimidin-4-one

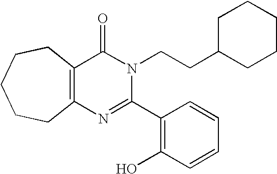

The title compound was prepared by substituting 2-oxo-cycloheptanecarboxylic acid ethyl ester for 2-oxo-cyclohexanecarboxylic acid ethyl ester in Example 168. ¹H NMR (400 MHz, CDCl₃): [symbol] 10.05 (s, 1H, OH), 7.40-7.34 (m, 2H), 7.06 (d, 1H), 6.96 (t, 1H), 4.13 (t, 2H), 2.88-2.79 (m, 4H), 1.90-1.82 (m, 2H), 1.75-1.51 (m, 11H), 1.25-1.10 (m, 4H), 0.90-0.80 (m, 2H). MS (m/z): 367.2 (M+H).

### Example 177

### Preparation of 2-(2-hydroxyphenyl)-3-(2-phenylethyl)-6-(phenylmethyl)-5,6,7,8-tetrahydropyrido[4,3-d]pyrimidin-4(3H)-one

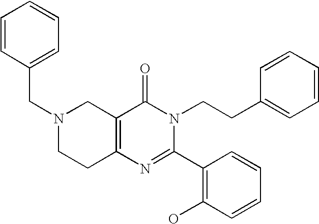

### a. 6-Benzyl-2-(2-methoxy-phenyl)-5,6,7,8-tetrahydro-3H-pyrido[4,3-d]pyrimidin-4-one

Dissolved 2-(methyloxy)benzenecarboximidamide (300 mg, 2.0 mmol) in MeOH/dioxane (20 mL/7 mL) and cooled to 0° C. 25% NaOCH₃ in MeOH (1.39 mL) was then added and stirred for 15 mins. 1-Benzyl-4-oxo-piperidine-3-carboxylic acid ethyl ester hydrochloride salt (893 mg, 3.0 mmol) was introduced and the reaction mixture was heated to reflux for 2 h. After removing the solvent, to the residue was added 10 mL H₂O and acetic acid was used to adjust pH=7-8. Extracted by CH₂Cl₂ (3×100 mL). The organic layer was dried over Na₂SO₄. Purified by Biotage (10% to 95% ethylacetate/hexane, with 1% MeOH) to give product 1.21 g as white solid in 87% yield.

### b. 2-[2-(Methyloxy)phenyl]-3-(2-phenylethyl)-6-(phenylmethyl)-5,6,7,8-tetrahydropyrido[4,3-d]pyrimidin-4(3H)-one

6-Benzyl-2-(2-methoxy-phenyl)-5,6,7,8-tetrahydro-3H-pyrido[4,3-d]pyrimidin-4-one (200 mg, 0.58 mmol) was dissolved in dry DMF at 0° C. LiH (5.5 mg, 0.69 mmol) was added and stirred for 5 mins at this temperature. Then (2-bromoethyl)benzene (0.533 g, 2.88 mmol) was added and stirred at RT overnight. Diluted with Et₂O and dried over MgSO₄. Filtered, concentrated and purified by flash column chromatography (30% to 90% ethylacetate/hexane) to get the desired product 100 mg (Yield 30.8%).

### c. 2-(2-Hydroxyphenyl)-3-(2-phenylethyl)-6-(phenylmethyl)-5,6,7,8-tetrahydropyrido[4,3-d]pyrimidin-4(3H)-one

2-[2-(methyloxy)phenyl]-3-(2-phenylethyl)-6-(phenylmethyl)-5,6,7,8-tetrahydropyrido[4,3-d]pyrimidin-4(3H)-one (90 mg, 0.20 mmol) was dissolved in dichlormethane at −60° C., BBr₃ (2.4 mL, 2.4 mmol) was introduced to the reaction mixture and stirred at this temperature for 1 h and then stirred at RT for 2 days. The crude mixture was purified on column (2% MeOH/dichlormethane) and then biotage (60% to 90% ethylacetate/hexane) to give product as white solid (55 mg 63% yield). ¹H NMR (400 MHz, CDCl₃): [symbol] 7.33-6.69 (m, 14H), 4.08 (t, 2H), 3.71 (s, 2H), 3.53 (s, 2H), 2.77 (t, 2H), 2.61 (m, 4H). MS (m/z): 438.4 (M+H).

### Example 178

### Preparation of 2-Methylpropyl 2-(2-hydroxyphenyl)-4-oxo-3-(2-phenylethyl)-3,5,7,8-tetrahydropyrido[4,3-d]pyrimidine-6(4H)-carboxylate

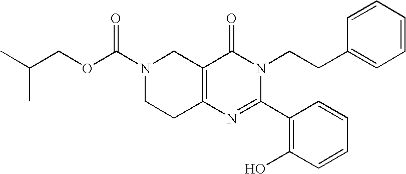

The title compound was prepared by substituting isobutyl chloroformate for ethylchloroformate in Example 161: ¹H NMR (400 MHz, CDCl₃): [symbol] 8.60(br, 1H), 7.29 (m, 1 H), 7.20 (m, 4H), 6.88 (m, 4H), 4.47 (S, 2H), 4.18 (t, 2H), 4.88 (s, 2H), 3.70 (t, 2H), 2.87 (t, 2H), 2.66 (t, 2H), 1.91 (m, 1H), 0.96 (d, 6H). MS (m/z): 448.4 (M+H).

### Example 179

### Preparation of 2-(3-Fluoro-2-hydroxyphenyl)-6-methyl-5-[5-(2-methyl-1,3-thiazol-4-yl)-2-thienyl]-3-(2-phenylethyl)-4(3H)-pyrimidinone

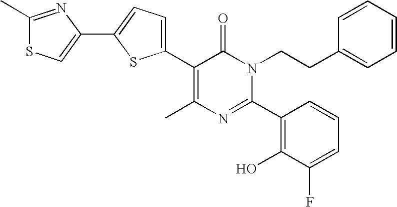

### a. 2-{3-Fluoro-2-[(phenylmethyl)oxy]phenyl}-6-methyl-5-[5-(2-methyl-1,3-thiazol-4-yl)-2-thienyl]-3-(2-phenylethyl)-4(3H)-pyrimidinone

A solution of 5-bromo-2-{3-fluoro-2-[(phenylmethyl)oxy]phenyl}-6-methyl-3-(2-phenylethyl)-4(3H)-pyrimidinone (0.3 g, 0.61 mmol) of example 11, 4-(5-bromo-2-thienyl)-2-methyl-1,3-thiazole (0.158 g, 0.61 mmol), hexamethyldistannane (0.13 mL, 0.61 mmol), Pd(PPh₃)₄ (0.070 g, 0.061 mmol) in 10 mL dioxane was degassed for 10 min and then heated at 90° C. for 16 h. The reaction mixture was concentrated and the crude product was purified on flash Silica gel column (EtOAc/hexanes) to give product (0.2 g) in yield 55%. MS (ES) m/e 594[M+H].

### b. 2-(3-Fluoro-2-hydroxyphenyl)-6-methyl-5-[5-(2-methyl-1,3-thiazol-4-yl)-2-thienyl]-3-(2-phenylethyl)-4(3H)-pyrimidinone

2-{3-Fluoro-2-[(phenylmethyl)oxy]phenyl}-6-methyl-5-[5-(2-methyl-1,3-thiazol-4-yl)-2-thienyl]-3-(2-phenylethyl)-4(3H)-pyrimidinone (0.1 g, 0.168 mmol) was taken up in glacial acetic acid (20 mL). To this was added 10% Pd/C. This mixture was placed under hydrogen atmosphere at 48 psi and shaken for 48 h. The reaction mixture was filtered through a bed of celite and concentrated. The crude residue was taken up in dichloromethane and washed with NaHCO₃ and brine. The organic layer was dried, filtered and concentrated. The crude residue was purified by chromatography on silica gel to afford the desired product (0.003 g) in 3.5% yield. MS (ES) m/e 504[M+H]⁺.

### Example 180

### Preparation of 2-[2-(hydroxy)phenyl]-3-(2-phenylethyl)-5,6,7,8-tetrahydropyrido[3,2-d]pyrimidin-4(3H)-one

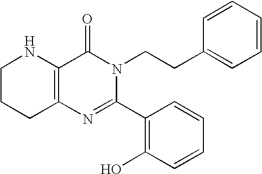

### a. 1,1-Dimethylethyl 2-[2-(methyloxy)phenyl]-4-oxo-4,6,7,8-tetrahydropyrido[3,2-d]pyrimidine-5(1H)-carboxylate

A suspension of 1-(1,1-dimethylethyl) 2-methyl 3-oxo-1,2-piperidinedicarboxylate (J. Med. Chem. 1989, 32, 2116-2128) (0.78 g, 3.04 mmol) and 2-(methoxy)benzenecarboxamidine (0.68 g, 1.5 eq.) in dry toluene (10 mL) was stirred at 100° C. for 3 h. The reaction mixture was concentrated and the residue was purified by flash column chromatography on silica gel (hexane/EtOAc 3:1) to give 0.95 g of the product.

### b. 1,1-Dimethylethyl 2-[2-(methyloxy)phenyl]-4-oxo-3-(2-phenylethyl)-4,6,7,8-tetrahydropyrido[3,2-d]pyrimidine-5(3H)-carboxylate

A suspension of pyrimidinone 1,1-dimethylethyl 2-[2-(methyloxy)phenyl]-4-oxo-4,6,7,8-tetrahydropyrido[3,2-d]pyrimidine-5(1H-carboxylate (0.95 g, 2.66 mmol), NaH (222 mg, 1.2 eq. 60% suspension in mineral oil) and LiBr (1.14 g, 5 eq.) in DMF was stirred at RT for 20 min. Phenethyl bromide (1.70 mL, 2 eq.) was added dropwise and the resulting mixture was stirred at RT overnight. The reaction mixture was diluted with EtOAc, which was washed with H₂O and brine. The organic layer was dried (Na₂SO₄), concentrated, and the residue was purified by flash column chromatography on silica gel (hexane/EtOAc 2:1) to give 0.40 g of the O-alkylated product (33%) and 663 mg of the desired N-alkylated product (55%).

### c. 2-[2-(hydroxy)phenyl]-3-(2-phenylethyl)-5,6,7,8-tetrahydropyrido[3,2-d]pyrimidin-4(3H)-one

BBr₃ (4.8 mL, 6.0 eq., 1M/CH₂Cl₂) was added to a solution of 1,1-dimethylethyl 2-[2-(methyloxy)phenyl]-4-oxo-3-(2-phenylethyl)-4,6,7,8-tetrahydropyrido[3,2-d]pyrimidine-5(3H)-carboxylate (0.37 g, 0.80 mmol) in CH₂Cl₂ (4 mL) at −78 C. Yellow precipitate was formed. The reaction was warmed up to RT and stirred overnight. The reaction mixture was treated with saturated Na₂CO₃ aqueous solution and the organic layer was washed with H₂O, brine, and dried (Na₂SO₄). Removal of the solvent and purification of the residue by flash column chromatography on silica gel (hexane/EtOAc 1:1) afforded 150 mg of white solid (54%). ¹H NMR (400 MHz, CDCl₃): [symbol] 9.94 (br, 1H, OH), 7.22-7.14 (m, 5H), 6.92-6.88 (m, 3H), 6.78 (d, 1H, J=8.2 Hz), 4.54 (br, 1H, NH), 4.25 (t, 2H, J=7.9 Hz), 3.50-3.33 (m, 2H), 2.86 (t, 2H, J=7.9 Hz), 2.58 (t, 2H, J=6.4 Hz) 2.01-1.95 (m, 2H). MS (m/z): 348.2 (M+H).

### Example 181

### Preparation of 2-(2-hydroxyphenyl)-5-methyl-3-(2-phenylethyl)-5,6,7,8-tetrahydropyrido[3,2-a]pyrimidin-4(3H)-one

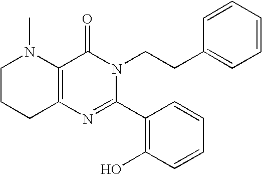

NaCNBH₃ was added to a mixture 2-[2-(hydroxy)phenyl]-3-(2-phenylethyl)-5,6,7,8-tetrahydropyrido[3,2-d]pyrimidin-4(3H)-one (41 mg, 012 mmol), aq. HCHO (0.11 mL, 12 eq., 37%). The resulting mixture was stirred at RT overnight and taken up into DCM, which was washed with saturated NaHCO₃ aqueous solution, brine, and dried (Na₂SO₄). Removal of the solvent and purification of the residue by flash column chromatography on silica gel (hexane/EtOAc 1:1) afforded 38.2 mg of yellow solid (88%). ¹H NMR (400 MHz, CDCl₃): δ 7.24-7.13 (m, 5H), 7.02-6.82 (m, 4H), 4.22 (t, 2H, J=7.8 Hz), 3.11-3.07 (m, 2H), 3.04 (s, 3H), 2.88 (t, 2H, J=7.8 Hz), 2.58 (t, 2H, J=6.4 Hz), 1.93-1.86 (m, 2H). MS (m/z): 362.2 (M+H).

### Example 182

### Preparation of 5-ethyl-2-[2-hydroxyphenyl]-3-(2-phenylethyl)-5,6,7,8-tetrahydropyrido[3,2-d]pyrimidin-4(3H)-one

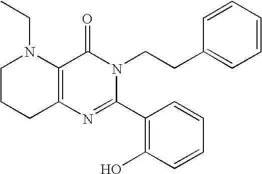

### a. 2-[2-(Methyloxy)phenyl]-3-(2-phenylethyl)-5,6,7,8-tetrahydropyrido[3,2-d]pyrimidin-4(3H)-one

A solution of 1,1-dimethylethyl 2-[2-(methyloxy)phenyl]-4-oxo-3-(2-phenylethyl)-4,6,7,8-tetrahydropyrido[3,2-d]pyrimidine-5(3H)-carboxylate (0.16 g, 0.35 mmol) of Example 180b and TFA (1 mL) in CH₂Cl₂ (3 mL) was stirred at RT for 4 hr, poured onto ice-cold saturated Na₂CO₃ aqueous solution. The resulting mixture was extracted with CH₂Cl₂ and the combined organic layers were washed with brine, dried (Na₂SO₄) and concentrated. The residue was purified by flash column chromatography on silica gel (hexane/EtOAc 1:1) to give 0.11 g of the title compound (88%).

### b. 5-Ethyl-2-[2-hydroxyphenyl]-3-(2-phenylethyl)-5,6,7,8-tetrahydropyrido[3,2-d]pyrimidin-4(3H)-one

A solution of 2-[2-(methyloxy)phenyl]-3-(2-phenylethyl)-5,6,7,8-tetrahydropyrido[3,2-d]pyrimidin-4(3H)-one (90 mg, 0.25 mmol) and CH₃CHO (70 □L, 5 eq.) in CH₂Cl₂ (2.5 mL) was stirred at RT for 1 hr. NaBH(OAc)₃ (79 mg, 1.5 eq.) was added and the resulting mixture was stirred at RT overnight. The reaction was quenched with saturated NaHCO₃ aqueous solution and the organic layer was separated, dried (Na₂SO₄) and concentrated. The residue was purified by flash column chromatography on silica gel (hexane/EtOAc 1:1) to give 77.5 milligrams of product. Subsquent demethylation utilizing the procedure detailed previously produced the product. ¹H NMR (400 MHz, CDCl₃): [symbol] 9.80 (br, 1H, OH), 7.11-7.05 (m, 5H), 6.81-6.77 (m, 3H), 6.68 (d, 1H, J=8.1 Hz), 4.11-4.07 (m, 2H), 3.31 (q, 2H, J=7.0 Hz), 3.05-3.02 (m, 2H), 2.77 (t, 2H, J=7.4 Hz), 2.48 (t, 2H, J=6.4 Hz), 1.78-1.28 (m, 2H), 1.13 (t, 3H, J=7.0 Hz). MS (m/z): 376.2 (M+H).

### Example 183

### Preparation of 1,1-dimethylethyl 2-(2-hydroxyphenyl)-4-oxo-3-(2-phenylethyl)-3,4,5,7-tetrahydro-6H-pyrrolo[3,4-d]pyrimidine-6-carboxylate

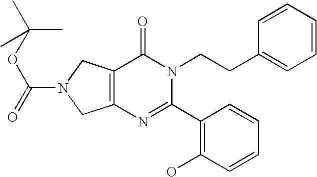

### a. 1,1-Dimethylethyl 4-oxo-2-[2-({[(phenylmethyl)oxy]carbonyl}oxy)phenyl]-1,4,5,7-tetrahydro-6H-pyrrolo[3,4-d]pyrimidine-6-carboxylate

The title compound was prepared by following the procedures outlined in Example 180 except substituting 4-oxo-pyrrolidine-1,3-dicarboxylic acid 1-tert-butyl ester 3-ethyl ester (Tetrahedron Lett. 2002, 43(17), 3243-3246) for 1-(1,1-dimethylethyl) 2-methyl 3-oxo-1,2-piperidinedicarboxylate.

### b. 1,1-Dimethylethyl 2-(2-hydroxyphenyl)-4-oxo-3-(2-phenylethyl)-3,4,5,7-tetrahydro-6H-pyrrolo[3,4-d]pyrimidine-6-carboxylate

A suspension of 1,1-dimethylethyl-4-oxo-3-(2-phenylethyl)-2-[2-({[(phenylmethyl)oxy]carbonyl}oxy)phenyl]-3,4,5,7-tetrahydro-6H-pyrrolo[3,4-d]pyrimidine-6-carboxylate (78 mg, 0.15 mmol) and Pd/C (10 mg, 10%) in EtOH was stirred under a balloon of H₂ at RT for 3 hr. The reaction mixture was filtered through a pad of celite, which was rinsed with CH₂Cl₂. The combined filtrates were concentrated and the residue was purified by flash column chromatography on silica gel (hexane/EtOAc 2:1) to give 49 mg of the product (76%): ¹H NMR (400 MHz, CDCl₃): [symbol] 8.69 (br, 1H, OH), 7.34-7.30 (m, 1H), 7.19-7.17 (m, 3H), 7.05-7.03 (m, 1H), 6.97-6.89 (m, 4H), 4.63-4.52 (m, 4H), 4.20-4.15 (m, 2H), 2.89-2.85 (m, 2H), 1.52, 1.50 (s, 9H, rotamers): MS (m/z): 434.4(M+H).

### Example 184

### Preparation of 5-(2-methyl propyl-2-yl-2-(2-hydroxy-phenyl)-6-methyl-3-(2-phenethyl)-3H-pyrimidin-4-one

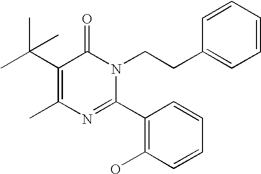

### a. Ethyl 2-acetyl-3,3-dimethylbutanoate

To the mixture of 3-oxo-butyric acid ethyl ester (19.5 g, 150 mmol) and 2-bromo-2-methylpropane (6.85 g, 50 mmol) in 5 mL nitromethane under Ar at 0° C., was added silver perchlorate (10.4 g, 50 mmol) in 20 mL nitromethane. The mixture was stirred at 0° C. for 3 hr. The solid was filtered off and the filtrate was concentrated. The crude residue was purified on flash column chromatography (10% EtOAc/Hexanes) to provide 3.5 g (38%) of the title compound.

### b. 5-(2-Methyl propyl-2-yl)-2-(2-hydroxy-phenyl)-6-methyl-3-(2-phenethyl)-3H-pyrimidin-4-one

The title compound was prepared by following the general procedures of Example 26 except substituting ethyl 2-acetyl-3,3-dimethylbutanoate for ethyl 2-chloro-3-oxobutanoate. ¹H NMR (400 MHz, CDCl₃): [symbol] 10.25(br, 1H), 2.40-7.35 (m, 2H), 7.23-7.20 (m, 3H), 7.03-6.92 (m, 4H), 4.48 (t, 2H), 3.03 (t, 2H), 2.49 (s, 3H), 1.53 (s, 9H). MS (m/z): 363.4 (M+H).

### Example 185

### Preparation of 5-[2-(3-fluorophenyl)ethyl]-6-(2-hydroxyphenyl)-1-methyl-1,5-dihydro-4H-pyrazolo[3,4-d]pyrimidin-4-one

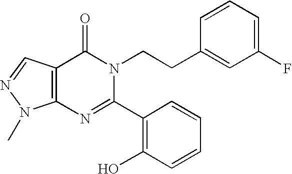

### a. N-[2-(3-Fluorophenyl)ethyl]-1-methyl-5-nitro-1H-pyrazole-4-carboxamide

To 1-Methyl-5-nitro-1H-pyrazole-4-carboxylic acid (500 mg, 2.9 mmol) was added CH₂Cl₂ 50 mL and (COCl)₂ (0.92 mL, 10.5 mmol) and the mixture was heated to reflux for 3 hr. Excess (COCl)₂ and the solvent were removed on rotavapor. The residue was dissolved in 50 mL CH₂Cl₂ and 3-fluorophenethylamine (730 mg, 5.22 mmol) in pyridine (1 mL) was added and stirred at RT overnight. The reaction mixture was diluted with CH₂Cl₂ and washed with H₂O, 1N HCl and brine (100 mL each) and then purified by flash column chromatography (50% EA/Hexane). To provide 550 mg of a white solid. Yield 65%.

### b. 5-Amino-N-[2-(3-fluorophenyl)ethyl]-1-methyl-1H-pyrazole-4-carboxamide

N-[2-(3-fluorophenyl)ethyl]-1-methyl-5-nitro-1H-pyrazole-4-carboxamide (300 mg, 1.03 mmol) and zinc powder (2.0 g, 30.8 mmol) were mixed in 2 mL HOAc and 2 mL THF. The resulted mixture was stirred at RT for 1.5 hr. The mixture was filtered through celite and evaporated. The residue was diluted in EtOAc and washed with 1N NaOH and brine. Dried over Na₂SO₄, filtered and concentrated to provide 0.25 g of the title compound

### c. N-[2-(3-Fluorophenyl)ethyl]-1-methyl-5-[({2-[(phenylmethyl)oxy]phenyl}carbonyl)amino]-1H-pyrazole-4-carboxamide

5-amino-N-[2-(3-fluorophenyl)ethyl]-1-methyl-1H-pyrazole-4-carboxamide (200 mg, 0.76 mmol) and 2-benzyloxy-benzoyl chloride (226 mg, 0.92 mmol) were mixed in THF 50 mL and 2 mL pyridine. The mixture was stirred overnight and THF was removed and the residue was diluted in EtOAc and washed with H₂O, 1N HCl, sat. NaHCO₃ and brine. Organic layer was concentrated and purified on flash column chromatography (50% EtOAc/Hexane), provided 350 mg of the title compound.

### d. 5-[2-(3-Fluorophenyl)ethyl]-6-(2-hydroxyphenyl)-1-methyl-1,5-dihydro-4H-pyrazolo[3,4-d]pyrimidin-4-one

To the solution of N-[2-(3-fluorophenyl)ethyl]-1-methyl-5-[({2-[(phenylmethyl)oxy]phenyl}carbonyl)amino]-1H-pyrazole-4-carboxamide (50 mg, 0.11 mmol) in toluene was added catalytic amount of pTsOH and refluxed for 2 days. Removed solvent and the residue was purified on flash column chromatography (30% EtOAc/Hexane) to provide 15 mg of the title compound: ¹H NMR (400 MHz, CDCl₃): [symbol] 8.13(s, 1H), 7.49-7.42 (m, 1H), 7.26-7.05 (m, 4H), 6.92-6.87 (m, 1H), 6.65 (d, 1H), 6.53 (d, 1H), 4.33 (t, 2H), 3.99 (s, 3H), 2.90-2.86 (t, 2H). MS(m/z): 365.2(M+H).

### Example 186

### Preparation of 5-ethyl-2-(3-fluoro-2-hydroxyphenyl)-3-[2-(3-fluorophenyl)ethyl]-6-phenyl-4(3H-pyrimidinone

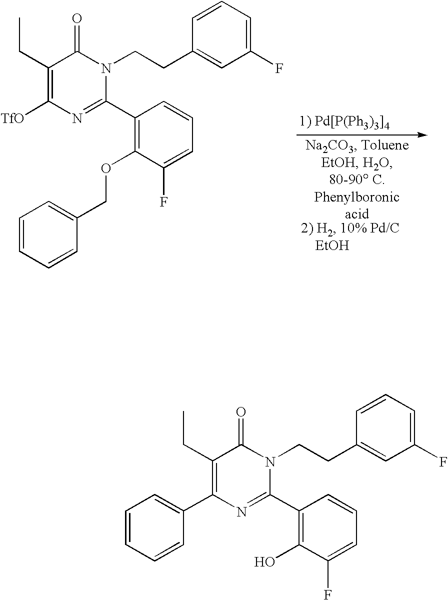

### a. 5-Ethyl-2-(3-fluoro-2-hydroxyphenyl)-3-[2-(3-fluorophenyl)ethyl]-6-phenyl-4(3H)-pyrimidinone

To the solution of 5-ethyl-1-[2-(3-fluorophenyl)ethyl]-6-oxo-2-{2-[(phenylmethyl)oxy]phenyl}-1,6-dihydro-4-pyrimidinyl trifluoromethanesulfonate of Example 27 (81 mg, 0.14 mmol) in 3 mL toluene was added Pd(PPh₃)₄ (16 mg, 0.014 mmol) and phenylboronic acid (35 mg, 0.29 mmol) in 0.5 mL EtOH followed by aqueous Na₂CO₃ (215 mg in 0.5 mL H₂O) at RT. The mixture was then stirred vigorously for 5 min and heated to 90° C. for 3 h. After which another portion of reagents (0.1 eq. Pd(PPh₃)₄, 2.1 eq. boronic acid and 1.0 eq. Na₂CO₃) were added and the reaction mixture refluxed overnight. Concentration and purification of the residue by flash column chromatography (0% to 50% EtOAc/Hexane) gave 52 mg of the title compound. Subsequent catalytic hydrogenolysis provided the title compound. ¹H NMR (400 MHz, CDCl₃): [symbol] 8.85 (br, 1H, OH), 7.47-7.43 (m, 4H), 7.20-7.10 (m, 4H), 6.93-6.65 (m, 4H), 4.40 (t, 2H), 3.02 (t, 2H), 2.64 (q, 2H), 1.24 (t, 3H).

### Example 187

### Preparation of 6-[3,4-bis(methyloxy)phenyl]-5-ethyl-2-(3-fluoro-2-hydroxyphenyl)-3-[2-(3 fluorophenyl ethyl]-4(3H)-pyrimidinone

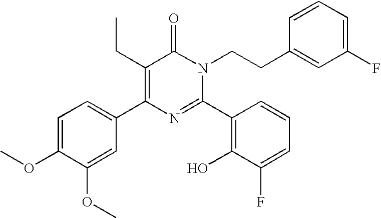

The title compound was prepared by following the general procedures of Example 186 except substituting 3,4-dimethoxyphenyl boronic acid for phenyl boronic acid. ¹H NMR (400 MHz, CDCl₃): [symbol] 9.10 (br, 1H, OH), 7.20-6.60 (m, 10H), 4.34 (t, 2H), 3.93 (s, 1H), 3.92 (s, 1H), 2.98 (t, 2H), 2.68 (q, 2H), 1.25 (t, 3H). MS (m/z): 493.4 (M+H).

### Example 188

### Preparation of 5-ethyl-2-(3-fluoro-2-hydroxyphenyl)-3-[2-(3-fluorophenyl)ethyl]-6-(3-nitrophenyl)-4(3H)-pyrimidinone

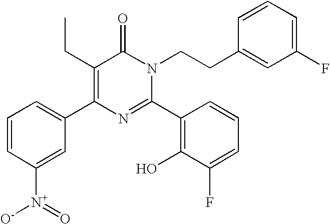

The title compound was prepared by following the general procedures of Example 186 except substituting 3-nitrophenyl boronic acid for phenyl boronic acid. ¹H NMR (400 MHz, CDCl₃): [symbol] 8.74 (t, 1H), 8.39-8.38 (m, 1H), 7.85-7.83 (m, 2H), 7.63 (t, 1H), 7.19-7.17 (m, 2H), 6.97-6.89 (m, 3H), 6.71 (d, 1H), 6.59 (d, 1H), 4.25 (t, 2H), 2.95 (t, 2H), 2.59 (q, 2H), 1.26 (t, 3H). MS (m/z): 434.4 (M+H).

### Example 189

### Preparation of 5-ethyl-3-[2-(3-fluorophenyl)ethyl]-2-(2-hydroxyphenyl)-6-(1-pyrrolidinyl)-4(3H)-pyrimidinone

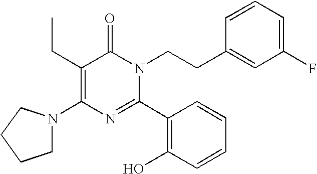

To the solution of 5-ethyl-1-[2-(3-fluorophenyl)ethyl]-6-oxo-2-{2-[(phenylmethyl)oxy]phenyl}-1,6-dihydro-4-pyrimidinyl trifluoromethanesulfonate of Example 28 (30 mg, 0.07 mmol) in dry dioxane was added pyrrolidine (7.7 uL, 0.08 mmol) and Cs₂CO₃ (31 mg, 0.1 mmol). The reaction mixture was heated at 105° C. overnight. Upon completion the reaction was concentrated and purified on flash column chromatography (0 to 50% EtOAc/Hexane) to provide 25.5 mg of 5-ethyl-3-[2-(3-fluorophenyl)ethyl]-2-{2-[(phenylmethyl)oxy]phenyl}-6-(1-pyrrolidinyl)-4(3H)-pyrimidinone. This compound was deprotected utilizing the BBr₃ procedure outlined previously to provide the title compound: ¹H NMR (400 MHz, CDCl₃): δ 9.81(br, 1H), 7.22(m, 2H), 7.08 (m, 1H), 6.90 (d, 1H), 6.78 (m, 2H), 6.67(d, 1H), 6.55 (d, 1H), 4.30 (t, 2H), 2.92 (t, 2H), 2.68 (q, 2H), 1.92 (m, 4H), 1.22 (m, 4H), 1.19 (t, 3H) MS (m/z): 408.4 (M+H).

### Example 190

### Preparation of 6-(dimethylamino)-5-ethyl-3-[2-(3-fluorophenyl)ethyl]-2-(2-hydroxyphenyl)-4(3H)-pyrimidinone

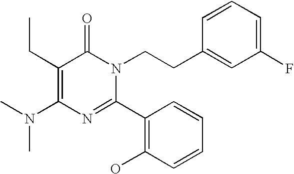

The title compound was prepared by following the general procedures of Example 189 except substituting dimethylamine for pyrrolidine: ¹H NMR (400 MHz, CDCl₃): δ 9.68(br, 1H), 7.22 (t, 1H), 7.20 (d, 2H), 7.01 (m, 1H), 6.91-6.46 (m, 4H), 4.29 (t, 2H), 2.95 (s, 6H), 2.88 (t, 2H), 2.52 (q, 2H), 1.08 (t, 3H). MS (m/z): 382.4 (M+H).

### Example 191

### Preparation of 5-ethyl-3-[2-(3-fluorophenyl)ethyl]-2-(2-hydroxyphenyl)-6-(methylamino)-4(3H)-pyrimidinone

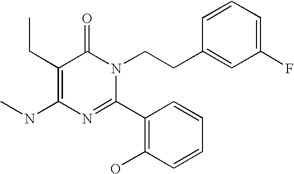

The title compound was prepared by following the general procedures of Example 189 except methylamine for pyrrolidine: ¹H NMR (400 MHz, CDCl₃): δ 7.31-6.72 (m, 8H), 4.31 (t, 2H), 2.99 (s, 3H), 2.96 (t, 2H), 2.43 (q, 2H), 1.09 (t, 3H). MS (m/z): 368.2 (M+H).

### Example 192

### Preparation of 5-cyclopentyl-3-[2-(3-fluorophenyl)ethyl]-2-(2-hydroxyphenyl)-6-methyl-4(3H)-pyrimidinone

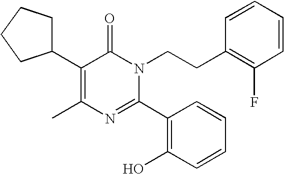

The title compound was prepared by substituting ethyl 2-cyclopentyl-3-oxobutanoate for ethyl 2-chloro-3-oxobutanoate and 2-fluoro-(2-bromoethyl)benzene for (2-bromoethyl)benzene in Example 26. ¹H NMR (400 MHz, CDCl₃): δ 7.31(m, 1H), 7.18 (m, 2H), 6.95 (m, 5H), 4.34 (t, 2H), 3.11 (m, 1H), 2.99 (t, 2H), 2.39 (s, 3H), 2.05-1.60 (m, 8H). MS (m/z): 393.2 (M+H).

### Example 193

### Preparation of 2-(2-hydroxyphenyl)-6-methyl-5-(2-methylpropyl)-3-(2-phenylethyl)-4(3H)-pyrimidinone

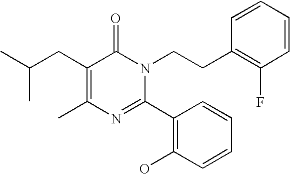

The title compound was prepared by substituting 2-(2-methyl-propyl)-3-oxo-butyric acid ethyl ester for ethyl 2-chloro-3-oxobutanoate and 2-fluoro-(2-bromoethyl)benzene for (2-bromoethyl)benzene in Example 26: ¹H NMR: 9.70(br, 1H), 7.06 (m, 2H), 6.90-6.66 (m, 6H), 4.07 (t, 2H), 2.88 (t, 2H), 2.38 (d, 2H), 2.18 (s, 3H), 1.88 (m, 1H), 0.85 (d, 6H). MS (m/z): 381.2 (M+H).

### Example 194

### Preparation of 2-(2-hydroxyphenyl)-6-methyl-5-(2-methylpropyl)-3-[2-(2-thienyl)ethyl]-4(3H)-pyrimidinone

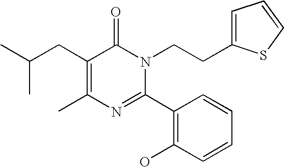

The title compound was prepared by substituting 2-(2-methyl-propyl)-3-oxo-butyric acid ethyl ester for ethyl 2-chloro-3-oxobutanoate and 2-thiophene-2-yl-ethyl bromide for (2-bromoethyl)benzene in Example 26. ¹H NMR (400 MHz, CDCl₃): δ 7.30(t, 1H), 7.02 (d, 1H), 6.96-6.79 (m, 4H), 6.66 (t, 1H), 4.37 (t, 2H), 3.28 (t, 2H), 2.49 (d, 2H), 2.38 (s, 3H), 2.02 (m, 1H), 0.95 (d, 6H). MS (m/z): 369.0 (M+H).

### Example 195

### Preparation of 5-Ethyl-2-(2-hydroxy-phenyl)-6-Ethyl-3-phenylethyl-3H-pyrimidin-4-one

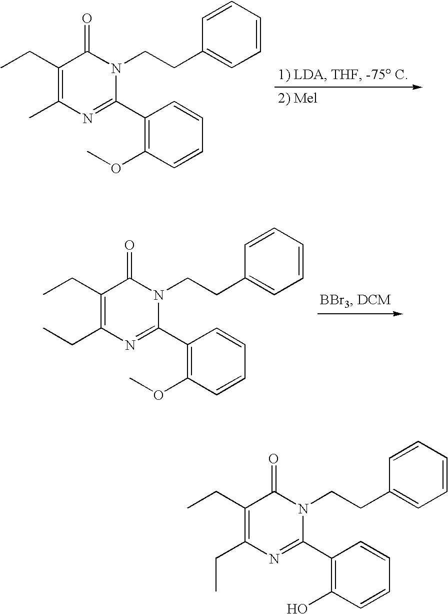

### a. 5-Ethyl-6-methyl-2-[2-(methyloxy)phenyl]-3-(2-phenylethyl)-4(3H)-pyrimidinone

The title compound was prepared by substituting ethyl 2-ethyl-3-oxobutanoate for ethyl 2-chloro-3-oxobutanoate in Example 26.

### b. 5-Ethyl-2-(2-hydroxy-phenyl)-6-Ethyl-3-phenylethyl-3H-pyrimidin-4-one

To the solution of 5-ethyl-6-methyl-2-[2-(methyloxy)phenyl]-3-(2-phenylethyl)-4(3H)-pyrimidinone (0.2 g, 0.48 mmol) in THF was cooled to −75° C. To this was added 2M LDA (0.27 mL. 0.53 mmol) dropwise. After stirring for at least one hour methyl iodide (0.083 g, 0.53 mmol) was added by syringe. The reaction was stirred overnight and quenched by NH₄Cl, extracted with EtOAc. EtOAc layer was washed with brine. Aqueous layer was back extracted with EtOAc. EtOAc layers were combined, dried over Na₂SO₄, filtered and concentrated in vacuo. The residue was purified by flash chromatography (hexane/EtOAc=4:1) to yield 5,6-diethyl-2-[2-(methyloxy)phenyl]-3-(2-phenylethyl)-4(3H)-pyrimidinone (0.145 g). This material was converted to the title compound using BBr₃ as previously detailed: ¹H NMR: 7.22(m, 2H), 7.02 (m, 3H), 6.90 (m, 4H), 4.35 (t, 2H), 3.08 (t, 2H), 1.28 (t, 3H), 1.20 (t, 3H). MS (m/z): 349.4 (M+H).

### Example 196

### Preparation of 5-Ethyl-2-(2-hydroxy-phenyl)-6-Propyl-3-phenylethyl-3H-pyrimidin-4-one

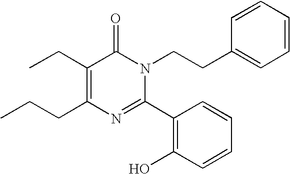

The title compound was prepared by substituting ethyl iodide for methyl iodide in Example 195: ¹H NMR: 9.90(br, 1H), 7.31-7.18 (m, 5H), 6.91 (m, 4H), 4.28 (t, 2H), 2.59 (q, 2H), 2.51 (t, 2H), 1.61 (q, 2H), 1.12 (t, 3H), 0.99 (t, 3H). MS (m/z): 363.4 (M+H).

### Example 197

### Preparation of 5-Ethyl-2-(3-fluoro-2-hydroxy-phenyl)-6-(2-phenylethyl)-3-(2-fluoro-phenylethyl)-3H-pyrimidin-4-one

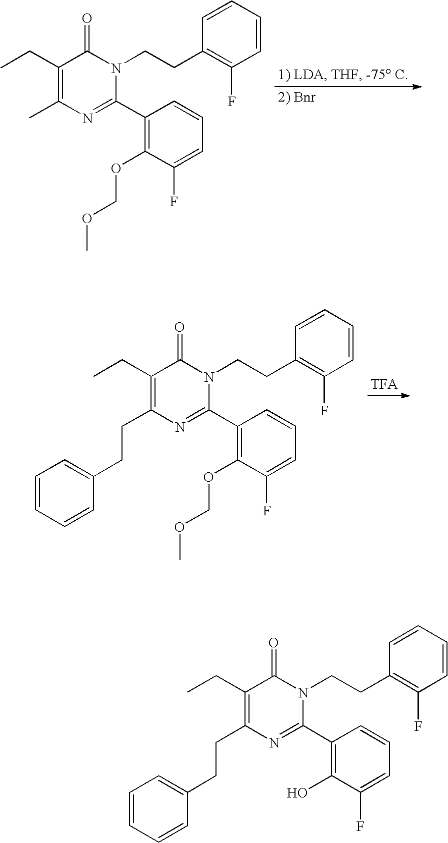

To the solution of 5-ethyl-2-(3-fluoro-2-{[(methyloxy)methyl]oxy}phenyl)-3-[2-(2-fluorophenyl)ethyl]-6-methyl-4(3H)-pyrimidinone (0.3 g, 0.725 mmol) of Example 45 in THF was cooled to −75° C. To this was added 2M LDA (0.38 mL, 0.76 mmol) dropwise. After stirring for at least one hour benzyl bromide (0.086 mL, 0.725 mmol) was added by syringe. Upon completion of the reaction, it was quenched by NH₄Cl, extracted with EtOAc. EtOAc layer was washed with brine. Aqueous layer was backextracted with EtOAc. EtOAc layers were combined, dried over Na₂SO₄, filtered and concentrated in vacuo. The residue was purified by flash chromatography (20% hexane/EtOAc) to yield 5,6-diethyl-2-[2-(methyloxy)phenyl]-3-(2-phenylethyl)-4(3H)-pyrimidinone (0.186 g) in 77% yield. This material was converted to the title compound using TFA as previously detailed: 1H NMR: 7.29-7.16 (m, 7H), 6.99-6.79 (m, 5H), 4.31 (t, 2H), 3.00 (t, 2H), 2.93 (m, 2H), 2.84 (m, 2H), 2.52 (q, 2H), 1.10 (t, 2H). MS (m/z): 461.4 (M+H).

### Example 198

### Preparation of 5-Ethyl-2-(3-fluoro-2-hydroxy-phenyl)-6-propyl-3-(2-fluoro-phenylethyl)-3H-pyrimidin-4-one

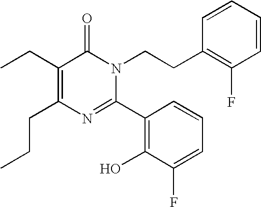

The title compound was prepared by substituting ethylbromide for benzylbromide in Example 197: ¹H NMR: 7.21-7.17 (m, 2H), 6.99-6.88 (m, 5H), 4.38 (t, 2H), 3.05 (t, 2H), 2.62 (m, 4H), 1.71 (q, 2H), 1.20 (t, 3H), 1.01 (t, 3H). MS (m/z): 399.4 (M+H).

### Example 199

### Preparation of 5-Ethyl-2-(3-fluoro-2-hydroxy-phenyl)-6-(3-phenyl-propyl)-3-(2-fluoro-phenylethyl)-3H-pyrimidin-4-one

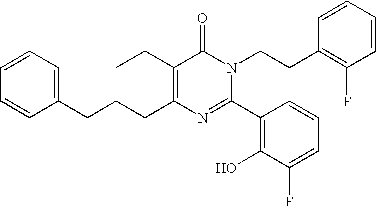

The title compound was prepared by substituting phenethyl bromide for benzylbromide in Example 197: ¹H NMR: 7.31-7.20 (m, 7H), 6.99-6.88 (m, 5H), 4.34 (t, 2H), 3.04 (t, 2H), 2.76-2.54 (m, 6H), 2.03 (m, 2H), 1.14 (t, 3H). MS (m/z): 475.5 (M+H).

### Example 200

### Preparation of 5-Ethyl-2-(3-fluoro-2-hydroxy-phenyl)-6-butyl-3-(2-fluoro-phenylethyl)-3H-pyrimidin-4-one

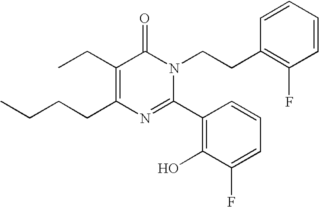

The title compound was prepared by substituting propylbromide for benzylbromide in Example 197: ¹H NMR: 7.13(m, 2H), 6.94-6.81 (m, 5H), 4.31 (t, 2H), 2.99 (t, 2H), 2.55 (m, 4H), 1.55 (m, 2H), 1.34 (m, H), 1.13 (t, 3H), 0.88 (t, 3H). MS (m/z): 413.4 (M+H).

### Example 201

### Preparation of 5-Ethyl-2-(3-fluoro-2-hydroxy-phenyl)-6-(2-methyl-propyl)-3-(2-fluoro-Phenylethyl)-3H-pyrimidin-4-one

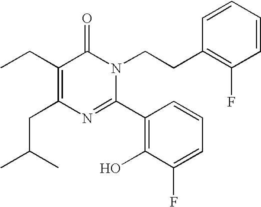

The title compound was prepared by substituting isoproylbromide for benzylbromide in Example 197: ¹H NMR: 7.19-7.11 (m, 2H), 7.02-6.84 (m, 5H), 4.44 (t, 2H), 3.04 (t, 2H), 2.64 (q, 4H), 2.48 (d, 2H), 2.08 (m, 1H), 1.16 (t, 3H), 0.98 (d, 6H). MS (m/z): 413.2 (M+H).

### Example 202

### Preparation of 5-Ethyl-2-(3-fluoro-2-hydroxy-phenyl)-6-(3-methyl-butyl)-3-(2-fluoro-phenylethyl)-3H-pyrimidin-4-one

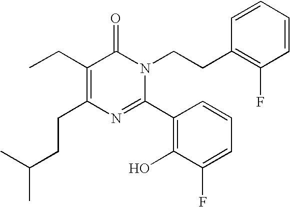

The title compound was prepared by substituting 3-methylbutyl bromide for benzylbromide in Example 197: ¹H NMR: 7.16-7.11(m, 2H), 7.98-6.84 (m, 5H), 4.35 (t, 2H), 3.02(t 2H), 2.61 (m, 4H), 1.62(m, 1H), 1.50(m, 2H), 1.20 (t, 3H), 0.98 (d, 6H). MS (m/z): 427.4 (M+H).

### Example 203

### Preparation of 5-Ethyl-2-(3-fluoro-2-hydroxy-phenyl)-6-(2-cyclobutyl-ethyl)-3-(2-fluoro-phenylethyl)-3H-pyrimidin-4-one

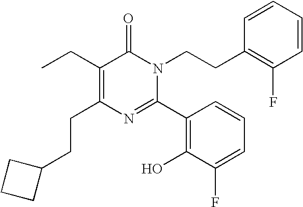

The title compound was prepared by substituting cyclobutylmethyl bromide for benzylbromide in Example 197: ¹H NMR: 7.21-7.11 (m, 2H), 7.98-6.81 (m, 5H), 4.37 (t, 2H), 3.06 (t, 2H), 2.61-2.47 (m, 4H), 2.30 (m, 1H), 1.96-1.58 (m, 6H), 1.22 (t, 3H). MS (m/z): 439.4 (M+H).

### Example 204

### Preparation of 2-(3-Fluoro-2-hydroxyphenyl)-6-methyl-3-(2-phenylethyl)-5-(3-thienyl)-4(3H)-pyrimidinone

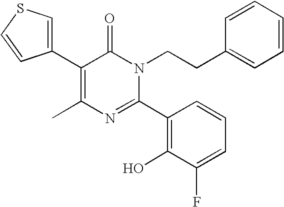

The title compound was prepared by substituting thiophene-3-boronic acid for 6-quinolinylboronic acid in Example 13. MS (m/z): 407.2 [M+H]⁺.

### Example 205

### Preparation of 5-ethyl-2-(4-fluoro-2-hydroxyphenyl)-6-methyl-3-(2-phenylethyl)-4(3H)-pyrimidinone

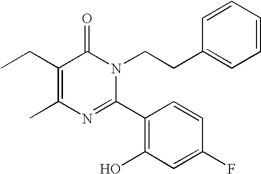

### a. 2-Ethyl-3-oxo-N-(2-phenylethyl)butanamide

Ethyl 2-ethylacetoacetate (2.0 mL, 12.4 mmol) and phenethylamine (0.78 mL, 6.19 mmol) in ethanol (0.2 mL) were heated to 180° C. in a microwave for 20 min. The reaction mixture was purified via column chromatography (2-60% ethyl acetate:hexane) to produce 0.860 g (60%) of the title compound: LCMS (m/z): 234.2 (M+H).

### b. 5-Ethyl-2-(4-fluoro-2-hydroxyphenyl)-6-methyl-3-(2-phenylethyl)-4(3H)-pyrimidinone

4-Fluoro-2-hydroxybenzamide (0.200 g, 1.29 mmol) was added to 2-ethyl-3-oxo-N-(2-phenylethyl)butanamide (0.150 g, 0.644 mmol) in titanium isopropoxide (3.0 mL, 10.2 mmol) and heated at 150° C. for 4 days. The reaction was diluted with CH₂Cl₂ and 1N HCl and stirred at room temperature for 3 h. The layers were separated and the aqueous layer extracted two times with CH₂Cl₂. The combined organic layers were dried over Na₂SO₄, filtered, and concentrated. Prep HPLC (20-95% CH₃CN:H₂O) afforded 0.063 g (28%) of the title compound: LCMS (m/z): 353.2 (M+H).

### Example 206

### Preparation of 2-(3-Fluoro-2-hydroxyphenyl)-6-methyl-3-(2-phenylethyl)-5-(5-phenyl-2-thienyl)-4(3H)-pyrimidinone

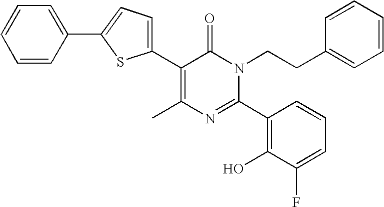

The title compound was prepared by substituting 5-phenylthienylboronic acid for 6-quinolinylboronic acid in Example 13. MS (m/z): 483.2 [M+H]⁺.

### Example 207

### Preparation of 2-(2-hydroxyphenyl)-6-methyl-5-(5-methyl-2-thienyl)-3-(2-phenylethyl)-4(3H)-pyrimidinone

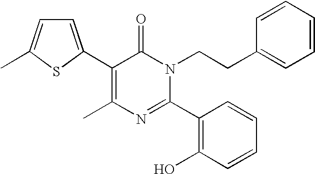

### a. 6-Methyl-5-(5-methyl-2-thienyl)-3-(2-phenylethyl)-2-{2-[(phenylmethyl)oxy]phenyl}-4(3H)-pyrimidinone

To a solution of 5-bromo-6-methyl-3-(2-phenylethyl)-2-{2-[(phenylmethyl)oxy]phenyl}-4(3H)-pyrimidinone (0.862 g, 1.81 mmol) in toluene (9.0 mL), ethanol (0.1 mL), and H₂O (0.1 mL) was added sodium carbonate (0.385 g, 3.63 mmol), 5-methylthiophene boronic acid (0.516 g, 3.63 mmol), and bis(tri-t-butylphosphine)palladium (0.145 g, 0.284 mmol). The resulting reaction mixture was heated at 110° C. for 3 h. The reaction was cooled to room temperature, filtered through a Celite-plugged filter frit, washed with CH₃OH and CH₂Cl₂, and concentrated. Column chromatography (1-35% ethyl acetate:hexane) provided 0.750 g (84%) of the title compound: LCMS (m/z): 493.2 (M+H).

### b. 2-(2-Hydroxyphenyl)-6-methyl-5-(5-methyl-2-thienyl)-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenyl methyl)oxy]phenyl}-4(3H)-pyrimidinone with 6-methyl-5-(5-methyl-2-thienyl)-3-(2-phenylethyl)-2-{2-[(phenylmethyl)oxy]phenyl}-4(3H)-pyrimidinone: LCMS (m/z): 403.2 (M+H).

### Example 208

### Preparation of 2-(2-hydroxyphenyl)-6-methyl-5-(5-methyl-3-thienyl)-3-(2-phenylethyl)-4(3H)-pyrimidinone

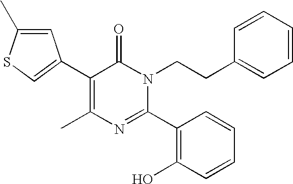

### a. 6-Methyl-5-(5-methyl-3-thienyl)-3-(2-phenylethyl)-2-{2-[(phenylmethyl)oxy]phenyl}-4(3H)-pyrimidinone

The title compound was prepared following the same procedure as Example 206d substituting 2-methyl-5-(tributylstannanyl)-1,3-thiazole with tributyl(5-methyl-3-thienyl)stannane: LCMS (m/z): 493.2 (M+H).

### b. 2-(2-Hydroxyphenyl)-6-methyl-5-(5-methyl-3-thienyl)-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 6-methyl-5-(5-methyl-3-thienyl)-3-(2-phenylethyl)-2-{2-[(phenylmethyl)oxy]phenyl}-4(3H)-pyrimidinone: LCMS (m/z): 403.2 (M+H).

### Example 209

### Preparation of 2-(3-fluoro-2-hydroxyphenyl-6-methyl-5-(5-methyl-3-thienyl)-3-(2-phenylethyl)-4(3H)-pyrimidinone

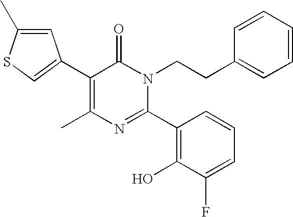

### a. 2-{3-Fluoro-2-[(phenylmethyl)oxy]phenyl}-6-methyl-5-(5-methyl-3-thienyl)-3-(2-phenylethyl)-4(3H)-pyrimidinone

The title compound was prepared following the same procedure as Example 206d substituting 5-bromo-6-methyl-3-(2-phenylethyl)-2-{2-[(phenylmethyl)oxy]phenyl}-4(3H)-pyrimidinone with 5-bromo-2-{3-fluoro-2-[(phenylmethyl)oxy]phenyl}-6-methyl-3-(2-phenylethyl)-4(3H)-pyrimidinone and 2-methyl-5-(tributylstannanyl)-1,3-thiazole with tributyl(5-methyl-3-thienyl)stannane. LCMS (m/z): 511.2 (M+H).

### b. 2-(3-fluoro-2-hydroxyphenyl)-6-methyl-5-(5-methyl-3-thienyl)-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 2-{3-fluoro-2-[(phenylmethyl)oxy]phenyl}-6-methyl-5-(5-methyl-3-thienyl)-3-(2-phenylethyl)-4(3H)-pyrimidinone: LCMS (m/z): 421.2 (M+H).

### Example 210

### Preparation of 5-(4,5-dimethyl-2-thienyl)-2-(2-hydroxyphenyl)-6-methyl-3-(2-phenylethyl)-4(3H)-pyrimidinone

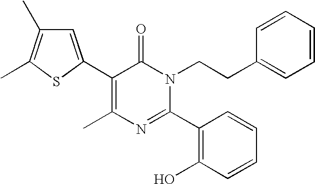

### a. 5-(4,5-Dimethyl-2-thienyl)-6-methyl-3-(2-phenylethyl)-2-{2-[(phenylmethyl)oxy]phenyl}-4(3H)-pyrimidinone

The title compound was prepared following the same procedure as Example 206d substituting 2-methyl-5-(tributylstannanyl)-1,3-thiazole with tributyl(4,5-dimethyl-2-thienyl)stannane: LCMS (m/z): 507.2 (M+H).

### b. 5-(4,5-Dimethyl-2-thienyl)-2-(2-hydroxyphenyl)-6-methyl-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 5-(4,5-dimethyl-2-thienyl)-6-methyl-3-(2-phenylethyl)-2-{2-[(phenylmethyl)oxy]phenyl}-4(3H)-pyrimidinone: LCMS (m/z): 417.0 (M+H).

### Example 211

### Preparation of 2-(2-hydroxyphenyl)-6-methyl-5-[5-(1,3-oxazol-5-yl)-2-thienyl]-3-(2-phenylethyl)-4(3H)-pyrimidinone

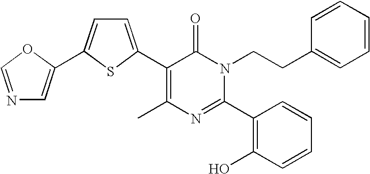

### a. 6-Methyl-5-[5-(1,3-oxazol-5-yl)-2-thienyl]-3-(2-phenylethyl)-2-{2-[(phenylmethyl)oxy]phenyl}-4(3H)-pyrimidinone

A solution of hexamethylditin (0.630 g, 1.92 mmol) in 1,4-dioxane (15 mL) was purged with N₂ for 15-20 min. 5-(5-Bromo-2-thienyl)-1,3-oxazole (0.450 g, 1.96 mmol) and bis(tri-t-butylphosphine)palladium (0.117 g, 0.229 mmol) were added and the reaction heated at 100° C. for 3 h. The reaction was cooled to room temperature over 30 min, 5-bromo-6-methyl-3-(2-phenylethyl)-2-{2-[(phenylmethyl)oxy]phenyl}-4(3H)-pyrimidinone (0.908 g, 1.91 mmol) was added, and the reaction heated at 100° C. for 48 h. The reaction was cooled, filtered through a Celite-plugged filter frit, washed with CH₃OH and CH₂Cl₂, and concentrated. Column chromatography (1-50% ethyl acetate:hexane) yielded 0.310 g (30%) of the title compound: LCMS (m/z): 546.2 (M+H).

### b. 2-(2-Hydroxyphenyl)-6-methyl-5-[5-(1,3-oxazol-5-yl)-2-thienyl]-3-(2-phenylethyl)-4(3H)=pyridinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 6-methyl-5-[5-(1,3-oxazol-5-yl)-2-thienyl]-3-(2-phenylethyl)-2-{2-[(phenylmethyl)oxy]phenyl}-4(3H)-pyrimidinone: LCMS (m/z): 456.0 (M+H).

### Example 212

### Preparation of 2-(2-hydroxyphenyl)-6-methyl-5-(4-methyl-2-thienyl)-3-(2-phenylethyl)-4(3H)-pyrimidinone

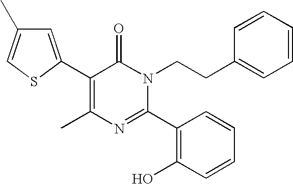

### a. 6-methyl-5-(4-methyl-2-thienyl-3-(2-phenylethyl)-2-{2-[(phenylmethyl)oxy]phenyl}-4(3H)-pyrimidinone

The title compound was prepared following the same procedure as Example 207a substituting 5-methylthiophene boronic acid with 4-methylthiophene boronic acid: LCMS (m/z): 493.2 (M+H).

### b. 2-(2-hydroxyphenyl)-6-methyl-5-(4-methyl-2-thienyl)-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 6-methyl-5-(4-methyl-2-thienyl)-3-(2-phenylethyl)-2-{2-[(phenylmethyl)oxy]phenyl}-4(3H)-pyrimidinone: LCMS (m/z): 403.2 (M+H).

### Example 213

### Preparation of 2-(3-Fluoro-2-hydroxyphenyl)-6-methyl-5-(2-methyl-1,3-thiazol-5-yl)-3-(2-phenylethyl)-4(3H)-pyrimidinone

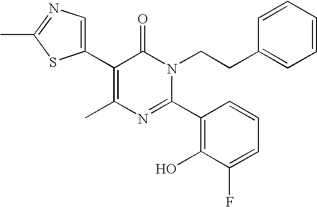

### a. 2-methyl-5-(tributylstannanyl)-1,3-thiazole

To a solution of 2-methyl-1,3-thiazole (5 g, 0.05 moles) in dry diethyl ether (60 mL) at −78° C. was added 1.6 M n-butyllithium (31.51 mL) in hexanes. Upon stirring for 1 h at −78° C. n-tributyltin chloride (16.32 mL, 0.061 moles) was added and the dry ice bath was removed. The reaction was slowly allowed to warm to RT overnight. The reaction mixture was filtered and concentrated and the crude residue was purified by distillation. ¹H NMR (400 MHz, CDCl₃) □ ppm 0.92 (t, J=7.8 Hz, 9H), 1.10-1.13 (m, 6H), 1.29-1.38 (m, 6H), 1.52-1.60 (m, 6H), 2.77 (s, 3H), 7.58 (d, J=6.57 Hz, 1H).

### b. 2-(3-Fluoro-2-hydroxyphenyl)-6-methyl-5-(2-methyl-1,3-thiazol-5-yl)-3-(2-phenylethyl)-4(3H)-pyrimidinone

To a solution containing 5-bromo-2-{3-fluoro-2-[(phenylmethyl)oxy]phenyl}-6-methyl-3-(2-phenylethyl)-4(3H)-pyrimidinone (0.2 g, 4.10 mmol, Example 11) in a sealed tube in deoxygenated dioxane was added Pd(t-Bu₃P)₂ (0.021 g, 0.41 mmol), cesium fluoride (0.14 g, 8.90 mmol) and 2-methyl-5-(tributylstannanyl)-1,3-thiazole (0.47 mL, 12.2 mmol) was added sequentially. The reaction was sealed and heated to 100° C. for 16 h and concentrated. The crude residue was filtered and concentrated. The crude material was purified by chromatography on silica gel (Biotage, 0-50% ethyl acetate/hexane) to afford the desired product (0.148 g) in 71% yield.

### c. 2-(3-Fluoro-2-hydroxyphenyl)-6-methyl-5-(2-methyl-1,3-thiazol-5-yl)-3-(2-phenylethyl)-4(3H)-pyrimidinone

2-{3-Fluoro-2-[(phenylmethyl)oxy]phenyl}-6-methyl-5-(2-methyl-1,3-thiazol-5-yl)-3-(2-phenylethyl)-4(3H)-pyrimidinone (0.148 g, 0.29 mmol) was placed in a round bottom flask equipped with a stir bar. To this was added HBr in acetic acid (5 mL) and stirred until starting material is all consumed. The reaction was quenched with saturated NaHCO₃ and extracted with dichloromethane. The combined organic layers were dried over Na₂SO₄, filtered and concentrated. The crude residue was purified by HPLC to afford the title compound (0.067 g) in 55% yield. MS (m/z): 422.2 [M+H]⁺.

### Example 214

### Preparation of 2-(2-hydroxyphenyl)-6-methyl-3-(2-phenylethyl)-5-[5-(1H-tetrazol-5-yl)-2-thienyl]-4(3H)-pyrimidinone

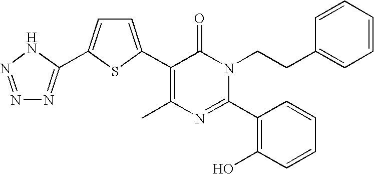

### a. 6-Methyl-3-(2-phenylethyl)-2-{2-[(phenylmethyl)oxy]phenyl}-5-[5-(1H-tetrazol-5-yl)-2-thienyl]-4(3H)-pyrimidinone

Ammonium chloride (0.024 g, 0.449 mmol) and sodium azide (0.029 g, 0.446 mmol) were added to 5-(4-methyl-6-oxo-1-(2-phenylethyl)-2-{2-[(phenylmethyl)oxy]phenyl}-1,6-dihydro-5-pyrimidinyl)-2-thiophenecarbonitrile (0.215 g, 0.427 mmol) in DMF (1.0 mL) and heated at 80° C. for 4 days. The reaction was cooled to room temperature, diluted with ethyl acetate, and washed with 0.5N HCl and brine. The organic layer was dried over Na₂SO₄, filtered, and concentrated to provide 0.210 g (90%) of the title compound: LCMS (m/z): 547.2 (M+H).

### b. 2-(2-Hydroxyphenyl)-6-methyl-3-(2-phenylethyl)-5-[5-(1H-tetrazol-5-yl)-2-thienyl]-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 6-methyl-3-(2-phenylethyl)-2-{2-[(phenylmethyl)oxy]phenyl}-5-[5-(1H-tetrazol-5-yl)-2-thienyl]-4(3H)-pyrimidinone: LCMS (m/z): 457.0 (M+H).

### Example 215

### Preparation of 5-[5-(Aminomethyl)-2-thienyl]-2-(2-hydroxyphenyl)-6-methyl-3-(2-phenylethyl)-4(3H)-pyrimidinone

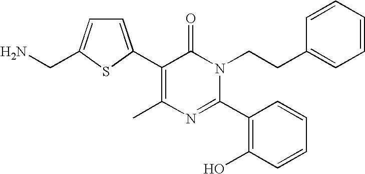

5-(4-Methyl-6-oxo-1-(2-phenylethyl)-2-{2-[(phenylmethyl)oxy]phenyl}-1,6-dihydro-5-pyrimidinyl)-2-thiophenecarbonitrile (0.630 g, 1.25 mmol) of Example 213 in methanol (15 mL) was purged with N₂ for 10-15 min. 10% Pd/C (0.500 g) was added and the reaction mixture stirred under H₂ (balloon pressure) for 3 days. The reaction was filtered through a Celite-plugged filter frit, washed with CH₃OH and CH₂Cl₂, and concentrated. Column chromatography (0-9% CH₃OH:CH₂Cl₂) afforded 0.123 g (24%) of the title compound: LCMS (m/z): 418.0 (M+H).

### Example 216

### Preparation of 2-(2-hydroxyphenyl)-6-methyl-5-{5-[(methylamino)methyl]-2-thienyl}-3-(2-phenylethyl)-4(3H)-pyrimidinone

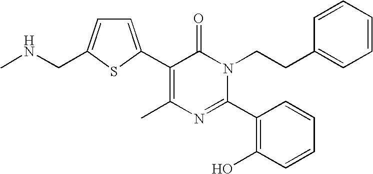

A solution of 5-[5-(aminomethyl)-2-thienyl]-2-(2-hydroxyphenyl)-6-methyl-3-(2-phenylethyl)-4(3H)-pyrimidinone (0.055 g, 0.132 mmol) of Example 215 in methanol (1.4 mL) under N₂ was cooled to 0° C. Formaldehyde (37% aqueous, 0.18 mL, 2.22 mmol) and sodium cyanoborohydride (0.032 g, 0.509 mmol) were added. The reaction was stirred at 0° C. for 5 min and then warmed to room temperature for 5 days. The reaction mixture was diluted with H₂O and extracted two times with CH₂Cl₂. The combined organic layers were dried over Na₂SO₄, filtered, and concentrated. Column chromatography (0-8% CH₃OH:CH₂Cl₂) provided 0.023 g (40%) of the title compound: LCMS (m/z): 432.2 (M+H).

### Example 217

### Preparation of 5-[5-(hydroxymethyl)-2-thienyl]-2-(2-hydroxyphenyl)-6-methyl-3-(2-phenylethyl)-4(3H)-pyrimidinone

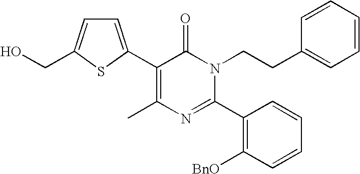

### a. 5-(4-Methyl-6-oxo-1-(2-phenylethyl)-2-{2-[(phenylmethyl)oxy]phenyl}-1,6-dihydro-5-pyrimidinyl)-2-thiophenecarboxylic acid

Sodium hydroxide (10%, 21 mL) was added to 5-(4-methyl-6-oxo-1-(2-phenylethyl)-2-{2-[(phenylmethyl)oxy]phenyl}-1,6-dihydro-5-pyrimidinyl)-2-thiophenecarbonitrile (0.813 g, 1.62 mmol) in ethanol (25 mL) and the reaction heated at reflux for 3.5 h. The reaction was cooled and acidified to pH ˜3 with 6N HCl. The aqueous layer was extracted two times with ethyl acetate. The combined organic layers were washed with brine, dried over Na₂SO₄, filtered, and concentrated. The title compound was isolated in 99% yield (0.840 g) and carried to the next step without further purification: LCMS (m/z): 523.0 (M+H).

### b. 5-[5-(Hydroxymethyl)-2-thienyl]-6-methyl-3-(2-phenylethyl)-2-{2-[(phenylmethyl)oxy]phenyl}-4(3H)-pyrimidinone

Sulfuric acid (0.22 mL) was added to 5-(4-methyl-6-oxo-1-(2-phenylethyl)-2-{2-[(phenyl methyl)oxy]phenyl}-1,6-dihydro-5-pyrimidinyl)-2-thiophenecarboxylic acid (0.250 g, 0.479 mmol) in ethanol (23 mL)under N₂ and heated at reflux for 40 h. The reaction was concentrated in vacuo upon cooling. The residue was diluted with sat. NaHCO₃ and extracted two times with CH₂Cl₂. The combined organic layers were washed with brine, dried over Na₂SO₄, filtered, and concentrated. The crude product was then dissolved in CH₂Cl₂ under N₂ and cooled to 0° C. DIBAL (1.0M in toluene, 1.9 mL, 1.91 mmol) was added and the reaction stirred at 0° C. for 1.5 h before warming to room temperature for 3 days. Acetone (3 mL), H₂O (6 mL), and Rochelle's salt solution (12 mL) were added and the reaction stirred for 1 h. The reaction mixture was then extracted four times with CH₂Cl₂. The combined organic layers were washed with brine, dried over Na₂SO₄, filtered, and concentrated. Column chromatography (0-10% CH₃OH:CH₂Cl₂) provided 0.111 g (46% over two steps) of the title compound: LCMS (m/z): 509.2 (M+H).

### c. 5-[5-(Hydroxymethyl)-2-thienyl]-2-(2-hydroxyphenyl)-6-methyl-3-(2-phenylethyl)-4(3H)-pyrimidinone

The title compound was prepared following the same procedure as Example 206e substituting 6-methyl-5-(2-methyl-1,3-thiazol-5-yl)-3-(2-phenylethyl)-2-{2-[(phenylmethyl)oxy]phenyl}-4(3H)-pyrimidinone with 5-[5-(hydroxymethyl)-2-thienyl]-6-methyl-3-(2-phenylethyl)-2-{2-[(phenylmethyl)oxy]phenyl}-4(3H)-pyrimidinone: LCMS (m/z): 419.2 (M+H).

### Example 218

### Preparation of 2-(3-Fluoro-2-hydroxyphenyl)-6-methyl-3-(2-phenylethyl)-5-(4, 5,6,7-tetrahydro-1-benzothien-2-yl)-4(3H)-pyrimidinone

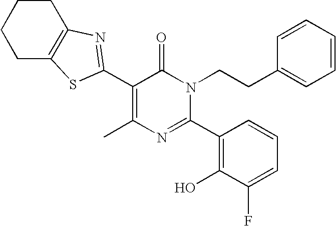

### a. 2-(3-Fluoro-2-hydroxyphenyl)-6-methyl-3-(2-phenylethyl)-5-(4,5,6,7-tetrahydro-1,3-benzothiazol-2-yl)-4(3H)-pyrimidinone

A solution of 5-bromo-2-{3-fluoro-2-[(phenylmethyl)oxy]phenyl}-6-methyl-3-(2-phenylethyl)-4(3H)-pyrimidinone (0.5 g, 1.02 mmol) of Example 11, 2-bromo-4,5,6,7-tetrahydro-1,3-benzothiazole (0.22 g, 1.02 mmol), hexamethyldistannane (0.21 mL, 1.02 mmol), Pd(tBu₃P)₂ (0.052 g, 0.102 mmol) in dioxane was degassed for 10 min and then heated for 48 h at 90° C. in a sealed tube. The reaction mixture was filtered through a bed of celite and concentrated and the crude product was purified on flash Silica gel column (0-40% EtOAc/hexanes) to give the product (0.1 g, MS (ES) m/e 551 [M+H]⁺) along with a side product, 2-{3-fluoro-2-[(phenylmethyl)oxy]phenyl}-6-methyl-3-(2-phenylethyl)-5-(trimethylstannanyl)-4(3H)-pyrimidinone (0.15 g) MS (ES) m/e 578[M+H]⁺.

### b. 2-(3-Fluoro-2-hydroxyphenyl)-6-methyl-3-(2-phenylethyl)-5-(4,5,6,7-tetrahydro-1,3-benzothiazol-2-yl)-4(3H)-pyrimidinone

A solution of 2-{3-fluoro-2-[(phenylmethyl)oxy]phenyl}-6-methyl-3-(2-phenylethyl)-5-(4,5,6,7-tetrahydro-1,3-benzothiazol-2-yl)-4(3H)-pyrimidinone (0.1 g, 0.181 mmol) in HBr (48% in acetic acid; 0.7 mL, 3.62 mmol) was stirred at room temperature for 3 h and additional HBr (0.5 mL) was added. After the starting material is all consumed, the reaction mixture was diluted with DCM and washed with Sat. NaHCO₃. The organic layers were dried over sodium sulfate, filtered, concentrated and purified by Biotage purification system to give the title compound as white solid (61 mg) in 73% yield. MS (ES) m/e 462.2[M+H]⁺.

### Example 219

### Preparation of 2-(3-Fluoro-2-hydroxyphenyl)-6-methyl-3-(2-phenylethyl)-5-(2-phenyl-1,3-thiazol-5-yl)-4(3H)-pyrimidinone

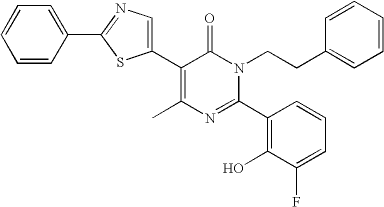

### a. 2-{3-Fluoro-2-[(phenylmethyl)oxy]phenyl}-6-methyl-3-(2-phenylethyl)-5-(2-phenyl-1,3-thiazol-5-yl)-4(3H)-pyrimidinone

To a solution containing 2-{3-fluoro-2-[(phenylmethyl)oxy]phenyl}-6-methyl-3-(2-phenylethyl)-5-(trimethylstannanyl)-4(3H)-pyrimidinone (0.15 g, 0.26 mmol) a by-product of example 218 step a and 5-bromo-2-phenyl-1,3-thiazole (0.063 g, 0.26 mmol) in deoxygenated dioxane was added Pd(tBu₃P)₂ (0.013 g, 0.026 mol) and cesium fluoride (0.087 g, 0.572 mmol) and refluxed overnight. The crude residue is filtered through a bed of celite and diluted with dichloromethane and washed with saturated aqueous potassium fluoride, water and brine. The organic layer was separated, dried over Na₂SO₄, filtered and concentrated. The crude material was purified by reverse phase HPLG (no TFA) to afford the desired product (0.03 g). Subsequent deprotection using HBr as detailed in example 1 provided the product (0.013 g) in 52% yield. MS (m/z): 484.2[M+H]⁺.

### Example 220

### Preparation of 2-(3-Fluoro-2-hydroxyphenyl)-5-(4-hydroxyphenyl-6-methyl-3-(2-phenylethyl)-4(3H)-pyrimidinone

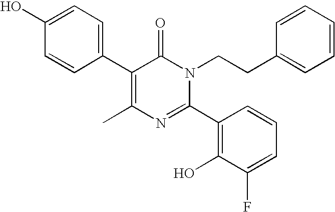

The title compound was prepared by BBr₃ deprotection of Example 75 as previously detailed. MS (m/z): 417[M+H]⁺.

### Example 221

### Preparation of 2-(3-Fluoro-2-hydroxyphenyl-5-(2-hydroxyphenyl)-6-methyl-3-(2-phenylethyl)-4(3H)-pyrimidinone

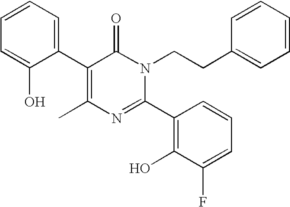

The title compound was prepared by BBr₃ deprotection of Example 94 as previously detailed. MS (m/z): 417[M+H]⁺.

### Example 222

### Preparation of 2-(3-Fluoro-2-hydroxyphenyl)-5-(3-hydroxyphenyl)-6-methyl-3-(2-phenylethyl)-4(3H)-pyrimidinone

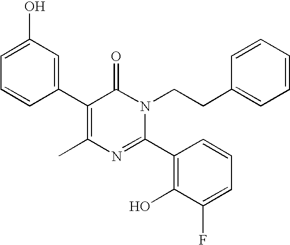

The title compound was prepared by BBr₃ deprotection of Example 95 as previously detailed. MS (m/z): 417[M+H]⁺.

### Example 223

### Preparation of 2-(2-Hydroxyphenyl)-6-methyl-5-(2-methylpropyl)-3-[2-(1-piperidinyl)ethyl]-4(3H)-pyrimidinone

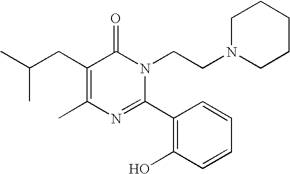

### a. 2-acetyl-4-methyl-N-[2-(1-piperidinyl)ethyl]pentanamide

To a solution of methyl 2-acetyl-4-methyl-4-pentanoate (2.0 g, 0.012 mol) in dimethoxyethane was added [2-(1-piperidinyl)ethyl]amine (0.83 mL, 5.81 mmol). This mixture was heated in a microwave at 180° C. for 20 minutes whereupon it was concentrated and purified by flash column chromatography (0-100% EtOAc/hexanes) to provide 1.1 g of the product

### b. 2-(3-fluoro-2-hydroxyphenyl)-6-methyl-5-(2-methylpropyl)-3-(2-phenylethyl)-4(3H)-pyrimidinone

To a solution of 2-acetyl-4-methyl-N-[2-(1-piperidinyl)ethyl]pentanamide (1.1 g, 4.10 mmol) and salicylamide (1.12 g, 8.2 mmol) and Ti(O-i-Pr)₄ (18 mL, 49.2 mmol). This mixture 6 was heated to reflux for 48 hours whereupon it was cooled to room temperature and concentrated. The residue was diluted with CH₂Cl₂ and washed 6NHC). The organic layer was concentrated and the residue purified by flash column chromatography (0-100% EtOAc/hexanes) to provide the title compound (0.193 g). MS (EI) 370(M+H)⁺.

### Example 224

### Preparation of 5-Ethyl-3-[2-(2-fluorophenyl)ethyl]-2-(3-hydroxyphenyl)-6-methyl-4(3H)-pyrimidinone

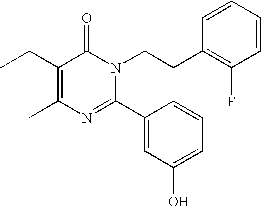

The title compound was prepared following procedures outlined in example 1 except substituting 2-ethyl-N-[2-(2-fluorophenyl)ethyl]-3-oxobutanamide for 2-acetyl-4-methyl-N-(2-phenylethyl)pentanamide and 3-methoxybenzamide for 2-fluoro-3-methoxybenzamide. MS (m/z). 353[M+H]⁺.

### Example 225

### Preparation of 2-(2-Hydroxyphenyl)-6-methyl-5-[5-(5-methyl-1,3,4-oxadiazol-2-yl)-2-thienyl]-3-(2-Phenylethyl)-4(3H)-pyrimidinone

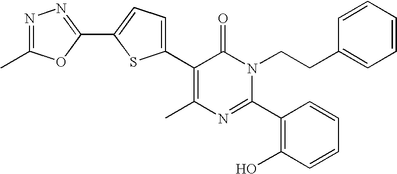

### a. 2-{3-Fluoro-2-[(phenylmethyl)oxy]phenyl}-6-methyl-5-[5-(2-methyl-1,3-thiazol-4-yl)-2-thienyl]-3-(2-phenylethyl)-4(3H)-pyrimidinone

A solution of 5-bromo-6-methyl-3-(2-phenylethyl)-2-{2-[(phenylmethyl)oxy]phenyl}-4(3H)-pyrimidinone (0.3 g, 0.63 mmol, Example 20), 2-(5-bromo-2-thienyl)-5-methyl-1,3,4-oxadiazole (0.155 g, 0.63 mmol), hexamethyldistannane (0.13 mL, 0.63 mmol), Pd(PPh₃)₄ (0.073 g, 0.063 mmol) in dioxane was degassed for 10 min and then heated at 90° C. for 16 h. The reaction mixture was concentrated and the crude product was purified on flash Silica gel column (EtOAc/hexanes) to give desired product (0.03 g) in yield. MS (ES) m/e 561[M+H]⁺.

### b. 2-(2-Hydroxyphenyl)-6-methyl-5-[5-(5-methyl-1,3,4-oxadiazol-2-yl)-2-thienyl]-3-(2-phenylethyl)-4(3H)-pyrimidinone

2-{3-Fluoro-2-[(phenylmethyl)oxy]phenyl}-6-methyl-5-[5-(2-methyl-1,3-thiazol-4-yl)-2-thienyl]-3-(2-phenylethyl)-4(3H)-pyrimidinone (0.03 g, 0.053 mmol) was taken up in glacial acetic acid (10 mL). To this was added 10% Pd/C. This mixture was placed under hydrogen atmosphere at 43 psi and shaken for 16 h. The reaction mixture was filtered through a bed of celite and concentrated. The crude residue was purified by chromatography on silica gel to afford the desired product. MS (ES) m/e 471[M+H]⁺.

### Example 226

### Preparation of 5-(2,3-Dihydro-1,4-benzodioxin-6-yl)-2-(2-hydroxyphenyl)-6-[(methyloxy)methyl]-3-(2-Phenylethyl)-4(3H)-pyrimidinone

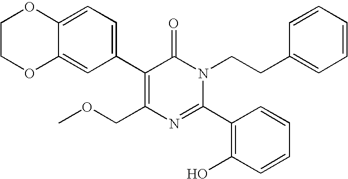

### a. 5-Bromo-6-[(methyloxy)methyl]-3-(2-phenylethyl)-2-{2-[(phenylmethyl)oxy]phenyl}-4(3H)-pyrimidinone

5-Bromo-6-[(methyloxy)methyl]-3-(2-phenylethyl)-2-{2-[(phenylmethyl)oxy]phenyl}-4(3H)-pyrimidinone (12.0 g, 0.028 moles) of Example 38 was taken up in glacial acetic acid (200 mL). To this was added bromine (1.4 mL, 0.028 moles) dropwise by a syringe. Reaction was stirred for 16 h. Ethyl acetate was added and acetic acid was washed with saturated sodium carbonate. The organic layer was further washed with brine and dried (MgSO₄). Solid was filtered off and organic layer was concentrated. The crude product was triturated with hexanes to obtain the desired product (2.05 g). MS (m/z): 507 [M+H]⁺.

### b. 5-(2,3-Dihydro-1,4-benzodioxin-6-yl)-6-[(methyloxy)methyl]-3-(2-phenylethyl)-2-{2-[(phenylmethyl)oxy]phenyl}-4(3H)-pyrimidinone

To a solution of 5-bromo-6-[(methyloxy)methyl]-3-(2-phenylethyl)-2-{2-[(phenylmethyl)oxy]phenyl}-4(3H)-pyrimidinone (0.505 g, 0.001 moles) in dioxane (6 mL) was added 1,4-benzodioxane-6-boronic acid (0.36 g, 0.002 mmoles) dissolved in solvent mixture of 1 mL ethanol and 0.5 mL of aqueous sodium carbonate (0.21 g, 0.002 moles) in a microwave vessel. Pd(PPh₃)₄ (0.17 g, 0.15 mmol) was added to this and irradiated to 150° C. for 3000 seconds. The reaction mixture was filtered through syringe filter (Acrodisc CR25 mm with 0.2 □m PTFE membrane). The filtrate was diluted with EtOAc and washed with brine, separated, dried over sodium sulfate, filtered, concentrated in vacuo and the residue was purified by chromatography on silica gel (30% ethyl acetate/hexane) to afford the desired product (0.3 g). MS (m/z): 563 [M+H]⁺. Subsequent deprotection with HBr in acetic acid as detailed previously produced the title compound. MS (m/z): 473 [M+H]⁺

### Example 227

### Preparation of 2-(2-Hydroxyphenyl)-6-[(methyloxy)methyl]-5-(4-methyl-2-thienyl)-3-(2-phenylethyl)-4(3H)-pyrimidinone

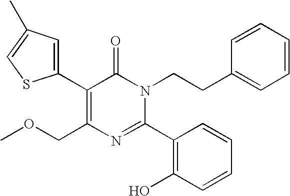

The title compound was prepared following procedures outlined in example 225 except substituting (4-methyl-2-thienyl)boronic acid for 1,4-benzodioxane-6-boronic acid. MS (m/z): 433 [M+H]⁺.

### Example 228

### Preparation of 2-(2-Hydroxyphenyl)-6-[(methyloxy)methyl]-5-(5-methyl-2-thienyl)-3-(2-phenylethyl)-4(3H)-pyrimidinone

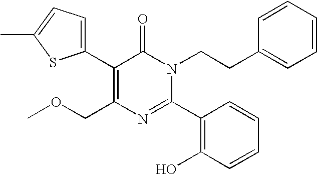

The title compound was prepared following procedures outlined in example 226 except substituting (5-methyl-2-thienyl)boronic acid for 1,4-benzodioxane-6-boronic acid. MS (m/z): 433 [M+H]⁺.

### Example 229

### Preparation of 5-Bromo-6-[(dimethylamino)methyl]-2-(2-hydroxyphenyl)-3-(2-phenylethyl)-4(3H)-pyrimidinone

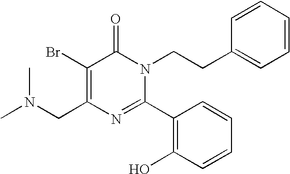

### a. 5-Bromo-2-(2-hydroxyphenyl)-6-[(methyloxy)methyl]-3-(2-phenylethyl)-4(3H)-pyrimidinone

The title compound was prepared upon deprotection of example 226a using BBr₃ as detailed previously.

### b. 5-Bromo-6-(bromomethyl)-2-(2-hydroxyphenyl)-3-(2-phenylethyl)-4(3H)-pyrimidinone

To a cold solution of 5-bromo-2-(2-hydroxyphenyl)-6-[(methyloxy)methyl]-3-(2-phenylethyl)-4(3H)-pyrimidinone (1.46 g, 0.0036 mol) and CBr₄ (1.52 g, 0.0046 mol) in DCM (30 mL) was added triphenylphosphine (1.43 g, 0.0054 mol). The reaction was stirred overnight while slowly warmed to RT. The reaction mixture was concentrated and purified by chromatography on silica gel (30% ethyl acetate/hexane) to afford the desired product (0.9 g) in 56% yield.

### c. 5-Bromo-6-[(dimethylamino)methyl]-2-(2-hydroxyphenyl)-3-(2-phenylethyl)-4(3H)-pyrimidinone

To a solution of 5-bromo-6-(bromomethyl)-2-(2-hydroxyphenyl)-3-(2-phenylethyl) 4(3H)-pyrimidinone (0.232 g, 0.51 mmol) and N,N-dimethylamine hydrochloride (0.203 g, 0.0025 mol) in 10 mL of DMF was added cesium carbonate (0.98 g, 0.003 mol) and stirred at RT for 16 h. The reaction was concentrated and diluted with water and extracted with DCM. The organic layer was washed with brine, dried (MgSO4) and concentrated. The crude residue was triturated with hexanes/ether mixtures to give the title compound (0.09 g) in 42% yield. MS (m/z): 428 [M+H]⁺.

### Example 230

### Preparation of 6-[Dimethylamino)methyl]-2-(2-hydroxyphenyl)-3-(2-phenylethyl)-4(3H)-pyrimidinone

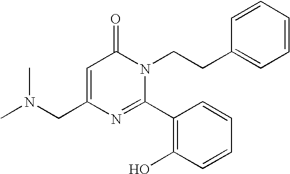

The title compound was prepared upon catalytic hydrogenolysis of example 229. MS (m/z): 350 [M+H]⁺.

### Example 231

### Preparation of 2-(2-Hydroxyphenyl)-3-(2-Phenylethyl)-5,6,7,8,9,10-hexahydrocycloocta[d]pyrimidin-4(3H)-one

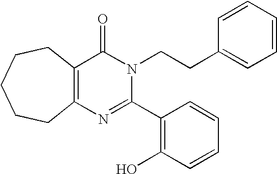

### a. 2-(Methyloxy)benzenecarboximidamide

To a cold solution of anhydrous ether was introduced under Argon, LiHMDS (150 ml, 150 mmol) and stirred for 5 min. 2-Methoxy-benzonitrile (8 g, 60 mmol) was then added and the mixture was stirred at room temperature for 3 days. When all the starting material is consumed the reaction mixture was concentrated and 200 mL of cold 1N HCl was added and stirred for additional 0.5 h. The aqueous layer was extracted with diethyl ether then adjusted the pH of the aqueous layer to 13 by addition of 6N NaOH. The 2-(methyloxy)benzenecarboximidamide free base was extracted with dichloromethane. The combined organic layers were dried over Na₂SO₄ and concentrated to give crude product (9.4 g) which was carried to the next without purification.

### b. 2-[2-(Methyloxy)phenyl]-5,6,7,8,9,10-hexahydrocycloocta[d]pyrimidin-4(1H)-one

To a solution of 2-(methyloxy)benzenecarboximidamide (3.78 g, 0.025 mol) in 250 mL of solvent mixture of MeOH/Dioxane (1/1) was added NaOMe (2.72 g) and stirred for 15 min. Ethyl 2-oxo-1-cyclooctanecarboxylate (5.0 g, 0.025 mol) was introduced and the reaction mixture was heated to reflux for 16 h. Upon completion the reaction mixture was concentrated, diluted with dichloromethane and added dilute HCl. The dichloromethane layer was separated and washed with brine, dried over Na₂SO₄. Upon filteration and concentration the crude product was purified by FCC (30% ethylacetate/hexane) to give product (3.81 g) in 53% yield.

### c. 2-[2-(Methyloxy)phenyl]-3-(2-phenylethyl)-5,6,7,8,9,10-hexahydrocycloocta[d]pyrimidin-4(3H)-one

To a solution of 2-[2-(Methyloxy)phenyl]-5,6,7,8,9,10-hexahydrocycloocta[d]pyrimidin-4(1H)-one (1.95 g, 6.86 mmol) in dry DMF was added LiH (0.11 g, 13.7 mmol) and lithium bromide (1.79 g, 20.6 mmol) and stirred for 10 min at room temperature. Then (2-bromoethyl)benzene (6.35 g, 34.3 mmol) was added and stirred overnight. The reaction mixture was quenched by addition of ice and 6N HCl. This mixture was extracted with EtOAc and the organic layer was washed with aqueous NaHCO₃, brine and dried over Na₂SO₄. The sodium sulfate was filtered and concentrated. The crude product is purified by FCC (30% ethyl acetate/hexane) to give product (1.60 g) in 60% yield.

### d. 2-(2-Hydroxyphenyl)-3-(2-phenylethyl)-5,6,7,8,9,10-hexahydrocycloocta[d]pyrimidin-4(3H)-one

2-[2-(Methyloxy)phenyl]-3-(2-phenylethyl)-5,6,7,8,9,10-hexahydrocycloocta[d]pyrimidin-4(3H)-one (1.60 g, 4.12 mmol) in 10 mL of dichloromethane was cooled to 0° C. 1M dichloromethane solution of BBr₃ (21 mL, 20.6 mmol) was then added and let the reaction mixture warmed to RT while stirring continued for 12 h. The reaction mixture was then diluted with dichloromethane and aqueous NaHCO₃ was then added. Organic layer was separated and washed with H₂O, brine and dried over Na₂SO₄. Filtered, concentrated and purified by purified by chromatography on silica gel (Biotage, 0-20% ethyl acetate/hexane) to afford pure compound (1.43 g) in 93% yield. MS (mjz): 375.4 [M+H]⁺.

### Example 232

### Preparation of 5-(4,5-Dimethyl-1,3-thiazol-2-yl)-2-(3-fluoro-2-hydroxyphenyl)-6-methyl-3-(2-phenylethyl)-4(3H)-pyrimidinone

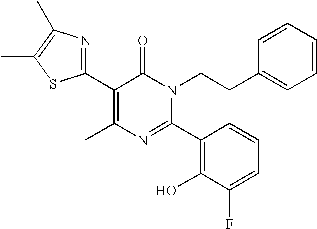

The title compound was prepared according to the procedure of Example 97 except substituting 4,5-dimethyl-2-(tributylstannanyl)-1,3-thiazole for 2-(tributylstannanyl)-1,3-thiazole. MS (m/z): 436.2 [M+H]⁺

### Example 233

### Preparation of 2-(3-Fluoro-2-hydroxyphenyl)-6-methyl-5-(4-methyl-1,3-thiazol-2-yl) 3-(2-phenylethyl)-4(3H)-pyrimidinone

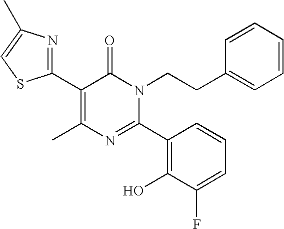

The title compound was prepared according to the procedure of Example 97 except substituting 4-methyl-2-(tributylstannanyl)-1,3-thiazole for 2-(tributylstannanyl)-1,3-thiazole. MS (m/z): 422.0 [M+H]⁺

### Example 234

### Preparation of 5-(1,3-Benzodioxol-5-yl)-2-(3-fluoro-2-hydroxyphenyl)-6-methyl-3-(2-phenylethyl)-4(3H)-pyrimidinone

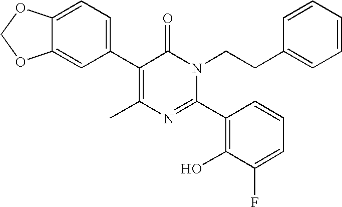

The title compound was prepared according to the procedure of Example 74 except substituting 3,4-methylenedioxyphenyl boronic acid for 4-(N,N-dimethylamino)phenylboronic acid. MS (m/z): 445.0 [M+H]⁺.

### Example 235

### Preparation of 3-(2,3-Dihydro-1H-inden-2-yl)-2-(2-hydroxyphenyl)-6-methyl-5-(5-methyl-2-thienyl)-4(3H)-pyrimidinone

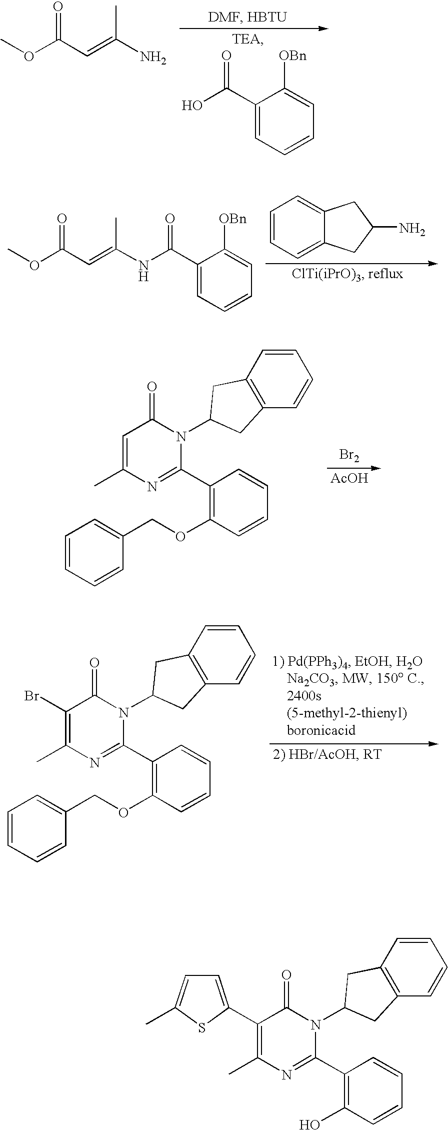

### a. Methyl (2Z)-3-[({2-[(phenylmethyl)oxy]phenyl}carbonyl)amino]-2-butenoate

To a solution of 2-[(phenylmethyl)oxy]benzoic acid (11.88 g, 0.052 mol) in 1,2-DCE was added HATU (20.64 g, 0.054 mol) and TEA (6.66 mL, 0.043 mol). The reaction mixture stirred for 1 h. At this time, methyl (2Z)-3-amino-2-butenoate (5.0 g, 0.043 mol) was added followed by another portion of TEA (6.66 mL, 0.043 mol). The reaction was heated to reflux for 16 h. The reaction mixture was cooled and diuted with EtOAc and washed successively with 1N HCl, 5% NaHCO₃ and brine. Dried over sodium sulfate, filtered and concentrated in vacuo. The residue was purified by Biotage purification system (0-50% ethyl acetate/hexane) to afford the title compound as two geometric isomers in 1:6.87 ratio (6.63 g, 47%).

### b. 5-Bromo-3-(2,3-dihydro-1H-inden-2-yl)-6-methyl-2-{2-[(phenylmethyl)oxy]phenyl}-4(3H)-pyrimidinone

To 2,3-dihydro-1H-inden-2-amine (6.66 g, 0.05 mol) in toluene was added chlorotriisopropoxytitanium (13.03 g, 0.05 mol) and stirred for 30 min. Methyl (2E)-3-[({2-[(phenylmethyl)oxy]phenyl}carbonyl)amino]-2-butenoate (3.25 g, 0.01 mol) was added and the reaction was heated to reflux. Upon completion the reaction was concentrated in vacuo, then diluted with EtOAc, washed with 1N HCl, sat. (NH₄)₂CO₃, and brine. EtOAc layer was dried over Na₂SO₄, filtered and concentrated. The residue was purified by flash chromatography (0-50% EtOAc/hexane) to afford the product (1.27 g) in 31% yield. Subsequent bromination as detailed previously gave the title compound.

### c. 3-(2,3-Dihydro-1H-inden-2-yl)-2-(2-hydroxyphenyl)-6-methyl-5-(5-methyl-2-thienyl)-4(3H)-pyrimidinone

The title compound was prepared according to the procedures of Example 74 except substituting 5-bromo-3-(2,3-dihydro-1H-inden-2-yl)-6-methyl-2-{2-[(phenylmethyl)oxy]phenyl}-4(3H)-pyrimidinone for 5-bromo-2-{3-fluoro-2-[(phenylmethyl)oxy]phenyl}-6-methyl-3-(2-phenylethyl)-4(3H)-pyrimidinone and

5-methylthiophene-2-boronic acid for 4-(N,N-dimethylamino)phenylboronic acid. Debenzylation using HBr in acetic acid as detailed previously afforded the product. MS (m/z): 415.2 [M+H]⁺.

### Example 236

### Preparation of 3-[1-(2,3-Dihydro-1H-inden-2-yl)-2-(2-hydroxyphenyl)-4-methyl-6-oxo-1,6-dihydro-5-pyrimidinyl]benzonitrile

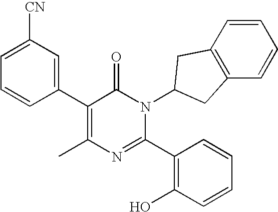

The title compound was prepared according to the procedures of Example 235 except substituting 3-cyanophenylboronic acid for 5-methylthiophene-2-boronic acid. Subsequent catalytic hydrogenolysis provided the title compound. MS (m/z): 420.2 [M+H]⁺

### Example 237

### Preparation of 3-(2,3-Dihydro-1H-inden-2-yl)-5-(4,5-dimethyl-1,3-thiazol-2-yl)-2-(2-hydroxyphenyl)-6-methyl-4(3H)-pyrimidinone

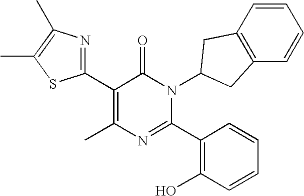

To a solution of 5-bromo-2-{3-fluoro-2-[(phenylmethyl)oxy]phenyl}-6-methyl-3-(2-phenylethyl)-4(3H)-pyrimidinone (0.20 g, 0.41 mmol) of Example 234b in dioxane was added cesium fluoride (0.137 g, 0.89 mmol). After 10 min. of deoxygenation, bis(tri-t-phosphine)palladium (0.021 g, 0.041 mmol) and 4,5-dimethyl-2-(tributylstannanyl)-1,3-thiazole (0.197 g, 0.49 mmol) were added. The mixture in sealed vessel was heated to 100° C. overnight. The reaction mixture was filtered through celite and diluted with ethyl acetate. The filtrate was washed with 10% w/v potassium fluoride, separated, dried over sodium sulfate, filtered, concentrated in vacuo and the residue purified by flash chromatography to afford the desired product (0.152 g, 71% yield). HBr deprotection yielded the title compound (0.039 g). MS (m/z): 430.2 [M+H]⁺.

### Example 238

### Preparation of 3-(2-Cyclohexylethyl)-2-(3-fluoro-2-hydroxyphenyl)-6-methyl-5-(5-methyl-2-thienyl)-4(3H)-pyrimidinone

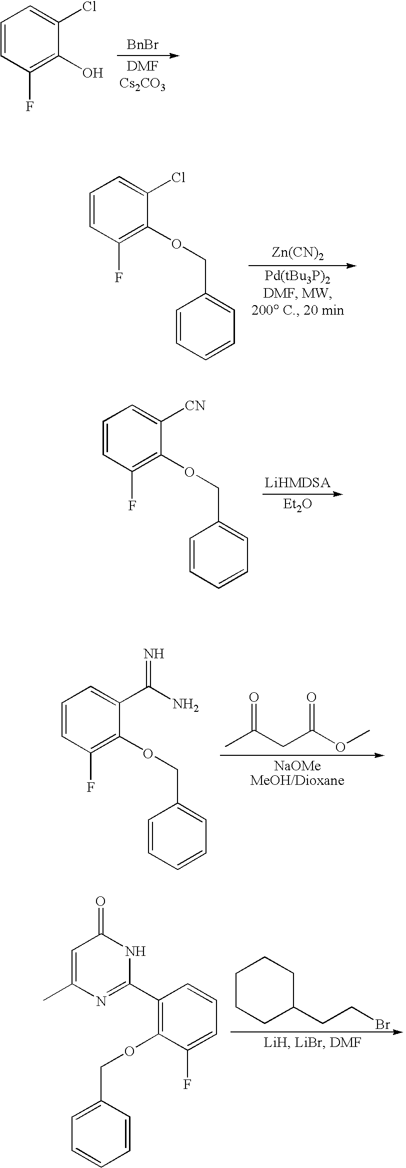

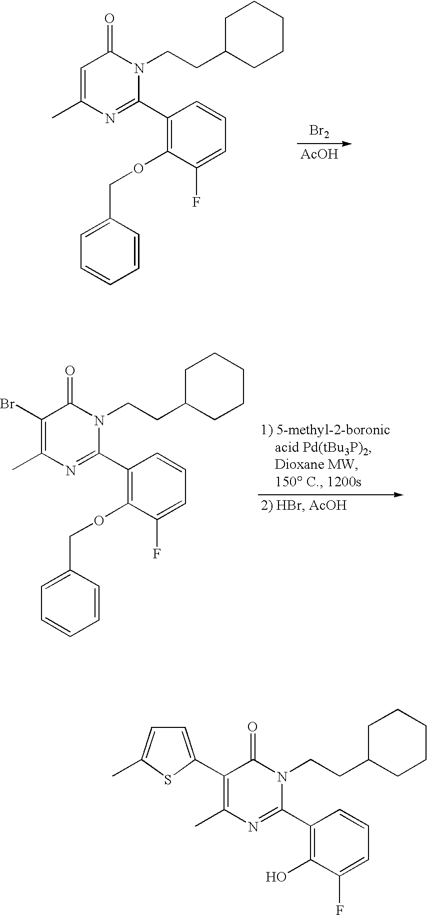

### a. 3-Fluoro-2-[(phenylmethyl)oxy]benzenecarboximidamide

The title compound is prepared following procedure outlined in example 35 except substituting benzyl bromide for methyl iodide.

### b. 2-{3-Fluoro-2-[(phenylmethyl)oxy]phenyl}-6-methyl-4(1H)-pyrimidinone

NaOMe (8.52 g, 0.158 mol) was added to a 0° C. solution of 3-fluoro-2-[(phenylmethyl)oxy]benzenecarboximidamide (17.51 g, 0.072 mol) and methylacetoacetate (9.99 g, 0.086 mol) in methanol and 1,4-dioxane mixture. The resulting mixture was refluxed overnight. The solvents were removed and the residue was diluted with H₂O and quenched with NH₄Cl. The layers were separated and the aqueous layer was extracted with dichlormethane 3 times. The combined organic portions were dried over Na₂SO₄ and purified by flash column chromatography to give 19.37 g of product in 87% yield.

### c. 3-(2-Cyclohexylethyl)-2-{3-fluoro-2-[(phenylmethyl)oxy]phenyl}-6-methyl-4(3H)-pyrimidinone

To a solution of 2-{3-fluoro-2-[(phenylmethyl)oxy]phenyl}-6-methyl-4(1H)-pyrimidinone (5.0 g, 16.12 mmol) in DMF were added lithium hydride (0.384 g, 48.30 mmol), lithium bromide (4.20 g, 48.3 mmol), and 2-cyclohexylethyl bromide (15.4 g, 80.6 mmol). Upon stirring overnight at room temperature, the reaction was quenched with saturated ammonium chloride, extracted with ethyl acetate. The combined organic layers were washed with brine, dried over sodium sulfate, filtered, concentrated in vacuo and the residue purified by flash chromatography (0-30% ethyl acetate/hexane) to afford the desired product (2.46 g, 36%).

### d. 5-Bromo-3-(2-cyclohexylethyl)-2-{3-fluoro-2-[(phenylmethyl)oxy]-phenyl}-6-methyl-4(3H)-pyrimidinone

3-(2-Cyclohexylethyl)-2-{3-fluoro-2-[(phenylmethyl)oxy]phenyl}-6-methyl-4(3H)-pyrimidinone (2.46 g, 5.85 mmol) was taken up in glacial acetic acid (60 mL). To this was added bromine (0.40 mL, 7.78 mmol) dropwise by a syringe. Reaction was stirred for 16 h. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was purified by chromatography on silica gel (Biotage) using ethylacetate and hexane mixtures (10-50%) to obtain the desired product (2.72 g) in 93% yield.

### e. 3-(2-Cyclohexylethyl)-2-(3-fluoro-2-hydroxyphenyl)-6-methyl-5-(5-methyl-2-thienyl)-4(3H)-pyrimidinone

To a solution of 5-bromo-3-(2-cyclohexylethyl)-2-{3-fluoro-2-[(phenylmethyl)oxy]phenyl}-6-methyl-4(3H)-pyrimidinone (0.462 g, 0.92 mmol) in dioxane was added 5-methylthiopheneboronic acid (0.263 g, 1.85 mmol) dissolved in solvent mixture of 0.5 mL ethanol and 0.5 mL of dioxane, and 0.5 mL of aqueous sodium carbonate (0.196 g, 1.85 mmol) in a microwave reaction vessel and irradiated to 150° C. for 2400 seconds. The reaction mixture was filtered through syringe filter (Acrodisc CR25 mm with 0.2 ␣m PTFE membrane). The filtrate was diluted with EtOAc and washed with brine, separated, dried over sodium sulfate, filtered, concentrated in vacuo and the residue was purified by chromatography on silica gel (Biotage, 20% ethyl acetate/hexane) to afford the desired product. Subsequent debenzylation using hydrobromic acid in acetic acid as detailed previously afforded the title compound. MS (m/z): 427.2 [M+H]⁺.

### Example 239

### Preparation of 3-(2-Cyclohexylethyl)-5-(4,5-dimethyl-1,3-thiazol-2-yl)-2-(3-fluoro-2-hydroxyphenyl)-6-methyl-4(3H)-pyrimidinone

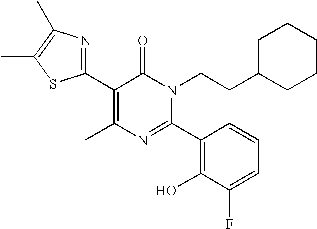

The title compound was prepared according to the procedures of Example 97 except substituting 5-bromo-3-(2-cyclohexylethyl)-2-{3-fluoro-2-[(phenylmethyl)oxy]phenyl}-6-methyl-4(3H)-pyrimidinone for 5-bromo-2-{3-fluoro-2-[(phenylmethyl)oxy]phenyl}-6-methyl-3-(2-phenylethyl)-4(3H)-pyrimidinone and 4,5-dimethyl-2-(tributylstannanyl)-1,3-thiazole for 2-(tributylstannanyl)-1,3-thiazole. Subsequent debenzylation using hydrobromic acid in acetic acid as detailed previously afforded the title compound. MS (m/z): 442.4 [M+H]⁺.

### Example 240

### Preparation of 3-(2-Cyclohexylethyl)-2-(3-fluoro-2-hydroxyphenyl)-6-methyl-5-(2-methyl-1,3-thiazol-5-yl)-4(3H)-pyrimidinone

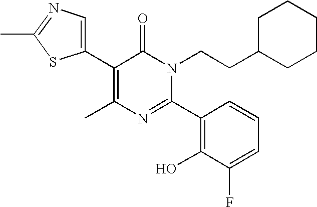

The title compound was prepared according to the procedures of Example 97 except substituting 2-methyl-5-(tributylstannanyl)-1,3-thiazole for 2-(tributylstannanyl)-1,3-thiazole. Subsequent debenzylation using hydrobromic acid in acetic acid as detailed previously afforded the title compound. MS (m/z): 428.0 [M+H]⁺.

### Example 241

### Preparation of 2-(3-Fluoro-2-hydroxyphenyl)-6-methyl-5-(5-methyl-2-thienyl)-3-[2-(2-thienyl)ethyl]-4(3H)-pyrimidinone

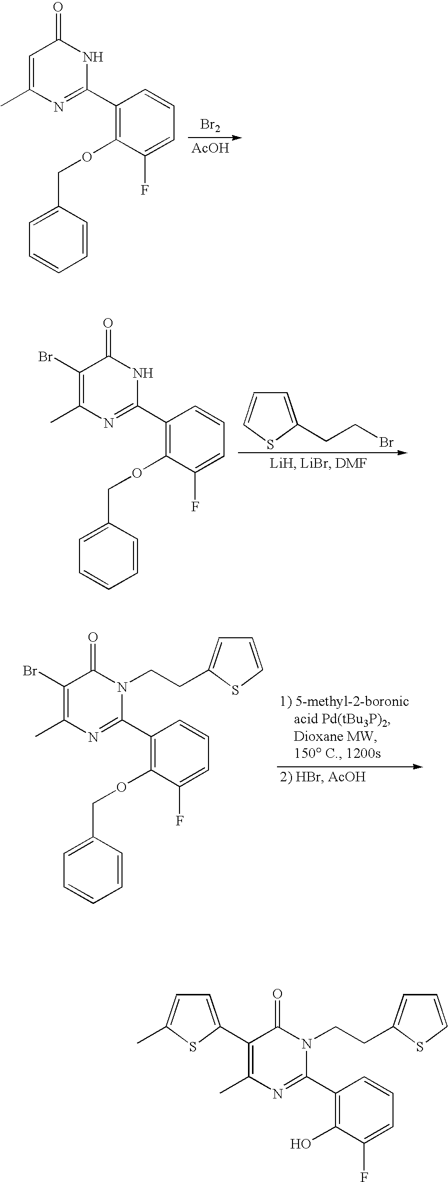

The title compound was prepared according to the procedures of Example 238 except substituting 2-(2-bromoethyl)thiophene for 2-(2-bromoethyl)cyclohexane. The debenzylation occurred during the Suzuki coupling reaction further eliminating the need for the deprotection step. MS (m/z): 427.0 [M+H]⁺.

### Example 242

### Preparation of 5-(4,5-Dimethyl-1,3-thiazol-2-yl)-2-(3-fluoro-2-hydroxyphenyl)-6-methyl-3-[2-(2-thienyl)ethyl]-4(3H)-pyrimidinone

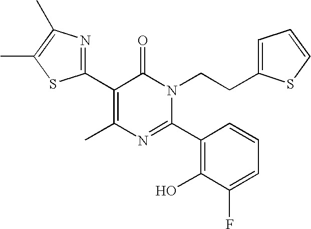

The title compound was prepared according to the procedure of Example 97 except substituting 4,5-dimethyl-2-(tributylstannanyl)-1,3-thiazole for 2-(tributylstannanyl)-1,3-thiazole and 5-bromo-2-{3-fluoro-2-[(phenylmethyl)oxy]phenyl}-6-methyl-3-[2-(2-thienyl)ethyl]-4(3H)-pyrimidinone for 5-bromo-2-{3-fluoro-2-[(phenylmethyl)oxy]phenyl}-6-methyl-3-(2-phenylethyl)-4(3H)-pyrimidinone. Subsequent debenzylation using catalytic hydrogenolysis afforded the product. MS (m/z): 442.2 [M+H]⁺.

### Example 243

### Preparation of 2-(3-Fluoro-2-hydroxyphenyl)-6-methyl-5-(5-methyl-2-thienyl)-3-[2-(tetrahydro-2H-pyran-4-yl)ethyl]-4(3H)-pyrimidinone

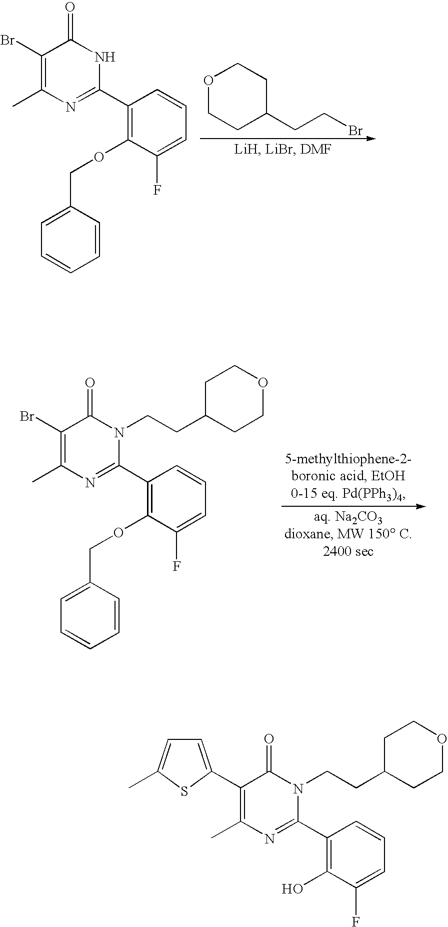

The title compound was prepared according to the procedures of Example 238 except substituting 4-(2-bromoethyl)tetrahydro-2H-pyran for 2-(2-bromoethyl)cyclohexane. The debenzylation occurred during the Suzuki coupling reaction further eliminating the need for the deprotection step. MS (m/z): 429.0 [M+H]⁺.

### Example 244

### Preparation of 2-(3-Fluoro-2-hydroxyphenyl)-3-[2-(2-fluorophenyl)ethyl]-6-methyl-5-(5-methyl-2-thienyl)-4(3H)-pyrimidinone

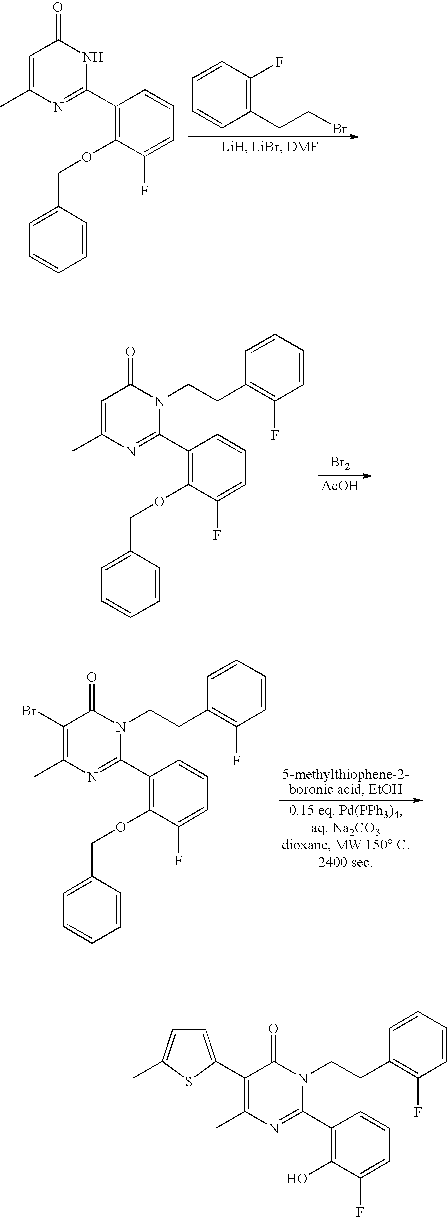

The title compound was prepared according to the procedures of Example 238 except substituting 1-(2-bromoethyl)-2-fluorobenzene for 2-(2-bromoethyl)cyclohexane. The debenzylation occurred during the Suzuki coupling reaction further eliminating the need for the deprotection step. MS (m/z): 439.2 [M+H]⁺.

### Example 245

### Preparation of 2-(3-Fluoro-2-hydroxyphenyl)-3-[2-(3-fluorophenyl)ethyl]-6-methyl-5-(5-methyl-2-thienyl)-4(3H)-pyrimidinone

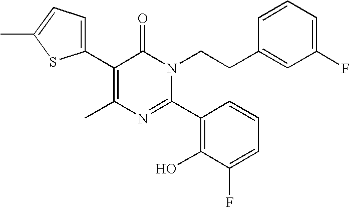

The title compound was prepared according to the procedures of Example 238 except substituting 1-(2-bromoethyl)-3-fluorobenzene for 2-(2-bromoethyl)cyclohexane. The debenzylation occurred during the Suzuki coupling reaction further eliminating the need for the deprotection step. MS (m/z): 439.2 [M+H]⁺.

### Example 246

### Preparation of 2-(3-Fluoro-2-hydroxyphenyl)-3-[2-(4-fluorophenyl)ethyl]-6-methyl-5-(5-methyl-2-thienyl)-4(3H)-pyrimidinone

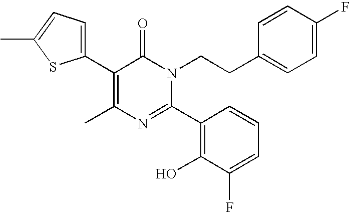

The title compound was prepared according to the procedures of Example 238 except substituting 1-(2-bromoethyl)-4-fluorobenzene for 2-(2-bromoethyl)cyclohexane. The debenzylation occurred during the Suzuki coupling reaction further eliminating the need for the deprotection step. MS (m/z): 439.2 [M+H]⁺.

### Example 247

### Preparation of 2-(3-Fluoro-2-hydroxyphenyl)-6-methyl-5-(3-methyl-2-thienyl)-3-(2-phenylethyl)-4(3H)-pyrimidinone

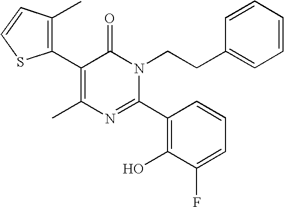

The title compound was prepared according to the procedures of Example 13 except substituting 3-methylthiophene-2-boronic acid for 6-quinolinylboronic acid. Subsequent debenzylatlion using hydrobromic acid in acetic acid afforded the title compound. MS (m/z): 421.2 [M+H]⁺.

### Example 248

### Preparation of 5-(1-Benzothien-2-yv)-3-(2,3-dihydro-1H-inden-2-yl)-2-(2-hydroxyphenyl)-6-methyl-4(3H)-pyrimidinone

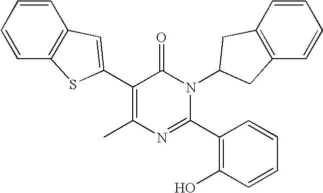

The title compound was prepared according to the procedures of Example 235 except substituting benzothiophene-2-boronic acid for 5-methylthiophene-2-boronic acid. Subsequent catalytic hydrogenolysis provided the title compound. MS (m/z): 451.2 [M+H]⁺

### Example 249

### Preparation of 2-(3-Fluoro-2-hydroxyphenyl)-6-methyl-5-(2-methylpropyl)-3-(2-phenyl)ethyl)-4(3H)-pyrimidinone

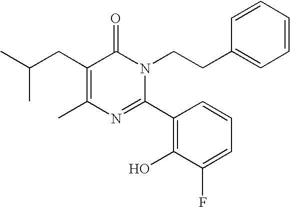

### a. 2-Acetyl-4-methylpentanoate

To a suspension of NaOMe (12.78 g, 0.24 mol) in dry methanol (430 mL) was added methyl acetoacetate (25 g, 0.22 mol) and stirred for 15 minutes and heated to gentle reflux. 1-bromo-2-methylpropane (29.5 g, 0.22 mol) added in portions within two hours and heated continued overnight. The reaction was concentrated and dilute with NH₄Cl and extracted with diethylether. The ether layer was dried and concentrated. The residue was purified by flash column chromatography (10% EtOAc/hexanes) to provide 2 g (5%) of the title compound.

### b. 2-{3-Fluoro-2-[(phenylmethyl)oxy]phenyl}-6-methyl-5-(2-methylpropyl)-4(1H)-pyrimidinone

### a. 2-Chloro-6-fluorophenyl phenylmethyl ether

To a solution of sodium methoxide (3.08 g) was added 3-fluoro-2-[(phenylmethyl)oxy]benzenecarboximidamide (3.95 g, 1.6 mmol). This mixture was maintained at room temperature for 15 minutes whereupon methyl 2-acetyl-4-methylpentanoate (2.23 g, 13 mmol) was added. This mixture was refluxed overnight whereupon it was cooled to room temperature and quenched with NH4Cl. 2-Chloro-6-fluoro phenol (2.0 g, 13.6 mmol) was dissolved in 68 ml DMF. To this solution was added Cs₂CO₃ (6.67 g, 20.5 mmol) and benzyl bromide (1.78 ml, 15 mmol) sequentially and stirred for 12 hr. The residue reaction mixture was diluted with EtOAc and washed with brine. (3×100 mL). The aqueous organic layer was reextracted with EtOAc and the combined organic layers were dried over Na₂SO₄, filtered and concentrated. The residue was purified by flash column chromatography (30% EtOAc/hexanes) to provide 0.9 g (19%) of the title compound to give 2.97 g of product in 92% yield.

### b. 2-{b. 3-fluoroFluoro-2-[(phenylmethyl)oxy]phenyl}-6-methyl-5-(2-methylpropyl)-3-(2-phenylethyl)-4(3H)-pyrimidinonebenzonitrile

To a solution of 2-{3-fluoro-2-[(phenylmethyl)oxy]phenyl}-6-methyl-5-(2-methylpropyl)-4(1H)-pyrimidinone (0.9 g, 2.46 mmol) in DMF (25 mL) was added lithium hydride (0.039 g, 4.91 mmol) and lithium bromide (0.64 g, 7.37 mmol). This mixture was stirred at room temperature for 15 minutes whereupon phenethylbromide (2.27 g, 12.3 mmol) was added. This mixture was maintained at room temperature for 12 hours whereupon it was diluted with EtOAc, washed with brine (3×'s) and concentrated. Column chromatography of the residue (25% EtOAc/hexanes) provided 0.323 g (28%) of the title compound.

### c. To the solution of 1-Benzyloxy-2-(3-Fluoro-2-hydroxyphenyl)chloro-6-methyl-5-(2-methylpropyl)-3-(2-phenylethyl)-4(3H)-pyrimidinone

To a fluoro-benzene (200 mg, 0° C. solution of 2-{3-fluoro-2-[(phenylmethyl)oxy]phenyl}-6-methyl-5-(2-methylpropyl)-3-(2-phenylethyl)-4(3H)-pyrimidinone (0.323 g, 0.68.42 mmol) in 8 ml dry DMF was added BBr₃ (2.0 mL of 1 M DCM solution, 2.06 mmol). This Zn(CN)₂ (110 mg, 0.93 mmol) and Pd(t-Bu₃P)₂ (86 mg, 0.08 mmol) and the mixture was allowed to warm to room temperature overnight whereupon methanol was added and the mixture concentrated. Column chromatography of the residue (0-30% EtOAc/hexanes) provided 0.22 g (85%) of the title compound. MS (EI) 381.2 (M+H)⁺.placed in microwave reactor (150° C., 20 min). The reaction mixture was diluted with EtOAc and washed with brine. Organic layer was dried over Na₂SO₄, filtered and concentrated. The crude product was purified by flash column chromatography (0 to 20% EtOAc/Hexane) to produce the desired product (0.8 g) in 83% in yield.

### d. 3-Fluoro-2-[(phenylmethyl)oxy]benzenecarboximidamide

3-Fluoro-2-[(phenylmethyl)oxy]benzonitrile (3.88 g, 0.017 mol) was added to a 0° C. solution of LiHMDS (43 mL, 1M in hexanes) in anhydrous Et₂O (34 m) under N₂. After warming to room temperature, the mixture stirred for three to four days. The resulting reaction mixture was cooled to 0° C. and quenched by the addition of 1N HCl. The layers were separated and the aqueous phase was extracted 2 times with Et₂O. The aqueous layer was cooled in an ice-bath, adjusted to pH 12 with 6N NaOH, and extracted 3 times with dichlormethane. The organic portions were pooled, dried over Na₂SO₄, and concentrated to a brown oil which solidified to a brown solid under vacuum (3.95 g, 95% yield):

### e. 2-Acetyl-4-methylpentanoate

To a suspension of NaOMe (13.45 g, 0.237 mol) in dry methanol (430 mL) was added methyl acetoacetate (25 g, 0.215 mol) and stirred for 15 minutes and heated to gentle reflux. 1-bromo-2-methylpropane (29.5 g, 0.215 mol) added in portions within two hours and heated continued overnight. The reaction was concentrated and dilute with NH₄Cl and extracted with diethylether. The ether layer was dried and concentrated. The residue was purified by flash column chromatography (10% EtOAc/hexanes) to provide 2.01 g of the title compound.

### f. 2-{3-Fluoro-2-[(phenylmethyl)oxy]phenyl}-6-methyl-5-(2-methylpropyl)-4(1H)-pyrimidinone

To a solution of 3-fluoro-2-[(phenylmethyl)oxy]benzenecarboximidamide (3.95 g) in methanol and dioxane solvent mixture (108 mL/22 mL) at 0° C. was added sodium methoxide (3.08 g). This mixture was stirred for 15 minutes whereupon methyl 2-acetyl-4-methylpentanoate (2.23 g) was added. This mixture was refluxed overnight whereupon it was cooled to room temperature and quenched with NH₄Cl. The residue was diluted with EtOAc and washed with brine. The aqueous layer was reextracted with EtOAc and the combined organic layers were dried, filtered and concentrated. The residue was purified by flash column chromatography (30% EtOAc/hexanes) to provide 0.9 g (19%) of the title compound.

### g. 2-{3-Fluoro-2-[(phenylmethyl)oxy]phenyl}-6-methyl-5-(2-methylpropyl)-3-(2-phenylethyl)-4(3H)-pyrimidinone

To a solution of 2-{3-fluoro-2-[(phenylmethyl)oxy]phenyl}-6-methyl-5-(2-methylpropyl)-4(1H)-pyrimidinone (0.9 g, 2.46 mmol) in DMF (25 mL) was added lithium hydride (0.039 g, 4.91 mmol) and lithium bromide (0.64 g, 7.37 mmol). This mixture was stirred at room temperature for 15 minutes whereupon phenethylbromide (2.27 g, 12.3 mmol) was added. This mixture was maintained at room temperature for 12 hours whereupon it was diluted with EtOAc, washed with brine (×3) and concentrated. Column chromatography of the residue (25% EtOAc/hexanes) provided 0.323 g (28%) of the title compound.

### h. 2-(3-Fluoro-2-hydroxyphenyl)-6-methyl-5-(2-methylpropyl)-3-(2-phenylethyl)-4(3H)-pyrimidinone

To a 0° C. solution of 2-{3-fluoro-2-[(phenylmethyl)oxy]phenyl}-6-methyl-5-(2-methylpropyl)-3-(2-phenylethyl)-4(3H)-pyrimidinone (0.323 g, 0.68 mmol) in was added BBr₃ (2.0 mL of 1 M DCM solution, 2.04 mmol). This mixture was allowed to warm to room temperature overnight whereupon methanol was added and the mixture concentrated. Column chromatography of the residue (0-30% EtOAc/hexanes) provided 0.22 g (85%) of the title compound. MS (EI) 381.2 (M+H)⁺.

### Example 250

### Preparation of 2-(2-Hydroxyphenyl)-5,5-dimethyl-3-(2-phenylethyl)-5,6,7,8-tetrahydro-4(3H)-quinazolinone

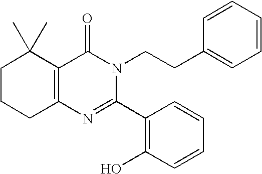

### a. 2-(2-hydroxyphenyl)-5,5-dimethyl-5,6,7,8-tetrahydro-4(1H)-quinazolinone

To a solution of sodium methoxide (38.3 mL of a 0.5 M solution in methanol) was added 2-(methyloxy)benzenecarboximidamide (1.29 g, 8.59 mmol). This mixture was maintained at room temperature for 15 minutes whereupon methyl 2,2-dimethyl-6-oxocyclohexanecarboxylate (1.6 g, 8.7 mmol) was added. This mixture was refluxed overnight whereupon it was cooled to room temperature and concentrated. The residue was diluted with CH₂Cl₂ and the pH adjusted to 3. The aqueous layer was extracted with CH₂Cl₂ and the combined organic layers were dried, filtered and concentrated. The residue was purified by flash column chromatography (0-75% EtOAc/hexanes) to provide 2.2 g (89%) of the title compound: MS (EI) 285.2 (M+H)⁺.

### b. 5,5-dimethyl-2-[2-(methyloxy)phenyl]-3-(2-phenylethyl)-5,6,7,8-tetrahydro-4(3H)-quinazolinone

To a solution of 2-(2-hydroxyphenyl)-5,5-dimethyl-5,6,7,8-tetrahydro-4(1H)-quinazolinone (0.4 g, 1.41 mmol) in DMF (14 mL) was added lithium hydride (13.5 mg, 1.7 mmol). This mixture was stirred at room temperature for 15 minutes whereupon phenethylbromide (0.23 mL, 0.17 mmol) was added. This mixture was maintained at room temperature for 12 hours whereupon it was diluted with EtOAc, washed with brine (3×'s) and concentrated. Column chromatography of the residue (10-50% EtOAc/hexanes) provided 0.21 g (39%) of the title compound: MS (EI) 389.2 (M+H)⁺.

### c. 2-(2-hydroxyphenyl)-5,5-dimethyl-3-(2-phenylethyl)-5,6,7,8-tetrahydro-4(3H)-quinazolinone

To a 0° C. solution of 5,5-dimethyl-2-[2-(methyloxy)phenyl]-3-(2-phenylethyl)-5,6,7,8-tetrahydro-4(3H)-quinazolinone (0.20 g, 0.51 mmol) in CH₂Cl₂ (5 mL) was added BBr₃ (2.5 mL of a 0.5 M solution in CH₂Cl₂). This mixture was allowed to warm to room temperature overnight whereupon methanol was added and the mixture concentrated. Column chromatography of the residue (0-30% EtOAc/hexanes) provided 0.15 g (76%) of the title compound: MS (EI) 375.2 (M+H)⁺.

### Example 251

### Preparation of 5-Chloro-2-(2-hydroxyphenyl)-6-methyl-3-(2-phenylethyl-4(3H)-pyrimidinone

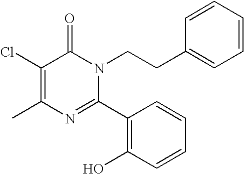

The title compound was prepared following the procedures described in Example 1a-c except substituting ethyl 2-chloro-3-oxobutanoate ethyl for 2-acetyl-4-methyl-4-pentenoate of step 1 ale and 2-hydroxybenzenecarboxamidemethoxybenzenecarboxamide for 3-fluoro-2-hydroxybenzenecarboxamide[(phenylmethyl)oxy]benzenecarboximidamide: MS (EI) 341.4 (M+H)⁺

### Example 252

### Preparation of 3-[2-(3-Fluorophenyl)ethyl]-2-(2-hydroxyphenyl)-5,6, 7,8-tetrahydro-4(3H)-quinazolinone

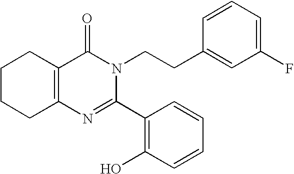

The title compound was prepared following the procedures described in Example 2a-c except substituting ethyl 2-oxocyclohexanecarboxylate for methyl 2,2-dimethyl-6-oxocyclohexanecarboxylate in step 2a and 1-(2-bromoethyl)-3-fluorobenzene for phenethylbromide in step 2b: MS (EI) 365.2 (M+H)⁺

### Example 253

### Preparation of 2-(3-Fluoro-2-hydroxyphenyl)-6-methyl-5-(5-methyl-2-thienyl)-3-(2-phenylethyl)-4(3H)-pyrimidinone

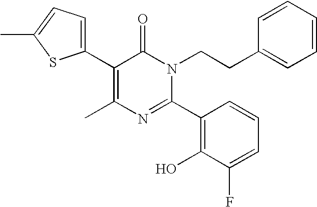

### a. Phenylmethyl 3-fluoro-2-[(phenylmethyl)oxy]benzoate

3-Fluoro-2-hydroxybenzoic acid (10 g, 0.064 moles) was dissolved in dry DMF (128 mL). To this was added potassium carbonate (18.5 g, 0.14 moles) and benzyl bromide (16.74 mL, 0.14 moles) sequentially. Reaction was stirred overnight at ambient temperature. Reaction was filtered and diluted with EtOAc. This was washed successively with 5% HCl and saturated sodium chloride solution (×3). Organic layer was dried over sodium sulfate and concentrated to give the product (21.9 g) in quantitative yield.

### b. 3-Fluoro-2-[(phenylmethyl)oxy]benzoic acid

A solution of phenylmethyl 3-fluoro-2-[(phenylmethyl)oxy]benzoate (20 g, 0.059 moles) in methanol (150 mL) and water (50 mL) was treated with 50% (w/w) NaOH (9.5 mL) and stirred overnight. The ethanol was removed in vacuo and the aqueous layer was diluted with water (10 mL) and then extracted with ether (2×100 mL). The aqueous layer was collected and the acidity was adjusted to pH˜4 with 3N HCl. The aqueous layer was extracted with EtOAc and the organic layer were washed with brine. The Organic layer was dried over sodium sulfate and concentrated to give the product (14.4 g) in 98.6% yield.

### c. 3-Fluoro-2-[(phenylmethyl)oxy]benzamide

To a solution of 3-fluoro-2-[(phenylmethyl)oxy]benzoic acid (11.3 g, 0.046 moles) was taken up in dry THF (34 mL) cooled to 0° C. To this was added TEA (6.66 mL, 0.046 moles) and ethyl chloroformate (5.03 mL, 0.046 moles) and stirred for 20 minutes. Ammonia solution (30% aq. NH₄OH, 28 mL) in THF (15 mL) was then added to the reaction and stirred for 30 min and then concentrated. The solid residue was then partition with dichloromethane and water. The aqueous was then washed again with dichloromethane and the combined organics were washed with saturated sodium hydrogen carbonate solution, brine, dried and concentrated to produce the product (11.2 g) in 99% yield.

### d. 3-Fluoro-2-hydroxybenzamide

3-Fluoro-2-[(phenylmethyl)oxy]benzamide (1.0 g, 4.07 mmol) was taken up in ethanol. To this was added 10% Pd/C (0.10 g). This mixture was placed under Hydrogen atmosphere (balloon) and stirred overnight. The reaction mixture was filtered through a bed of celite and concentrated to afford the desired product (0.61 g) in 97% yield. MS (m/z): 156.2 [M+H]⁺.

### e. 3-Oxo-N-(2-phenylethyl)butanamide

Diketene (10.0 g, 0.12 moles) was taken up in anhydrous ether (237 mL). To this was added phenethylamine (14.93 mL, 0.12 moles). After addition of amine was complete the reaction was heated to reflux for 3 h. The crude mixture was concentrated and purified by biotage purification system using EtOAc/hexanes (1:1) to give 22.78 grams in 93% yield.

### f. 2-(3-Fluoro-2-hydroxyphenyl)-6-methyl-3-(2-phenylethyl)-4(3H)-pyrimidinone

The 3-oxo-N-(2-phenylethyl)butanamide (10 g, 0.049 moles) was placed in 500 mL round bottom flask. To this was added titanium isopropoxide (214 mL, 0.73 moles). While the reaction is stirring 3-fluoro-2-hydroxybenzamide (11.42 g, 0.098 moles) was added in portions, a condenser was placed and the reaction was heated to reflux (oil bath temperature=150° C.). The 2-hydroxy-3-fluorobenzamide dissolved slowly and gave brown homogenous solution upon some time at elevated temperatures. Reaction was run for 36 h and cooled to ambient temperature and diluted with dichloromethane. 3N HCl was slowly added until all the solid that was initially formed has dissolved. Organic layer was separated, dried over sodium sulfate and filtered and concentrated and purified by crashing out from EtOAc/hexanes mixture (6.79 g, 43%).

### g. 2-{3-Fluoro-2-[(phenylmethyl)oxy]phenyl}-6-methyl-3-(2-phenylethyl)-4(3H)-pyrimidinone

The 2-(3-fluoro-2-hydroxyphenyl)-6-methyl-3-(2-phenylethyl)-4(3H)-pyrimidinone (6.0 g, 0.019 moles) was dissolved in dry DMF (92 mL). To this was added potassium carbonate (3.83 g, 0.028 moles) and benzyl bromide (2.64 mL, 0.028 moles) sequentially. Reaction was warmed to 60° C. and stirred overnight. Reaction mixture was cooled to ambient temperature and washed with H₂O and brine (×3). Organic layer was dried over sodium sulfate and concentrated and purified by biotage purification system using EtOAc/hexanes (0-50%) to give the product (7.12 g) in 93% yield.

### h. 5-Bromo-2-{3-fluoro-2-[(phenylmethyl)oxy]phenyl}-6-methyl-3-(2-phenylethyl)-4(3H)-pyrimidinone

The 2-{3-fluoro-2-[(phenylmethyl)oxy]phenyl}-6-methyl-3-(2-phenylethyl)-4(3H)-pyrimidinone (6.0 g, 0.0145 moles) was taken up in glacial acetic acid (100 mL). To this was added bromine (0.74 mL, 0.0145 moles) dropwise by a syringe. Reaction was stirred for 3 h. Additional amount of bromine (1 eq.) was added and stirred overnight. Ethyl acetate was added and acetic acid was washed with saturated sodium bicarbonate. The organic layer was further washed with saturated solution of sodium hydrogensulfite/sodium metabisulfite and dried over sodium sulfate. Sodium sulfate was filtered off and organic layer was concentrated. The crude product was purified by chromatography on silica gel (Biotage) using ethylacetate and hexane mixtures (0-50%) to obtain the desired product (7.06 g) in 98% yield. MS (m/z): 495.2 [M+H]⁺.

### i. 2-(3-Fluoro-2-hydroxyphenyl)-6-methyl-5-(5-methyl-2-thienyl)-3-(2-phenylethyl)-4(3H)-pyrimidinone

To a solution of 5-bromo-2-{3-fluoro-2-[(phenylmethyl)oxy]phenyl}-6-methyl-3-(2-phenylethyl)-4(3H)-pyrimidinone (0.20 g, 0.41 mmol) in dioxane (5 mL) was added 5-methyl-2-thiopheneboronic acid (0.12 g, 0.81 mmol), 0.5 mL ethanol, and 0.5 mL aqueous sodium carbonate (0.086 g, 0.81 mmol) in a microwave reaction vessel. After 10 min. of deoxygenation, tetrakis(triphenylphosphine)palladium (0.047 g, 0.041 mmol) was added. The mixture in sealed vessel was irradiated to 150° C. for 2400 seconds. The reaction mixture was filtered through syringe filter (Acrodisc CR25 mm with 0.2 μm PTFE membrane). The vessel and filter were washed with dichloromethane. The dichloromethane was concentrated and the residue was purified by flash chromatography (0-40% ethyl acetate/hexane) to afford the title compound (0.14 g, 79%). MS (m/z): 421.2 [M+H]⁺. ¹H NMR (400 MHz, CDCl₃) δ ppm 2.50 (s, 3H), 2.57 (s, 3H), 3.01 (t, J=7.4 Hz, 2H), 4.30 (t, J=7.4 Hz, 2H), 6.80 (s, 1H), 6.92-7.19 (s, 6H), 7.20 7.28 (m, 3H), 9.00 (brs, 1H). Anal. Calcd. for C₂₄H₂₁FN₂O₂S: C, 67.91; H, 4.84; N, 6.62. Found: C, 68.55; H, 5.03; N, 6.66.

### Alternative Synthetic Route:

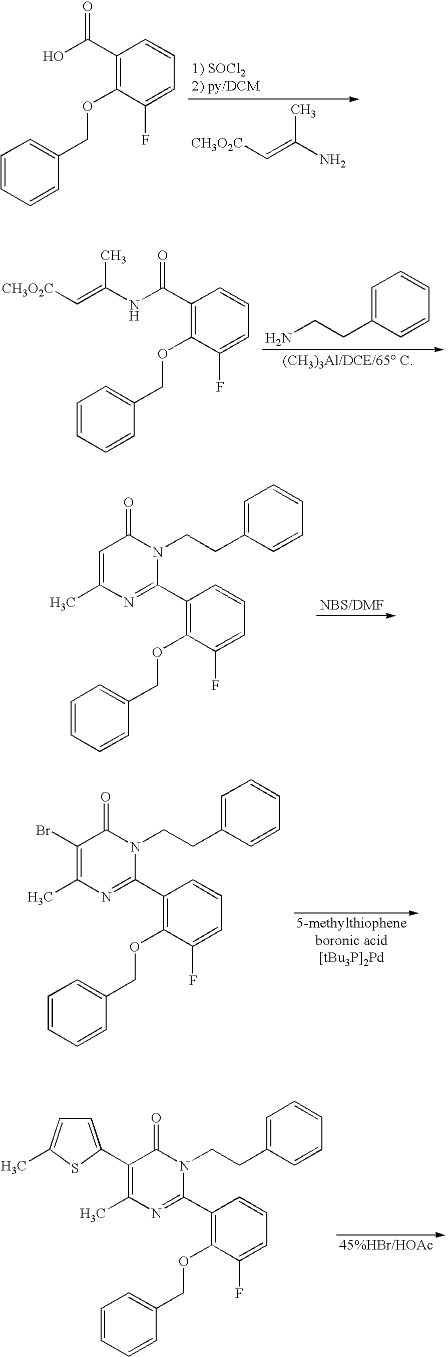

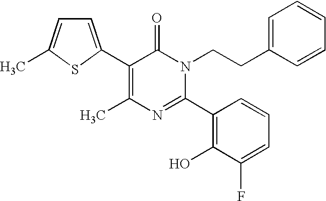

### a. Phenylmethyl 3-fluoro-2-[(phenylmethyl)oxy]benzoate

3-Fluoro-2-hydroxybenzoic acid (210 g, 1.345 moles) was dissolved in dry DMF (2 L) and added to a 5 L 3-necked flask. Powdered potassium carbonate (390 g, 2.82 moles, 2.1 equiv.) was added in portions to control the gas evolution, and then benzyl bromide (506 g, 2.96 moles, 2.2 equiv.) was added to this suspension. The reaction was mechanically stirred at ambient temperature for 16 h; filtered using a sintered glass funnel, and then the filtrate was diluted with ethyl acetate (3 L). This solution was washed successively with 5% HCl and saturated sodium chloride solution (3×1 L). The organic layer was dried over sodium sulfate and concentrated to give the product (429.9 g) in 95% yield.

### b. 3-fluoro-2-[(phenylmethyl)oxy]benzoic acid

A solution of phenylmethyl 3-fluoro-2-[(phenylmethyl)oxy]benzoate (429 g, 1.275 moles) in methanol (800 mL) and water (300 mL) was treated with 50% (w/w) NaOH solution (150 mL) and stirred at room temperature for 3 h. The methanol was removed in vacuo and the waxy residue was diluted with water (1.5 L) and then extracted with t-butyl methyl ether (2×500 mL). The aqueous layer was collected, cooled in an ice water bath, and the pH adjusted to pH 3 with conc. HCl (−200 mL) while stirring. The precipitate was collected by filtration and the aqueous layer was extracted with EtOAc (3×500 mL). The combined organic layers were used to dissolve the filtered precipitate, and then this solution was washed with brine. The organic layer was dried over sodium sulfate and concentrated to give 3-fluoro-2-[(phenylmethyl)oxy]benzoic acid (301 g, 1.222 mol) in 95.9% yield.

### c. Methyl (2E,Z)-3-{[2-benzyloxy)-3-fluorobenzoyl]amino}but-2-enoate:

A suspension of 3-fluoro-2-[(phenylmethyl)oxy]benzoic acid (301 g, 1.222 mol) in thionyl chloride (713 mL, 9.78 mol, 8 equiv.) was heated at reflux for 1.5 h. The reaction was cooled, and then the excess thionyl chloride was evaporated using a rotary evaporator. The residue was azeotroped with toluene (4×600 mL) and then dichloromethane (1×600 mL). The resulting acid chloride was dissolved in dichloromethane (500 mL) and added drop wise to a solution of methyl 3-aminocrotonate (141 g, 1.222 mol) and pyridine (178 mL, 2.2 mol, 1.8 equiv.) in dichloromethane (1.8 L) and then stirred at room temperature for 4.5 h. The reaction was then quenched with ice-cooled 3N aqueous HCl solution and extracted with dichloromethane (3×250 mL). The combined organic layers were washed sequentially with water (1 L), saturated sodium bicarbonate solution (1 L), and with brine (1 L). The solvent was removed in vacuo, and the residue was chromatographed on 2.5 kg of silica gel eluted with a gradient of chloroform/hexanes (50:50) to chloroform/hexanesl ethyl acetate (45:45:10). The fractions that corresponded to product were combined, then concentrated to provide GSK1507280A (320 g, 0.933 mole, 74% yield) as a mixture of (E,Z-) isomers.

### d. 2-{3-fluoro-2-[(phenylmethyl)oxy]phenyl}-6-methyl-3-(2-phenylethyl)-4(3H)-pyrimidinone

A solution of phenethylamine (142 mL, 1.2 mol, 3 equiv.) in 1,2-dichloroethane (1 L) was cooled to 0° C. A solution of trimethylaluminum in toluene (565 mL, 1.13 mol, 3 equiv.) was added dropwise. The ice bath was removed and the mixture was mechanically stirred at room temperature for 45 min, and then recooled to 0° C. A solution of GSK1507280A (129.2 g, 0.377 mol) in 1,2-dichloroethane (350 mL) was added under nitrogen over 45 min. and the resulting mixture was stirred at room temperature for 30 min, then heated at 65° C. for 2 h. The reaction mixture was cooled to room temperature and was quenched by adding the mixture portionwise with stirring to ice water adjusted to pH 3 with 3N aqueous HCl solution. The aqueous phase was extracted with dichloromethane (3×300 mL). The combined organic layers were washed with ice-cooled 3N aqueous HCl solution (500 mL), water (500 mL), brine (500 mL), and dried over sodium sulfate. The dried solution was concentrated in vacuo to provide GSK1511986A (126.4 g, 0.305 mol) in 81% yield.

### e. 5-bromo-2-{3-fluoro-2-[(phenylmethyl)oxy]phenyl}-6-methyl-3-(2-phenylethyl)-4(3H)-pyrimidinone

A solution of GSK1511986A (201.7 g, 0.487 mol) in N,N-dimethylformamide (400 mL) was cooled to 0° C. and a solution of N-bromosuccinimide (173.4 g, 0.974 mol, 2 equiv.) in N,N-dimethylformamide (400 mL) was added dropwise. The reaction mixture was warmed to room temperature and stirred at room temperature for 4 h. The DMF was removed in vacuo, then the residue crystallized form 2-propanol (600 mL) to provide GSK970293A (177.8 g, 0.36 mol) in 74% yield after crystallization and column chromatography on silica gel.

### f. 2-{3-Fluoro-2-[(phenylmethyl)oxy]phenyl}-6-methyl-5-(5-methyl-2-thienyl)-3-(2-phenylethyl)-4(3H)-pyrimidinone

A suspension of 5-bromo-2-{3-fluoro-2-[(phenylmethyl)oxy]phenyl}-6-methyl-3-(2-phenylethyl)-4(3H)-pyrimidinone (70 g, 0.142 mol) 5-methyl-thiopheneboronic acid (40 g, 0.282 mol, Frontier Scientific), grounded sodium carbonate (30 g, 0.282 mol), water (7 mL), ethanol (7 mL) in toluene (800 mL) in a 2-L 3-neck round bottom flask was degassed for 10 min with nitrogen. (t-Bu₃P)₂P (10.8 g, 21 mmol) was added to the suspension. The resulting reaction mixture was placed in an oil bath preheated at 100° C. under nitrogen. After stirring for 1 h, the black suspension was filtered through a bed of Celite. The filtrate was concentrated and the residue was azeotroped with 3× toluene to give the titled GSK125064641A (70 g) in 95% crude yield.

### g. 2-(3-Fluoro-2-hydroxyphenyl)-6-methyl-5-(5-methyl-2-thienyl)-3-(2-phenylethyl)-4(3H)-pyrimidinone

To the crude 2-{3-fluoro-2-[(phenylmethyl)oxy]phenyl}-6-methyl-5-(5-methyl-2-thienyl)-3-(2-phenylethyl)-4(3H)-pyrimidinone (78 g) was added 45% HBr/HOAc (550 mL). The reaction mixture was stirred at RT for 5 h. The dark mixture was quenched into ice-water (3 L) and pH carefully was adjusted to 4 by adding 50% NaOH. The aqueous phase was extracted well with DCM, dried over Na₂SO₄, and filtered. The filtrate was concentrated to give the desired crude product GSK728817A (78 g). Biotage silica gel (750 cartridge) purification using DCM, 2% hexane (60)/EtOAc (30)/MeOH (10) in DCM as the mobile phase gave pure GSK728817A (45 g) in 67% isolated over-all yield in 2 steps. To remove trace of Pd metal, a sample (1 g)was dissolved in EtOH (10 mL) was heated to reflux for 18 h in the presence of Darco G060 100 mesh (0.5 g). The cooled suspension was filtered and concentrated to dryness to yield GSK728817A (0.8 g). A sample (78 g) was dissolved in MTBE (650 mL) and placed in a 1 L round bottom flask. The MTBE solution was concentrated in the Buchie to about 350 mL and heptane (100 mL) was added. The resulting crystalline suspension was sonicated and filtered to yield 61 g of pure product.

Novel intermediates of the present invention involve compounds of formula (VII), (VIII), (IX), and (X):

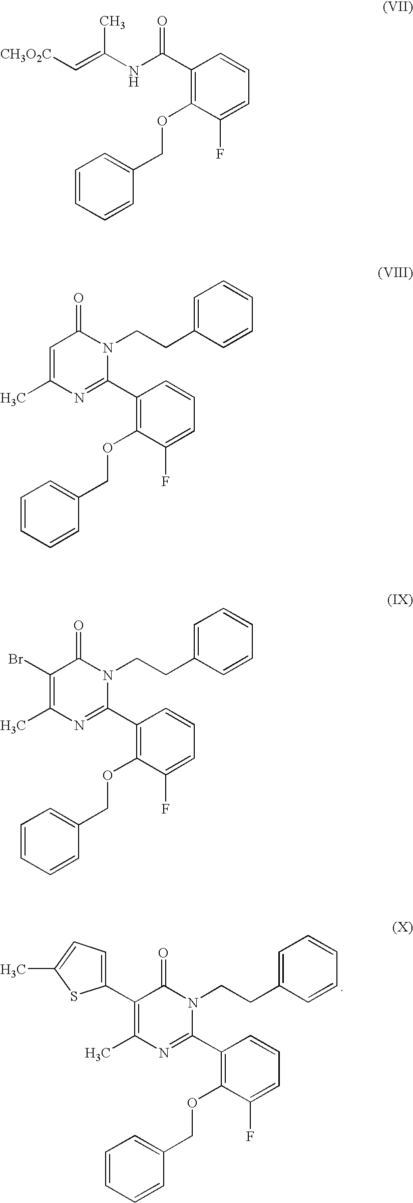

A novel synthetic step disclosed by the present invention includes the cyclization of an enamide according to structure (VII)

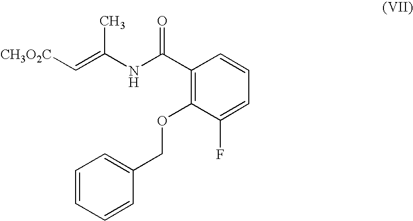

with phenethylamine and trimethylaluminum, in toluene, to yield a pyrimidinone according to structure (VIII).

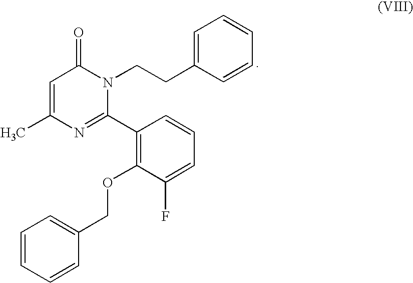

### Example 254

### Preparation of 2-(3-Fluoro-2-hydroxyphenyl-6-methyl-3-(2-phenylethyl)-5-(2-thienyl)-4(3H)-pyrimidinone

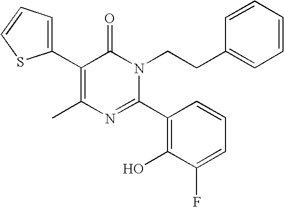

### a. 2-(3-Fluoro-2-hydroxyphenyl)-6-methyl-3-(2-phenylethyl)-5-(2-thienyl)-4(3H)-pyrimidinone

To a solution containing 5-bromo-2-{3-fluoro-2-[(phenylmethyl)oxy]phenyl}-6-methyl-3-(2-phenylethyl)-4(3H)-pyrimidinone (1.0 g, 2.02 mol) of Example 253 h in deoxygenated dioxane was added Pd(tBu₃P)₂ (0.10 g, 0.20 mol), cesium fluoride (0.67 g, 4.5 mol) and tributyl(2-thienyl)stannane (0.6 mL, 2.22 mol) was added sequentially. The reaction was heated to 90° C. for 16 h and concentrated. The crude residue is diluted with dichloromethane and washed with saturated aqueous potassium fluoride, water and brine. The organic layer was separated, dried over Na₂SO₄, filtered and concentrated. The crude material was purified by chromatography on silica gel (Biotage, 0-50% ethyl acetate/hexane) to afford the desired product (0.81 g) in 81% yield.

### b. 2-(3-Fluoro-2-hydroxyphenyl)-6-methyl-3-(2-phenylethyl)-5-(2-thienyi)-4(3H)-pyrimidinone

The 2-(3-fluoro-2-hydroxyphenyl)-6-methyl-3-(2-phenylethyl)-5-(2-thienyl)-4(3H)-pyrimidinone (0.81 g, 1.63 mmol) was placed in a round bottom flask equipped with a stir bar and a condenser. To this was added HBr (45%) in acetic acid (10 mL), water (1.0 mL) and stirred for 5 h. The reaction was quenched with saturated NaHCO₃ and extracted with dichloromethane. The combined organic layers were dried over Na₂SO₄, filtered and concentrated. The crude residue was purified by chromatography on silica gel (Biotage, 0-50% ethyl acetate/hexane) to afford the desired product (0.61 g) in 91% yield. MS (m/z): 407.2 [M+H]⁺. ¹H NMR (400 MHz, CDCl₃) δ ppm 2.51 (s, 3H), 3.02 (t, J=7.6 Hz, 2H), 4.31 (t, J=7.6 Hz, 2H), 6.95-6.98 (m, 4H), 7.06-7.26 (m, 6H), 7.52 (d, J=1.06 Hz, 1H), 8.50 (brs, 1H).

### Example 255

### Preparation of 3-[2-(3-Fluoro-2-hydroxyphenyl)-4-methyl-6-oxo-1-(2-phenylethyl)-1,6-dihydro-5-pyrimidinyl]benzonitrile

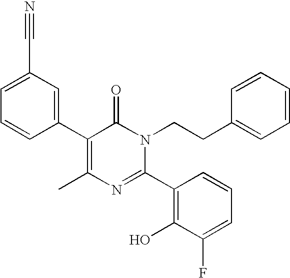

The title compound was prepared by substituting 3-cyanophenylboronic acid for 5-methyl-2-thiopheneboronic acid in Example 253 (h). MS (m/z): 426.2 [M+H]⁺. ¹H NMR (400 MHz, CDCl₃) δ ppm 2.26 (s, 10H), 2.98 (t, J=7.7 Hz, 2H), 4.26 (t, J=7.7 Hz, 2H), 6.94-7.09 (m, 4H), 7.22-7.28 (m, 3H), 7.60-7.72 (m, 5H), 7.9 (brs, 1H). Anal. Calcd. for C₂₆H₂₀FN₃O₂: C, 72.43; H, 4.54; N, 9.66. Found: C, 73.40; H, 4.74; N, 9.88.

### Example 256

### Preparation of 5-(2,3-Dihydro-1,4-benzodioxin-6-yl)-2-(3-fluoro-2-hydroxyphenyl)-6-methyl-3-(2-phenylethyl)-4(3H)-pyrimidinone

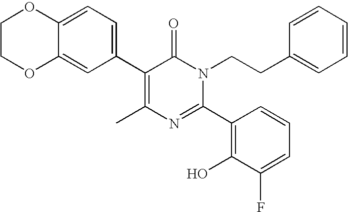

The title compound was prepared by substituting 2,3-dihydro-benzo[1,4]dioxine-6-boronic acid for 5-methyl-2-thiopheneboronic acid in Example 253(h). MS (m/z): 459.4 [M+H]⁺. ¹H NMR (400 MHz, CDCl₃) δ ppm 2.26 (s, 3H), 3.00 (t, J=7.7 Hz, 2H), 4.28 (t, J=7.8 Hz, 2H), 4.32 (s, 4H), 6.83-7.25 (m, 10H), 8.7 (brs, 1H). Anal. Calcd. for C₂₇H₂₃FN₂O₄: C, 69.74; H, 4.95; N, 5.94. Found: C, 70.73; H, 5.06; N, 6.11.

### Example 257

### Preparation of 5-(3,5-Difluorophenyl)-2-(3-fluoro-2-hydroxyphenyl)-6-methyl-3-(2-phenylethyl) 4(3H)-pyrimidinone

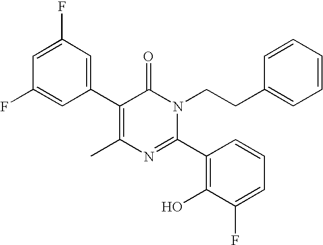

The title compound was prepared by substituting 3,5-difluorophenylboronic acid for 5-methyl-2-thiopheneboronic acid in Example 253(h). MS (m/z): 437.2 [M+H]⁺. 1H NMR (400 MHz, DMSO-d₆) δ ppm 2.15 (s, 3H), 2.76 (t, J=7.8 Hz, 2H), 3.96 (t, J=7.8 Hz, 2H), 6.81-7.303 (m, 11H), 10.7 (brs, 1H).

### Example 258

### Preparation of 2-(3-Fluoro-2-hydroxyphenyl)-6-methyl-5-(4-methyl-2-thienyl)-3-(2-phenylethyl)-4(3H)pyrimidinone

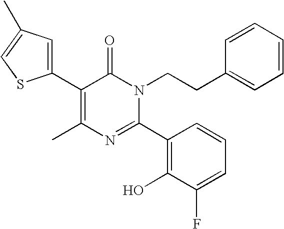

The title compound was prepared by substituting 4-methyl-2-thiopheneboronic acid for 5-methyl-2-thiopheneboronic acid in Example 253(h). MS (m/z): 421.2 [M+H]⁺.

### Example 259

### Preparation of 5-(1-Benzothien-2-yl)-2-(3-fluoro-2-hydroxyphenyl)-6-methyl-3-(2-phenylethyl)-4(3H)-pyrimidinone

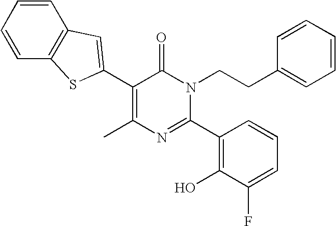

The title compound was prepared by substituting benzothiophene-2-boronic acid for 5-methyl-2-thiopheneboronic acid in Example 253(h). MS (m/z): 457.2 [M+H]⁺. Anal. Calcd. for C₂₇H₂₁FN₂O₂S: C, 70.69; H, 4.33; N, 6.20. Found: C, 71.03; H, 4.64; N, 6.14.

### Example 260

### Preparation of 2-(3-Fluoro-2-hydroxyphenyl)-6-methyl-3-(2-phenylethyl)-5-(2-thienyl)-4(3H)-pyrimidinone

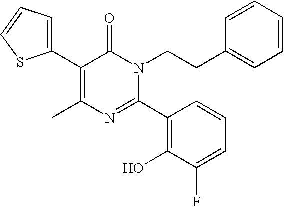

### a. 2-{3-Fluoro-2-[(phenylmethyl)oxy]phenyl}-6-methyl-3-(2-phenylethyl)-5-(2-thienyl)-4(3H)-pyrimidinone

To a solution containing 5-bromo-2-{3-fluoro-2-[(phenylmethyl)oxy]phenyl}-6-methyl-3-(2-phenylethyl)-4(3H)-pyrimidinone (1.0 g, 2.02 mol) of Example 5 h in dioxane was added Pd(tBu₃P)₂ (0.10 g, 0.20 mol), cesium fluoride (0.67 g, 4.5 mol) and tributyl(2-thienyl)stannane (0.6 mL, 2.22 mol) was added sequentially. The reaction was heated to 90° C. for 16 h. The reaction mixture was cooled to room temperature and the crude residue was purified by chromatography on silica gel (Biotage, 0-50% ethyl acetate/hexane) to afford the desired product (0.81 g) in 81% yield.

### b. 2-(3-Fluoro-2-hydroxyphenyl)-6-methyl-3-(2-phenylethyl)-5-(2-thienyl)-4(3H)-pyrimidinone:

The 2-{3-Fluoro-2-[(phenylmethyl)oxy]phenyl}-6-methyl-3-(2-phenylethyl)-5-(2-thienyl)-4(3H)-pyrimidinone (0.81 g, 1.63 mmol) was placed in a round bottom flask equipped with a stir bar and a condenser. To this was added HBr (45%) in acetic acid (10 mL), water and stirred for 5 h. The reaction was quenched with saturated NaHCO₃ and extracted with dichloromethane. The combined organic layers were dried over Na₂SO₄, filtered and concentrated. The crude residue was purified by chromatography on silica gel (Biotage, 0-50% ethyl acetate/hexane) to afford the desired product (0.61 g) in 91% yield. MS (m/z): 407.2 [M+H]⁺. ¹H NMR (400 MHz, CDCl₃) δ ppm 2.51 (s, 3H), 3.02 (t, J=7.6 Hz, 2H), 4.31 (t, J=7.6 Hz, 2H), 6.95-6.98 (m, 4H), 7.06-7.26 (m, 6H), 7.52 (d, J=1.06 Hz, 1H), 8.50 (brs, 1 H).

### Example 261

### Preparation of 5-(1,3-Benzothiazol-2-yl)-2-(3-fluoro-2-hydroxyphenyl)-6-methyl-3-(2-phenylethyl-4(3H)-pyrimidinone

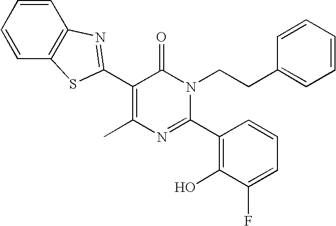

The title compound was prepared by the general procedure outlined in Example 11 substituting 2-tributylstannylbenzothiazole for tributyl(2-thienyl)stannane. MS (m/z): 458.2 [M+H]⁺.

### Example 262

### Preparation of 5-(1-Benzothien-2-yl)-2-(2-hydroxyphenyl)-6-methyl-3-(2-phenylethyl)-4(3H)-pyrimidinone

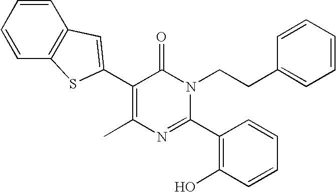

### a. 5-Bromo-6-methyl-3-(2-phenylethyl)-2-{2-[(phenylmethyl)oxy]phenyl}-4(3H)-pyrimidinone

The title compound was prepared by the general procedure outlined in Example 253f-h except substituting 2-hydroxybenzamide for 3-fluoro-2-hydroxybenzamide in Example 253(f). MS (m/z): 477.2 [M+H]⁺.

### b. 5-(1-Benzothien-2-yl)-6-methyl-3-(2-phenylethyl)-2-{2-[(phenylmethyl)oxy]phenyl}-4(3H)-pyrimidinone

To a solution of 5-bromo-6-methyl-3-(2-phenylethyl)-2-{2-[(phenylmethyl)oxy]phenyl}-4(3H)-pyrimidinone (0.60 g, 1.26 mmol) in dioxane (3 mL) was added benzothiophene-2-boronic acid (0.45 g, 2.53 mmol) dissolved 1.0 mL ethanol and 1.0 mL of dioxane, and 1.0 mL aqueous sodium carbonate (0.27 g, 2.53 mmol) in a microwave reaction vessel. This mixture was irradiated to 150° C. for 2000 seconds. The reaction mixture was filtered through syringe filter (Acrodisc CR25 mm with 0.2 μm PTFE membrane). The filtrate was diluted with EtOAc and washed with brine, separated, dried over sodium sulfate. Filtered, concentrated in vacuo and the residue was purified by chromatography on silica gel (Biotage, 0-40% ethyl acetate/hexane) to afford the desired product (0.53 g) in 79% yield.

### c. 5-(1-Benzothien-2-yl)-2-(2-hydroxyphenyl)-6-methyl-3-(2-phenylethyl)-4(3H)-pyrimidinone

5-(1-Benzothien-2-yl)-6-methyl-3-(2-phenylethyl)-2-{2-[(phenylmethyl)oxy]phenyl}-4(3H)-pyrimidinone (0.53 g, 1.0 mmol) was taken up in ethanol. To this was added 10% Pd/C (0.5 g). This mixture was placed under Hydrogen atmosphere in a parr vessel (60 psi) and shaken for 12 h. The reaction mixture was filtered through a bed of celite and concentrated and purified by chromatography on silica gel (Biotage, 0-40% ethyl acetate/hexane) to afford the desired product (0.31 g) in 71% yield. MS (m/z): 439.2 [M+H]⁺. ¹H NMR (400 MHz, CDCl₃) δ ppm 2.51 (s, 3H), 3.08 (t, J=7.6 Hz, 2H), 4.43 (t, J=7.6 Hz, 2H), 7.01-7.04 (m, 4H), 7.07-7.28 (m, 4H), 7.37-7.43 (m, 3H), 7.88-7.94 (s, 2H), 9.41 (s, 1H).

### Example 263

### Preparation of 2-(2-Hydroxyphenyl)-6-methyl-5-(2-methyl-1,3-thiazol-5-yl)-3-(2-phenylethyl)-413H)-pyrimidinone

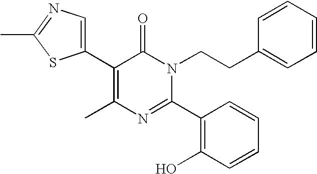

### a. 6-Methyl-5-(2-methyl-1,3-thiazol-5-yl)-3-(2-phenylethyl)-2-{2-[(phenylmethyl)oxy]-phenyl}-4(3H)-pyrimidinone

To a solution of 5-bromo-6-methyl-3-(2-phenylethyl)-2-{2-[(phenylmethyl)oxy]phenyl}-4(3H)-pyrimidinone (2.79 g, 5.87 mmol) of Example 15a in 1,4-dioxane (42 mL) was added cesium fluoride (1.96 g, 12.9 mmol) and (tEuaP)₂Pd (0.451 g, 0.88 mmol) and the reaction was purged with N₂ for 10 min. 2-Methyl-5-(tributylstannanyl)-1,3-thiazole (15.5 g, 40.1 mmol) was added and the reaction was heated at reflux for 20 h. The reaction was cooled and filtered through a Celite-plugged filter frit, washed with CH₃OH and CH₂Cl₂, and concentrated. Column chromatography (1-80% ethyl acetate:hexane) afforded the desired product (1.99 g, 69%): MS (m/z): 494.2 [M+H]⁺.

### b. 2-(2-Hydroxyphenyl)-6-methyl-5-(2-methyl-1,3-thiazol-5-yl)-3-(2-phenylethyl)-4(3H)-pyrimidinone

A solution of 6-Methyl-5-(2-methyl-1,3-thiazol-5-yl)-3-(2-phenylethyl)-2-{2-[(phenylmethyl)oxy]-phenyl}-4(3H)-pyrimidinone (1.99 g, 4.03 mmol) in ethanol (36 mL) was purged with N₂. Pd/C (10%, 2.5 g) was added and the reaction stirred under balloon pressure of H₂ for 3 days. The reaction was filtered through a Celite-plugged filter frit, washed with CH₃OH and CH₂Cl₂, and concentrated. Column chromatography (5-100% ethyl acetate:hexane) produced the title compound (1.16 g, 71%): MS (m/z): 404.0 [M+H]⁺.

### Example 264

### Preparation of 5-[2-(2-Hydroxyphenyl)-4-methyl-6-oxo-1-(2-Phenylethyl)-1,6-dihydro-5-pyrimidinyl]-2-thiophenecarbonitrile

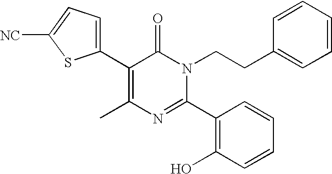

### a. 5-(4-Methyl-6-oxo-1-(2-phenylethyl)-2-{2-[(phenylmethyl)oxy]phenyl}-1,6-dihydro-5-pyrimidinyl)-2-thiophenecarbonitrile

A toluene (13 mL) solution of 5-bromo-6-methyl-3-(2-phenylethyl)-2-{2-[(phenylmethyl)oxy]phenyl}-4(3H)-pyrimidinone (2.0 g, 4.22 mmol) of Example 262(a) in a sealed tube was added (5-cyano-2-thienyl)boronic acid (1.29 g, 8.44 mmol), potassium phosphate (2.69 g, 12.66 mmol), tri(dibenzylideneacetone)dipalladium(0) (386 mg, 0.422 mmol) and 2-dicyclohexylphosphino-2′,6′-dimethyoxy-1,1′biphenyl (346 mg, 0.844 mmol) under nitrogen environment. The reaction vessel was tightly capped and heated for 100° C. degree overnight. The mixture was filtered through a pad of celite and concentrated. Column chromatography of the crude material (0-50% EtOAc/hexanes) provided 1.78 g (84%) of the desired compound: MS (EI) 504 (M+H)⁺.

### b. 5-[2-(2-Hydroxyphenyl)-4-methyl-6-oxo-1-(2-phenylethyl)-1,6-dihydro-5-pyrimidinyl]-2-thiophenecarbonitrile

A solution of 5-(4-methyl-6-oxo-1-(2-phenylethyl)-2-{2-[(phenylmethyl)oxy]phenyl}-1,6-dihydro-5-pyrimidinyl)-2-thiophenecarbonitrile (800 mg, 1.59 mmol) in HBr (48% in acetic acid; 4 mL, 23.8 mmol) was stirred at room temperature overnight. The reaction mixture was quenched with water and adjusted the pH to ˜7 with 6N NaOH. The aqueous layer was extracted with dichloromethane. Combined organic layers were dried over sodium sulfate, filtered, concentrated and purified by Biotage purification system using 0-90% of EtOAc/hexanes to give the title compound as white solid (540 mg, 82%). MS (EI) 414 (M+H)⁺.

### Example 265

### Preparation of 3-[2-(2-Hydroxyphenyl)-4-methyl-6-oxo-1-(2-Phenylethyl)-1,6-dihydro-5-pyrimidinyl]benzonitrile

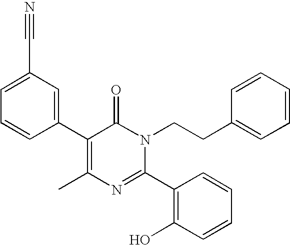

The title compound was prepared by substituting 3-cyanophenylboronic acid for benzothiophene-2-boronic acid in Example 262 (b). 13b. MS (ES) m/e 408[M+H]⁺.

### Example 267

### Preparation of 2-(3-Fluoro-2-hydroxyphenyl)-6-methyl-5-phenyl-3-(2-phenylethyl)-4(3H)-pyrimidinone

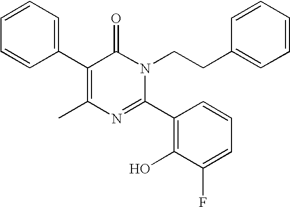

### a. 3-Oxo-2-phenyl-N-(2-phenylethyl)butanamide

To a solution of ethyl 3-oxo-2-phenylbutanoate (49 g, 0.238 moles) in DME was added phenethylamine (24 g, 0.198 mmol) in a microwave reaction vessel. Few drops of ethanol was added to the reaction mixture and irradiated to 180° C. for 1200 s. The reaction mixture was diluted with EtOAc and washed with 1N HCl. Organic layer was separated and dried over Na₂SO₄. Filtered, concentrated and purified by chromatography on silica gel to afford pure amide (17.26 g).

### b. (1Z)-1-M ethyl-3-oxo-2-phenyl-3-[(2-phenylethyl)amino]-1-propen-1-yl trifluoromethanesulfonate

To a solution of 3-oxo-2-phenyl-N-(2-phenylethyl)butanamide (17.26 g, 0.061 mol) in dry dichloromethane was cooled to −78° C. To this was added trifluoromethanesulfonic anhydride (12.36 mL, 0.073 mol) and triethyl amine (12.80 mL, 0.092 mol) sequentially and stirred while reaction warmed to 0° C. The reaction was concentrated and purified by chromatography on silica gel (Biotage, 0-40% ethyl acetate/hexane) to afford the trfilate (14.3 g) in 56% yield.

### c. 3-Fluoro-N-{(1Z)-1-methyl-3-oxo-2-phenyl-3-[(2-phenylethyl)amino]-1-propen-1-yl}-2-(methyloxy)benzamide

To a solution of (1Z)-1-methyl-3-oxo-2-phenyl-3-[(2-phenylethyl)amino]-1-propen-1-yl trifluoromethanesulfonate (13.1 g, 32 mmol) in dry deoxygenated dioxane was added 3-fluoro-2-hydroxybenzamide (5.49 g, 35 mmol), cesium carbonate (14.7 g, 45 mol), Pd₂(dba)₃ (0.74 g, 0.081 mmol) and xantophos (1.40 g, 2.4 mmol). The reaction was heated to reflux for 16 h. The cooled reaction mixture was filtered through a bed of celite and concentrated. Purification was purified by chromatography on silica gel (Biotage) to provide enamide (7.56 g) in 56% yield.

### d. 2-(3-Fluoro-2-hydroxyphenyl)-6-methyl-5-phenyl-3-(2-phenylethyl)-4(3H)-pyrimidinone

The 3-fluoro-N-((1Z)-1-methyl-3-oxo-2-phenyl-3-{[2-(2-thienyl)ethyl]amino}-1-propen-1-yl)-2-(methyloxy)benzamide (7.56 g, 0.018 mol) was dissolved in ethanol (100 mL). To this was added 20 mL of 25% (w/v) aqueous potassium hydroxide and refluxed for 16 h. The crude reaction mixture was acidified by 6N HCl to pH ˜1 and extracted with dichloromethane. The combined organic layers were washed with brine and concentrated. The crude residue was purified by chromatography on silica gel (Biotage) followed by recrystallization from EtOAc provided the desired product (6.32 g) in 88% yield. MS (m/z): 401.2 [M+H]⁺. ¹H NMR (400 MHz, CDCl₃) δ ppm 2.29 (s, 3H), 3.01 (t, J=7.8 Hz, 2H), 4.28 (t, J=7.8 Hz, 2H), 6.94-7.09 (m, 4H), 7.11-7.39 (m, 4H), 7.41-7.51 (m, 5H).

### Example 268

### Preparation of 2-(3-Fluoro-2-hydroxyphenyl)-6-methyl-3-(2-phenylethyl)-5-propyl-4(3H)-pyrimidinone

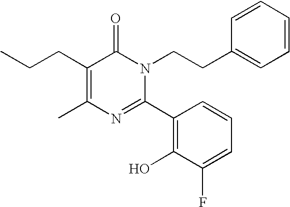

### a. 2-Acetyl-N-(2-phenylethyl)pentanamide

To a solution of ethyl 2-acetylpentenoate (8.0 g, 0.051 mol) in DME was added phenethylamine (5.17 g, 0.043 mol) in a microwave reaction vessel. Few drops of ethanol was added to the reaction mixture was irradiated to 180° C. for 1200 s. The reaction mixture was concentrated and purified by biotage to afford pure amide (5.1 g) along with some impure material (1.75 g). Catalytic hydrogenolysis of these batches individually separately then combining after purification resulted in a total of 6.3 grams of pure product in 50% for two steps.

### b. 2-(3-Fluoro-2-hydroxyphenyl)-6-methyl-3-(2-phenylethyl)-5-propyl-4(3H)-pyrimidinone

The 3-oxo-N-(2-phenylethyl)butanamide (6.2 g, 0.025 mol) was placed in 500 mL round bottom flask and added 251 mL of m-xylene followed by titanium isopropoxide (74 mL, 0.25 mol). While the reaction is stirring 3-fluoro-2-hydroxybenzamide (3.92 g, 0.025 mol) was added, a condenser was placed and the reaction was heated to reflux (oil bath temperature=150° C.). The 2-hydroxy-3-fluorobenzamide dissolved slowly and gave brown homogenous solution upon some time at elevated temperatures. Reaction was run for 36 h and cooled to ambient temperature and diluted with dichloromethane. 3N HCl was slowly added until all the solid that was initially formed has dissolved. Organic layer was separated and the aqueous layer was further extracted with dichloromethane. Combined organic layer were dried over sodium sulfate and filtered and concentrated. The crude reaction mixture was purified by EtOAc/hexanes and followed by MeOH in dichloromethane to give the pure product in 46% (4.21 g) yield. ¹H NMR (400 MHz, CDCl₃) δ ppm 1.04 (t, J=7.4 Hz, 2H), 1.55-1.61 (m, 2H), 2.27 (s, 3H), 2.52-2.56 (m, 2H), 2.88 (t, J=7.4 Hz, 2H), 4.17 (t, J=7.4 Hz, 2H), 6.85-6.89 (m, 5H), 7.04-7.19 (m, 3H), 9.98 (brs, 1H). MS (m/z): 367.2 [M+H]⁺.

### Example 269

### Preparation of 2-(3-Fluoro-2-hydroxyphenyl)-6-methyl-3-(2-phenylethyl)-5-(1H-pyrrol-1-yl)-4(3H)-pyrimidinone

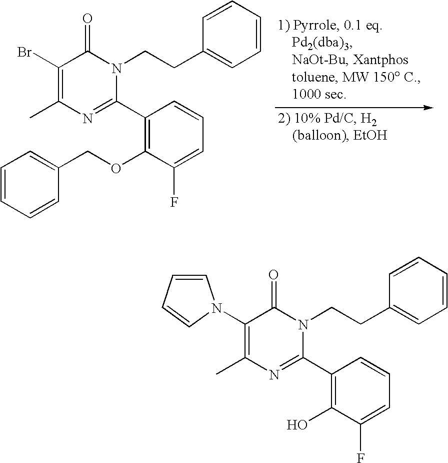

### a. 2-{3-Fluoro-2-[(phenylmethyl)oxy]phenyl}-6-methyl-3-(2-phenylethyl)-5-(1H-pyrrol-1-yl)-4(3H)-pyrimidinone

To a solution of 5-bromo-2-{3-fluoro-2-[(phenylmethyl)oxy]phenyl}-6-methyl-3-(2-phenylethyl)-4(3H)-pyrimidinone (0.3 g, 0.61 moles) in deoxygenated toluene (3.2 mL) was added xantophos (0.05 g, 0.091 mmol), Pd₂(dba)₃ (0.028 g, 0.03 mmol) and NaOtu (0.083 g, 0.85 mmols) in a microwave vessel. The reaction stirred for 5 min. and pyrrole (0.051 mL, 0.073 mmol) was added. The reaction vessel was capped and irradiated in Smith Synthesizer at 150° C. for 1000 s. The reaction mixture was concentrated and purified by chromatography on silica gel (Biotage) using EtOAc and hexane mixtures (5-30%) to obtain the desired product (0.11 g) in 38% yield.

### b. 2-(3-Fluoro-2-hydroxyphenyl)-6-methyl-3-(2-phenylethyl)-5-(1H-pyrrol-1-yl)-4(3H)-pyrimidinone

2-{3-fluoro-2-[(phenylmethyl)oxy]phenyl}-6-methyl-3-(2-phenylethyl)-5-(1H-pyrrol-1-yl)-4(3H)-pyrimidinone (0.601 g, 0.35 mmol) was taken up in ethanol. To this was added 10% Pd/C (0.10 g). This mixture was placed under hydrogen atmosphere at atmospheric pressure and stirred for 12 h. The reaction mixture was filtered through a bed of celite, concentrated and purified by chromatography on silica gel (Biotage) using EtOAc and hexane mixtures (5-30%) to obtain the desired product (0.41 g, 84%). MS (m/z): 390.2 (M+H)⁺.

### Parenteral Formulation

A pharmaceutical composition for parenteral administration is prepared by dissolving an appropriate amount of a compound of Formula (I) in polyethylene glycol with heating. This solution is then diluted with water for injections (to 100 mL). The solution is then rendered sterile by filtration through a 0.22 micron membrane filter and sealed in sterile containers.

All publications, including but not limited to patents and patent applications cited in this specification are herein incorporated by reference as if each individual publication were specifically and individually indicated to be incorporated by reference as though fully set forth.

1. A compound according to formula (I): 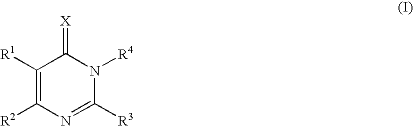
wherein:
X is O or S;
R¹ and R² are, independently, selected from the group consisting of H, halogen, CN, C₁₋₁₀alkyl, C₂₋₆alkenyl, cycloalkyl, cycloalkylC₁₋₆alkyl, aryl, arylC₁₋₆alkyl, heterocyclyl, heteroaryl, (CR₁₀R₁₁)ₓNR₅R₆, C(O)OR₅, C(O)NR₅R₆, NR₅C(O)R₆, (CR₁₀R₁₁)ₓOR₅ and NC(O)R₅, optionally substituted, except for H, halogen and CN, one to three times, independently, by halogen, CN, C₁₋₄alkyl, aryl, heteroaryl, C(O)OR₁₉, O—(CR₁₉R₂₀)_{q}—O, C(O)R₁₉, CF₃, OCF₃, NO₂, C(O)NR₁₉R₂₀, (CR₁₀R₁₁)_{z}—OR₁₉, (CR₁₀R₁₁)_{z}NR₁₉R₂₀, and (CR₁₀R₁₁)ₓS(O)ₘR₁₉;
or R¹ and R² together form an optionally substituted 5 to 8 membered ring, optionally containing one to three heteroatoms selected from N, O and S, wherein the optional substituents are selected, independently, at each occurrence, one to three times, from the group consisting of halogen, C₁₋₄alkyl, (CR₁₀R₁₁)_{z}—S(O)ₘR₅, (CR₁₀R₁₁)_{z}OR₅, (CR₁₀R₁₁)_{z}NR₅R₆, C(O)R₅ and C(O)OR₅;
or R¹ and R² together form an optionally substituted heteroaryl ring, wherein the optional substituents are selected, independently, at each occurrence, one to three times, from the group consisting of halogen, C₁₋₄alkyl, (CR₁₀R₁₁)_{z}—S(O)ₘR₅, (CR₁₀R₁₁)_{z}OR₅, (CR₁₀R₁₁)_{z}NR₅R₆, C(O)R₅ and C(O)OR₅;
or, when R¹ is NR₅R₆, R₅ and R₆ can join together to form a 5 to 7 membered ring, optionally substituted by C₁₋₄alkyl or halogen;
R₅ and R₆ represent, independently, at each occurrence, H, C₁₋₄alkyl, cycloalkyl, cycloalkylC₁₋₆alkyl, C₂₋₆alkenyl, heterocyclyl, heterocyclylC₁₋₆alkyl, aryl, arylC₁₋₆alkyl, heteroaryl or heteroarylC₁₋₆alkyl, wherein each moiety, except H, is optionally substituted, independently, one to three times, by halogen or C₁₋₄alkyl;
R₁₀ and R₁₁, represent, at each occurrence, independently, H or C₁₋₄alkyl;
R₁₉ and R₂₀ represent, independently, at each occurrence, H, C₁₋₄alkyl, cycloalkyl, cycloalkylC₁₋₆alkyl, C₂₋₆alkenyl, heterocyclyl, heterocyclylC₁₋₄alkyl, aryl, arylC₁₋₆alkyl, heteroaryl or a heteroarylC₁₋₆alkyl moiety, wherein each moiety, except H, may be substituted, independently, one to three times, by halogen or C₁₋₄alkyl;
R³ represents aryl or heteroaryl, optionally substituted, independently, one to three times, by C₁₋₄alkyl, halogen, CN or CF₃;
R⁴ is selected from the group consisting of cycloalkylC₁₋₄alkyl, heteroaryl, heterocyclyl, aryl, heteroarylC₁₋₂alkyl, heterocyclylC₁₋₂alkyl, cycloalkylC₂alkenyl, arylC₂alkenyl, heteroarylC₂alkenyl and heterocyclylC₂alkenyl, wherein each moiety is optionally substituted, independently, one to three times, by C₁₋₄alkyl, F, CF₃ or Cl;
m is 0, 1 or 2;
x is 0, 1, 2 or 3;
q is 1, 2 or 3; and
z is 0, 1, 2, 3 or 4;
or a pharmaceutically acceptable salt thereof.

2. A compound according to claim 1 wherein X is O.

3. A compound according to claim 1 wherein R¹ and R² are, independently, selected from the group consisting of H, I, Cl, Br, F, CN, methyl, ethyl, isobutyl, propyl, butyl, isopropyl, hexyl, 2-methylbutyl, 3-methylbutyl, 2-hydroxyethyl, 2-methyl-2-propenyl, 2-methyl-1-propenyl, 1-propenyl, cyclopentyl, cyclopropyl, cyclobutylethyl, cyclobutylmethyl, cyclopropylmethyl, phenyl, 2,4-difluorophenyl, 3,4-difluorophenyl, 4-fluorophenyl, 2-fluorophenyl, 3-fluorophenyl, 3-methylphenyl, 4-hydroxyphenyl, 2-cyanophenyl, 4-cyanophenyl, 4-trifluoromethylphenyl, 3-hydroxymethylphenyl, 3-hydroxyphenyl, 4-N,N-dimethylphenyl, 4-ethoxyphenyl, 4-biphenyl, 4-isopropoxyphenyl, 5-methylsulfonylphenyl, 3-ethoxyphenyl, 2-ethoxyphenyl, 3-cyanophenyl, 4-trifluoromethoxyphenyl, 3-trifluoromethoxyphenyl, 3-dimethylaminomethylphenyl, 3-N,N-dimethylbenzamidyl, 4-t-butylphenyl, 4-isopropylphenyl, 3-N,N-dimethylmethylphenyl, 3-nitrophenyl, carboxylic acid, pyrrolidinyl, morpholinyl, azetidinyl, phenpropyl, phenethyl, 3,4-dichlorophenethyl, ethylamino, methylethylisobutylamino, diethylamino, dimethylamino, 2,2-dimethylpropanamide, NH₂, N-N-dimethylamino, aniline, N-propyl, methylmethylether, benzylethylether, methylethylether, ethylether, isopropylether, N,2-dimethylpropanamide, 2-methylpropanamide, pyrazinyl, 3-pyridyl, 2-furyl, 3-furyl, 2-indanyl, 3-methyl-2-thienyl, 4-methyl-2-thienyl, 5-methyl-2-thienyl, 3-thienyl, 2-thienyl, 5-chloro-2-thienyl, 2-pyrrolyl, 1-methyl-2-pyrrolyl, 5-methyl-3-thienyl, 5-methylamino-2-thienyl, 5-hydroxymethyl-2-thienyl, 4,5-dimethyl-2-thienyl, 5-cyano-2-thienyl, 5-phenyl-2-thienyl, 2-methyl-1,3-thiazol-5-yl, 1,3-thiazol-2-yl, 5-acetyl-2-thienyl, 4,5-dimethyl-1,3-thiazol-2-yl, 4-methyl-1,3-thiazol-2-yl, 5-methyl-1,3,4-oxadiazol-2-yl, quinolinyl, 1,2,3,4-tetrahydroquinolinyl, 1,2,3,4-methyltetrahydroquinolinyl, 2,3-dihydro-1,4-benzodioxinyl, 3-benzothiophenyl, 1,3-benzodioxol-5-yl, 4-benzothienyl, 2-benzofuranyl, 4,5,6,7-tetrahydrobenzothienyl, 1-methylindol-5-yl, 5-(2-phenyl-1,3-thiazol-5-yl), 5-chloro-3-methyl-1-benzothien-2-yl, 2-benzothiophenyl, 1-methylindol-2-yl, and 5-(2-methyl-1,3-thiazol-4-yl)-2-thienyl.

4. A compound according to claim 1 wherein R⁴ is selected from the group consisting of phenylC₁₋₂alkyl, cyclohexylC₁₋₂alkyl, cyclopentylC₁₋₂alkyl, thienylC₁₋₂alkyl, pyranylC₁₋₂alkyl, indenylC₁₋₂alkyl and piperidinylC₁₋₂alkyl, optionally substituted, independently, once or twice, by F, CF₃ or Cl.

5. A compound according to claim 1 wherein:
R¹ is selected from the group consisting of an isobutyl, ethyl, phenyl, furanyl, quinolinyl, halogen, tetrahydroquinolinyl, pyrrolidinyl, thiophenyl, morpholinyl, cyclopentyl, isopropyl, amino, pyrazinyl, indolyl, thiazolyl, piperidinyl, N-acyl, benzothiophenyl and benzothiazolyl moiety, optionally substituted, independently, one to three times, by C₁₋₄alkyl or halogen; and
R² is selected from the group consisting of a methyl, methoxymethyl, piperidinyl, ethyl, methoxyethyl, benzyloxyethyl, phenyl, pyrrolidinyl, amino, alkylamino, propyl, phenethyl, phenpropyl, butyl, isobutyl, cyclobutylethyl, 3-methylbutyl, dimethylaminomethyl, piperidinylmethyl, and alkylaminomethyl moiety, optionally substituted, independently, one to three times, by C₁₋₄alkyl or halogen.

6. A compound according to claim 1 wherein R³ represents an optionally substituted heteroaryl moiety.

7. A compound according to claim 1 wherein R³ represents an optionally substituted aryl moiety.

8. A compound according to claim 1 selected from the group consisting of:
2-(2-Fluoro-3-hydroxyphenyl)-6-methyl-5-(2-methylpropyl)-3-(2-phenylethyl)-4(3H)-pyrimidinone;
2-(3-Hydroxyphenyl)-6-methyl-5-(2-methylpropyl)-3-(2-phenylethyl)-4(3H)-pyrimidinone;
2-(2,3-Dihydroxyphenyl)-6-methyl-5-(2-methylpropyl)-3-(2-phenylethyl)-4(3H)-pyrimidinone;
6-Methyl-5-(2-methylpropyl)-3-(2-phenylethyl)-2-(1H-pyrrol-2-yl)-4(3H)-pyrimidinone;
6-Methyl-5-(2-methylpropyl)-3-(2-phenylethyl)-2-(2-thienyl)-4(3H)-pyrimidinone;
6-Methyl-5-(2-methylpropyl)-3-(2-phenylethyl)-2-(2-pyridinyl)-4(3H)-pyrimidinone;
2-(2-Furanyl)-6-methyl-5-(2-methylpropyl)-3-(2-phenylethyl)-4(3H)-pyrimidinone;
2-(1H-imidazol-2-yl)-6-methyl-5-(2-methylpropyl)-3-(2-phenylethyl)-4(3H)-pyrimidinone;
5-Ethyl-2-(2-fluoro-3-hydroxyphenyl)-3-[2-(3-fluorophenyl)ethyl]-6-methyl-4(3H)-pyrimidinone;
5-Ethyl-3-[2-(3-fluorophenyl)ethyl]-6-methyl-2-(1H-pyrrol-2-yl)-4(3H)-pyrimidinone;
5-Bromo-2-{3-fluoro-2-[(phenylmethyl)oxy]phenyl}-6-methyl-3-(2-phenylethyl)-4(3H)-pyrimidinone;
5-Bromo-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(6-quinolinyl)-4(3H)-pyrimidinone;
2-(3-Fluoro-2-hydroxyphenyl)-6-methyl-3-(2-phenylethyl)-5-(1,2,3,4-tetrahydro-6-quinolinyl)-4(3H)-pyrimidinone;
2-(3-Fluoro-2-hydroxyphenyl)-6-methyl-5-(1-methyl-1,2,3,4-tetrahydro-6-quinolinyl)-3-(2-phenylethyl)-4(3H)-pyrimidinone;
2-(3-Fluoro-2-hydroxyphenyl)-5-(2-furanyl)-6-methyl-3-(2-phenylethyl)-4(3H)-pyrimidinone;
2-(3-Fluoro-2-hydroxyphenyl)-6-methyl-5-phenyl-3-[2-(2-thienyl)ethyl]-4(3H)-pyrimidinone;
2-(3-Fluoro-2-hydroxyphenyl)-6-methyl-3-(2-phenylethyl)-5-(1-pyrrolidinyl)-4(3H)-pyrimidinone;
5-(5-Chloro-2-thienyl)-2-(3-fluoro-2-hydroxyphenyl)-6-methyl-3-(2-phenylethyl)-4(3H)-pyrimidinone;
5-bromo-6-methyl-3-(2-phenylethyl)-2-{2-[(phenylmethyl)oxy]phenyl}-4(3H)-pyrimidinone;
2-(2-Hydroxyphenyl)-3-(2-phenylethyl)-6-(1-piperidinylmethyl)-4(3H)-pyrimidinone;
2-(2-Hydroxyphenyl)-6-{[methyl(2-methylpropyl)amino]methyl}-3-(2-phenylethyl)-4(3H)-pyrimidinone;
2-(2-Hydroxyphenyl)-6-methyl-5-[(1-methylethyl)oxy]-3-(2-phenylethyl)-4(3H)-pyrimidinone;
5-(2-Furanyl)-2-(2-hydroxyphenyl)-6-methyl-3-(2-phenylethyl)-4(3H)-pyrimidinone;
2-(2-Hydroxyphenyl)-6-methyl-3-(2-phenylethyl)-5-(2-thienyl)-4(3H)-pyrimidinone;
2-(2-Hydroxyphenyl)-6-methyl-5-(4-morpholinyl)-3-(2-phenylethyl)-4(3H)-pyrimidinone;
5-Ethyl-2-(3-fluoro-2-hydroxyphenyl)-3-[2-(3-fluorophenyl)ethyl]-6-(1-piperidinyl)-4(3H)-pyrimidinone;
5-Ethyl-1-[2-(3-fluorophenyl)ethyl]-2-[2-(methyloxy)phenyl]-6-oxo-1,6-dihydro-4-pyrimidinecarboxylic acid;
5-Ethyl-2-(2-hydroxyphenyl)-6-methyl-3-[(E)-2-phenylethenyl]-4(3H)-pyrimidinone;
2-(3,6-Difluoro-2-hydroxyphenyl)-5-ethyl-3-[2-(3-fluorophenyl)ethyl]-6-methyl-4(3H)-pyrimidinone;
2-(3-Fluoro-2-hydroxyphenyl)-6-methyl-5-propyl-3-[2-(2-thienyl)ethyl]-4(3H)-pyrimidinone;
2-(2-Hydroxyphenyl)-5,5-dimethyl-3-[2-(2-thienyl)ethyl]-5,6,7,8-tetrahydro-4(3H)-quinazolinone;
3-[2-(2-Fluorophenyl)ethyl]-2-(2-hydroxyphenyl)-5,5-dimethyl-5,6,7,8-tetrahydro-4(3H)-quinazolinone;
2-(2-Hydroxyphenyl)-3-(2-phenylethyl)-3,5,6,7,8,9-hexahydro-4H-cyclohepta[d]pyrimidin-4-one;
2-(3-Fluoro-2-hydroxyphenyl)-3-(2-phenylethyl)-5,6,7,8-tetrahydro-4(3H)-quinazolinone;
5-Cyclopentyl-2-(3-fluoro-2-hydroxyphenyl)-6-methyl-3-(2-phenylethyl)-4(3H)-pyrimidinone;
5-(2,3-Dihydro-1,4-benzodioxin-6-yl)-6-methyl-3-(2-phenylethyl)-2-(2-thienyl)-4(3H)-pyrimidinone;
2-(2-Hydroxyphenyl)-6-[(methyloxy)methyl]-3-(2-phenylethyl)-4(3H)-pyrimidinone;
2-(2-Hydroxyphenyl)-6-[(methyloxy)methyl]-5-(2-methylpropyl)-3-(2-phenylethyl)-4(3H)-pyrimidinone;
2-(3-Fluoro-2-hydroxyphenyl)-6-methyl-5-[2-(methyloxy)ethyl]-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinethione;
2-(3-Fluoro-2-hydroxyphenyl)-6-methyl-5-phenyl-3-(2-phenylethyl)-4(3H)-pyrimidinethione;
2-(3-Fluoro-2-hydroxyphenyl)-6-methyl-5-(2-methylpropyl)-3-(2-phenylethyl)-4(3H)-pyrimidinethione;
3-(2,3-Dihydro-1H-inden-2-yl)-2-(2-hydroxyphenyl)-6-methyl-5-(1-methylethyl)-4(3H)-pyrimidinone;
5,6-Diethyl-2-(3-fluoro-2-hydroxyphenyl)-3-[2-(2-fluorophenyl)ethyl]-4(3H)-pyrimidinone;
6-(2-Cyclohexylethyl)-5-ethyl-2-(3-fluoro-2-hydroxyphenyl)-3-[2-(2-fluorophenyl)ethyl]-4(3H)-pyrimidinone;
6-[2-(3,4-Dichlorophenyl)ethyl]-5-ethyl-2-(3-fluoro-2-hydroxyphenyl)-3-[2-(2-fluorophenyl)ethyl]-4(3H)-pyrimidinone;
2-(3-Fluoro-2-hydroxyphenyl)-3-[2-(2-fluorophenyl)ethyl]-6-methyl-5-(2-methylpropyl)-4(3H)-pyrimidinone;
2-(3-Fluoro-2-hydroxyphenyl)-6-methyl-5-(2-methylpropyl)-3-[2-(2-thienyl)ethyl]-4(3H)-pyrimidinone;
2-(3-Fluoro-2-hydroxyphenyl)-3-[2-(4-fluorophenyl)ethyl]-6-methyl-5-(2-methylpropyl)-4(3H)-pyrimidinone;
2-(3-Fluoro-2-hydroxyphenyl)-3-[2-(3-fluorophenyl)ethyl]-6-methyl-5-(2-methylpropyl)-4(3H)-pyrimidinone;
2-(2-Hydroxyphenyl)-7-methyl-3-(2-phenylethyl)-3,5,6,7,8,9-hexahydro-4H-pyrimido[4,5-d]azepin-4-one;
7-acetyl-2-(2-hydroxyphenyl)-3-(2-phenylethyl)-3,5,6,7,8,9-hexahydro-4H-pyrimido[4,5-d]azepin-4-one;
2-(2-Hydroxyphenyl)-7-(methylsulfonyl)-3-(2-phenylethyl)-3,5,6,7,8,9-hexahydro-4H-pyrimido[4,5-d]azepin-4-one;
5-Bromo-2-(2-hydroxyphenyl)-6-methyl-3-(2-phenylethyl)-4(3H)-pyrimidinone;
2-(2-Hydroxyphenyl)-5-iodo-6-methyl-3-(2-phenylethyl)-4(3H)-pyrimidinone;
5-Chloro-3-(2-cyclohexylethyl)-2-(2-hydroxyphenyl)-6-methyl-4(3H)-pyrimidinone;
5-Chloro-2-(2-hydroxyphenyl)-6-methyl-3-[2-(2-thienyl)ethyl]-4(3H)-pyrimidinone;
5-Chloro-2-(2-hydroxyphenyl)-6-methyl-3-(2-phenylethyl)-4(3H)-pyrimidinethione;
5-Bromo-2-(3-hydroxyphenyl)-6-methyl-3-(2-phenylethyl)-4(3H)-pyrimidinone;
2-(3-Hydroxyphenyl)-6-methyl-5-phenyl-3-(2-phenylethyl)-4(3H)-pyrimidinone;
2-(2-hydroxyphenyl)-6-methyl-5-(phenylamino)-3-(2-phenylethyl)-4(3H)-pyrimidinone;
5-(1-Azetidinyl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(propylamino)-4(3H)-pyrimidinone;
2-(2-Fluoro-3-hydroxyphenyl)-6-methyl-5-phenyl-3-(2-phenylethyl)-4(3H)-pyrimidinone;
2-(2-Hydroxyphenyl)-6-methyl-3-(2-phenylethyl)-5-(3-thienyl)-4(3H)-pyrimidinone;
5-(3-Furanyl)-2-(2-hydroxyphenyl)-6-methyl-3-(2-phenylethyl)-4(3H)-pyrimidinone;
5-(4-Biphenylyl)-2-(2-hydroxyphenyl)-6-methyl-3-(2-phenylethyl)-4(3H)-pyrimidinone;
5-(1,3-Benzodioxol-5-yl)-2-(2-hydroxyphenyl)-6-methyl-3-(2-phenylethyl)-4(3H)-pyrimidinone;
5-(2-fluorophenyl)-2-(2-hydroxyphenyl)-6-methyl-3-(2-phenylethyl)-4(3H)-pyrimidinone;
2-(2-Hydroxyphenyl)-6-methyl-3-(2-phenylethyl)-5-[4-(trifluoromethyl)phenyl]-4(3H)-pyrimidinone;
5-(3-Fluorophenyl)-2-(2-hydroxyphenyl)-6-methyl-3-(2-phenylethyl)-4(3H)-pyrimidinone;
5-(2,4-Difluorophenyl)-2-(2-hydroxyphenyl)-6-methyl-3-(2-phenylethyl)-4(3H)-pyrimidinone;
5-[4-(Dimethylamino)phenyl]-2-(3-fluoro-2-hydroxyphenyl)-6-methyl-3-(2-phenylethyl)-4(3H)-pyrimidinone;
5-[4-(Ethyloxy)phenyl]-2-(3-fluoro-2-hydroxyphenyl)-6-methyl-3-(2-phenylethyl)-4(3H)-pyrimidinone;
5-(1-Benzothien-3-yl)-2-(3-fluoro-2-hydroxyphenyl)-6-methyl-3-(2-phenylethyl)-4(3H)-pyrimidinone;
5-(1-Benzothien-4-yl)-2-(3-fluoro-2-hydroxyphenyl)-6-methyl-3-(2-phenylethyl)-4(3H)-pyrimidinone;
2-[2-(3-Fluoro-2-hydroxyphenyl)-4-methyl-6-oxo-1-(2-phenylethyl)-1,6-dihydro-5-pyrimidinyl]benzonitrile;
4-[2-(3-Fluoro-2-hydroxyphenyl)-4-methyl-6-oxo-1-(2-phenylethyl)-1,6-dihydro-5-pyrimidinyl]benzonitrile;
5-[2-(Ethyloxy)phenyl]-2-(3-fluoro-2-hydroxyphenyl)-6-methyl-3-(2-phenylethyl)-4(3H)-pyrimidinone;
5-[3-(Ethyloxy)phenyl]-2-(3-fluoro-2-hydroxyphenyl)-6-methyl-3-(2-phenylethyl)-4(3H)-pyrimidinone;
5-(1-Benzofuran-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(1H-pyrrol-2-yl)-4(3H)-pyrimidinone;
2-(3-Fluoro-2-hydroxyphenyl)-5-[3-(hydroxymethyl)phenyl]-6-methyl-3-(2-phenylethyl)-4(3H)-pyrimidinone;
2-(3-Fluoro-2-hydroxyphenyl)-6-methyl-5-[3-(methylsulfonyl)phenyl]-3-(2-phenylethyl)-4(3H)-pyrimidinone;
2-(3-Fluoro-2-hydroxyphenyl)-6-methyl-3-(2-phenylethyl)-5-[3-(trifluoromethyl)phenyl]-4(3H)-pyrimidinone;
5-(3,4-Difluorophenyl)-2-(3-fluoro-2-hydroxyphenyl)-6-methyl-3-(2-phenylethyl)-4(3H)-pyrimidinone;
5-[4-(1,1-Dimethylethyl)phenyl]-2-(3-fluoro-2-hydroxyphenyl)-6-methyl-3-(2-phenylethyl)-4(3H)-pyrimidinone;
5-(5-Acetyl-2-thienyl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-{3-[(trifluoromethyl)oxy]phenyl}-4(3H)-pyrimidinone;
5-{3-[(Dimethylamino)methyl]phenyl}-2-(3-fluoro-2-hydroxyphenyl)-6-methyl-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]-N,N-dimethylbenzamide;
5-(4,5-Dimethyl-2-thienyl)-2-(3-fluoro-2-hydroxyphenyl)-6-methyl-3-(2-phenylethyl)-4(3H)-pyrimidinone;
5-[2-(3-Fluoro-2-hydroxyphenyl)-4-methyl-6-oxo-1-(2-phenylethyl)-1,6-dihydro-5-pyrimidinyl]-2-thiophenecarbonitrile;
2-(3-Fluoro-2-hydroxyphenyl)-6-methyl-5-(1-methyl-1H-pyrrol-2-yl)-3-(2-phenylethyl)-4(3H)-pyrimidinone;
2-(3-fluoro-2-hydroxyphenyl)-6-methyl-5-(1-methyl-1H-indol-2-yl)-3-(2-phenylethyl)-4(3H)-pyrimidinone;
2-(3-Fluoro-2-hydroxyphenyl)-6-methyl-3-(2-phenylethyl)-5-(1,3-thiazol-2-yl)-4(3H)-pyrimidinone;
2-(2-Hydroxyphenyl)-6-methyl-3-(2-phenylethyl)-5-(3-pyridinyl)-4(3H)-pyrimidinone;
2-(2-Hydroxyphenyl)-6-methyl-3-(2-phenylethyl)-5-(2-pyrazinyl)-4(3H)-pyrimidinone;
6-Methyl-2-[2-(methyloxy)phenyl]-3-(2-phenylethyl)-5-(2-thienyl)-4(3H)-pyrimidinone;
2-(2-Hydroxyphenyl)-6-methyl-5-phenyl-3-(2-phenylethyl)-4(3H)-pyrimidinone;
5-(4-Fluorophenyl)-2-(2-hydroxyphenyl)-6-methyl-3-(2-phenylethyl)-4(3H)-pyrimidinone;
2-(2-Hydroxyphenyl)-6-methyl-5-(3-methylphenyl)-3-(2-phenylethyl)-4(3H)-pyrimidinone;
2-(3-Fluoro-2-hydroxyphenyl)-6-methyl-5-(1-methyl-1H-indol-5-yl)-3-(2-phenylethyl)-4(3H)-pyrimidinone;
2-(3-Fluoro-2-hydroxyphenyl)-6-methyl-3-(2-phenylethyl)-5-{4-[(trifluoromethyl)oxy]phenyl}-4(3H)-pyrimidinone;
2-(3-Fluoro-2-hydroxyphenyl)-6-methyl-5-{4-[(1-methylethyl)oxy]phenyl}-3-(2-phenylethyl)-4(3H)-pyrimidinone;
2-(3-Fluoro-2-hydroxyphenyl)-6-methyl-3-(2-phenylethyl)-5-(6-quinolinyl)-4(3H)-pyrimidinone;
5-(2,3-Dihydro-1,4-benzodioxin-6-yl)-2-(2-hydroxyphenyl)-6-methyl-3-(2-phenylethyl)-4(3H)-pyrimidinone;
5-(5-Chloro-3-methyl-1-benzothien-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-[5-(1,3-oxazol-5-yl)-2-thienyl]-3-(2-phenylethyl)-4(3H)-pyrimidinone;
5-Fluoro-2-(2-hydroxyphenyl)-6-methyl-3-(2-phenylethyl)-4(3H)-pyrimidinone;
2-(2-Hydroxyphenyl)-6-methyl-5-(2-methylpropyl)-3-(2-phenylethyl)-4(3H)-pyrimidinone;
2-(2-Hydroxyphenyl)-6-methyl-5-(2-methyl-2-propen-1-yl)-3-(2-phenylethyl)-4(3H)-pyrimidinone;
5-(Cyclobutylmethyl)-6-methyl-2-[2-(methyloxy)phenyl]-3-(2-phenylethyl)-4(3H)-pyrimidinone;
5-(Cyclobutylmethyl)-2-(2-hydroxyphenyl)-6-methyl-3-(2-phenylethyl)-4(3H)-pyrimidinone;
2-(2-Hydroxyphenyl)-6,6-dimethyl-3-(2-phenylethyl)-4-a,5,6,7,8,8a-hexahydro-4(3H)-quinazolinone;
5-(Cyclopropylmethyl)-2-(3-fluoro-2-hydroxyphenyl)-6-methyl-3-(2-phenylethyl)-4(3H)-pyrimidinone;
5-Cyclopropyl-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(3-methylbutyl)-3-(2-phenylethyl)-4(3H)-pyrimidinone;
5-(2-Cyclohexylethyl)-2-(3-fluoro-2-hydroxyphenyl)-6-methyl-3-(2-phenylethyl)-4(3H)-pyrimidinone;
5-(Cyclohexylmethyl)-2-(2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(phenylmethyl)-4(3H)-pyrimidinone;
5-Amino-2-(2-hydroxyphenyl)-6-methyl-3-(2-phenylethyl)-4(3H)-pyrimidinone;
2-(2-Hydroxyphenyl)-6-methyl-3-(2-phenylethyl)-5-(1-piperidinyl)-4(3H)-pyrimidinone;
5-(Dimethylamino)-2-(2-hydroxyphenyl)-6-methyl-3-(2-phenylethyl)-4(3H)-pyrimidinone;
N-[2-(2-Hydroxyphenyl)-4-methyl-6-oxo-1-(2-phenylethyl)-1,6-dihydro-5-pyrimidinyl]-2,2-dimethylpropanamide;
N-[2-(2-Hydroxyphenyl)-4-methyl-6-oxo-1-(2-phenylethyl)-1,6-dihydro-5-pyrimidinyl]-2-methylpropanamide;
N-[2-(2-hydroxyphenyl)-4-methyl-6-oxo-1-(2-phenylethyl)-1,6-dihydro-5-pyrimidinyl]-N,2-dimethylpropanamide;
5-(Dipropylamino)-2-(2-hydroxyphenyl)-6-methyl-3-(2-phenylethyl)-4(3H)-pyrimidinone;
5-(Diethylamino)-2-(2-hydroxyphenyl)-6-methyl-3-(2-phenylethyl)-4(3H)-pyrimidinone;
5-(Ethylamino)-2-(2-hydroxyphenyl)-6-methyl-3-(2-phenylethyl)-4(3H)-pyrimidinone;
2-(2-Hydroxyphenyl)-5-(2-methylpropyl)-3-(2-phenylethyl)-6-propyl-4(3H)-pyrimidinone;
6-Ethyl-2-(2-hydroxyphenyl)-5-(2-methylpropyl)-3-(2-phenylethyl)-4(3H)-pyrimidinone;
6-Butyl-2-(2-hydroxyphenyl)-5-(2-methylpropyl)-3-(2-phenylethyl)-4(3H)-pyrimidinone;
2-(2-Hydroxyphenyl)-5-(2-methylpropyl)-3-(2-phenylethyl)-6-{2-[(phenylmethyl)oxy]ethyl}-4(3H)-pyrimidinone;
6-(2-Hydroxyethyl)-2-(2-hydroxyphenyl)-5-(2-methylpropyl)-3-(2-phenylethyl)-4(3H)-pyrimidinone;
6-[2-(methyloxy)ethyl]-5-(2-methyl-1-propen-1-yl)-3-(2-phenylethyl)-4(3H)-pyrimidinone;
2-(2-hydroxyphenyl)-6-[2-(methyloxy)ethyl]-5-(2-methylpropyl)-3-(2-phenylethyl)-4(3H)-pyrimidinone;
5-(dimethylamino)-2-(3-fluoro-2-hydroxyphenyl)-6-methyl-3-(2-phenylethyl)-4(3H)-pyrimidinone;
5-(Dimethylamino)-2-(2-fluoro-3-hydroxyphenyl)-6-methyl-3-(2-phenylethyl)-4(3H)-pyrimidinone;
6-Methyl-2,5-diphenyl-3-(2-phenylethyl)-4(3H)-pyrimidinone;
2-(2-Fluorophenyl)-6-methyl-5-phenyl-3-(2-phenylethyl)-4(3H)-pyrimidinone;
3-[2-(2-chlorophenyl)ethyl]-2-(2-hydroxyphenyl)-5,6,7,8-tetrahydro-4(3H)-quinazolinone;
3-[2-(3-fluorophenyl)ethyl]-2-(2-hydroxyphenyl)-5,5-dimethyl-5,6,7,8-tetrahydro-4(3H)-quinazolinone;
3-(2-cyclohexylethyl)-2-(2-hydroxyphenyl)-5,5-dimethyl-5,6,7,8-tetrahydro-4(3H)-quinazolinone;
3-[2-(3-fluorophenyl)ethyl]-2-(2-furanyl)-5,6,7,8-tetrahydro-4(3H)-quinazolinone;
3-[2-(3-fluorophenyl)ethyl]-2-(2-thienyl)-5,6,7,8-tetrahydro-4(3H)-quinazolinone;
ethyl 2-(2-hydroxyphenyl)-4-methyl-6-oxo-1-(2-phenylethyl)-1,6-dihydro-5-pyrimidinecarbonitrile;
Ethyl 2-(2-hydroxyphenyl)-4-methyl-6-oxo-1-(2-phenylethyl)-1,6-dihydro-5-pyrimidinecarboxylate;
2-(2-hydroxyphenyl)-6-methyl-5-(1-methylpropyl)-3-[2-(2-thienyl)ethyl]-4(3H)-pyrimidinone;
2-(2-hydroxyphenyl)-6-methyl-5-(1-methylpropyl)-3-(2-phenylethyl)-4(3H)-pyrimidinone;
2-(3-fluoro-2-hydroxyphenyl)-6-methyl-5-(1-methylpropyl)-3-(2-phenylethyl)-4(3H)-pyrimidinone;
5-butyl-2-(2-hydroxyphenyl)-6-methyl-3-(2-phenylethyl)-4(3H)-pyrimidinone;
2-(2-hydroxyphenyl)-6-methyl-5-pentyl-3-(2-phenylethyl)-4(3H)-pyrimidinone;
5-hexyl-2-(2-hydroxyphenyl)-6-methyl-3-(2-phenylethyl)-4(3H)-pyrimidinone;
5-butyl-2-(2-hydroxyphenyl)-6-methyl-3-[2-(2-thienyl)ethyl]-4(3H)-pyrimidinone;
2-(2-hydroxyphenyl)-6-methyl-5-pentyl-3-[2-(2-thienyl)ethyl]-4(3H)-pyrimidinone;
5-hexyl-2-(2-hydroxyphenyl)-6-methyl-3-[2-(2-thienyl)ethyl]-4(3H)-pyrimidinone;
2-(3-fluoro-2-hydroxyphenyl)-3-[2-(3-fluorophenyl)ethyl]-5,6,7,8-tetrahydro-4(3H)-quinazolinone;
2-(3-fluoro-2-hydroxyphenyl)-3-[2-(3-fluorophenyl)ethyl]-3,5,6,7,8,9-hexahydro-4H-cyclohepta[d]pyrimidin-4-one;
Ethyl 2-(2-hydroxyphenyl)-4-oxo-3-(2-phenylethyl)-3,5,7,8-tetrahydropyrido[4,3-d]pyrimidine-6(4H)-carboxylate;
(2-hydroxyphenyl)-6-(3-methylbutanoyl)-3-(2-phenylethyl)-5,6,7,8-tetrahydropyrido[4,3-d]pyrimidin-4(3H)-one;
5-ethyl-2-(2-hydroxyphenyl)-6-methyl-3-[2-(2-thienyl)ethyl]-4(3H)-pyrimidinone;
5-Isopropyl-2-(2-hydroxy-phenyl)-6-methyl-3-(2-thiophen-2-yl-ethyl)-3H-pyrimidin-4-one;
5-isopropyl-2-(2-hydroxy-phenyl)-6-methyl-3-(2-cyclohexyl-ethyl)-3H-pyrimidin-4-one;
5-Ethyl-2-(2-hydroxy-3-fluorophenyl)-6-methyl-3-(2-fluorophenylethyl)-3H-pyrimidin-4-one;
5-propenyl-2-(2-hydroxy-3-fluorophenyl)-6-methyl-3-(3-fluorophenylethyl)-3H-pyrimidin-4-one;
3-(2-cyclohexylethyl)-2-(2-hydroxyphenyl)-5,6,7,8-tetrahydro-4(3H)-quinazolinone;
3-(2-thiophen-2-yl-ethyl)-2-(2-hydroxy-phenyl)-5,6,7,8-tetrahydro-3H-quinazolin-4-one;
3-(2-thiophen-2-yl-ethyl)-2-(2-hydroxy-3-fluorophenyl)-5,6,7,8-tetrahydro-3H-quinazolin-4-one;
3-(2-thiophen-3-yl-ethyl)-2-(2-hydroxy-phenyl)-5,6,7,8-tetrahydro-3H-quinazolin-4-one;
3-(3-chlorophenethyl)-2-(2-hydroxy-phenyl)-5,6,7,8-tetrahydro-3H-quinazolin-4-one;
3-(2-cyclopentylethyl)-2-(2-hydroxy-phenyl)-5,6,7,8-tetrahydro-3H-quinazolin-4-one;
3-(3-trifluoromethylphenethyl)-2-(2-hydroxy-phenyl)-5,6,7,8-tetrahydro-3H-quinazolin-4-one;
2-(2-Hydroxyphenyl)-3-(2-phenylethyl)-5,6,8,9-tetrahydrooxepino[4,5-d]pyrimidin-4(3H)-one;
3-(2-Cyclohexyl-ethyl)-2-(2-hydroxy-phenyl)-3,5,6,7,89-hexahydro-cycloheptapyrimidin-4-one;
2-(2-hydroxyphenyl)-3-(2-phenylethyl)-6-(phenylmethyl)-5,6,7,8-tetrahydropyrido[4,3-d]pyrimidin-4(3H)-one;
2-Methylpropyl 2-(2-hydroxyphenyl)-4-oxo-3-(2-phenylethyl)-3,5,7,8-tetrahydropyrido[4,3-d]pyrimidine-6(4H)-carboxylate;
2-(3-Fluoro-2-hydroxyphenyl)-6-methyl-5-[5-(2-methyl-1,3-thiazol-4-yl)-2-thienyl]-3-(2-phenylethyl)-4(3H)-pyrimidinone;
2-[2-(hydroxy)phenyl]-3-(2-phenylethyl)-5,6,7,8-tetrahydropyrido[3,2-d]pyrimidin-4(3H)-one;
2-(2-hydroxyphenyl)-5-methyl-3-(2-phenylethyl)-5,6,7,8-tetrahydropyrido[3,2-d]pyrimidin-4(3H)-one;
5-ethyl-2-[2-hydroxyphenyl]-3-(2-phenylethyl)-5,6,7,8-tetrahydropyrido[3,2-d]pyrimidin-4(3H)-one;
1,1-dimethylethyl 2-(2-hydroxyphenyl)-4-oxo-3-(2-phenylethyl)-3,4,5,7-tetrahydro-6H-pyrrolo[3,4-d]pyrimidine-6-carboxylate;
5-(2-methyl propyl-2-yl)-2-(2-hydroxy-phenyl)-6-methyl-3-(2-phenethyl)-3H-pyrimidin-4-one;
5-[2-(3-fluorophenyl)ethyl]-6-(2-hydroxyphenyl)-1-methyl-1,5-dihydro-4H-pyrazolo[3,4-d]pyrimidin-4-one;
5-ethyl-2-(3-fluoro-2-hydroxyphenyl)-3-[2-(3-fluorophenyl)ethyl]-6-phenyl-4(3H)-pyrimidinone;
6-[3,4-bis(methyloxy)phenyl]-5-ethyl-2-(3-fluoro-2-hydroxyphenyl)-3-[2-(3-fluorophenyl)ethyl]-4(3H)-pyrimidinone;
5-ethyl-2-(3-fluoro-2-hydroxyphenyl)-3-[2-(3-fluorophenyl)ethyl]-6-(3-nitrophenyl)-4(3H)-pyrimidinone;
5-ethyl-3-[2-(3-fluorophenyl)ethyl]-2-(2-hydroxyphenyl)-6-(1-pyrrolidinyl)-4(3H)-pyrimidinone;
6-(dimethylamino)-5-ethyl-3-[2-(3-fluorophenyl)ethyl]-2-(2-hydroxyphenyl)-4(3H)-pyrimidinone;
5-ethyl-3-[2-(3-fluorophenyl)ethyl]-2-(2-hydroxyphenyl)-6-(methylamino)-4(3H)-pyrimidinone;
5-cyclopentyl-3-[2-(3-fluorophenyl)ethyl]-2-(2-hydroxyphenyl)-6-methyl-4(3H)-pyrimidinone;
2-(2-hydroxyphenyl)-6-methyl-5-(2-methylpropyl)-3-(2-phenylethyl)-4(3H)-pyrimidinone;
2-(2-hydroxyphenyl)-6-methyl-5-(2-methylpropyl)-3-[2-(2-thienyl)ethyl]-4(3H)-pyrimidinone;
5-Ethyl-2-(2-hydroxy-phenyl)-6-Ethyl-3-phenylethyl-3H-pyrimidin-4-one;
5-Ethyl-2-(2-hydroxy-phenyl)-6-Propyl-3-phenylethyl-3H-pyrimidin-4-one;
5-Ethyl-2-(3-fluoro-2-hydroxy-phenyl)-6-(2-phenylethyl)-3-(2-fluoro-phenylethyl)-3H-pyrimidin-4-one;
5-Ethyl-2-(3-fluoro-2-hydroxy-phenyl)-6-propyl-3-(2-fluoro-phenylethyl)-3H-pyrimidin-4-one;
5-Ethyl-2-(3-fluoro-2-hydroxy-phenyl)-6-(3-phenyl-propyl)-3-(2-fluoro-phenylethyl)-3H-pyrimidin-4-one;
5-Ethyl-2-(3-fluoro-2-hydroxy-phenyl)-6-butyl-3-(2-fluoro-phenylethyl)-3H-pyrimidin-4-one;
5-Ethyl-2-(3-fluoro-2-hydroxy-phenyl)-6-(2-methyl-propyl)-3-(2-fluoro-phenylethyl)-3H-pyrimidin-4-one;
5-Ethyl-2-(3-fluoro-2-hydroxy-phenyl)-6-(3-methyl-butyl)-3-(2-fluoro-phenylethyl)-3H-pyrimidin-4-one;
5-Ethyl-2-(3-fluoro-2-hydroxy-phenyl)-6-(2-cyclobutyl-ethyl)-3-(2-fluoro-phenylethyl)-3H-pyrimidin-4-one;
5-Ethyl-2-(3-fluoro-2-hydroxy-phenyl)-6-(3,4-dichlorophenethyl)-3-(2-fluoro-phenylethyl)-3H-pyrimidin-4-one;
5-ethyl-2-(4-fluoro-2-hydroxyphenyl)-6-methyl-3-(2-phenylethyl)-4(3H)-pyrimidinone;
6-methyl-5-(2-methyl-1,3-thiazol-5-yl)-3-(2-phenylethyl)-2-{2-[(phenylmethyl)oxy]phenyl}-4(3H)-pyrimidinone;
2-(2-hydroxyphenyl)-6-methyl-5-(5-methyl-2-thienyl)-3-(2-phenylethyl)-4(3H)-pyrimidinone;
2-(2-hydroxyphenyl)-6-methyl-5-(5-methyl-3-thienyl)-3-(2-phenylethyl)-4(3H)-pyrimidinone;
2-(3-fluoro-2-hydroxyphenyl)-6-methyl-5-(5-methyl-3-thienyl)-3-(2-phenylethyl)-4(3H)-pyrimidinone;
5-(4,5-dimethyl-2-thienyl)-2-(2-hydroxyphenyl)-6-methyl-3-(2-phenylethyl)-4(3H)-pyrimidinone;
2-(2-hydroxyphenyl)-6-methyl-5-[5-(1,3-oxazol-5-yl)-2-thienyl]-3-(2-phenylethyl)-4(3H)-pyrimidinone;
2-(2-hydroxyphenyl)-6-methyl-5-(4-methyl-2-thienyl)-3-(2-phenylethyl)-4(3H)-pyrimidinone;
5-[2-(2-hydroxyphenyl)-4-methyl-6-oxo-1-(2-phenylethyl)-1,6-dihydro-5-pyrimidinyl]-2-thiophenecarbonitrile;
2-(2-hydroxyphenyl)-6-methyl-3-(2-phenylethyl)-5-[5-(1H-tetrazol-5-yl)-2-thienyl]-4(3H)-pyrimidinone;
5-[5-(Aminomethyl)-2-thienyl]-2-(2-hydroxyphenyl)-6-methyl-3-(2-phenylethyl)-4(3H)-pyrimidinone;
2-(2-hydroxyphenyl)-6-methyl-5-{5-[(methylamino)methyl]-2-thienyl}-3-(2-phenylethyl)-4(3H)-pyrimidinone;
5-[5-(hydroxymethyl)-2-thienyl]-2-(2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(4,5,6,7-tetrahydro-1-benzothien-2-yl)-4(3H)-pyrimidinone;
2-(3-Fluoro-2-hydroxyphenyl)-6-methyl-3-(2-phenylethyl)-5-(2-phenyl-1,3-thiazol-5-yl)-4(3H)-pyrimidinone;
2-(3-Fluoro-2-hydroxyphenyl)-5-(4-hydroxyphenyl)-6-methyl-3-(2-phenylethyl)-4(3H)-pyrimidinone;
2-(3-Fluoro-2-hydroxyphenyl)-5-(2-hydroxyphenyl)-6-methyl-3-(2-phenylethyl)-4(3H)-pyrimidinone;
2-(3-Fluoro-2-hydroxyphenyl)-5-(3-hydroxyphenyl)-6-methyl-3-(2-phenylethyl)-4(3H)-pyrimidinone;
2-(2-Hydroxyphenyl)-6-methyl-5-(2-methylpropyl)-3-[2-(1-piperidinyl)ethyl]-4(3H)-pyrimidinone;
5-Ethyl-3-[2-(2-fluorophenyl)ethyl]-2-(3-hydroxyphenyl)-6-methyl-4(3H)-pyrimidinone;
2-(2-Hydroxyphenyl)-6-methyl-5-[5-(5-methyl-1,3,4-oxadiazol-2-yl)-2-thienyl]-3-(2-phenylethyl)-4(3H)-pyrimidinone;
5-(2,3-Dihydro-1,4-benzodioxin-6-yl)-2-(2-hydroxyphenyl)-6-[(methyloxy)methyl]-3-(2-phenylethyl)-4(3H)-pyrimidinone;
2-(2-Hydroxyphenyl)-6-[(methyloxy)methyl]-5-(4-methyl-2-thienyl)-3-(2-phenylethyl)-4(3H)-pyrimidinone;
2-(2-Hydroxyphenyl)-6-[(methyloxy)methyl]-5-(5-methyl-2-thienyl)-3-(2-phenylethyl)-4(3H)-pyrimidinone;
5-Bromo-6-[(dimethylamino)methyl]-2-(2-hydroxyphenyl)-3-(2-phenylethyl)-4(3H)-pyrimidinone;
6-[(Dimethylamino)methyl]-2-(2-hydroxyphenyl)-3-(2-phenylethyl)-4(3H)-pyrimidinone;
2-(3-Fluoro-2-hydroxyphenyl)-6-methyl-5-(5-methyl-3-thienyl)-3-(2-phenylethyl)-4(3H)-pyrimidinone;
5-(4,5-Dimethyl-1,3-thiazol-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(4-methyl-1,3-thiazol-2-yl) 3-(2-phenylethyl)-4(3H)-pyrimidinone;
5-(1,3-Benzodioxol-5-yl)-2-(3-fluoro-2-hydroxyphenyl)-6-methyl-3-(2-phenylethyl)-4(3H)-pyrimidinone;
3-(2,3-Dihydro-1H-inden-2-yl)-2-(2-hydroxyphenyl)-6-methyl-5-(5-methyl-2-thienyl)-4(3H)-pyrimidinone;
3-[1-(2,3-Dihydro-1H-inden-2-yl)-2-(2-hydroxyphenyl)-4-methyl-6-oxo-1,6-dihydro-5-pyrimidinyl]benzonitrile;
3-(2,3-Dihydro-1H-inden-2-yl)-5-(4,5-dimethyl-1,3-thiazol-2-yl)-2-(2-hydroxyphenyl)-6-methyl-4(3H)-pyrimidinone;
3-(2-Cyclohexylethyl)-2-(3-fluoro-2-hydroxyphenyl)-6-methyl-5-(5-methyl-2-thienyl)-4(3H)-pyrimidinone;
3-(2-Cyclohexylethyl)-5-(4,5-dimethyl-1,3-thiazol-2-yl)-2-(3-fluoro-2-hydroxyphenyl)-6-methyl-4(3H)-pyrimidinone;
3-(2-Cyclohexylethyl)-2-(3-fluoro-2-hydroxyphenyl)-6-methyl-5-(2-methyl-1,3-thiazol-5-yl)-4(3H)-pyrimidinone;
2-(3-Fluoro-2-hydroxyphenyl)-6-methyl-5-(5-methyl-2-thienyl)-3-[2-(2-thienyl)ethyl]-4(3H)-pyrimidinone;
5-(4,5-Dimethyl-1,3-thiazol-2-yl)-2-(3-fluoro-2-hydroxyphenyl)-6-methyl-3-[2-(2-thienyl)ethyl]-4(3H)-pyrimidinone;
2-(3-Fluoro-2-hydroxyphenyl)-6-methyl-5-(5-methyl-2-thienyl)-3-[2-(tetrahydro-2H-pyran-4-yl)ethyl]-4(3H)-pyrimidinone;
2-(3-Fluoro-2-hydroxyphenyl)-3-[2-(2-fluorophenyl)ethyl]-6-methyl-5-(5-methyl-2-thienyl)-4(3H)-pyrimidinone;
2-(3-Fluoro-2-hydroxyphenyl)-3-[2-(3-fluorophenyl)ethyl]-6-methyl-5-(5-methyl-2-thienyl)-4(3H)-pyrimidinone;
2-(3-Fluoro-2-hydroxyphenyl)-3-[2-(4-fluorophenyl)ethyl]-6-methyl-5-(5-methyl-2-thienyl)-4(3H)-pyrimidinone;
2-(3-Fluoro-2-hydroxyphenyl)-6-methyl-5-(3-methyl-2-thienyl)-3-(2-phenylethyl)-4(3H)-pyrimidinone;
2-(2-Hydroxyphenyl)-3-(2-phenylethyl)-5,6,7,8,9,10-hexahydrocycloocta[d]pyrimidin-4(3H)-one;
5-(1-Benzothien-2-yl)-3-(2,3-dihydro-1H-inden-2-yl)-2-(2-hydroxyphenyl)-6-methyl-4(3H)-pyrimidinone;
2-(3-Fluoro-2-hydroxyphenyl)-6-methyl-5-(2-methylpropyl)-3-(2-phenylethyl)-4(3H)-pyrimidinone;
2-(2-Hydroxyphenyl)-5,5-dimethyl-3-(2-phenylethyl)-5,6,7,8-tetrahydro-4(3H)-quinazolinone;
5-Chloro-2-(2-hydroxyphenyl)-6-methyl-3-(2-phenylethyl)-4(3H)-pyrimidinone;
3-[2-(3-Fluorophenyl)ethyl]-2-(2-hydroxyphenyl)-5,6,7,8-tetrahydro-4(3H)-quinazolinone;
2-(3-Fluoro-2-hydroxyphenyl)-6-methyl-5-(5-methyl-2-thienyl)-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]benzonitrile;
5-(2,3-Dihydro-1,4-benzod ioxin-6-yl)-2-(3-fluoro-2-hydroxyphenyl)-6-methyl-3-(2-phenylethyl)-4(3H)-pyrimidinone;
5-(3,5-Difluorophenyl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-5-(4-methyl-2-thienyl)-3-(2-phenylethyl)-4(3H)-pyrimidinone;
5-(1-Benzothien-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(3-thienyl)-4(3H)-pyrimidinone;
2-(3-Fluoro-2-hydroxyphenyl)-6-methyl-3-(2-phenylethyl)-5-(5-phenyl-2-thienyl)-4(3H)-pyrimidinone;
2-(3-Fluoro-2-hydroxyphenyl)-6-methyl-3-(2-phenylethyl)-5-(2-thienyl)-4(3H)-pyrimidinone;
5-(1,3-Benzothiazol-2-yl)-2-(3-fluoro-2-hydroxyphenyl)-6-methyl-3-(2-phenylethyl)-4(3H)-pyrimidinone;
5-(1-Benzothien-2-yl)-2-(2-hydroxyphenyl)-6-methyl-3-(2-phenylethyl)-4(3H)-pyrimidinone;
2-(2-Hydroxyphenyl)-6-methyl-5-(2-methyl-1,3-thiazol-5-yl)-3-(2-phenylethyl)-4(3H)-pyrimidinone;
5-[2-(2-Hydroxyphenyl)-4-methyl-6-oxo-1-(2-phenylethyl)-1,6-dihydro-5-pyrimidinyl]-2-thiophenecarbonitrile;
3-[2-(2-Hydroxyphenyl)-4-methyl-6-oxo-1-(2-phenylethyl)-1,6-dihydro-5-pyrimidinyl]benzonitrile;
2-(3-Fluoro-2-hydroxyphenyl)-6-methyl-5-phenyl-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinone;
2-(3-Fluoro-2-hydroxyphenyl)-6-methyl-5-(2-methyl-1,3-thiazol-5-yl)-3-(2-phenylethyl)-4(3H)-pyrimidinone; and
2-(3-Fluoro-2-hydroxyphenyl)-6-methyl-3-(2-phenylethyl)-5-(1H-pyrrol-1-yl)-4(3H)-pyrimidinone;
or a pharmaceutically acceptable salt thereof.

9. A compound according to claim 1 of formula (II): 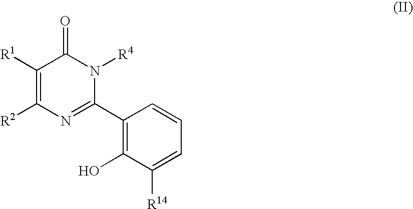
wherein:
R¹ and R² are, independently, selected from the group consisting of H, halogen, C₁₋₈alkyl, aryl, heterocyclyl, and heteroaryl, optionally substituted, except for H and halogen, one to three times, independently, by halogen, CN, C₁₋₄alkyl, aryl, heteroaryl,
—O—(CH₂)ₙ—O, CF₃, and OCF₃;
or R¹ and R² together form a 5 to 8 membered ring, optionally containing one to three heteroatoms selected from N, O and S, optionally substituted, independently, once or twice, by methyl;
R¹⁴ represents F or H;
R⁴ is represents arylC₁₋₂alkyl, optionally substituted, independently, one to three times, by F, CF₃ or Cl; and
n is 1,2, or 3;
or a pharmaceutically acceptable salt thereof.

10. A compound according to claim 9 wherein R¹ is selected from the group consisting of chloro, propyl, isobutyl, 2-thienyl, 5-methyl-2-thienyl, 3-cyano-2-thienyl, 4-methyl-2-thienyl, 3-cyano-2-thienyl, 2-cyanophenyl, 3-cyanophenyl, 3,5-difluorophenyl, dihydrobenzodioxyl, benzothienyl, benzothiazolyl, 2-methylthiazolyl, N-pyrrolyl and 2-methylthiazolyl.

11. A compound according to claim 9 wherein R¹ and R² form a cyclohexyl ring, optionally substituted, independently, once or twice by methyl.

12. A compound according to claim 9 wherein R² is methyl.

13. A compound according to claim 9 wherein R⁴ is 3-fluorophenethyl.

14. A compound according to claim 9 wherein R¹⁴ is F.

15. A compound according to claim 9 selected from the group consisting of:
2-(3-fluoro-2-hydroxyphenyl)-6-methyl-5-(2-methylpropyl)-3-(2-phenylethyl)-4(3H)-pyrimidinone;
2-(2-hydroxyphenyl)-5,5-dimethyl-3-(2-phenylethyl)-5,6,7,8-tetrahydro-4(3H)-quinazolinone;
5-chloro-2-(2-hydroxyphenyl)-6-methyl-3-(2-phenylethyl)-4(3H)-pyrimidinone;
3-[2-(3-Fluorophenyl)ethyl]-2-(2-hydroxyphenyl)-5,6,7,8-tetrahydro-4(3H)-quinazolinone;
2-(3-Fluoro-2-hydroxyphenyl)-6-methyl-5-(5-methyl-2-thienyl)-3-(2-phenylethyl)-4(3H)-pyrimidinone;
3-[2-(3-Fluoro-2-hydroxyphenyl)-4-methyl-6-oxo-1-(2-phenylethyl)-1,6-dihydro-5-pyrimidinyl]benzonitrile;
5-(2,3-dihydro-1,4-benzodioxin-6-yl)-2-(3-fluoro-2-hydroxyphenyl)-6-methyl-3-(2-phenylethyl)-4(3H)-pyrimidinone;
5-(3,5-Difluorophenyl)-2-(3-fluoro-2-hydroxyphenyl)-6-methyl-3-(2-phenylethyl)-4(3H)-pyrimidinone;
2-(3-fluoro-2-hydroxyphenyl)-6-methyl-5-(4-methyl-2-thienyl)-3-(2-phenylethyl)-4(3H)-pyrimidinone;
5-(1-benzothien-2-yl)-2-(3-fluoro-2-hydroxyphenyl)-6-methyl-3-(2-phenylethyl)-4(3H)-pyrimidinone;
2-(3-Fluoro-2-hydroxyphenyl)-6-methyl-3-(2-phenylethyl)-5-(2-thienyl)-4(3H)-pyrimidinone;
5-(1,3-Benzothiazol-2-yl)-2-(3-fluoro-2-hydroxyphenyl)-6-methyl-3-(2-phenylethyl)-4(3H)-pyrimidinone;
5-(1-Benzothien-2-yl)-2-(2-hydroxyphenyl)-6-methyl-3-(2-phenylethyl)-4(3H)-pyrimidinone;
2-(2-Hydroxyphenyl)-6-methyl-5-(2-methyl-1,3-thiazol-5-yl)-3-(2-phenylethyl)-4(3H)-pyrimidinone;
5-[2-(2-Hydroxyphenyl)-4-methyl-6-oxo-1-(2-phenylethyl)-1,6-dihydro-5-pyrimidinyl]-2-thiophenecarbonitrile;
3-[2-(2-hydroxyphenyl)-4-methyl-6-oxo-1-(2-phenylethyl)-1,6-dihydro-5-pyrimidinyl]benzonitrile;
2-(3-Fluoro-2-hydroxyphenyl)-6-methyl-5-phenyl-3-(2-phenylethyl)-4(3H)-pyrimidinone;
2-(3-Fluoro-2-hydroxyphenyl)-6-methyl-3-(2-phenylethyl)-5-propyl-4(3H)-pyrimidinone; and
2-(3-Fluoro-2-hydroxyphenyl)-6-methyl-3-(2-phenylethyl)-5-(1H-pyrrol-1-yl)-4(3H)-pyrimidinone;
or a pharmaceutically acceptable salt thereof.

16. A compound according to claim 9 which is 2-(3-Fluoro-2-hydroxyphenyl)-6-methyl-5-(5-methyl-2-thienyl)-3-(2-phenylethyl)-4(3H)-pyrimidinone; or a pharmaceutically acceptable salt thereof.

17. A pharmaceutical composition comprising a compound according to claim 1, and a pharmaceutically acceptable carrier or diluent.

18. A method of antagonizing a calcium receptor, which comprises administering to a subject in need thereof, an effective amount of a compound according to claim 1.

19. A method of treating a disease or disorder characterized by an abnormal bone or mineral homeostasis, which comprises administering to a subject in need of treatment thereof an effective amount of a compound of claim 1.

20. A method according to claim 19 wherein the abnormal bone or mineral homeostasis disease or disorder is selected from the group consisting of osteosarcoma, periodontal disease, fracture healing, osteoarthritis, rheumatoid arthritis, Paget's disease, humoral hypercalcemia, malignancy and osteoporosis.

21. A method according to claim 20 wherein the bone or mineral disease or disorder is osteoporosis.

22. A method of increasing serum parathyroid levels which comprises administering to a subject in need of treatment an effective amount of a compound of claim 1.

23. A method according to claim 20 wherein the compound according to formula (I) is co-administered with an anti-resorptive agent.

24. A method according to claim 23 wherein the anti-resorptive agent is selected from the group consisting of estrogen, 1α,25-(OH)₂D₃, 1α-(OH)D₃, calcitonin, selective estrogen receptor modulators, vitronectin receptor antagonists, V-H+-ATPase inhibitors, src SH₂ antagonists, bisphosphonates and cathepsin K inhibitors.

25. A method of synthesizing a compound according to claim 1 comprising the step of cyclizing an enamide according to structure (III) 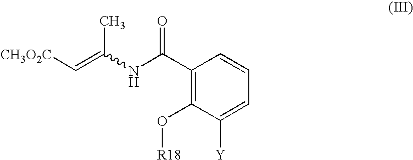
with R⁴NH₂ and trimethylaluminum to yield a pyrimidinone according to (IV): 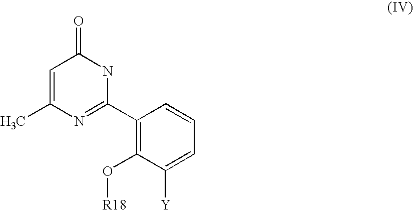
wherein:
R⁴ is selected from the group consisting of phenethyl, 3-fluorophenethyl, 4-fluorophenethyl, 2-fluorophenethyl, 2-thienylethyl, phenylethyl, dihydroindenyl, cyclohexylethyl, 3-chlorophenethyl, 3-trifluoromethylphenethyl, cyclopentylethyl and tetrahydropyranylethyl;
Y is a displacing group selected from the group consisting of F, Cl, Br and I; and
R¹⁸ is a protecting group selected from C₁₋₂alkyl, benzyl and acetyl.

26. An intermediate according to formula (III). 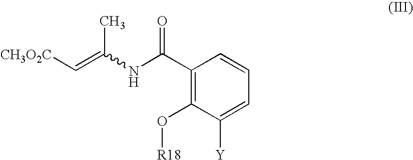
wherein:
R¹⁸ is C₁₋₂alkyl, benzyl or acetyl; and
Y is a displacing group selected from F, Cl, Br and I.

27. An intermediate according to formula (IV). 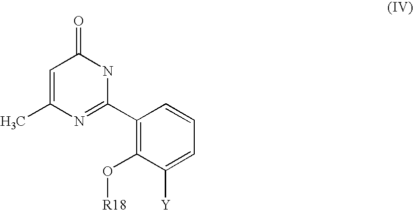
wherein:
R⁴ is selected from the group consisting of phenethyl, 3-fluorophenethyl, 4-fluorophenethyl, 2-fluorophenethyl, 2-thienylethyl, phenylethyl, dihydroindenyl, cyclohexylethyl, 3-chlorophenethyl, 3-trifluoromethylphenethyl, cyclopentylethyl and tetrahydropyranylethyl;
Y is a displacing group selected from F, Cl, Br and I; and
R¹⁸ is C₁₋₂alkyl, benzyl or acetyl.

28. An intermediate according to formula (V) 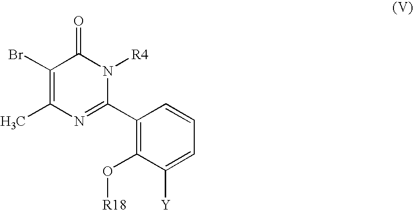
wherein:
R⁴ is selected from the group consisting of phenethyl, 3-fluorophenethyl, 4-fluorophenethyl, 2-fluorophenethyl, 2-thienylethyl, phenylethyl, dihydroindenyl, cyclohexylethyl, 3-chlorophenethyl, 3-trifluoromethylphenethyl, cyclopentylethyl and tetrahydropyranylethyl;
Y is a displacing group selected from F, Cl, Br and I; and
R¹⁸ is C₁₋₂alkyl, benzyl or acetyl.

29. An intermediate according to formula (VI). 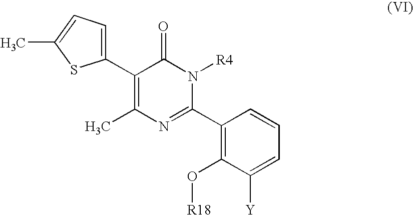
wherein:
R⁴ is selected from the group consisting of phenethyl, 3-fluorophenethyl, 4-fluorophenethyl, 2-fluorophenethyl, 2-thienylethyl, phenylethyl, dihydroindenyl, cyclohexylethyl, 3-chlorophenethyl, 3-trifluoromethylphenethyl, cyclopentylethyl and tetrahydropyranylethyl;
Y is a displacing group selected from F, Cl, Br and I; and
R¹⁸ is C₁₋₂alkyl, benzyl or acetyl.
